(19) United States
(12) Patent Application Publication
(10) Pub. No.: US 2023/0149560 A1
(43) Pub. Date: May 18, 2023

(54) LIPID COMPOSITIONS FOR DELIVERY OF STING AGONIST COMPOUNDS AND USES THEREOF

(71) Applicants: Massachusetts Institute of Technology, Cambridge, MA (US); Millennium Pharmaceuticals, Inc., Cambridge, MA (US)

(72) Inventors: Darrell J. IRVINE, Arlington, MA (US); Eric DANE, Cambridge, MA (US); Masanori OKANIWA, Cambridge, MA (US)

(21) Appl. No.: 17/919,952

(22) PCT Filed: Apr. 20, 2021
(86) PCT No.: PCT/US2021/028190

§ 371 Date: Oct. 19, 2022

Related U.S. Application Data
(60) Provisional application No. 63/012,706, filed on Apr. 20, 2020.

(51) Int. Cl. A61K 47/69; A61K 47/54; A61K 47/60; A61K 31/7084

(52) U.S. Cl. CPC A61K 47/6909; A61K 31/7084; A61K 47/60; A61K 47/544

(57) ABSTRACT

The present disclosure provides lipid compositions comprising lipid particles (e.g., lipid nanodiscs), wherein the lipid particles (e.g., lipid nanodiscs) comprise a STING agonist amphiphile conjugate, a phospholipid and a PEG-lipid. The compositions are used in methods to induce or promote immune responses, such as immune responses useful for the treatment of cancer or infectious disease.

### CROSS-REFERENCE TO RELATED APPLICATIONS

This application claims the benefit of U.S. Provisional Pat. Application Serial No. 63/012,706, filed Apr. 20, 2020, the entire contents of which are incorporated herein by reference.

### BACKGROUND OF DISCLOSURE

The immune system plays a critical role in our health. The importance of understanding the function of the immune system and learning how to modulate immunity to protect against or treat disease and infection cannot be overstated. Compounds that can modulate both the innate and adaptive immune responses to fight infectious pathogens or cancer are needed.

Immunotherapy treatments have revolutionized the treatment of cancer patients, and approved checkpoint blockade and chimeric antigen receptor T cell therapies have revolutionized cancer care (see, e.g., Sharma, P. et al. Science 348, 56-61 (2015); Larson, R. C. et al. Nat Rev Cancer 21:145-161 (2021)). However, intensive efforts are focused on expanding the repertoire of clinically-viable immunostimulatory drugs to enable a greater fraction of patients to experience meaningful clinical benefit (Chen, D. S. et al. Nature 541, 321-330 (2017)). One important class of immunotherapy agents in development are drugs that activate innate immune danger sensors, receptors that may be expressed by host immune cells and/or cancer cells, including Toll-like receptors, RIG-I-like receptors, NOD-like receptors, and STING (see, e.g., Seelige, R., et al. Curr Opin Immunol 50, 1-8 (2018); Flood, B. A., et. al. Immunol Rev 290, 24-38 (2019)). Delivery of such “danger signals” to the tumor has the potential to induce an in situ vaccination, where dying tumor cells act as a source of tumor antigens that are taken up by dendritic cells, which in parallel become activated by the innate immune stimulators, leading to priming of an adaptive T cell response (Hammerich, L., et al. Mol Oncol 9, 1966-1981 (2015)).

Stimulator of Interferon Genes (STING) (also known as MITA and MPYS; encoded by TMEM173) is a receptor associated with the endoplasmic reticulum (ER) that plays an important role in innate immunity by controlling the transcription of numerous host defense genes, including type I interferons (IFNs) and pro-inflammatory cytokines, following the recognition of aberrant DNA species and/or cyclic dinucleotides (CDNs) in the cytosol of the cell. The sources of exogenous CDNs include the genome of invading pathogens, such as viruses, as well as CDNs secreted by certain bacteria following infection of the host (see, e.g., Ishikawa, H. et al. (2009) NATURE 461:788-792; Sauer, J. et al. (2011) INFECT IMMUN. 79:688-694).

The STING signaling pathway has also been shown to have immunostimulatory effects that contribute to activation of radiation-induced or spontaneous anti-tumor T cell responses (see, e.g., Woo, S. et al. (2014) IMMUNITY 41:830-842; Deng, L. et al. (2014) IMMUNITY 41:843-852). Activation of STING occurs when tumor-derived DNA triggers cyclic-GMP-AMP synthase to produce cGAMP, the endogenous ligand of STING. STING undergoes a conformation change in response to ligand binding, resulting in activation of a downstream signaling cascade via recruitment of serine/threonine-protein kinase (TBK1), phosphorylation of the interferon regulatory transcription factor IRF3, and production of type I interferon (IFN) and other pro-inflammatory cytokines. The production of type I IFNs stimulates cross-presentation of tumor antigens by dendritic cells (DCs) and subsequent mobilization and priming of tumor-specific T cells.

The role of STING signaling in facilitating anti-tumor T cell responses has motivated development of STING agonists for use in treating cancer (see, e.g., Corrales, L. et al. Cell Reports 11:1018 (2015); Sivick, K. E. et al.Cell Reports 25:3074 (2018); Demaria, O. et al. Proc National Acad Sci 112:15408 (2015); Yang, H. et al. J Clin Invest 129:4350 (2019)). Several reports indicate that administration of CDNs can promote anti-tumor immune responses in animal models. For example, CDNs administered by intratumoral injection have been shown to promote rapid tumor shrinkage and immune cell activation in syngeneic preclinical tumor models (Corrales, et al (2015) CELL REPORTS, 11:1018). Additionally, intratumoral administration of CDNs has also been shown to promote tumor eradication and activation of adaptive immunity that is sufficient for rejection of subsequent tumor challenge (Sivick, et al (2018) CELL REPORTS, 25:3074). However, though local administration of CDNs has therapeutic benefit for promoting tumor eradication, not all tumors are amenable to local administration (e.g., by intratumoral injection). Moreover, systemic administration of CDNs has been shown to be ineffective for inducing tumor shrinkage or anti-tumor immunity, as CDNs are hydrophilic small molecules with short serum half-life (see, e.g., Shae, D. et al (2019) Nat Nanotech 14:269-278; Cheng, N. et al (2018) JCI Insight 3(22)). CDNs are also membrane impermeable and susceptible to rapid degradation by nucleases, making them unsuitable as agents that could be administered systemically for tumor delivery (see, e.g., Flood, B. A., et al. Immunol Rev 290, 24-38 (2019)). Systemic administration of CDNs also has a risk of treatment-associated toxicity due to systemic inflammatory effects.

Thus, there remains a need for strategies to deliver STING agonists that produce desirable innate and adaptive immune responses.

### SUMMARY OF DISCLOSURE

The present disclosure provides lipid compositions for delivery of STING agonists for use in methods of treating, preventing, or ameliorating a disease, such as a cancer or an infectious disease. In one aspect, the disclosure provides lipid nanodiscs comprising a STING agonist amphiphile conjugate, a phospholipid, and a polyethylene glycol (PEG)-lipid, and compositions thereof, for use in the methods of the disclosure, including administering the lipid nanodiscs or a composition thereof to a subject in need. The disclosure also provides STING agonist amphiphile conjugates comprising a STING agonist covalently-linked to a polymer-modified lipid, optionally via a linker, and methods for making the STING agonist amphiphile conjugates and lipid nanodiscs and compositions comprising the same as described herein. In some aspects, the disclosure provides methods of treating, preventing, or ameliorating cancer or an infectious disease in a subject, the method comprising administration (e.g., systemic administration) of a STING agonist amphiphile conjugate or lipid composition thereof described herein. In some aspects, the lipid composition comprises a lipid particle comprising the STING agonist amphiphile conjugate.

Accordingly, in some aspects, the disclosure provides a lipid composition comprising a STING agonist amphiphile conjugate, wherein the STING agonist amphiphile conjugate comprises an agonist of STING covalently linked to a polymer-modified lipid, optionally via a linker. In some aspects, the lipid composition comprises the STING agonist amphiphile conjugate and a phospholipid. In some aspects, the lipid composition comprises the STING agonist amphiphile conjugate, a phospholipid, and a polymer-modified lipid. In some aspects, the lipid composition comprises the STING agonist amphiphile conjugate, a phospholipid, and a polyethylene glycol (PEG)-lipid. In some aspects, the lipid composition comprises lipid particles, wherein a plurality of lipid particles are lipid nanodiscs. In some aspects, a plurality of lipid particles are liposomes (e.g., unilamellar liposomes, multilamellar liposomes). In some aspects, a plurality of lipid particles are spherical micelles. In some aspects, the lipid composition comprises lipid particles, wherein the particle fraction consisting of lipid nanodiscs is at least 0.4, 0.45, 0.5, 0.55, 0.6, 0.65, 0.70, 0.75, 0.8, 0.85, 0.90, 0.95 or higher as measured by, e.g., TEM or cryo-TEM. In some aspects, the particle fraction consisting of lipid naondiscs is at least 0.8, 0.85, 0.9, 0.95, or 0.99 as measured by, e.g., TEM or cryo-TEM.

In some aspects, the disclosure provides a lipid composition comprising a lipid particle comprising (i) a STING agonist amphiphile conjugate wherein the STING agonist amphiphile conjugate comprises an agonist of STING covalently linked to a polymer-modified lipid, optionally via a linker; (ii) a phospholipid; and (iii) a polyethylene glycol (PEG)-lipid. In some aspects, the lipid particle is a lipid nanodisc, wherein the lipid nanodisc has a diameter of between about 10 and about 100 nm, between about 10 nm and about 80 nm, between about 10 nm and about 60 nm, between about 10 and about 40 nm, or between about 20 and about 40 nm as measured by TEM, and wherein the lipid nanodisc has a height of about 4, 5, 6, 7, 8, 9, or 10 nm as measured by TEM. In some aspects, the lipid particle is a liposome (e.g., a unilamellar liposome, a multilamellar liposome), wherein the liposome has a diameter between about 10 nm and about 150 nm, between about 10 nm and about 130 nm, between about 20 nm and about 110 nm, between about 20 nm and about 90 nm, between about 40 nm and about 80 nm, or about 40, 50, 60, or 70 nm as measured by TEM. In some aspects, the lipid particle is a spherical micelle.

In some aspects, the disclosure provides a lipid nanodisc comprising (i) a STING agonist amphiphile conjugate, wherein the STING agonist amphiphile conjugate comprises an agonist of STING covalently linked to a polymer-modified lipid, optionally via a linker; (ii) a phospholipid; and (iii) a polyethylene glycol (PEG)-lipid. In some aspects, the lipid nanodisc is a disc-like micelle, a discoidal micelle, a bilayer disc, or a particle with disc morphology as measured by transmission electron microscopy (TEM). In some aspects, the lipid nanodisc has (i) a hydrodynamic diameter of about 10 nm to about 100 nm, about 20 nm to about 90 nm, about 30 nm to about 80 nm, about 30 nm to about 70 nm, about 30 nm to about 60 nm, about 30 nm to about 50 nm, or about 30 nm to about 40 nm as measured by dynamic light scattering (DLS); (ii) a diameter of about 10 nm to about 100 nm, about 20 nm to about 90 nm, about 30 nm to about 80 nm, about 30 nm to about 70 nm, about 30 nm to about 60 nm, about 30 nm to about 50 nm, about 30 nm to about 40 nm as measured by TEM; (iii) a height of about 5 nm to about 15 nm, about 6 nm, about 7 nm, about 8 nm, about 9 nm or about 10 nm as measured by TEM; and (iv) a combination of (i)-(iii). In some aspects, the lipid nanodisc comprises (i) a hydrodynamic diameter of about 10-15 nm, about 10-20 nm, about 15-20 nm, about 15-25 nm, about 20-25 nm, about 20-30 nm, about 25-30 nm, about 25-35 nm, about 30-35 nm, about 35-40 nm, about 35-45 nm, about 40-45 nm, about 40-50 nm, or about 45-50 nm as measured by DLS; (ii) a diameter of about 10-15 nm, about 10-20 nm, about 15-20 nm, about 15-25 nm, about 20-25 nm, about 20-30 nm, about 25-30 nm, about 25-35 nm, about 30-35 nm, about 35-40 nm, about 35-45 nm, about 40-45 nm, about 40-50 nm, or about 45-50 nm as measured by TEM; and/or (iii) a height of about 3, 4, 5, 6, 7, 8, 9, 10, 11 or 12 nm as measured by TEM. In some aspects, the lipid nanodisc comprises (i) a mean diameter between about 20 and about 30 nm; and (iii) a mean height between about 4 and 8 nm as measured by TEM. In some aspects, the lipid nanodisc remains assembled in the presence of serum albumin under physiological conditions.

In any of the foregoing or related aspects, the disclosure provides a STING agonist amphiphile conjugate comprising a STING agonist covalently-linked to a polymer-modified lipid, wherein the polymer-modified lipid comprises a polymer covalently-linked to a lipid that is a diacyl lipid. In some aspects, the diacyl lipid comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units. In some aspects, the diacyl lipid is selected from: a phosphatidylethanolamine (PE), 1,2-distearoyl-sn-glycero-3-phosphoethanolamine (DSPE), 1,2-dipalmitoyl-sn-glycero-3-phosphoethanolamine (DPPE), 1,2-dioleoyl-sn-glycero-3-phosphoethanolamine (DOPE), 1-palmitoyl-2-oleoyl-sn-glycero-3-phosphoethanolamine (POPE), or 1,2-dimyristoyl-sn-glycero-3-phosphoethanolamine (DMPE). In some aspects, the polymer is selected from: a hydrophilic polymer, a string of hydrophilic amino acids, a polysaccharide, or a combination thereof. In some aspects, the hydrophilic polymer comprises PEG, poly(propylene oxide) (PPO), poly(methacrylate), or a combination thereof. In some aspects, the polymer is a hyrophilic polymer. In some aspects, the hydrophilic polymer comprises “n” consecutive PEG units, wherein n is about 25 to about 230. In some aspects, the polymer-modified lipid comprises a hydrophilic polymer covalently-linked to a DSPE head-group, wherein the hydrophilic polymer comprises “n” consecutive PEG units, and wherein n is about 25 to about 230.

In any of the foregoing or related aspects, the disclosure provides a STING agonist amphiphile conjugate comprising a STING agonist covalently-linked to a polymer-modified lipid, wherein the STING agonist is a compound that binds to mouse STING receptor, human STING receptor, or both. In some aspects, the STING agonist increases or promotes production of one or more STING-dependent cytokines in a STING-expressing cell, wherein the STING-dependent cytokine is selected from: a cytokine that binds interferon receptor, interferon, type 1 interferon, IFN-α, IFN-β, IL-6, or TNF-α. In some aspects, the STING agonist is selected from: a cyclic dinucleotide (CDN) or a non-nucleotide small molecule, optionally wherein the non-nucleotide small molecule is an amidobenzimidazole (ABZI)-based compound or a di-ABZI-based compound.

In any of the foregoing or related aspects, the disclosure provides a STING agonist amphiphile conjugate comprising a STING agonist covalently-linked to a polymer-modified lipid, wherein the STING agonist is a CDN, and wherein the CDN comprises (i) a pyrimidine nucleotide base or analog thereof, a purine nucleotide base or analog thereof, or both; and (ii) a 2′,5′ phosphate bridge linkage, a 3′5′ phosphate bridge linkage, or both. In some aspects, the CDN is selected from: cyclic di-guanosine 5′-monophosphate (cyclic di-GMP), cyclic di-inosine monophosphate, cyclic di-adenosine 5′-monophosphate (cyclic di-AMP or CDA), cyclic GMP-AMP (cGAMP), cyclic[G(2′,5′)pA(3′,5′)p] (2′-3′ cGAMP), or cyclic[A(2′,5′)pA(3′,5′)p] (2′-3′ CDA). In some aspects, the CDN comprises at least one phosphate bridge linkage wherein a non-bridging oxygen atom is substituted with a sulfur atom, and wherein the CDN is covalently linked by the sulfur atom.

In any of the foregoing or related aspects, the disclosure provides a STING agonist amphiphile conjugate comprising a STING agonist covalently linked to a polymer-modified lipid via a linker. In some aspects, the linker comprises one or more cleavage elements, wherein the cleavage element is susceptible to cleavage. In some aspects, the cleavage is acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, disulfide reduction-based cleavage. In some aspects, the cleavage element is susceptible to cleavage by protease-induced cleavage. In some aspects, the linker comprises a cleavage element susceptible to cleavage by one or more proteases. In some aspects, the one or more proteases are intracellular proteases.

In some aspects, the disclosure provides a lipid composition comprising (i) a STING agonist amphiphile conjugate comprising a STING agonist covalently-linked to a polymer-modified lipid, optionally via a linker, wherein the polymer-modified lipid comprises a polymer covalently-linked to a lipid that is a diacyl lipid, optionally (ii) a phospholipid (e.g., HSPC), and optionally (iii) a polymer-modified lipid or a PEG-lipid (e.g., PEG2k-DSPE, PEG5k-DSPE).

In some aspects, the disclosure provides a lipid composition comprising (i) a STING agonist amphiphile conjugate comprising a STING agonist covalently-linked to a polymer-modified lipid via a linker, wherein the polymer-modified lipid comprises a polymer covalently-linked to a lipid that is a diacyl lipid (e.g., DSPE), wherein the linker comprises one or more cleavage elements, wherein the one or more cleavage elements are susceptible to cleavage by acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, disulfide reduction-based cleavage; optionally (ii) a phospholipid (e.g., HSPC); and optionally (iii) a polymer-modified lipid or a PEG-lipid (e.g., PEG2k-DSPE; PEG5k-DSPE).

In some aspects, the disclosure provides a lipid composition comprising (i) a STING agonist amphiphile conjugate comprising a STING agonist covalently-linked to a polymer-modified lipid via a linker, wherein the polymer-modified lipid comprises a polymer covalently-linked to a lipid that is a diacyl lipid, and wherein the linker comprises one or more cleavage elements susceptible to peptidase-induced cleavage or protease-induced cleavage; optionally (ii) a phospholipid (e.g., HSPC); and optionally (iii) a polymer-modified lipid or a PEG-lipid (e.g., PEG2k-DSPE; PEG5k-DSPE).

In some aspects, the disclosure provides a lipid composition comprising (i) a STING agonist amphiphile conjugate comprising a STING agonist covalently-linked to a polymer modified-lipid, optionally via a linker, wherein the polymer-modified lipid comprises a hydrophilic polymer covalently-linked to a diacyl lipid, wherein the hydrophilic polymer comprises “n” consecutive PEG, PEO, or poly(methacrylate) units, and wherein n is about 25 to about 230, optionally (ii) a phospholipid (e.g., HSPC), and optionally (iii) a polymer-modified lipid or a PEG-lipid (e.g., PEG2k-DSPE; PEG5k-DSPE).

In some aspects, the disclosure provides a lipid composition comprising (i) a STING agonist amphiphile conjugate comprising a STING agonist covalently-linked to a polymer modified-lipid via a linker, wherein the polymer-modified lipid comprises a hydrophilic polymer covalently-linked to a diacyl lipid, wherein the hydrophilic polymer comprises “n” consecutive PEG, PEO, or poly(methacrylate) units, wherein n is about 25 to about 230, wherein the linker comprises one or more cleavage elements, wherein the one or more cleavage elements are susceptible to cleavage by acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, disulfide reduction-based cleavage; optionally (ii) a phospholipid (e.g., HSPC); and optionally (iii) a polymer-modified lipid or a PEG-lipid (e.g., PEG2k-DSPE, PEG5k-DSPE).

In some aspects, the disclosure provides a lipid composition comprising (i) a STING agonist amphiphile conjugate comprising a STING agonist covalently-linked to a polymer modified-lipid via a linker, wherein the polymer-modified lipid comprises a hydrophilic polymer covalently-linked to a diacyl lipid, wherein the hydrophilic polymer comprises “n” consecutive PEG, PEO, or poly(methacrylate) units, wherein n is about 25 to about 230, and wherein the linker comprises one or more cleavage elements susceptible to peptidase-induced cleavage or protease-induced cleavage; optionally (ii) a phospholipid (e.g., HSPC); and optionally (iii) a polymer-modified lipid or a PEG-lipid (e.g., PEG2k-DSPE, PEG5k-DSPE).

In any of the foregoing or related aspects, the STING agonist is a CDN. In some aspects, the STING agonist is a non-nucleotide small molecule. In some aspects, the STING agonist is a an amidobenzimidazole (ABZI)-based compound or a di-ABZI-based compound.

In some aspects, the disclosure provides a lipid composition comprising (i) a STING agonist amphiphile conjugate comprising a STING agonist covalently-linked to a polymer-modified lipid, optionally via a linker, (a) wherein the STING agonist is a CDN, wherein the CDN comprises at least one phosphate bridge linkage, wherein the at least one phosphate bridge linkage comprises a non-bridging oxygen atom substituted with a sulfur atom, wherein the CDN is covalently linked to the polymer-modified lipid by the sulfur atom; wherein (b) the polymer-modified lipid comprises a polymer covalently-linked to a lipid that is a diacyl lipid (e.g., DSPE); optionally (ii) a phospholipid (e.g., HSCP), and optionally (iii) a polymer-modified lipid or a PEG-lipid (e.g., PEG2k-DSPE, PEG5k-DSPE).

In some aspects, the disclosure provides a lipid composition comprising (i) a STING agonist amphiphile conjugate comprising a STING agonist covalently-linked to a polymer-modified lipid via a linker, wherein (a) the STING agonist is a CDN, wherein the CDN comprises at least one phosphate bridge linkage, wherein the at least one phosphate bridge linkage comprises a non-bridging oxygen atom substituted with a sulfur atom, wherein the CDN is covalently linked to the polymer-modified lipid by the sulfur atom; wherein (b) the linker comprises one or more cleavage elements, wherein the one or more cleavage elements are susceptible to cleavage by acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, disulfide reduction-based cleavage; wherein (c) the polymer-modified lipid comprises a polymer covalently-linked to a lipid that is a diacyl lipid (e.g., DSPE); optionally (ii) a phospholipid (e.g., HSPC), and optionally (iii) a polymer-modified lipid or a PEG-lipid (e.g., PEG2k-DSPE, PEG5k-DSPE).

In some aspects, the disclosure provides a lipid composition comprising (i) a STING agonist amphiphile conjugate comprising a STING agonist covalently-linked to a polymer-modified lipid via a linker, wherein (a) the STING agonist is a CDN, wherein the CDN comprises at least one phosphate bridge linkage, wherein the at least one phosphate bridge linkage comprises a non-bridging oxygen atom substituted with a sulfur atom, wherein the CDN is covalently linked to the polymer-modified lipid by the sulfur atom; wherein (b) the linker comprises one or more cleavage elements, wherein the one or more cleavage elements are susceptible to cleavage by peptidase-induced cleavage or protease-induced cleavage; wherein (c) the polymer-modified lipid comprises a polymer covalently-linked to a lipid that is a diacyl lipid (e.g., DSPE); optionally (ii) a phospholipid (e.g., HSCP), and optionally (iii) a polymer-modified lipid or a PEG-lipid (e.g., PEG2k-DSPE, PEG5k-DSPE).

In some aspects, the disclosure provides a lipid composition comprising (i) a STING agonist amphiphile conjugate comprising a STING agonist covalently-linked to a polymer-modified lipid, optionally via a linker, (a) wherein the STING agonist is a CDN, wherein the CDN comprises at least one phosphate bridge linkage, wherein the at least one phosphate bridge linkage comprises a non-bridging oxygen atom substituted with a sulfur atom, wherein the CDN is covalently linked to the polymer-modified lipid by the sulfur atom; wherein (b) the polymer-modified lipid comprises a hydrophilic polymer covalently-linked to a diacyl lipid, wherein the hydrophilic polymer comprises “n” consecutive PEG, PEO, or poly(methacrylate) units, wherein n is about 25 to about 230; optionally (ii) a phospholipid (e.g., HSPC), and optionally (iii) a polymer-modified lipid or a PEG-lipid (e.g., PEG2k-DSPE, PEG5k-DSPE).

In some aspects, the disclosure provides a lipid composition comprising (i) a STING agonist amphiphile conjugate comprising a STING agonist covalently-linked to a polymer-modified lipid via a linker, wherein (a) the STING agonist is a CDN, wherein the CDN comprises at least one phosphate bridge linkage, wherein the at least one phosphate bridge linkage comprises a non-bridging oxygen atom substituted with a sulfur atom, wherein the CDN is covalently linked to the polymer-modified lipid by the sulfur atom; wherein (b) the linker comprises one or more cleavage elements, wherein the one or more cleavage elements are susceptible to cleavage by acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, disulfide reduction-based cleavage; wherein (c) the polymer-modified lipid comprises a hydrophilic polymer covalently-linked to a diacyl lipid, wherein the hydrophilic polymer comprises “n” consecutive PEG, PEO, or poly(methacrylate) units, wherein n is about 25 to about 230; optionally (ii) a phospholipid (e.g., HSPC), and optionally (iii) a polymer-modified lipid or a PEG-lipid (e.g., PEG2k-DSPE, PEG5k-DSPE).

In some aspects, the disclosure provides a lipid composition comprising (i) a STING agonist amphiphile conjugate comprising a STING agonist covalently-linked to a polymer-modified lipid via a linker, wherein (a) the STING agonist is a CDN, wherein the CDN comprises at least one phosphate bridge linkage, wherein the at least one phosphate bridge linkage comprises a non-bridging oxygen atom substituted with a sulfur atom, wherein the CDN is covalently linked to the polymer-modified lipid by the sulfur atom; wherein (b) the linker comprises one or more cleavage elements, wherein the one or more cleavage elements are susceptible to cleavage by peptidase-induced cleavage or protease-induced cleavage; wherein (c) the polymer-modified lipid comprises a hydrophilic polymer covalently-linked to a diacyl lipid, wherein the hydrophilic polymer comprises “n” consecutive PEG, PEO, or poly(methacrylate) units, wherein n is about 25 to about 230; optionally (ii) a phospholipid (e.g., HSPC), and optionally (iii) a polymer-modified lipid or a PEG-lipid (e.g., PEG2k-DSPE, PEG5k-DSPE).

In any of the foregoing or related aspects, the CDN comprises a pyrimidine nucleotide base or analog thereof. In some aspects, the CDN comprises two a pyrimidine nucleotide bases or analog thereof. In some aspects, the CDN comprises a purine nucleotide base or analog thereof. In some aspects, the CDN comprises two purine nucleotide bases or analog thereof. In some aspects, the CDN comprises a pyrimidine nucleotide base or analog thereof and a purine nucleotide base or analog thereof. In some apsects, the CDN is selected from: cyclic di-guanosine 5′-monophosphate (cyclic di-GMP), cyclic di-inosine monophosphate, cyclic di-adenosine 5′-monophosphate (cyclic di-AMP or CDA), cyclic GMP-AMP (cGAMP), cyclic[G(2′5′)pA(3′,5′)p] (2′-3′ cGAMP), or cyclic[A(2′,5′)pA(3′,5′)p] (2′-3′ CDA).

In any of the foregoing or related aspects, the disclosure provides a STING agonist amphiphile conjugate comprising the formula (XIV):

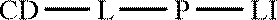

or a pharmaceutically acceptable salt thereof, wherein CD is a group represented by any one of Formula (XX) - (XXIX), vida infra, L is a linker; P is a polymer; and LI is a diacyl lipid.

In any of the foregoing or related aspects, the disclosure provides a STING agonist amphiphile conjugate comprising the formula (XIV):

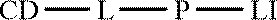

or a pharmaceutically acceptable salt thereof, wherein CD is a group represented by any one of Formula (XX-A), Formula (XXI-A), Formula (XXII-A), Formula (XXIII-A), Formula (XXIV-A), Formula (XXV-A), Formula (XXVI-A), Formula (XXVII-A), Formula (XXVIII-A), or Formula (XXIX-A), vida infra, L is a linker; P is a polymer; and LI is a diacyl lipid.

In any of the foregoing or related aspects, the disclosure provides a STING agonist amphiphile conjugate comprising the formula (XIV), wherein CD is a group represented by any one of Formula (XX)-(XXIX), (XX-A), (XXI-A), (XXII-A), (XXIII-A), (XXIV-A), (XXV-A), (XXVI-A), (XXVII-A), or (XXVIII-A), (XXIX-A), and wherein L is -X³-T-Z-Q-.

In some aspects, the disclosure provides a STING agonist amphiphile comprising the formula (XXX) or (XXXI), vida infra.

In some aspects,
X³ is —(CH₂)ₒ—,

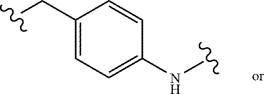

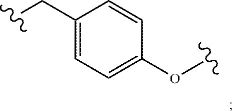

o is 1, 2, or 3; or X³ is absent;
T is a peptide, or is absent;
Z is a spacer;
Q is a heterobifunctional group or heterotrifunctional group, or Q is absent. In some aspects,
X³ is

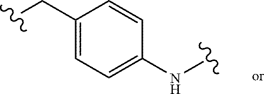

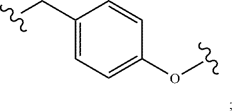

T is

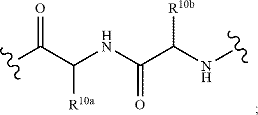

R^{10a} and R^{10b} are independently selected from the group consisting of hydrogen and optionally substituted C₁₋₆ alkyl;
Z is

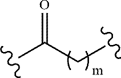

or —(CH₂CH₂O)ⱼ—;
m is 1, 2, 3, 4, 5, or 6;
j is 1, 2, 3, 4, 5, 6, 7, 8, 9, or 10;
Q is a heterobifunctional group selected from:

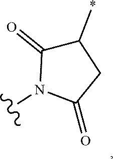

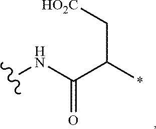

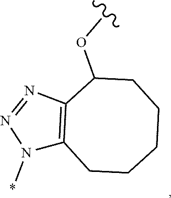

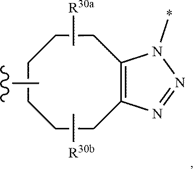

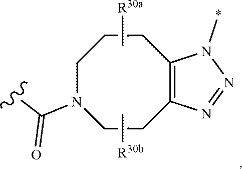

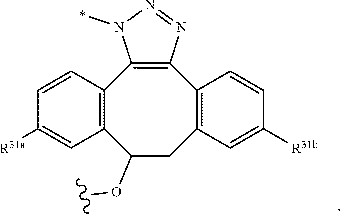

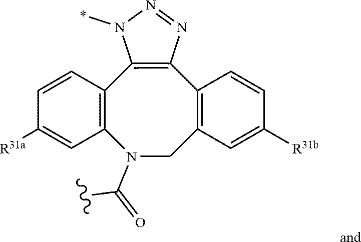

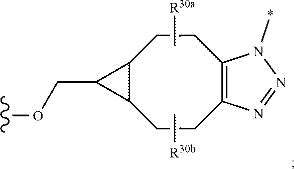

R²⁹ is hydrogen or C₁₋₆ alkyl; R^{30a} and R^{30b} are independently selected from the group consisting of hydrogen, C₁₋₆ alkyl, halo, —C(═O)OR²⁹, —NH₂, C₁₋₆ alkoxy, —CN, —NO₂, and —OH; R^{31a} and R^{31b} are independently selected from the group consisting of hydrogen, C₁₋₆ alkyl, halo, —C(═O)OR²⁹, —NH₂, —N(CH₃)₂, C₁₋₆ alkoxy, —CN, —NO₂, and —OH; and * indicates the attachment point to a carbon atom, nitrogen atom, oxygen atom, or sulfur atom of P;
P is —Z²—(CH₂CH₂O)ₙ—Z³—;
Z^{Z} is

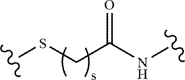

or —(CH₂CH₂O)—;
n is 25-230;
Z³ is —C(O)— or C₁₋₆ alkyl;
s is 1, 2, 3, 4, 5, or 6;
LI is a compound according to Formula (LI-1):

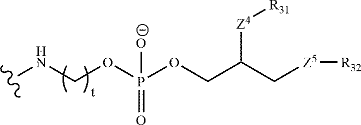

t is 1, 2, 3, 4, 5, 6, 7, 8, 9, or 10;
Z⁴ and Z⁵ are each independently selected from the group consisting of —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
R₃₁ and R₃₂ are each independently an optionally substituted alkyl, alkenyl, or alkynyl group comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units; and
R^{N} is selected from hydrogen, C₁₋₆ alkyl, or a nitrogen protecting group.

In any of the foregoing or related aspects, the STING agonist amphiphile conjugate of the disclosure comprises the formula ((XXXII) or formula (XXXII-A),

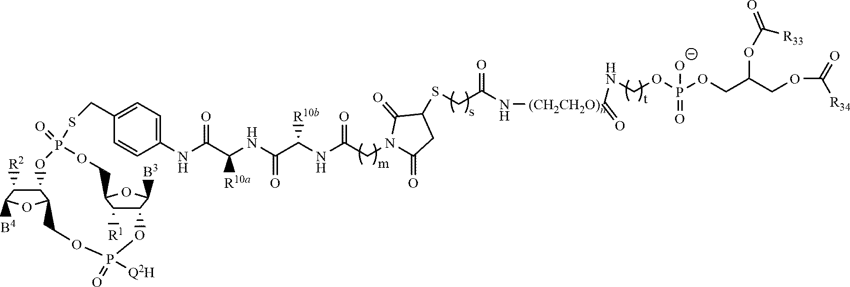

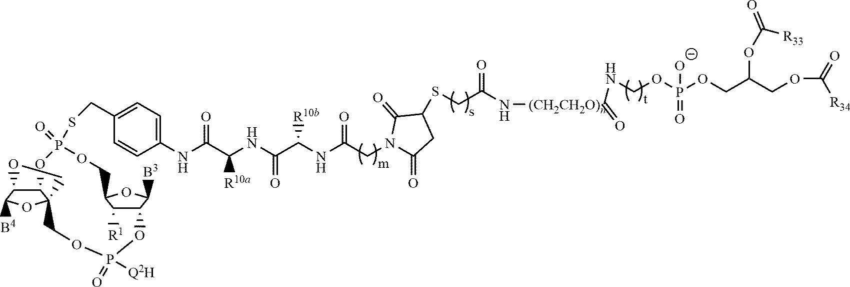

or a pharmaceutically acceptable salt thereof;
R^{10a} and R^{10b} are independently C₁₋₃ alkyl;
n is 25-230;
m is 2, 3, 4, or 5;
s is 1, 2, 3, 4, 5, or 6;
t is 1, 2, 3, 4, 5, or 6; and
R₃₃ and R₃₄ are each independently an optionally substituted alkyl, alkenyl, or alkynyl group comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units.

In any of the foregoing or related aspects, the STING agonist amphiphile conjugate of the disclosure comprises the formula ((XXXII) or formula (XXXII-A),

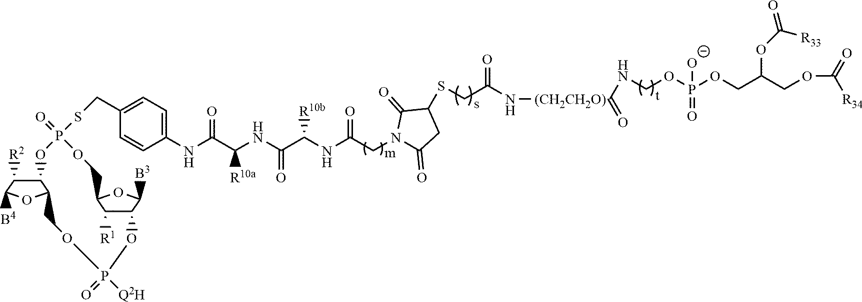

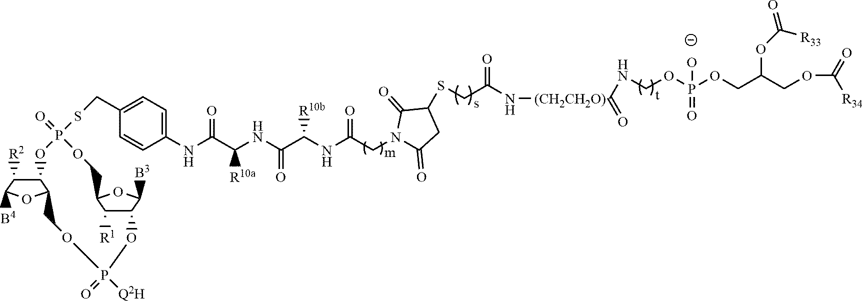

or a pharmaceutically acceptable salt thereof;
R^{10a} and R^{10b} are independently C₁₋₃ alkyl;
n is 25-230;
m is 1, 2, 3, 4, 5, 6, 7, 8, 9, or 10;
s is 1, 2, 3, 4, 5, or 6;
t is 1, 2, 3, 4, 5, or 6; and
R₃₃ and R₃₄ are each independently an optionally substituted alkyl, alkenyl, or alkynyl group comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units. In some aspects, m is 5.

In any of the foregoing or related aspects, the STING agonist amphiphile conjugate of the disclosure is selected from: (i) the formula (XIV), wherein CD is a group represented by any one of Formula (XX)-(XXIX), (XX-A), (XXI-A), (XXII-A), (XXIII-A), (XXIV-A), (XXV-A), (XXVI-A), (XXVII-A), or (XXVIII-A), (XXIX-A); and (ii) any one of the formulas (XXX), (XXXI), (XXXII), (XXXII-A), wherein B³ is a group represented by formula (B³-A) or formula (B³-B):

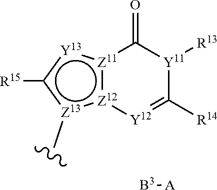

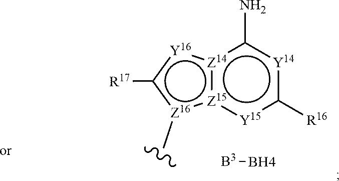

R¹³, R¹⁴, R¹⁵, R¹⁶ and R¹⁷ are each independently a hydrogen atom or a substituent;
Y¹¹, Y¹², Y¹³, Y¹⁴, Y¹⁵ and Y¹⁶ are each independently N or CR^{1a};
Z¹¹, Z¹², Z¹³, Z¹⁴, Z¹⁵ and Z¹⁶ are each independently N or C;
R^{1a} is a hydrogen atom or a substituent;
B⁴ is a group represented by formula (B⁴-A) or formula (B⁴-B):

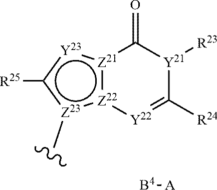

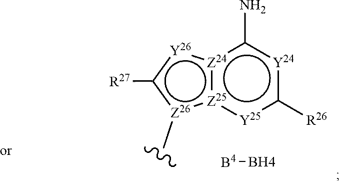

R²³, R²⁴, R²⁵, R²⁶ and R²⁷ are each independently a hydrogen atom or a substituent;
Y²¹, Y²², Y²³, Y²⁴, Y²⁵ and Y²⁶ are each independently N or CR^{2a};
Z²¹, Z²², Z²³, Z²⁴, Z²⁵ and Z²⁶ are each independently N or C; and
R^{2a} is a hydrogen atom or a substituent;
provided that
   i) at least one of Y¹¹, Y¹², Y¹³, Y¹⁴, Y¹⁵ and Y¹⁶ is C^{R1a},
   ii) at least one of Y²¹, Y²², Y²³, Y²⁴, Y²⁵ and Y²⁶ is C^{R2a}, or
   iii) at least one of Z¹³, Z¹⁶, Z²³ and Z²⁶ is C;
X¹ and X² are each independently an oxygen atom or a sulfur atom; and
Q¹, Q², Q3 and Q⁴ are each independently an oxygen atom or a sulfur atom, or a salt thereof.

In any of the foregoing or related aspects, the STING agonist amphiphile conjugate of the disclosure is selected from: (i) the formula (XIV), wherein CD is a group represented by any one of Formula (XX)-(XXIX), (XX-A), (XXI-A), (XXII-A), (XXIII-A), (XXIV-A), (XXV-A), (XXVI-A), (XXVII-A), or (XXVIII-A), (XXIX-A); and (ii) any one of the formulas (XXX), (XXXI), (XXXII), (XXXII-A), wherein at least one of B³ or B⁴ is

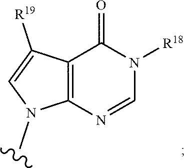

R¹⁸ is hydrogen or C₁₋₆ alkyl;
R¹⁹ is a halogen atom;
X¹ and X² are each independently an oxygen atom or a sulfur atom; and
Q¹, Q², Q3 and Q⁴ are each independently an oxygen atom or a sulfur atom, or a salt thereof. In some aspects, R¹⁹ is a fluoro atom. In some aspects, R¹⁸ is hydrogen. In some aspects, R¹⁸ is methyl. In some aspects, X¹ and X² are O.

In any of the foregoing or related aspects, the disclosure provides a STING agonist amphiphile selected from: (i) the formula (XIV), wherein CD is a group represented by any one of Formula (XX)-(XXIX), (XX-A), (XXI-A), (XXII-A), (XXIII-A), (XXIV-A), (XXV-A), (XXVI-A), (XXVII-A), or (XXVIII-A), (XXIX-A); and (ii) any one of the formulas (XXX), (XXXI), (XXXII), (XXXII-A), wherein B³ is

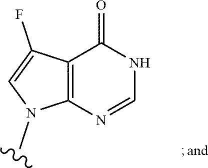

B⁴ is

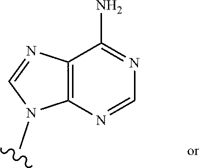

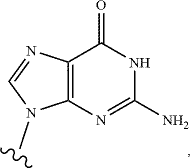

or a pharmaceutically acceptable salt thereof; X¹ and X² are each independently an oxygen atom or a sulfur atom; and Q¹, Q², Q3 and Q⁴ are each independently an oxygen atom or a sulfur atom, or a salt thereof. In some aspects, X¹ and X² are O.

In any of the foregoing or related aspects, the disclosure provides a STING agonist amphiphile selected from: (i) the formula (XIV), wherein CD is a group represented by any one of Formula (XX)-(XXIX), (XX-A), (XXI-A), (XXII-A), (XXIII-A), (XXIV-A), (XXV-A), (XXVI-A), (XXVII-A), or (XXVIII-A), (XXIX-A); and (ii) any one of the formulas (XXX), (XXXI), (XXXII), (XXXII-A), wherein B⁴ is

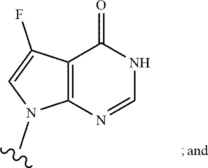

B³ is

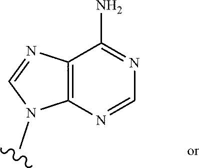

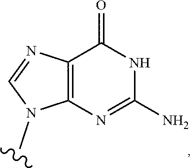

or a pharmaceutically acceptable salt thereof; X¹ and X² are each independently an oxygen atom or a sulfur atom; and Q¹, Q², Q³ and Q⁴ are each independently an oxygen atom or a sulfur atom, or a salt thereof. In some aspects, X¹ and X² are O.

In some aspects, the disclosure provides a STING agonist amphiphile conjugate that is CDN-PEG-Lipid as shown in FIG. 1B.

In some aspects, the disclosure provides a pharmaceutical composition comprising a STING agonist amphiphile conjugate described herein, or a pharmaceutically acceptable salt thereof, and a pharmaceutically acceptable carrier.

In some aspects, the disclosure provides a method of inducing or enhancing an immune response in a subject with cancer, comprising administering to a subject in need thereof a STING agonist amphiphile conjugate described herein, or a pharmaceutically acceptable salt thereof.

In some aspects, the disclosure provides a method of treating cancer comprising administering to a subject in need thereof a STING agonist amphiphile conjugate described herein, or a pharmaceutically acceptable salt thereof, or a pharmaceutical composition described herein.

In some aspects, the disclosure provides a vaccine comprising a STING agonist amphiphile conjugate of described herein, or a pharmaceutically acceptable salt thereof, and an antigen. In some aspects, the antigen is an infectious disease antigen. In some aspects, the antigen is a cancer antigen. In some aspects, the vaccine comprises a pharmaceutically acceptable carrier for mucosal administration. In some aspects, the vaccine comprises a pharmaaceutically acceptable carrier for parenteral administration, optionally subcutaneous administration, intramuscular administration, or intravenous administration.

In some aspects, the disclosure provides a method of immunizing a subject, the method comprising administering a vaccine described herein.

In any of the foregoing or related aspects, the disclosure provides a lipid nanodisc comprising a STING agonist amphiphile conjugate described herein, a PEG-lipid, and a phospholipid, wherein the PEG-lipid is selected from: a PEG-modified DSPE, a PEG-modified DOPE, a PEG-modified DPPE, a PEG-modified DMPE, a PEG-modified POPE, a PEG-modified ceramide, or a mixture thereof. In some aspects, the PEG comprises about 25 to about 230 consecutive PEG units, or wherein the PEG is has an average molecular weight of about 1,000 daltons, about 2,000 daltons, about 3,000 daltons, about 4,000 daltons, about 5,000 daltons, about 6,000 daltons, about 7,000 daltons, about 8,000 daltons, about 9,000 daltons, or about 10,000 daltons. In some aspects, the PEG-lipid is selected from: PEG2000-DSPE, PEG5000-DSPE, PEG7500-DSPE, PEG10000-DSPE or a mixture thereof.

In any of the foregoing or related aspects, the disclosure provides a lipid nanodisc comprising a STING agonist amphiphile conjugate described herein, a PEG-lipid, and a phospholipid, wherein the phospholipid is selected from: a glycerophospholipid, a sphingophospholipid, a phosphatidylcholine (PC), a phosphatidylethanolamine (PE), a phosphotidylserine (PS), a phosphatidylinositol (PI), a phosphatidylglycerol (PG), a phosphatidic acid (PA), or mixtures thereof. In some aspects, the phospholipid is selected from: DSPE, DPPE, DOPE, DMPE, POPE, soy L-α-phosphatidylcholine (soy PC), hydrogenated soy PC (HSPC), 1,2-dipalmitoyl-sn-glycero-3-phosphocoline (DPPC), 1-palmitoyl-2-oleoyl-sn-glycero-3-phosphocholine (POPC), 1,2-diastearoyl-sn-glycero-3-phosphocholine (DSPC), 1,2-dimyristoyl-sn-glycero-3-phosphocholine (DMPC), 1-palmitoyl-2-oleoyl-sn-glycero-3-ethylphosphocholine, egg PC, 1-myristoyl-2-stearoyl-sn-glycero-3-phosphocholine (MSPC), 1-palmitoyl-2-oleoyl-sn-glycero-3-phospho-L-serine (POPS), L-α-phosphatidylinositol, a sphingomyelin, or mixtures thereof. In some aspects, the phospholipid is DOPC.

In any of the foregoing or related aspects, the disclosure provides a lipid nanodisc comprising a STING agonist amphiphile conjugate described herein, a PEG-lipid, and a phospholipid, wherein the lipid nanodisc comprises (i) about 5-10 mol% or about 5 mol%, about 6 mol%, about 7 mol%, about 8 mol%, about 9 mol% or about 10 mol% STING agonist amphiphile conjugate; (ii) about 10-30 mol %, about 15-25 mol%, about 15-20 mol%, or about 15 mol%, about 16 mol%, about 17 mol%, about 18 mol%, about 19 mol%, or about 20 mol% PEG-Lipid; and (iii) at least about 85-45 mol%, about 80-50 mol%, about 75-55 mol%, about 70-60 mol%, or about 55 mol%, about 60 mol%, about 65 mol%, about 70 mol%, about 75 mol%, or about 80 mol% phospholipid. In some aspects, the lipid nanodisc comprises about 5-10 mol% STING agonist amphiphile conjugate, about 20 mol% PEG-Lipid, and at least about 50 mol% phospholipid. In some aspects, the lipid nanodisc further comprises a cationic lipid or sterol. In some aspects, the lipid nanodisc comprises about 10-30 mol%, about 10-20 mol%, about 10 mol%, about 15 mol%, about 20 mol%, about 25 mol%, or about 30 mol% cationic lipid. In some aspects, the cationic lipid is 1,2-dipalmitoyl-3-trimethylammonium-propane (16:0 TAP).

In some aspects, the disclosure provides a pharmaceutical composition comprising a lipid nanodisc described herein and a pharmaceutically acceptable carrier. In some aspects, the pharmaceutical composition further comprises a unilamellar liposome, a multilamellar liposome, a spherical micelle, or any combination thereof. In some aspects, the pharmaceutical composition has a particle fraction consisting of lipid nanodisc particles that is at least 0.4, 0.45, 0.5, 0.55, 0.6, 0.65, 0.70, 0.75, 0.8, 0.85, or 0.90 as measured by TEM or cryo-TEM.

In some aspects, the disclosure provides a lipid composition comprising (i) a STING agonist amphiphile conjugate described herein, (ii) a PEG-lipid, wherein the PEG-lipid is selected from: a PEG-modified DSPE, a PEG-modified DOPE, a PEG-modified DPPE, a PEG-modified DMPE, a PEG-modified POPE, a PEG-modified ceramide, or a mixture thereof, and wherein the PEG comprises about 25 to about 230 consecutive PEG units, and (iii) a phospholipid selected from: a glycerophospholipid, a sphingophospholipid, a phosphatidylcholine (PC), a phosphatidylethanolamine (PE), a phosphotidylserine (PS), a phosphatidylinositol (PI), a phosphatidylglycerol (PG), a phosphatidic acid (PA), or mixtures thereof. In some aspects, the PEG-lipid is PEG2000-DSPE. In some aspects, the PEG-lipid is PEG5000-DSPE.

In some aspects, the disclosure provides a lipid composition comprising (i) about 5-10 mol% of a STING agonist amphiphile conjugate described herein, (ii) about 15-25 mol% PEG-Lipid; and (iii) at least about 50 mol% phospholipid, wherein the lipid composition comprises a plurality of lipid particles that are lipid nanodiscs (e.g., a particle fraction consisting of lipid naondiscs that is at least 0.8, 0.85, 0.9, 0.95, or 0.99). In some aspects, the lipid composition further comprises a cationic lipid (e.g., 1,2-dipalmitoyl-3-trimethylammonium-propane (16:0 TAP).

In some aspects, the disclosure provides a lipid composition comprising (i) about 5-10 mol% of a STING agonist amphiphile conjugate described herein, (ii) about 15-25 mol% PEG-Lipid; (iii) at least about 50 mol% phospholipid, and (iv) about 15-30 mol% cationic lipid (e.g., 1,2-dipalmitoyl-3-trimethylammonium-propane (16:0 TAP), wherein the lipid composition comprises a plurality of lipid particles that are lipid nanodiscs (e.g., a particle fraction consisting of lipid naondiscs that is at least 0.8, 0.85, 0.9, 0.95, or 0.99).

In some aspects, the disclosure provides a lipid composition comprising (i) about 1-5 mol% of a STING agonist amphiphile conjugate described herein, (ii) about 1-5 mol% PEG-Lipid; and (iii) at least about 50-60% phospholipid, wherein the lipid composition comprises a plurality of lipid particles that are liposomes (e.g., particle fraction consisting of liposomes of at least 0.8, 0.85, 0.9, 0.95, or 0.99). In some aspects, the lipid composition further comprises a structural lipid (e.g., cholesterol).

In some aspects, the lipid composition comprises (i) about 1-5 mol% of a STING agonist amphiphile conjugate described herein, (ii) about 1-5 mol% PEG-Lipid; (iii) at least about 50-60 mol% phospholipid, and (iv) about 30-40 mol% structural lipid (e.g., cholesterol), wherein the lipid composition comprises a plurality of lipid particles that are liposomes (e.g., a particle fraction consisting of liposomes that is at least 0.8, 0.85, 0.9, 0.95, or 0.99).

. In some aspects, the disclosure provides a pharmaceutical composition comprising a lipid composition described herein and a pharmaceutically acceptable carrier.

In some aspects, the disclosure provides a method of inducing or enhancing an immune response in a subject with cancer, comprising administering to a subject in need thereof a lipid composition described herein. In some aspects, the disclosure provides a method of inducing or enhancing an immune response in a subject with cancer, comprising administering to a subject in need thereof a pharmaceutical composition described herein.

In some aspects, the disclosure provides a method of treating cancer comprising administering to a subject in need thereof a lipid composition described herein. In some aspects, the disclosure provides a method of treating cancer comprising administering to a subject in need thereof a pharmaceutical composition described herein.

In some aspects, the disclosure provides a vaccine comprising a lipid composition described herein, and an antigen. In some aspects, the disclosure provides a vaccine comprising a pharmaceutical composition described herein, and an antigen. In some aspects, the antigen is an infectious disease antigen. In some aspects, the antigen is a cancer antigen. In some aspects, the vaccine comprises a pharmaceutically acceptable carrier for mucosal administration. In some aspects, the vaccine comprises a pharmaaceutically acceptable carrier for parenteral administration, optionally subcutaneous administration, intramuscular administration, or intravenous administration.

In some aspects, the disclosure provides a method of immunizing a subject, the method comprising administering a vaccine described herein.

In some aspects, the disclosure provides a method for increasing serum half-life of a STING agonist in a subject, comprising administering to a subject in need thereof a lipid composition or a pharmaceutical composition thereof described herein, thereby increasing serum half-life of the STING agonist in the subject. In some aspects, the disclosure provides a method for increasing serum half-life of a STING agonist in a subject, comprising administering to a subject in need thereof a lipid composition or a pharmaceutical composition thereof described herein, wherein the lipid composition comprises a lipid particle comprising the STING agonist, and wherein the lipid particle has a mean diameter of greater than 10 n, wherein the diameter is of sufficient size to reduce clearance of the STING agonist from circulation. In some aspects, the disclosure provides a method for increasing serum half-life of a STING agonist in a subject, comprising administering to a subject in need thereof a lipid composition or a pharmaceutical composition thereof described herein, wherein the lipid composition comprises a lipid particle comprising the STING agonist, and wherein the lipid particle is of sufficient size to reduce clearance of the STING agonist from circulation. In some aspects, the disclosure provides a method for increasing serum half-life of a STING agonist in a subject, comprising administering to a subject in need thereof a lipid nanodisc or a pharmaceutical composition described herein, wherein the lipid nanodisc comprises the STING agonist, and wherein the lipid nanodisc is of sufficient size to reduce clearance of the STING agonist from circulation. In some aspects, the administering comprises systemic administration. In some aspects, the serum half-life of the STING agonist is at least about 5, 6, 7, 8, 9, 10, 11, 12, 13, 14, 15, 16, 17, 18, 19, or 20 hours. In some aspects, the serum half-life of the STING agonist is about 5-10 hours, about 5-15 hours, about 5-20 hours, about 10-15 hours, about 10-20 hours, or about 5, 6, 7, 8, 9, 10, 11, 12, 13, 14, 15, 16, 17, 18, 19, or 20 hours. In some aspects, the serum half-life of the STING agonist is about 10 hours. In some aspects, the serum half-life of the STING agonist is about 11 hours. In some aspects, the serum half-life of the STING agonist is about 12 hours. In some aspects, the serum half-life of the STING agonist is about 13 hours.

In some aspects, the disclosure provides a method for increasing accumulation of STING agonist in a target tissue and/or target cell population in a subject, the method comprising administering to a subject in need thereof a lipid composition or pharmaceutical composition thereof described herein, thereby increasing the accumulation of the STING agonist. In some aspects, the target tissue is a tumor. In some aspects, the target tissue is tumor draining lymphoid tissue. In some aspects, the disclosure provides a method for increasing accumulation of STING agonist in a subject with a tumor, the method comprising administering to a subject in need thereof a lipid composition or pharmaceutical composition thereof described herein, wherein the lipid composition comprises a lipid particle comprising the STING agonist, wherein the accumulation occurs in the tumor and/or tumor lymphoid tissue, and wherein the lipid particle is of sufficient size and/or shape to promote targeting of STING agonist to the tumor upon administration. In some aspects, the disclosure provides a method for increasing accumulation of STING agonist in a subject with a tumor, comprising administering to a subject in need thereof a lipid nanodisc or a pharmaceutical composition described herein, wherein the lipid nanodisc comprises the STING agonist, wherein the accumulation occurs in the tumor and/or tumor lymphoid tissue, and wherein the lipid nanodisc is of sufficient size and/or shape to promote targeting of STING agonist to the tumor upon administration. In some aspects, the administering comprises systemic administration. In some aspects, the tumor is a solid tumor. In some aspects, the accumulation in the tumor is at least about 1%, at least about 2%, at least about 3%, at least about 4%, at least about 5%, at least about 10%, or at least about 15% of injected dose per gram of tumor tissue. In some aspects, the accumulation in the tumor occurs in one or more vascular regions of the tumor. In some aspects, the accumulation in the tumor occurs in one or more extravascular regions of the tumor. In some aspects, the accumulation occurs in at least one cell subset in the tumor microenvironment. In some aspects, the at least one cell subset comprises tumor cells, tumor endothelial cells, tumor myeloid cells, dendritic cells, or a combination thereof. In some aspects, the at least one cell subset comprises tumor cells. In some aspects, the at least one cell subset comprises tumor myeloid cells. In some aspects, the at least one cell subset comprises tumor endothelial cells. In some aspects, the accumulation occurs in at least one additional tissue, optionally wherein the at least one additional tissue is liver and/or spleen. In some aspects, the accumulation is increased relative to a control lipid particle comprising the STING agonist, optionally wherein the control lipid particle is a spherical particle or a liposome.

In some aspects, the disclosure provides a method for increasing tumor accumulation of STING agonist in a subject with a tumor, the method comprising administering to a subject in need thereof a lipid composition or pharmaceutical composition thereof described herein, thereby increasing the accumulation of STING agonist the tumor. In some aspects, the disclosure provides a method for increasing tumor accumulation of STING agonist in a subject with a tumor, the method comprising administering to a subject in need thereof a lipid composition described herein, or pharmaceutical composition thereof, wherein the lipid composition comprise lipid particles comprising the STING agonist, wherein the lipid particles have a mean diameter of about 10-30 nm, a mean height of about 5-6 nm, and disc-like morphology as measured by, e.g., TEM, and wherein the lipid particles are of sufficient size and/or shape to promote lipid particle accumulation in the tumor following administration, thereby increasing tumor accumulation of STING agonist in the tumor. In some aspects, the administering comprises systemic administration. In some aspects, the tumor is a solid tumor. In some aspects, the accumulation in the tumor is at least about 1%, 2%, 3%, 4%, 5%, about 10%, or about 15% of injected dose per gram of tumor tissue. In some aspects, the accumulation in the tumor occurs in one or more vascular regions of the tumor. In some aspects, the accumulation in the tumor occurs in one or more extravascular regions of the tumor.

In some aspects, the disclosure provides a method for increasing accumulation of STING agonist in one or more target cell populations in a subject with a tumor, the method comprising administering to a subject in need thereof a lipid composition or pharmaceutical composition thereof described herein, and wherein the one or more target cell population are in the tumor, thereby increasing the accumulation of STING agonist in the one or more target cell populations. In some aspects, the disclosure provides a method for increasing accumulation of STING agonist in one or more target cell populations in a subject with a tumor, the method comprising administering to a subject in need thereof a lipid composition described herein, or pharmaceutical composition thereof, wherein the lipid composition comprise lipid particles comprising the STING agonist, wherein the lipid particles have a mean diameter of about 10-30 nm, a mean height of about 5-6 nm, and disc-like morphology as measured by, e.g., TEM, and wherein the lipid particles are of sufficient size and/or shape to promote lipid particle accumulation in the tumor following administration, thereby increasing accumulation in one or more target cell populations in a subject with a tumor. In some aspects, the administering comprsises systemic administration. In some aspects, the one or more target cell populations are selected from: tumor cells, tumor endothelial cells, tumor myeloid cells, dendritic cells, or a combination thereof. In some aspects, the one or more target cell populations comprises tumor cells. In some aspects, the one or more target cell populations comprises tumor myeloid cells. In some aspects, the one or more target cell populations tumor endothelial cells. In some aspects, the one or more target cell populations comprises dendritic cells.

In some aspects, the disclosure provides a method of activating, enhancing, or promoting a response by an immune cell in a subject, comprising administering to the subject a STING agonist amphiphile conjugate described herein, or pharmaceutical composition thereof, thereby activating, enhancing, or promoting a response by an immune cell. In some aspects, the disclosure provides a method of activating, enhancing, or promoting a response by an immune cell in a subject, comprising administering to the subject a lipid composition described herein, or pharmaceutical composition thereof, thereby activating, enhancing, or promoting a response by an immune cell. In some aspects, the disclosure provides a method of activating, enhancing, or promoting a response by an immune cell in a subject, comprising administering to a subject in need thereof a lipid nanodisc or a pharmaceutical composition described herein. In some aspects, the administering comprises systemic administration. In some aspects, the response by the immune cell is a STING-induced response and/or a type I IFN-induced response. In some aspects, the immune cell is a lymphoid cell selected from an innate lymphoid cell, a T cell, a B cell, an NK cell, or a combination thereof. In some aspects, the immune cell is a cytotoxic T cell. In some aspects, the immune cell is an antigen presenting cell selected from a monocyte, a macrophage, a dendritic cell, or a combination thereof. In some aspects, the immune cell is a dendritic cell. In some aspects, the immune cell is a cross-presenting dendritic cell. In some aspects, the response by the immune cell comprises cytokine production, antibody production, priming of tumor antigen-specific immune cells, increased effector function and/or cytotoxicity, or a combination thereof. In some aspects, the immune cell is a dendritic cell, and the response by the immune cell comprises (i) increased production of IFN-α, IFN-β, IL-6, and/or TNF-α, (ii) increased priming of tumor antigen-specific immune cells, or (iii) a combination of (i)-(ii). In some aspects, the response by the immune cell occurs in a tumor microenvironment and/or a tumor lymphoid tissue.

In some aspects, the disclosure provides a method inducing or enhancing an anti-tumor immune response in a subject with cancer, comprising administering to the subject a STING agonist amphiphile conjugate described herein, or a pharmaceutical composition thereof, thereby inducing or enhancing an anti-tumor immune response in the subject. In some aspects, the disclosure provides a method inducing or enhancing an anti-tumor immune response in a subject with cancer, comprising administering to the subject a lipid composition described herein, or a pharmaceutical composition thereof, thereby inducing or enhancing an anti-tumor immune response in the subject. In some aspects, the disclosure provides a method of inducing or enhancing an anti-tumor immune response in a subject with cancer, comprising administering to a subject in need thereof a lipid nanodisc or a pharmaceutical composition described herein. In some aspects, the administering comprises systemic administration. In some aspects, the anti-tumor immune response comprises a STING-induced immune response and/or a type I interferon-induced immune response. In some aspects, the anti-tumor immune response comprises increased production of one or more inflammatory cytokines. In some aspects, the one or more inflammatory cytokines are selected from: a type I INF, IFN-α, IFN-β, IL-6, TNF-α, and any combination thereof. In some aspects, the anti-tumor immune response comprises increased tumor necrosis. In some aspects, the tumor necrosis is measured by increased necrotic tumor cells and/or necrotic tumor endothelial cells. In some aspects, the anti-tumor immune response comprises internalization of lipid particles by at least one cell subset in the tumor microenvironment. In some aspects, the at least one cell subset comprises tumor cells, tumor endothelial cells, myeloid cells, dendritic cells, or a combination thereof. In some aspects, the at least one cell subset comprises tumor cells. In some aspects, the at least one cell subset comprises tumor endothelial cells. In some aspects, the at least one cell subset comprises myeloid cells, optionally CD1 1b⁺CD11c⁻ or CD11b⁻CD1 1c⁺ cells. In some aspects, the at least on cell subset comprises dendritic cells. In some aspects, the anti-tumor immune response comprises (i) increased production of IFN-α, IFN-β, IL-6, and/or TNF-α, (ii) increased priming of tumor antigen-specific immune cells, (i) increased expression of one of more activation markers; and/or (ii) increased trafficking to tumor draining lymphoid tissue by dendritic cells. In some aspects, the dendritic cells are cross-presenting dendritic cells. In some aspects, the anti-tumor immune response comprises increased infiltration of one or more immune cell subsets into the tumor microenvironment and/or tumor lymphoid tissue. In some aspects, the one or more immune cell subsets comprises effector cells. In some aspects, the effector cells are cytotoxic T cells.

In some aspects, the disclosure provides a method of treating cancer comprising administering to a subject in need thereof a STING agonist amphiphile conjugate described herein or a pharmaceutical composition thereof, thereby treating cancer in the subject. In some aspects, the disclosure provides a method of treating cancer comprising administering to a subject in need thereof a lipid composition described herein or a pharmaceutical composition thereof, thereby treating cancer in the subject. In some aspects, the disclosure provides a method of treating cancer comprising administering to a subject in need thereof a lipid nanodisc or a pharmaceutical composition described herein. In some aspects, the administering comprises systemic administration. In some aspects, the cancer is selected from: melanoma, leukemia, lymphoma, lung cancer, breast cancer, prostate cancer, ovarian cancer, colon cancer, mesothelioma, renal cell carcinoma, and brain cancer.

In any of the foregoin or related aspects, the systemic administration is intravenous administration.

In any of the foregoing or related aspects, the disclosure provides a method of treating cancer comprising administering to a subject in need thereof a lipid composition described herein or a pharmaceutical composition described herein, wherein the route of administration of the lipid compostion or the pharmaceutical composition is intravenous.

In any of the foregoing or related aspects, the disclosure provides a method of treating cancer comprising administering to a subject in need thereof a lipid nanodisc or a pharmaceutical composition described herein, wherein the route of administration of a lipid nanodisc or a pharmaceutical composition described herein is intravenous.

In any of the foregoing or related aspects, the method further comprises administering one or more additional therapies selected from: an immune checkpoint inhibitor or an immunogenic cell death inducer. In some aspects, the immune checkpoint inhibitor is an antibody or antigen binding portion thereof that binds to PD-1, PD-L1, CTLA-4, or LAG3.

In some aspects, the disclosure provides a kit comprising a container comprising a lipid nanodisc described herein, and an optional pharmaceutically acceptable carrier, or a pharmaceutical composition described herein, and a package insert comprising instructions for administration of the lipid nanodisc or pharmaceutical composition, for treating or delaying progression of cancer or reducing or inhibiting tumor growth in a subject in need thereof.

In some aspects, the disclosure provides a kit comprising a container comprising a lipid nanodisc described herein, and an optional pharmaceutically acceptable carrier, or a pharmaceutical composition described herein, and a package insert comprising instructions for administration of the lipid nanodisc or pharmaceutical composition, alone or in combination with another agent, for treating or delaying progression of cancer or reducing or inhibiting tumor growth in a subject in need thereof.

In some aspects, the disclosure provides a kit comprising a container comprising a STING agonist amphiphile conjugate described herein, or a pharmaceutically acceptable salt thereof, and an optional pharmaceutically acceptable carrier, and a package insert comprising instructions for administration of the STING agonist amphiphile conjugate, for treating or delaying progression of cancer or reducing or inhibiting tumor growth in a subject in need thereof.

In some aspects, the disclosure provides a kit comprising a container comprising the STING agonist amphiphile conjugate described herein, or a pharmaceutically acceptable salt thereof, and a package insert comprising instructions for administration of the STING agonist amphiphile conjugate, in combination with another agent, for treating or delaying progression of cancer or reducing or inhibiting tumor growth in a subject in need thereof.

In some aspects, the disclosure provides use of STING agonist amphiphile conjugate described herein, or a pharmaceutically acceptable salt thereof, and an optional pharmaceutically acceptable carrier, for the manufacture of a medicament for treating or delaying progression of cancer or reducing or inhibiting tumor growth in a subject in need thereof.

In some aspects, the disclosure provides a kit comprising a container comprising a lipid composition described herein, and an optional pharmaceutically acceptable carrier, and a package insert comprising instructions for administration of the lipid composition, for treating or delaying progression of cancer or reducing or inhibiting tumor growth in a subject in need thereof.

In some aspects, the disclosure provides a kit comprising a container comprising a lipid composition described herein, and a package insert comprising instructions for administration of lipid composition, in combination with another agent, for treating or delaying progression of cancer or reducing or inhibiting tumor growth in a subject in need thereof.

In some aspects, the disclosure provides use of a lipid composition described herein, and an optional pharmaceutically acceptable carrier, for the manufacture of a medicament for treating or delaying progression of cancer or reducing or inhibiting tumor growth in a subject in need thereof.

In some aspects, the disclosure provides a kit comprising a container comprising a pharmaceutical composition described herein, and a package insert comprising instructions for administration of the pharmaceutical composition, for treating or delaying progression of cancer or reducing or inhibiting tumor growth in a subject in need thereof.

In some aspects, the disclosure provides a kit comprising a container comprising the pharmaceutical composition described herein, and a package insert comprising instructions for administration of the pharmaceutical composition, in combination with another agent, for treating or delaying progression of cancer or reducing or inhibiting tumor growth in a subject in need thereof.

In some aspects, the disclosure provides use of pharmaceutical composition described herein, for the manufacture of a medicament for treating or delaying progression of cancer or reducing or inhibiting tumor growth in a subject in need thereof.

In some aspects, the disclosure provides use of a lipid nanodisc described herein, and an optional pharmaceutically acceptable carrier, or a pharmaceutical composition described herein, for the manufacture of a medicament for treating or delaying progression of cancer or reducing or inhibiting tumor growth in a subject in need thereof.

In some aspects, the disclosure provides a vaccine comprising a lipid nanodisc or a pharmaceutical composition described herein, and an antigen, optionally wherein the antigen is conjugated to the lipid nanodisc. In some aspects, the antigen is a tumor-associated antigen. In some aspects, the vaccine is formulated for mucosal or parenteral administration.

In some aspects, the disclosure provides a method of immunizing a subject comprising administering a vaccine described herein.

### BRIEF DESCRIPTION OF DRAWINGS

FIGS. 1A-1B provide schematics depicting the chemical structures of the Linker (intermediate-3) and parent CDN used to generate CDN-Linker for conjugation to a PEG-lipid (FIG. 1A) and the chemical structure of an exemplary CDN-PEG-Lipid conjugate having PEG2k (FIG. 1B).

FIGS. 2A-2B provide graphs measuring STING-induced luciferase activity (FIG. 2A) and quantity of intracellular soluble CDN resulting from cellular uptake (FIG. 2B) in a mouse macrophage STING reporter cell line as a function of CDN concentration following in vitro treatment with parent CDN or exemplary CDN-PEG-Lipid having PEG2k.

FIG. 3A provides a schematic of a lipid nanodisc (LND) comprising hydrogenated soy L-α-phosphatidylcholine (HSPC), PEG-Lipid (with PEG-5k), and CDN-PEG-Lipid (with PEG-2k), referred to herein as “LND-CDN” or “LND-formulated CDN”.

FIG. 3B provides a representative negative stain transmission electron micrograph of LND-CDN particles (scale bar represents 200 nm).

FIG. 3C provides a graph quantifying LDN-CDN particle diameter as measured by transmission electron microscopy.

FIGS. 4A-4B provide graphs depicting area (FIG. 4A) and Day 13 weight (FIG. 4B) of MC38 tumors in mice following administration of a single intravenous (iv) dose of parent CDN or LND-formulated CDN-PEG-Lipid (LND-CDN) as compared to untreated tumors in mice receiving an intratumoral (it) injection of phosphate buffered saline (PBS).

FIGS. 5A-5B provide graphs depicting tumor area (FIG. 5A) and Kaplan-Meier survival (FIG. 5B) of MC38-tumor bearing mice following administration on day 7 post-tumor inoculation of a single intravenous dose of 5 nmol or 100 nmol parent CDN, 100 nmol known STING agonist ADU-S100, or LND-formulated CDN-PEG-Lipid (LND-CDN) at a dose of 5 nmol CDN as compared to untreated mice receiving an injection of PBS.

FIGS. 5C-5D provide graphs depicting tumor area (mean±s.e.m) (FIG. 5C) and Kaplan-Meier survival (FIG. 5D) of MC38-tumor bearing mice following intravenous administration of LND-CDN on day 10 post-tumor inoculation at a dose of 5 nmol CDN per mouse (n=10). Control mice were administered saline only (n=5). Statistical comparisons among tumor areas in FIG. 5C tested using an unpaired, two-tailed Student’s t-test. Statistical comparisons between survival curves in FIG. 5D were performed using a log-rank (Mantel-Cox) test

FIG. 5E provides a graph quantifying response to MC38 tumor re-challenge in the mice from FIG. 5D that rejected tumors (n = 9 animals). Re-challenge with MC38 cells was performed on the opposite flank at 90 days following the initial tumor inoculation. Shown is tumor growth (mean±s.e.m) that was assessed 20 days later. Control animals were naive age-matched control mice (n = 5) given the same MC38 tumor challenge. Statistical comparisons among tumor areas was performing using an unpaired, two-tailed Student’s t-test

FIGS. 6A-6B provide graphs depicting tumor area (FIG. 6A) and Kaplan-Meier survival (FIG. 6B) of B16F10-tumor bearing mice following administration of a single intravenous dose of 5 nmol parent CDN or LND-formulated CDN-PEG-Lipid (LND-CDN) as compared to untreated mice receiving an injection of PBS.

FIGS. 6C-6D provide graphs depicting tumor area (mean±s.e.m) (FIG. 6C) and Kaplan-Meier survival (FIG. 6D) of mice bearing orthotopic 4T1 tumors following administration of a single intravenous dose of parent CDN (200 nmol per mouse; n=10) or LND-CDN (10 nmol CDN per mouse; n=10) on day 7 post tumor-inoculation. Control mice received saline only (n=10). Statistical comparisons among tumor areas in FIG. 6C was performed by an ordinary one-way ANOVA with Tukey’s multiple comparisons test. Statistical comparisons between survival curves in FIG. 6D was performed using a log-rank (Mantel-Cox) test.

FIGS. 6E-6F provide graphs depicting tumor area (mean±s.e.m) (FIG. 6E) and Kaplan-Meier survival (FIG. 6F) of mice bearing TC-1 tumors following administration of a single intravenous dose of LND-CDN (5 nmol CDN per mouse; n=7) on day 7 post tumor-inoculation. Control mice received saline only (n=6). Statistical comparisons was performed as in FIGS. 6C-6D.

FIG. 7A provides a graph quantifying infiltration of immune cells in tumors isolated from MC38-tumor bearing mice following intravenous administration of LND-CDN (5 nmol CDN per mouse; n=5) or parent CDN (5 nmol per mouse; n=5) on day 7 post-tumor inoculation. Tumors were harvested six days following administration and infiltrating CD8+ T cells (left panel), CD4⁺ T cells (middle panel), and NK cells (right panel) were assessed by flow cytometry as a percentage of total live cells. Bars represent means and error bars show s.e.m. Statistical comparisons were made using an unpaired, two-tailed Student’s t-test.

FIG. 7B provides a graph depicting average tumor area (mean ±s.e.m) over time in MC38-tumor bearing mice that were administered an anti-CD8α monoclonal antibody (aCD8a) for depletion of CD8+ T cells or an isotype antibody control (Iso IgG2b) on days 6, 8, 11, and 15 post tumor-inoculation and administration of a single intravenous dose of LND-formulated CDN-PEG-Lipid (LND-CDN) (5 nmol CDN per mouse) on day 7 post tumor-inoculation (n=5 animals/group). Control mice were administered saline only (n=5). Statistical analysis of tumor growth was performed using one-way ANOVA with Tukey’s multiple comparisons test.

FIG. 7C provides a graph depicting average tumor area (mean ±s.e.m) over time in MC38-tumor bearing mice that were administered an anti-NK1.1 monoclonal antibody (αNK1.1) for depletion of NK cells or an isotype antibody control (Iso IgG2a) on days 6, 8, 11, and 15 post tumor inoculation and further administered a single intravenous dose of LDN-CDN (5 nmol CDN per mouse; n=5) on day 7 post tumor inoculation. Control mice were administered saline only (n=5). Statistical analysis performed as in FIG. 7B.

FIG. 7D provides a graph quantifying tumor area (left panel; mean ±s.e.m) and Kaplan-Meier survival (right panel) of Batf3^{-/-} mice bearing MC38 flank tumors following administration of a single intravenous dose of LND-CDN (5 nmol CDN per animal; n=5) on day 7 post-tumor inoculation. Control mice were administered saline only (n=5). Statistical analysis to compare tumor area was performed using an unpaired, two-tailed Student’s t-test and to compare survival curves was performed using a log-rank (Mantel-Cox) test.

FIG. 7E provides a graph depicting tumor area in MC38-tumor bearing mice that were administered PBS or a single intravenous dose of LND-formulated CDN-PEG-Lipid (LND-CDN) alone or in combination with an anti-TNFα monoclonal antibody (aTNFa) for neutralization of TNFα.

FIG. 7F provides a graph depicting tumor area in MC38-tumor bearing mice that were administered PBS or a single intravenous dose of LND-formulated CDN-PEG-Lipid (LND-CDN) alone or in combination with an anti-IFN1 monoclonal antibody (aIFNR1) for blockade of the type 1 interferon receptor (IFNAR-1).

FIGS. 7G-7H provide graphs depicting tumor area (mean ±s.e.m) (FIG. 7G) and survival (FIG. 7H) of MC38-tumor bearing mice that were administered an anti-TNFα, anti-IFNR-1, or anti-IFNy monoclonal antibody, or a combination of the antibodies, at days 6 and 7 post tumor inoculation and a single intravenous dose of LND-CDN (5 nmol per mouse; n=5) on day 7 post tumor inoculation. Control mice were administered saline only (n=5). Statistical comparisons among tumor areas in FIG. 7G were performed using one-way ANOVA with Tukey’s multiple comparisons test and survival curves in FIG. 7H were compared using a log-rank (Mantel-Cox) test.

FIG. 8A (top panel) provides representative images of liposome particles formulated with HSPC, cholesterol, PEG-lipid (DSPE-PEG5k-OMe) and CDN-PEG-lipid with PEG2k (particles referred to as “liposome-CDN” herein) and (bottom panel) provides a graph showing quantification of liposome-CDN particle diameter as measured by cryoTEM.

FIG. 8B provides dose response curves showing STING activation as measured by bioluminescence reporter in RAW-ISG reporter cells as a function of CDN concentration following in vitro treatment for 24 hours with parent CDN, LND-CDN, or liposome-CDN.

FIG. 9A provides a graph quantifying percentage of particles that diffused across a membrane with the indicated pore size. LND-CDN or liposome-CDN were added to a diffusion chamber at 0.50 µM (CDN concentration) that was separated from a receiving chamber by the membrane. Shown is the percentage of total particles detected in the receiving chamber after 24 hours (mean±s.e.m.).

FIGS. 9B-9D provide results from fluorescent imaging of MC38 tumor spheroids that were incubated with fluorescently-labeled LND-CDN (n=5 spheroids) or liposome-CDN (n=6 spheroids) at a concentration of 5.0 µM CDN for 24 hours, followed by washing and imaging of particle penetration by confocal microscopy. FIG. 9B provides representative images depicting particle fluorescence at an optical plane proximal the center of a tumor spheroid that received the LND-CDN (left panel) or the liposome-CDN (right panel). FIG. 9C provides quantification of mean particle fluorescence as a function of radial distance from the tumor spheroid center. FIG. 9D provides quantification of mean fluorescence measured in the central 100 µm radius core of the tumor spheroid (region denoted by dotted circle in FIG. 9B), with statistical comparison performed using an unpaired, two-tailed Student’s t-test.

FIGS. 10A-10B provide graphs depicting tumor area (FIG. 10A) and Kaplan-Meier survival (FIG. 10B) of MC38-tumor bearing mice following administration of a single intravenous dose of LND-formulated CDN-PEG-Lipid (LND-CDN; 5 nmol CDN per mouse; n=10) or liposome-formulated CDN-PEG-Lipid (liposome-CDN; 5 nmol CDN per mouse; n=10) as compared to mice receiving an injection of PBS (n=9).

FIGS. 11A-11B provide graphs depicting the percent of injected dose per gram of tissue (% ID per g) in tumor (FIG. 11A) and tumor draining lymph node (TDLN) (FIG. 11B) tissue at 4 hours and 24 hours following intravenous injection of fluorescently-labeled LND particles formulated with CDN-PEG-Lipid (LND) or liposome particles formulated with CDN-PEG-Lipid (Liposome-CDN) in MC38-tumor bearing mice.

FIG. 12A provides a graph showing the percent of injected dose (% ID) quantified in blood plasma isolated from C57Bl/6 mice that were injected intravenously with Cy5-labeled cGAMP (top panel), Cy5-labeled LND-CDN (bottom left panel), or Cy5-labeled liposome-CDN (bottom right panel). Each were administered a dose of 5.0 nmol CDN per mouse (n=3 animals per group). % ID was determined by fluorescence measurements over time. Dotted lines show two-phase decay curve fits.

FIGS. 12B-12C provide graphs showing the % ID quantified in indicated tissues isolated from MC38-tumor bearing mice that received a single intravenous injection of Cy5-labeled cGAMP, Cy5-labeled LND-CDN, or Cy5-labeled liposome CDN (5 nmol CDN per animal for each; n=4 per group) on day 10 post tumor inoculation. Shown is the organ-level biodistribution (mean±s.e.m.) determined from fluorescence measurements on digested tissues at 24 hours post-injection. Statistical comparisons were evaluated using an ordinary one-way ANOVA with Tukey’s multiple comparisons test.

FIG. 12D provides quantification of mean tumor fluorescent intensity (±s.e.m) of tumor regions measured by cryofluorescence tomography in MC38-tumor bearing mice that received a single intravenous injection of LND-CDN, or liposome-CDN labeled with a near-infrared dye (5 nmol CDN per animal; n=4 per group) on day 10 post tumor inoculation. Control mice were untreated. Cryofluorescence tomography was performed with 50 µm serial sections. Shown is the mean fluorescence intensities summed from 3 tumor regions of interest per mouse (one at the tumor center, one 1 mm dorsal, and one 1 mm ventral). Statistical comparisons were tested using an ordinary one-way ANOVA with Tukey’s multiple comparisons test.

FIG. 12E provides analysis of fluorescence images of tumors that were isolated from MC38-tumor bearing mice administered a single intravenous injection of Cy5-labeled LND-CDN or Cy5-labeled liposome-CDN on day 10 post tumor inoculation (n=3 per group). Control mice were untreated (n=3). At 24 hours following the injection, the mice received an intravenous injection of high MW FITC-dextran (cyan) to label vasculature and tumors were harvested 10 minutes later for histology and confocal imaging to measure particle and dextran fluorescence. Shown is the quantification of the percentage of extravascular tumor area having particle fluorescence (left panel) and the average fluorescence intensity of the extravascular tumor area (right panel). Each point represents 1 mouse and is the average of 2 unique tumor cross-sections. Statistical comparisons were tested using an ordinary one-way ANOVA with Tukey’s multiple comparisons test.

FIG. 13A provides graphs quantifying IFN-β, IL-6, and TNF-α in tumor lysates as measured by bead-based ELISA. The lysates were prepared from tumors obtained from MC38-tumor bearing mice at 4 hours following administration of a single intravenous injection of parent CDN, LND-CDN or liposome-CDN (5 nmol CDN per mouse). Statistical comparisons were performed using one-way ANOVA with Tukey’s multiple comparisons test.

FIG. 13B provides a graph quantifying the number of live tumor cells per milligram of tumor tissue as measured by flow cytometry. The tumors were obtained from MC38-tumor bearing mice at 24 hours following administration of a single intravenous injection of parent CDN, LND-CDN or liposome-CDN (5 nmol CDN per mouse). Control animals were untreated. Statistical analysis performed as in FIG. 13A.

FIGS. 14A-14C provide graphs depicting the total percentage of tumor cells (FIG. 14A), tumor endothelial cells (FIG. 14B), or tumor immune cells (FIG. 14C) in MC38 tumors that were Cy5 positive, wherein the tumors were excised from untreated mice or mice administered Cy5-labeled PEG2k polymer (PEG2k-Cy5) or Cy5-labeled lipid particles (micelles, LNDs, or liposomes).

FIGS. 14E-14H provides graphs showing flow cytometry analysis of tumors isolated from MC38-tumor bearing mice at 24 hours following administration of an intravenous injection of Cy5-labeled particles (LND or liposome). Control mice were untreated. Shown is the percentage of total cells that were particle-positive (left panel) and their mean particle fluorescence intensity (right panel). The cell subsets analyzed that were analyzed were tumor endothelial cells (FIG. 14E), CD11b⁺CD11c⁻ myeloid cells (FIG. 14F), CD11c⁺CD11b⁻ myeloid cells (FIG. 14G), and CD45⁻ non-endothelial cells (FIG. 14H). Statistical analysis was performed using one-way ANOVA with Tukey’s multiple comparisons test.

FIGS. 15A-15F provide graphs showing flow cytometry analysis of DCs in TDLNs that were obtained from mice bearing MC38-ZsGreen tumors, in which the TDLNs were harvested at 1, 2, or 3 days following administration of a single intravenous injection of Cy5-labeled LND-CDN or liposome-CDN (5 nmol CDN; n=5 per group). Control mice received saline only (n=5). FIG. 15A provides a representative flow cytometry plot of uptake of tumor antigen (ZsGreen) and Cy5-labeled particle by DCs. The DCs were obtained from TDLNs harvested at 2 days following administration. FIG. 15B shows the mean (± s.e.m) percentage of DCs that were positive for Cy5-particles. FIG. 15C shows the quantification of the area under the curve (AUC) for FIG. 15B. FIG. 15D shows mean (± s.e.m.) percentages of DCs that were double-positive for Zsgreen tumor antigen and Cy5-particles. FIG. 15E shows the quantification of the AUC for FIG. 15D. FIG. 15F shows analysis of DCs obtained from mice administered LND-CDN particles to determine the percentage of antigen-negative DCs and of antigen-positive DCs that were particle-positive. Statistical comparisons among cell percentages and AUCs were tested using an ordinary one-way ANOVA with Tukey’s multiple comparisons test.

FIGS. 15G-15H provide graphs showing flow cytometry analysis of DCs in TDLNs obtained from mice bearing MC38-ZsGreen tumors, in which the TDLNs were harvested at 1, 2, or 3 days following administration of a single intravenous injection of Cy5-labeled LND-CDN or liposome-CDN (5 nmol CDN; n=5 per group). Control mice received saline only (n=5). FIG. 15G shows representative flow cytometry plot quantifying uptake of tumor antigen (ZsGreen) and expression of CD86 activation marker by DCs. The DCs were obtained from TDLNs harvested at day 3 following administration. FIG. 15H shows a graph quantifying the mean (± s.e.m) percentage of DCs obtained on day 3 that were double-positive for tumor antigen (ZsGreen) and CD86. Statistical comparisons among cell percentages were tested using an ordinary one-way ANOVA with Tukey’s multiple comparisons test.

FIG. 15I provides a graph quantifying IFNy ELISPOT of splenocytes harvested from MC38-tumor bearing mice at 14 days following administration of a single intravenous injection of LND-CDN or liposome-CDN (5 nmol CDN; n=10 per group) to quantify tumor antigen-specific T cells. Control mice received PBS only. Statistical comparisons among groups were tested using Brown-Forsythe and Welch’s ANOVA tests with Dunnett’s T3 multiple comparisons test.

FIGS. 16A-16B provide graphs quantifying mean (± s.e.m.) tumor area (FIG. 16A) and Kaplan-Meier survival (FIG. 16B) of MC38 tumor bearing mice that received a single intratumoral injection of LND-CDN or liposome-CDN (5 nmol; n=5 per group). Control mice received PBS only. Statistical comparisons among tumor growth FIG. 16A (day 18) were tested using an ordinary one-way ANOVA with Tukey’s multiple comparisons test. Statistical comparisons between survival curves in FIG. 16B were performed using a log-rank (Mantel-Cox) test.

### DETAILED DESCRIPTION

### Overview

The present disclosure is based, at least in part, on the surprising discovery that systemic delivery of STING agonist induces a potent anti-tumor immune response, provided the STING agonist is effectively targeted to the tumor with a lipid composition of the disclosure. For example, it was found that systemic delivery of STING agonist using lipid nanodisc carriers as described herein, was highly effective for inducing rapid tumor shrinkage and long-term survival in multiple tumor models. Indeed, formulation of the STING agonist in a lipid nanodisc carrier was found to result in potent therapeutic efficacy following a single intravenous dose. Without being bound by theory, it is believed that these results were due, at least in part, by the lipid nanodisc effectively targeting the STING agonist to tumor and tumor draining lymphatic tissues. In contrast, systemic administration of unformulated (i.e., soluble) STING agonist had limited therapeutic effect. As described herein, systemic delivery of the STING agonist using a lipid nanodisc of the disclosure was found to extend the serum half-life of the STING agonist by more than 10-fold in a pre-clinical animal model compared to unformulated STING agonist. Accordingly, and without being bound by theory, while unformulated STING agonist is rapidly cleared from circulation, systemic delivery of STING agonist by lipid nanodiscs provides for improved STING agonist potency by extending its circulation half-life.

Surprisingly, it was also discovered that systemic delivery of STING agonist using a liposome carrier had minimal effect on tumors. It was further demonstrated that the lipid nanodiscs of the disclosure are more effective than liposomes for targeting tumor tissue, as well as inducing uptake by tumor and immune cells in the tumor microenvironment. As described herein, the lipid nanodisc and the liposome carriers were each found to be effective for reaching tumor vasculature following injection into the circulatory system. Moreover, systemic delivery of STING agonist by either lipid nanodisc or liposome was found to trigger STING-mediated tumor necrosis. However, it was demonstrated that the lipid nanodiscs have improved accumulation within tumor tissue following systemic administration, as well as improved penetration into extravascular areas of the tumor. Without being bound by theory, the lipid nanodiscs are amenable to substantial deformations, such as in response to weak forces, that allows for passage through narrow constrictions, e.g., the dense ECM matrix of the tumor microenvironment. Accordingly, and without being bound by theory, the small size, high aspect ratio, and/or deformable morphology of the lipid nanodiscs of the disclosure mediates effective penetration and accumulation in tumor tissues following systemic administration.

Additionally, it was demonstrated that the systemic delivery of STING agonist using a lipid nanodisc of the disclosure resulted in improved particle uptake by tumor cells and tumor immune cells following systemic administration, e.g., as compared to liposomes. In particular, the lipid nanodiscs were found to induce effective particle uptake and STING-induced activation of tumor dendritic cells. Moreover, as demonstrated herein, systemic delivery of STING agonist formulated using a lipid nanodisc of the disclosure resulted in robust priming of tumor-specific CD8+ T cells. Without being bound by theory, delivery of STING agonist using a lipid nanodisc of the disclosure promotes effective T cell activation by triggering release of tumor antigens through, e.g., STING-mediated necrosis of tumor cells; promoting effective uptake of tumor antigens by dendritic cells; and inducing STING activation of dendritic cells, thereby mediating, e.g., efficient priming of a tumor-specific CD8+ T cell immune response. Thus, lipid nanodiscs were shown to be superior carriers for systemic delivery of STING agonist to tumors, thereby enabling effective STING activation and priming of an anti-tumor immune response. Without being bound by theory, one or more characteristics of the lipid nanodiscs of the disclosure including their lipid composition (e.g., lipid components, component ratio), stability, size, shape, and/or STING agonist release efficiency are thought to contribute to effective targeting of STING agonist to tumors and activation of an anti-tumor immune response.

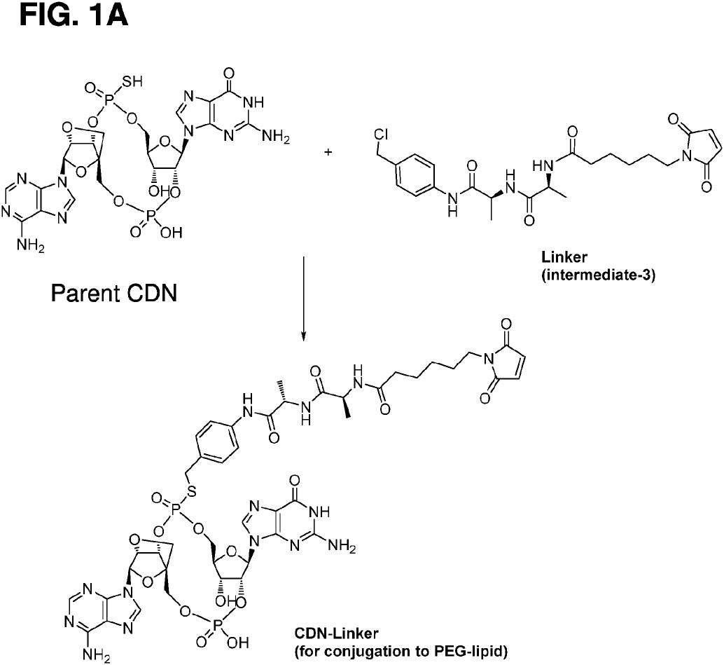

As described herein, the disclosure provides lipid nanodiscs comprising lipid aggregates with disc-like morphology comprising a phospholipid and a PEG-lipid (also referred to as LNDs). The disclosure also provides STING agonist amphiphile conjugates to incorporate STING agonist into lipid nanodiscs. The STING agonist amphiphile conjugates of the disclosure comprise a STING agonist covalently linked to a polymer-modified lipid, optionally via a linker. In some embodiments, the STING agonist is a CDN and the polymer-modified lipid is PEG-lipid, wherein the STING agonist amphiphile conjugate comprises a CDN covalently linked to a PEG-lipid, such as by a peptide linker susceptible to cleavage by intracellular proteases (see, e.g., CDN-PEG-Lipid). In some aspects, the STING agonist amphiphile conjugate is CDN-PEG-Lipid as shown in FIG. 1B. As described herein, STING agonist amphiphile conjugates comprising a protease-cleavable linker were shown to induce an order-of magnitude more potent STING activation in in vitro studies compared to unconjugated agonist, indicating the conjugates are effective for intracellular delivery and release of the agonist to induce STING activation. Furthermore, as described herein, lipid compositions were developed that both provide lipid nanodiscs comprising a STING agonist amphiphile conjugate and have desirable characteristics (e.g., size, morphology, stability, lipid composition, efficient release of STING agonist) for (i) systemic delivery of STING agonist to tumor and tumor lymphoid tissues, and/or (ii) intracellular delivery of STING agonist to target cell populations within the tumor microenvironment. Indeed, as described herein, systemic delivery of lipid nanodiscs induced potent anti-tumor immune responses dependent on CD8+ T cells and STING-induced immunostimulatory cytokines.

### Lipid Compositions of the Disclosure

In some embodiments, the disclosure provides lipid compositions comprising a STING agonist amphiphile conjugate, a phospholipid, and a polyethylene glycol (PEG)-modified lipid (i.e., PEG-lipid), wherein the STING agonist amphiphile conjugate, phospholipid, and PEG-lipid self-assemble to form one or more lipid particles comprising the STING agonist amphiphile conjugate, the phospholipid, and the PEG-lipid. In some embodiments, the one or more lipid particles are selected from: a liposome, a lipid nanodisc, and a spherical micelle. In some embodiments, the one or more lipid particles are lipid nanodiscs.

In some embodiments, the lipid composition of the disclosure comprises a plurality of lipid particles having a diameter between about 10 nm and about 100 nM. In some embodiments, the lipid composition of the disclosure comprises a plurality of lipid particles having a diameter between about 20 nm and about 100 nM. In some embodiments, the lipid composition of the disclosure comprises a plurality of lipid particles having a diameter between about 20 nm and about 80 nM. In some embodiments, the lipid composition of the disclosure comprises a plurality of lipid particles having a diameter between about 20 nm and about 60 nM. In some embodiments, the lipid composition of the disclosure comprises a plurality of lipid particles having a diameter between about 20 nm and about 40 nM. In some embodiments, the lipid composition of the disclosure comprises a plurality of lipid particles having a diameter between about 20 nm and about 30 nM. In some embodiments, the lipid composition of the disclosure comprises a plurality of lipid particles having a diameter between about 30 nm and about 70 nM. In some embodiments, the lipid composition of the disclosure comprises a plurality of lipid particles having a diameter between about 50 nm and about 70 nM. Methods for measuring lipid particle diameter are further described herein.

In some embodiments, the lipid compositions of the disclosure comprise a plurality of lipid particles that are lipid nanodiscs. In some embodiments, the lipid composition of the disclosure comprises a plurality of lipid particles that have disc-like morphology. In some embodiments, the lipid composition of the disclosure comprises a plurality of lipid particles that are discoid micelles. In some embodiments, the lipid compositions of the disclosure comprise a plurality of lipid particles that are spherical micelles. In some embodiments, the lipid compositions of the disclosure comprise a plurality of lipid particles that are liposomes.

In some embodiments, the disclosure provides lipid compositions comprising a STING agonist amphiphile conjugate, a phospholipid, and a polyethylene glycol (PEG)-modified lipid (i.e., PEG-lipid), wherein the STING agonist amphiphile conjugate, phospholipid, and PEG-lipid self-assemble to form lipid nanodiscs. In some embodiments, the lipid nanodiscs comprise one or more characteristics that contribute to effective delivery of a STING agonist to a target tissue and/or target cell population following systemic administration (e.g., intravenous injection). As used herein, “systemic administration” refers to a route of administration of a substance to a subject that introduces the substance into the circulatory system. Non-limiting examples of systemic administration include intravenous injection and intraperitoneal injection. In some embodiments, the lipid nanodisc comprises one or more characteristics that enable effective delivery of STING agonist to a target tissue and/or target cell population following intravenous injection. In some embodiments, the one or more characteristics of the lipid nanodiscs of the disclosure are selected from (i) particle size (e.g., particle diameter of about 20 nm, about 25 nm, about 30 nm, about 35 nm, about 40 nm, about 45 nm, about 50 nm, about 55 nm, about 60 nm, about 65 nm); (ii) particle shape or morphology (e.g., disc-like or discoidal morphology); (iii) particle stability (e.g., stability in serum under physiological conditions, e.g., resistance to phase separation); (iv) composition and/or ratio of lipid components; (v) efficient release of STING agonist (e.g., via cleavage of the STING agonist amphiphile conjugate linker to release the STING agonist or a cleavage product thereof); or (vi) a combination of (i)-(v). In some embodiments, the one or more characteristics include small size (e.g., particle diameter less than about 100, 90, 80, 70, 60, or 50 nm), high aspect ratio (e.g., ratio of particle diameter to particle height that is about 10, 9, 8, 7, 6, 5, 4, or 3 to 1), and/or deformable morphology.

### I. Lipid Particles

In some embodiments, lipid compositions of the disclosure demonstrate phase transition behavior, wherein particle formation transitions from a dispersed lamellar phase (i.e., liposomes) to a micellar phase of spherical micelles via the formation of discoidal or disc-like micelles (i.e., lipid nanodiscs) (see, e.g., Johnsson, et al (2003) BIOPHYSICAL J. 85:3839). In some embodiments, the phase transition behavior is dependent on the molar ratio of PEG-lipid, wherein transition from dispersed lamellar phase to discoidal micellar phase (lipid nanodisc) to spherical micellar phase occurs when the molar ratio of PEG-lipid exceeds certain thresholds. For example, in some embodiments, a phase transition from dispersed lamellar phase (liposome) to discoidal micellar phase (lipid nanodisc) occurs when the molar ratio of PEG-lipid exceeds approximately 5 mol% to about 10 mol%; and the phase transition from a discoidal micellar phase (lipid nanodisc) to a spherical micellar phase occurs when the molar ratio of PEG-lipid exceeds approximately 40 mol% to approximately 70 mol%. In some embodiments, the phase transition from a discoidal micellar phase (lipid nanodisc) to a spherical micellar phase occurs when the molar ratio of PEG-lipid exceeds approximately 25 mol%, 30 mol%, 35 mol%, 40 mol%, or higher.

In some embodiments, the disclosure provides lipid compositions comprising a STING agonist amphiphile conjugate, a phospholipid, and a PEG-lipid, wherein the molar ratio of PEG-lipid exceeds the threshold for phase transition from a dispersed lamellar phase to a micellar phase. In some embodiments, the molar ratio of PEG-lipid is above the threshold for phase transition from a dispersed lamellar phase to a discoidal micellar phase. In some embodiments, the molar ratio of PEG-lipid is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2.0-fold above the threshold. In some embodiments, the threshold for phase transition from a dispersed lamellar phase to a micellar phase or a discoidal micellar phase is about 4 mol%, 5 mol%, 6 mol%, 7 mol%, 8 mol%, 9 mol%, or 10 mol%.

In some embodiments, the molar ratio of PEG-lipid is below the threshold for phase transition from a discoidal micellar phase to a spherical micellar phase. In some embodiments, the molar ratio of PEG-lipid is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.5-fold, or at least 3.0-fold below the threshold. In some embodiments, the threshold for phase transition from a discoidal micellar phase to a spherical micellar phase is about 40 mol%, about 45 mol%, about 50 mol%, about 55 mol%, about 60 mol%, about 65 mol%, or about 70 mol%. In some embodiments, the threshold for phase transition from a discoidal micellar phase to a spherical micellar phase is about 25 mol%, about 30 mol%, about 35 mol%, or about 40 mol%.

In some embodiments, the disclosure provides a lipid composition comprising a molar ratio of PEG-lipid, wherein self-assembly of the STING agonist amphiphile conjugate, the phospholipid and the PEG-lipid favors formation of discoidal micelles (i.e., lipid nanodiscs). In some embodiments, the lipid composition comprises a plurality of lipid particles that are discoidal micelles or lipid nanodiscs. In some embodiments, the molar ratio of PEG-lipid favors formation of lipid particles comprising STING agonist amphiphile conjugate, the phospholipid and the PEG-lipid, wherein at least 50%, at least 55%, at least 60%, at least 65%, at least 70%, at least 75%, at least 80%, at least 85%, at least 90%, at least 95%, or higher of the lipid particles formed are discoidal micelles (i.e., lipid nanodiscs). In some embodiments, less than about 40%, about 35%, about 30%, about 25%, about 20%, about 15%, about 10%, about 9%, about 8%, about 7%, about 6%, about 5%, about 4%, about 3%, about 2%, about 1%, or lower of the particles formed are non-lipid nanodisc particles (e.g., liposomes, spherical micelles, amorphous lipid aggregates).

In some embodiments, the molar ratio of PEG-lipid is about 10-30 mol%, about 15-25 mol%, about 15-20 mol%, or about 15 mol%, about 16 mol%, about 17 mol%, about 18 mol%, about 19 mol%, or about 20 mol% PEG-Lipid.

In some embodiments, the disclosure provides lipid compositions comprising a STING agonist amphiphile conjugate, a phospholipid, and a PEG-lipid, wherein the molar ratio of PEG-lipid is lower than the threshold for phase transition from a dispersed lamellar phase to a micellar phase, thereby providing a lipid composition comprising a plurality of lipid particles that are liposomes. In some embodiments, the molar ratio of PEG-lipid is less than about 10%, 9%, 8%, 7%, 6%, or 5%. In some embodiments the lipid composition comprises a molar ratio of STING agonist amphiphile conjugate that is about 1%, a molar ratio of PEG-lipid that is about 1-9%, and a molar ratio of phospholipid that is about 90-95%, wherein the lipid composition comprises a plurality of lipid particles that are liposomes. In some embodiments, the lipid composition comprises a molar ratio of STING agonist amphiphile conjugate that is about 1%, a molar ratio of PEG-lipid that is about 4%, and a molar ratio of phospholipid that is about 95%, wherein the lipid composition comprises a plurality of lipid particles that are liposomes. In some embodiments, the liposomes have a particle diameter between about 10 nm and about 150 nm. In some embodiments, the liposomes have a particle diameter between about 40 nm and about 100 nm. In some embodiments, the liposomes have a particle diameter of about 65 nm.

### II. Delivery of STING Agonist

The present disclosure is based, at least in part, on the discovery that lipid compositions of the disclosure were particularly effective for systemic delivery of STING agonist in a manner that generates a potent anti-tumor immune response. As described herein, the lipid compositions of the disclosure (e.g., lipid nanodisc compositions) provide an anti-tumor immune response that is effective for reducing or eliminating tumor burden and/or promoting long-term survival following systemic administration in multiple pre-clinical tumor models. Furthermore, the lipid compositions (e.g., lipid nanodisc compositions) were found to deliver STING agonist in a manner that extend serum half-life of the STING agonist, e.g., as compared to systemic administration of a control STING agonist (e.g., unformulated and/or unconjugated STING agonist), thereby improving in vivo potency. The lipid compositions (e.g., lipid nanodisc compositions) were also demonstrated to induce substantial STING-induced tumor necrosis, thereby providing, e.g., tumor antigens for eliciting a tumor-specific immune response. Additionally, the lipid compositions (e.g., lipid nanodisc compositions) were demonstrated to induce effective uptake of STING agonist and tumor antigen by dendritic cells in the tumor microenvironment, thereby providing, e.g., optimal activation of dendritic cells and priming of tumor antigen-reactive CD8+ T cells. Accordingly, in some aspects, the disclosure provides lipid compositions that deliver STING agonist in manner that extends its serum half-life and provides robust STING-induced anti-tumor immune responses.

In some aspects, the disclosure provides lipid compositions comprising a lipid particle (e.g., lipid nanodisc) comprising a STING agonist amphiphile conjugate, wherein the lipid particle has disc-like morphology, small size (e.g., diameter of about 15-50 nm), high aspect ratio (e.g., particle diameter to particle height that is about 10, 9, 8, 7, 6, 5, 4, or 3 to 1), and/or deformable morphology to enable efficient deliver to a target tissue (e.g., tumor) following systemic administration. In some embodiments, the disclosure provides lipid nanodiscs comprising a STING agonist amphiphile conjugate described herein, wherein the lipid nanodiscs promote delivery of STING agonist to a target tissue and/or a target cell population. In some embodiments, the target tissue is a tumor tissue and/or a lymphatic tissue. In some embodiments, the target cell population is a STING expressing cell population, such as a STING-expressing tumor cell population or immune cell population. In some embodiments, the target cell population comprises a STING-expressing antigen presenting cell. In some embodiments, the target cell population comprises a STING-expressing dendritic cell. In some embodiments, the target cell population comprises a STING-expressing cross-presenting dendritic cell. In some embodiments, systemic administration (e.g., intravenous injection) of the lipid nanodiscs is used for delivery of the STING agonist to a target tissue and/or target cell population.

In some embodiments, the disclosure provides lipid nanodiscs that promote delivery of STING agonist by increasing serum half-life of STING agonist. In some embodiments, the lipid nanodiscs increase serum half-life of STING agonist following systemic administration (e.g., intravenous administration). For example, serum half-life is increased compared to delivery of unformulated STING agonist. In some embodiments, serum half-life is increased compared to systemic administration of unformulated or unconjugated STING agonist. In some embodiments, serum half-life is increased by about 1 hour, about 2 hours, about 3 hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, 24 hours, about 30 hours, about 36 hours, about 42 hours, about 48 hours, or more compared to unformulated STING agonist. In some embodiments, the serum half-life of STING agonist following systemic administration of a lipid composition described herein (e.g., lipid composition comprising lipid nanodiscs) comprising the STING agonist is at least about 1-2 hours, 1-3 hours, 1-4 hours, 1-5 hours, 1-6 hours, 1-7 hours, 1-8 hours, 1-9 hours, 1-10 hours, 5-10 hours, 5-15 hours, 5-20 hours, 10-15 hours, 10-20 hours, or 10-30 hours. In some embodiments, the serum half-life of the STING agonist is at least about 1, 2, 3, 4, 5, 6, 7, 8, 9, 10, 11, 12, 13, 14, or 15 hours. In some embodiments, the serum half-life of the STING agonist is about 5 hours. In some embodiments, the serum half-life of the STING agonist is about 6 hours. In some embodiments, the serum half-life of the STING agonist is about 7 hours. In some embodiments, the serum half-life of the STING agonist is about 8 hours. In some embodiments, the serum half-life of the STING agonist is about 9 hours. In some embodiments, the serum half-life of the STING agonist is about 10 hours. In some embodiments, the serum half-life of the STING agonist is about 11 hours. In some embodiments, the serum half-life of the STING agonist is about 12 hours. In some embodiments, the serum half-life of the STING agonist is about 13 hours. In some embodiments, the serum half-life of the STING agonist is about 14 hours. In some embodiments, the serum half-life of the STING agonist is about 15 hours. Methods of measuring serum half-life are known in the art. For example, collection of blood samples at regular intervals following administration, and quantification by liquid chromatography, mass spectrometry, or a STING agonist functional assay such as those detailed below.

In some aspects, the disclosure provides lipid compositions comprising a lipid particle (e.g., lipid nanodisc) comprising a STING agonist amphiphile conjugate, wherein the lipid particle has one or more characteristics selected from: a disc-like morphology, small size (e.g., diameter of about 15-50 nm), high aspect ratio (e.g., particle diameter to particle height that is about 10, 9, 8, 7, 6, 5, 4, or 3 to 1), and deformable morphology, that enable increased accumulation in one or more target tissues (e.g., tumor and/or tumor draining lymphoid tissue) following systemic administration, e.g., as compared to a control lipid particle comprising the STING agonist (e.g., a spheroid particle or liposome comprising the STING agonist amphiphile conjugate). As used herein, “accumulation” refers to the total quantity or proportion of the injected dose that is present in a target tissue at a pre-determined time point following in vivo administration (e.g., systemic administration). Methods for measuring STING agonist and/or lipid particle accumulation in a target tissue (e.g., tumor and/or tumor draining lymphatic tissue) following administration to a subject are known in the art, and include, e.g., medical diagnostic imaging of a STING agonist and/or lipid particle labeled with a contrast agent, obtaining a tissue biopsy sample to quantify STING agonist and/or lipid particle. In some embodiments, the target tissue is tumor tissue. In some embodiments, the target tissue is tumor lymphoid tissue. In some embodiments, the target tissue is non-tumor tissue (e.g., liver).

In some aspects, the disclosure provides lipid compositions comprising a lipid particle (e.g., lipid nanodisc) comprising a STING agonist amphiphile conjugate, wherein the lipid particle has a disc-like morphology, small size (e.g., diameter of about 15-50 nm), and deformable morphology, that enable increased accumulation in tumor tissue following systemic administration, e.g., compared to a control lipid particle comprising the STING agonist (e.g., a spheroid particle or liposome comprising the STING agonist amphiphile conjugate). In some embodiments, the disclosure provides lipid nanodiscs that promote delivery of STING agonist by increasing accumulation of STING agonist in tumor and/or tumor draining lymphatic tissue. For example, accumulation of STING agonist is increased compared to delivery as unformulated STING agonist or delivery by a non-lipid nanodisc particle (e.g., STING agonist formulated as a liposome or spherical micelle).

In some aspects, the disclosure provides lipid compositions comprising a lipid particle (e.g., lipid nanodisc) comprising a STING agonist amphiphile conjugate, wherein the lipid particle has one or more characteristics selected from: a disc-like morphology, small size (e.g., diameter of about 15-50 nm), high aspect ratio (e.g., particle diameter to particle height that is about 10, 9, 8, 7, 6, 5, 4, or 3 to 1), and deformable morphology, that enable increased penetration of one or more target tissues (e.g., tumor and/or tumor draining lymphoid tissue) following systemic administration, e.g., compared to a control lipid particle comprising the STING agonist (e.g., a spheroid particle or liposome comprising the STING agonist amphiphile conjugate). As used herein, “tissue penetration” or “penetration” each refer to the total quantity or proportion of the injected dose (e.g., injected dose of STING agonist and/or injected dose of lipid particle) that passes from the vasculature into the extravasculature regions of the target tissue (e.g., tumor) following injection into the circulatory system of a subject.

In some aspects, the disclosure provides lipid compositions comprising a lipid particle (e.g., lipid nanodisc) comprising a STING agonist amphiphile conjugate, wherein the lipid particle has disc-like morphology, small size (e.g., diameter of about 15-50 nm), high aspect ratio (e.g., particle diameter to particle height that is about 10, 9, 8, 7, 6, 5, 4, or 3 to 1), and deformable morphology, and wherein the lipid particle has increased tumor penetration following systemic administration, e.g., compared to a control lipid particle comprising the STING agonist (e.g., a spheroid particle or liposome comprising the STING agonist amphiphile conjugate). As used herein, “tumor penetration” refers to the total quantity or proportion of the injected dose (e.g., injected dose of STING agonist and/or injected dose of lipid particle) that passes from the tumor vasculature in the extravasculature regions of the tumor tissue following injection into the circulatory system of a subject having a tumor. Methods for measuring tissue penetration or tumor penetration are known in the art and described in the Examples provided herein, and include, e.g., histology. In some embodiments, tumor penetration is evaluated as the percentage of total lipid particle (e.g., lipid nanodisc) in the tumor that is present in the extravascular areas of the tumor, e.g., as measured by histology. In some embodiments, at least about 5, 10, 15, 20, 25, 30, 35, 40, 45, or 50% of the total lipid particle (e.g., lipid nanodisc) in the tumor is in extravascular areas of the tumor. In some embodiments, about 10% of the total lipid particle (e.g., lipid nanodisc) in the tumor tissue is in extravascular areas of the tumor. In some embodiments, about 15% of the total lipid particle (e.g., lipid nanodisc) in the tumor tissue is in extravascular areas of the tumor. In some embodiments, about 20% of the total lipid particle (e.g., lipid nanodisc) in the tumor tissue is in extravascular areas of the tumor. In some embodiments, about 25% of the total lipid particle (e.g., lipid nanodisc) in the tumor tissue is in extravascular areas of the tumor. In some embodiments, about 30% of the total lipid particle (e.g., lipid nanodisc) in the tumor tissue is in extravascular areas of the tumor.

In some embodiments, the lipid nanodiscs promote delivery of STING agonist by increasing tumor penetration of the STING agonist. Without being bound by theory, the lipid nanodiscs of the disclosure have improved tumor penetration, e.g., as compared to a control lipid particle comprising the STING agonist (e.g., spheroid particle or liposome), due to having a small size, high aspect ratio, and/or deformable morphology that allows for translocation across narrow pores present in the dense ECM of the tumor microenvironment. In some embodiments, tumor penetration is increased compared to delivery by non-lipid nanodisc particle (e.g., STING agonist formulated as a liposome or spherical micelle). In some embodiments, tumor penetration of STING agonist following systemic administration of a lipid nanodisc comprising the STING agonist is increased by at least about 1.1, 1.2, 1.3, 1.4, 1.5, 1.6, 1.7, 1.8, 1.9, 2, 2.5, 3, 3.5, 4, 4.5, or 5-fold relative to systemic delivery of a control lipid particle (e.g., STING agonist formulated as a spherical particle, a liposome, a micelle). In some embodiments, tumor penetration of STING agonist following systemic delivery of lipid nanodisc is increased by about 2-fold relative to systemic delivery of a control lipid particle (e.g., STING agonist formulated as a spherical particle, a liposome, a micelle). In some embodiments, tumor penetration of STING agonist following systemic delivery of lipid nanodisc is increased by about 3-fold relative to systemic delivery of a control lipid particle (e.g., STING agonist formulated as a spherical particle, a liposome, a micelle). In some embodiments, tumor penetration of STING agonist following systemic delivery of lipid nanodisc is increased by about 4-fold relative to systemic delivery of a control lipid particle (e.g., STING agonist formulated as a spherical particle, a liposome, a micelle). In some embodiments, tumor penetration of STING agonist following systemic delivery of lipid nanodisc is increased by about 5-fold relative to systemic delivery of a control lipid particle (e.g., STING agonist formulated as a spherical particle, a liposome, a micelle).

In some embodiments, the disclosure provides lipid nanodiscs that promote delivery of STING agonist by increasing STING agonist uptake by cells of the tumor microenvironment (e.g., tumor cells, immune cells, epithelial cells). For example, uptake is increased compared to delivery as unformulated STING agonist or STING agonist formulated as a non-lipid nanodisc particle (e.g., liposome, spherical micelle). In some embodiments, the lipid nanodiscs promote increased intracellular concentration of STING agonist in cells of the tumor microenvironment. For example, intracellular concentration of STING agonist is increased compared to delivery of STING agonist or STING agonist formulated as a non-lipid nanodisc particle (e.g., liposome, spherical micelle). In some embodiments, the lipid nanodiscs promote delivery of STING agonist by increasing STING activation by cells of the tumor microenvironment (e.g., tumor cells, immune cells, epithelial cells). For example, STING activation is increased compared to delivery as unformulated STING agonist or STING agonist formulated as a non-lipid nanodisc particle (e.g., liposome, spherical micelle). Methods of measuring cell uptake and/or intracellular concentration of STING agonist are known in the art and provided in the Example section herein. Methods of measuring STING activation are further described below.

Certain properties of lipid particle carriers are appreciated for their impact on drug delivery. For example, lipid particle carriers have been shown to increase serum half-life of loaded drug compounds and promote passive targeting of the drug compounds to tumor tissue by having sufficient size and/or shape to exploit the enhanced permeability and retention (EPR) effect (see, e.g., Golombek, et al (2018) ADV. DRUG DELIV. REV. 130:17-38). However, it is also known that tumor tissues present barriers that prevent penetration by large particles (e.g., particles with diameter of approximately 100 nm or higher, e.g., liposomes) (see, e.g., Stapleton, S. et al (2015) J CONTROLLED RELEASE 211:163-170). Such barriers include dense extracellular matrix (ECM) and high interstitial fluid pressure that hinder particle penetration into tumor tissue. Without being bound by theory, the lipid compositions of the disclosure provide lipid nanodiscs comprising a STING agonist amphiphile conjugate described herein that have optimal size, shape, aspect ratio, and/or deformability to enable systemic delivery of the STING agonist, wherein the systemic delivery provides one or more improved pharmacokinetic and/or biodistribution properties compared to systemic administration of unformulated STING agonist and/or a control lipid particle comprising the STING agonist (e.g., spherical particle, liposome). In some embodiments, the one or more improved pharmacokinetic and/or biodistribution properties is selected from: increased circulation half-life of the STING agonist, increased accumulation in the tumor microenvironment, increased penetration of extravascular areas of the tumor microenvironment, and increased STING agonist uptake by tumor cells and/or immune cells in the tumor microenvironment. In some embodiments, the one or more improved pharmacokinetic and/or biodistribution properties provides for an enhanced STING-induced anti-tumor immune response, as describe herein.

In some embodiments, the disclosure provides lipid nanodiscs of sufficient size (e.g., particle diameter greater than about 10 to about 15 nm, or about 10 nm to about 20 nm) to increase serum half-life of STING agonist following systemic administration (e.g., intravenous injection) compared to systemic administration of unformulated STING agonist. In some embodiments, the lipid nanodiscs are of sufficient size to promote passive targeting of STING agonist to tumor tissue by the EPR effect.

In some embodiments, disclosure provides lipid nanodiscs of sufficient size (e.g., particle diameter less than about 100 nm, about 90 nm, about 80 nm, about 70 nm, or about 60 nm) to enable increased accumulation of STING agonist in tumor tissue following systemic administration (e.g., intravenous injection). In some embodiments, the lipid nanodisc morphology (e.g., disc-like morphology) enables increased accumulation of STING agonist in tumor tissue following systemic administration. In some embodiments, accumulation of STING agonist in tumor tissue is increased by about 1.5-fold, about 2-fold, about 3-fold, about 4-fold, about 5-fold, about 6-fold, about 7-fold, about 8-fold, about 9-fold, about 10-fold, or higher compared to systemic administration of equivalent, unformulated STING agonist or STING agonist formulated as a non-lipid nanodisc particle (e.g., liposome particle). In some embodiments, the lipid nanodiscs have sufficient size (e.g., particle diameter less than about 100 nm, about 90 nm, about 80 nm, about 70 nm, or about 60 nm), shape (e.g., disc-like morphology), flexibility, and/or aspect ratio (e.g., ratio of particle diameter to height that is about 10, 9, 8, 7, 6, 5, 4, or 3 to 1) to penetrate ECM of tumor tissue to a greater extent that a control lipid particle (e.g., spherical particle, liposome). Accordingly, in some embodiments, the lipid nanodiscs provide increased penetration of STING agonist in extravascular areas of the tumor tissue as compared to a control lipid particle (e.g., spherical particle, liposome) comprising the STING agonist.

Spherical micelles have been shown to disassemble in the presence of serum proteins (see, e.g., Liu, H. et al (2014) NATURE 507:519-522). By contrast, and without being bound by theory, lipid nanodiscs are thought to be more resistant to disassembly by serum proteins. In some embodiments, the disclosure provides lipid nanodiscs with increased stability in the presence of serum albumin under physiological conditions. For example, disclosure provides lipid nanodiscs with increased stability compared to a spherical micelle. In some embodiments, the lipid nanodiscs of the disclosure remain assembled in the presence of serum albumin under physiological conditions. Methods of measuring particle stability in serum are known in the art, for example, as described by Lui, et al.

### III. Components of the Lipid Composition

### A. STING Agonist Amphiphile Conjugate

As described herein, the present disclosure provides lipid nanodiscs comprising a STING agonist amphiphile conjugate, wherein the conjugate comprises a STING agonist covalently linked to a polymer-modified lipid, optionally via a linker. In some embodiments, the STING agonist is a compound that (i) binds to STING receptor, and (ii) comprises a reactive moiety for covalent linkage. In some embodiments, the STING agonist is covalently linked directly to a polymer-modified lipid via the reactive moiety. In some embodiments, the STING agonist is covalently linked to a polymer-modified lipid via a linker, wherein the STING agonist is covalently-linked to the linker via the reactive moiety and the linker is covalently-linked to the polymer of the polymer-modified lipid. In some embodiments, the polymer-modified lipid comprises a polymer covalently-linked to a lipid, for example a diacyl lipid.

In some embodiments, a STING agonist amphiphile conjugate comprises the formula ST-L-P-LI, wherein: ST is a STING agonist that (i) binds to STING receptor, and (ii) comprises a reactive moiety for covalent linkage to L; wherein L is a linker that (i) comprises a reactive moiety for covalent linkage with ST, (ii) comprises a reactive moiety for covalent linkage with P, and optionally (iii) comprises one or more cleavage element; wherein P is a polymer, and wherein LI is a lipid.

In some embodiments, the STING agonist is a CDN. In some embodiments, the STING agonist is a compound represented by any one of Formula (XX)-(XXIX) as provided below. In some embodiments, the linker is represented by the formula -X³-T-Z-Q-, wherein X³ is a divalent radical that connects the STING agonist to the rest of the linker, or is absent, T is a peptide, or is absent, Z is a spacer, and Q is a heterobifunctional group or a heterotrifunctional group. By way of illustration, the following generic formula shows a STING agonist amphiphile conjugate of the disclosure having a para-aminobenzyl-based connector, an alanine-alanine-based dipeptide, a propanone-based spacer, and a succinimide thioether-based heterobifunctional group:

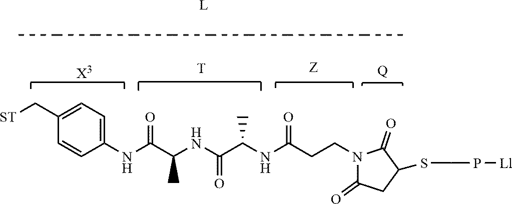

In some embodiments, the polymer is represented by the formula -Z²-(CH₂CH₂O)ₙ-Z³-, wherein Z² is a spacer for covalent attachment to the linker, n is 25-230, and Z³ is a spacer for covalent attachment to the head group of the lipid. By way of illustration, the following generic formula shows a STING agonist amphiphile conjugate of the disclosure having a para-aminobenzyl-based connector, an alanine-alanine-based dipeptide, a propanone-based spacer, a succinimide thioether-based heterobifunctional group, an ethane carboxamide spacer, ethylene glycol units (number of units denoted by “n”; e.g., n = 25-230), and an acyl group spacer:

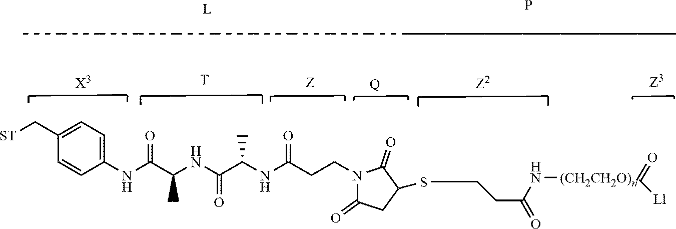

In some embodiments, the lipid is a diacyl lipid. In some embodiments, the lipid is a phospholipid comprising two fatty acid tails and a phosphate head group. In some embodiments, the phospholipid is a glycerophospholipid. In some embodiments, the phosphate is appended to the polymer via an ethanolamine spacer unit. In some embodiments, the lipid is represented by the formula (LI-I):

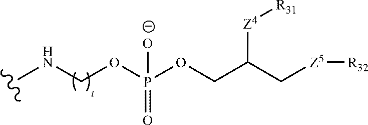

wherein t is 1, 2, 3, 4, 5, 6, 7, 8, 9, or 10, Z⁴ and Z⁵ are spacer groups for attachment to the carbon backbone, and R₃₁ and R₃₂ are aliphatic chains (e.g., optionally substituted alkyl, alkenyl, or alkynyl groups, e.g., optionally substituted C₁₋₃₀ alkyl, alkenyl, or alkynyl groups). By way of illustration, the following generic formula shows a STING agonist amphiphile conjugate of the disclosure having a para-aminobenzyl-based connector, an alanine-alanine-based dipeptide, a propanone-based spacer, a succinimide thioether-based heterobifunctional group, an ethane carboxamide spacer, n ethylene glycol units (e.g., n = 25-230), an acyl group spacer for attachment to a glycophospholipid with a phosphoethanolamine head group and acyl chains of x methylene units.

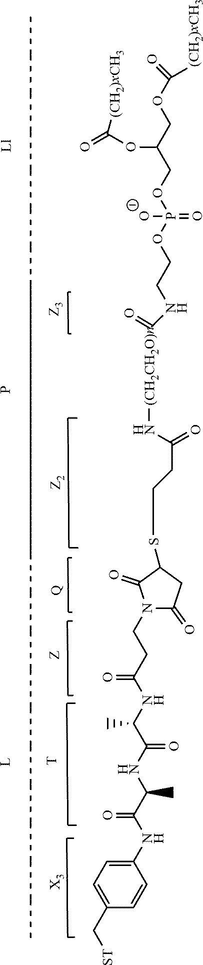

### (I) STING Agonist Compounds

In some embodiments, the STING agonist is a compound that binds to mouse and human STING. In some embodiments, the STING agonist is a compound that binds to human STING. Five haplotypes encoding human STING have been identified (WT, REF, HAQ, AQ, and Q allelles) which vary at amino acid positions 71, 230, 232, and 293 (Jin et al (2011), GENES IMMUN. 12:263-269; Yi, et al (2013) PLoS ONE 8:e77846). In some, the STING agonist is a compound that binds to human STING encoded by the WT, REF, HAQ, AQ, and/or Q allele. In some embodiments, the STING agonist is a compound that binds to human STING encoded by the WT allele. In some embodiments, the STING agonist is a compound that binds to human STING encoded by the REF allele. In some embodiments, the STING agonist is a compound that binds to human STING encoded by the HAQ allele. In some embodiments, the STING agonist is a compound that binds to human STING encoded by the AQ allele. In some embodiments, the STING agonist is a compound that binds to human STING encoded by the Q allele.

In some embodiments, the STING agonist activates or potentiates STING activity. Activation of STING results in a downstream signaling cascade involving TBK1 activation, IRF-3 phosphorylation, and production of inflammatory cytokines (see, e.g. Burdette, et al (2011) NATURE 478:515-518; Burdette and Vance, (2013) NAT. IMMUNOL. 14:19-26; Ishikawa and Barber, (2008) NATURE 455:674-678). In some embodiments, the STING agonist promotes or increases production of at least one cytokine and/or chemokine in a STING-expressing cell. For example, in some embodiments, production is increased by at least 1.1-fold, 1.2-fold, 1.3-fold, 1.4-fold, 1.5-fold, 1.6-fold, 1.7-fold, 1.8-fold, 1.9-fold, 2-fold, or higher compared to an untreated STING-expressing cell. In some embodiments, the inflammatory cytokine is an interferon or a cytokine that binds interferon receptor. In some embodiments, the inflammatory cytokine is a type 1 interferon, such as IFN-alpha or IFN-beta. In some embodiments, the inflammatory cytokine is a type 3 interferon. In some embodiments, the inflammatory cytokine is IFN-gamma. In some embodiments, the inflammatory cytokine is TNF-alpha, IL-6, IL-12, or IL-1B. In some embodiments, the STING activates production of at least one chemokine. In some embodiments, at least one chemokine is selected from CXCL10, CXCL9, CCL4, or CXCL11. Methods of measuring production of chemokines and/or cytokines in a cell are known in the art, including for example, ELISA, a multiplex bead array, or a multiplex surface array assay (e.g., Luminex).

In some embodiments, STING agonist activity is measured by an interferon stimulation assay, a reporter gene assay, a TANK binding kinase (TBK) 1 activation assay, a interferon-gamma-inducible protein (IP-10) assay, a STING Biochemical [3H]cGAMP competition assay, or other assays known to one skilled in the art for assessing STING activity. In some embodiments, the STING agonist promotes or increases STING activity in a STING-expressing cell line when measured in comparison to the STING-expressing cell line in the absence of the STING agonist (i.e., untreated cells). In some embodiments, the activity of the STING agonist is measured in a STING-expressing cell line and compared to activity in the same cell line with the STING pathway partially or wholly deleted (e.g., STING^{-/-} cells), wherein agonist activity is significantly elevated in the STING-expressing cell line compared to the cell line with the STING pathway partially or wholly deleted.

In some embodiments, the STING agonist promotes or increases STING activity in STING-expressing cells that further express a reporter gene (e.g., GFP, luciferase) under control of an interferon (IFN)-stimulated response element. In some embodiments, the STING agonist stimulates expression of a luciferase reporter gene controlled by a IFN-stimulated response element in a STING-expressing cell with an EC50 of at least 50 µM, 45 µM, 40 µM, 35 µM, 30 µM, 25 µM, 20 µM, 19 µM, 18 µM, 17 µM, 16 µM, 15 µM, 14 µM, 13 µM, 12 µM, 11 µM, 10 µM, 9 µM, 8 µM, 7 µM, 6 µM, 5 µM, 4 µM, 3 µM, 2 µM¸ 1 µM, or less. In some embodiments, the STING agonist stimulates expression of a luciferase reporter gene controlled by a IFN-stimulated response element in a STING-expressing cell to a level equal to or greater than the level of stimulation induced by cyclic di-guanosine 5′-monophosphate (cyclic di-GMP), cyclic di-inosine monophosphate, cyclic di-adenosine 5′-monophosphate (cyclic di-AMP or CDA), cyclic GMP-AMP (cGAMP), cyclic[G(2′,5′)pA(3′5′)p] (2′-3′ cGAMP), or cyclic[A(2′,5′)pA(3′,5′)p] (2′-3′ CDA).

In some embodiments, the STING agonist is a non-nucleotide small molecule that (i) binds to STING receptor, and (ii) comprises a reactive moiety for covalent linkage. STING agonists that are non-nucleotide small molecules are known in the art. For example, amidobenzimidazole (ABZI)-based compounds or diABZI-based compounds such as those disclosed by Ramanjulu, et al (2018) NATURE 564:439-443 and WO2019/069270, both of which are incorporated by reference herein in their entirety. In some embodiments, the STING agonist is an ABZI-based compound according to formula (I) as defined in WO2019/069270, wherein formula (I) is:

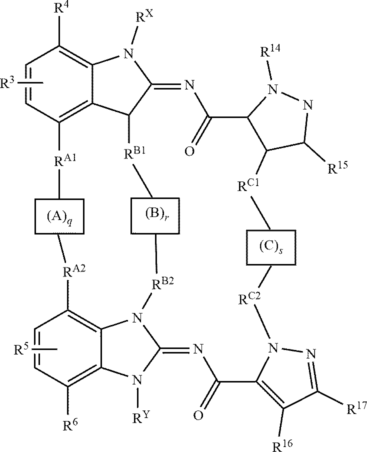

wherein q, r, s, A, B, C, R^{A1}, R^{A2}, R^{B1}, R^{B2}, R^{C1}, R^{C2}, R³, R⁴, R⁵, R⁶, R¹⁴, R¹⁵, R¹⁶, R¹⁷, R^{X}, and R^{Y} are as defined in WO2019/069270, or a tautomer thereof, or a salt thereof.

In some embodiments, an ABZI-based compound according to formula (I) as defined in WO2019/069270 is modified for covalent linkage to a polymer-modified lipid of the disclosure according to one of the following:
(i) q is 0; R^{A1} is -OR, -NR′R, -PO₃R, or -SR; and R^{A2} is as defined in WO2019/069270 for q is 0;
(ii) q is 0; R^{A2} is -OR, -NR′R, -PO₃R, or -SR; and R^{A1} is as defined in WO2019/069270 for q is 0;
(iii) r is 0; R^{B1} is -OR, -NR′R, -PO₃R, or -SR; and R^{B2} is as defined in WO2019/069270 for r is 0;
(iv) r is 0; R^{B2} is -OR, -NR′R, -PO₃R, or -SR; and R^{B1} is as defined in WO2019/069270 for r is 0;
(v) s is 0; R^{C1} is -OR, -NR′R, -PO₃R, or -SR; and R^{C2} is as defined in WO2019/06270 for s is 0;
(vi) s is 0; R^{C2} is -OR, -NR′R, -PO₃R, or -SR; and R^{C1} is as defined in WO2019/06270 for s is 0;
(vii) R³ is -CONR′R, CO₂R, or OR;
(viii) R⁵ is -CONR′R, CO₂R, or OR;
(ix) R⁴ is -OR, -PO₃R, -NR′R, -COR, -CO₂R, -N(R′)COR, -N(R′)SO₂R, -N(R′)SO₂(C₁-C₂alkyl)-NRR, -N(R′)CO(C₁-C₂alkyl)-NR′R, or a C₁₋₆ alkyl optionally substituted with -OR, -NR′R, -CO₂R, -CONR′R, -SO₂NR′R, or -OCONR′R;
(x) R⁶ is -OR, -PO₃R, -NR′R, -COR, -CO₂R, -N(R′)COR, -N(R′)SO₂R, -N(R′)SO₂(C₁-C₂alkyl)-NR′R, -N(R′)CO(C₁-C₂alkyl)-NR′R, or a C₁₋₄ alkyl substituted with -OR, -NR′R, -CO₂R, -CONR′R, -SO₂NR′R, or -OCONR′R;
(xi) R¹⁴ is a C₁₋₄ alkyl substituted with -OR, -NR′R, -CO₂R, -CONR′R, -SO₂NR′R, or -OCONR′R;
(xii) R¹⁵ is a C₁₋₄ alkyl substituted with -OR, -NR′R, -CO₂R, -CONR′R, -SO₂NR′R, or -OCONR′R;
(xiii) R¹⁶ is a C₁₋₄ alkyl substituted with -OR, -NR′R, -CO₂R, -CONR′R, -SO₂NR′R, or -OCONR′R;
(xiv) R¹⁷ is a C₁₋₄ alkyl substituted with -OR, -NR′R, -CO₂R, -CONR′R, -SO₂NR′R, or -OCONR′R;
(xv) R^{X} is is a C₁₋₄ alkyl substituted with -OR, -NR′R, -CO₂R, -CONR′R, -SO₂NR′R, or -OCONR′R; or
(xvi) R^{Y} is is a C₁₋₄ alkyl substituted with -OR, -NR′R, -CO₂R, -CONR′R, -SO₂NR′R, or -OCONR′R; wherein R is a linker providing covalent attachment to the polymer-modified lipid (a linker providing covalent attachment to an available carbon atom, oxygen atom, nitrogen atom, or sulfur atom of the polymer portion of the polymer-modified lipid); and R′ is a hydrogen atom or a substituent; wherein q+r+s = 1 or 2. In some embodiments, q is 1, and R^{A1}, A, and R^{A2} form a linking group as defined in WO2019/069270. In some embodiments, r is 1, and R^{B1}, B, and R^{B2} form a linking group as defined in WO2019/069270. In some embodiments, s is 1, and R^{C1}, C, and R^{C2} form a linking group as defined in WO2019/069270.

### Cyclic Dinucleotide (CDN) and Modified CDN Compounds

In some embodiments, the STING agonist is a nucleotide-based small molecule. In some embodiments, the STING agonist is a cyclic dinucleotide (CDN) or a modified CDN. In some embodiments, the STING agonist is a CDN comprising at least one purine base, or analog thereof. In some embodiments, the STING agonist is a CDN comprising at least one pyrimidine base, or analog thereof. In some embodiments, the STING agonist is a CDN comprising both purine bases, or analogs thereof. In some embodiments, the STING agonist is a CDN comprising both pyrimidine bases, or analogs thereof. In some embodiments, the STING agonist is a CDN comprising a purine base, or analog thereof, and a pyrimidine base, or analog thereof. In some embodiments, the STING agonist is a CDN comprising at least one adenine, or analog thereof. In some embodiments, the STING agonist is a CDN comprising at least one guanine, or analog thereof. In some embodiments, the STING agonist is a CDN comprising at least one inosine, or analog thereof. In some embodiments, the STING agonist is a CDN comprising at least one uridine, or analog thereof.

In some embodiments, the configuration of both phosphate bridge linkages of a CDN is a 2′-5′ linkage. In some embodiments, the configuration of both phosphate bridge linkages of a CDN is a 3′-5′ linkage. In some embodiments, the configuration of one phosphate bridge linkages of a CDN is a 3′-5′ linkage and the configuration of the other phosphate bridge linkage of the CDN is a 2′-5′ linkage. In some embodiments, a CDN with mixed linkage (a 2′-5′ phosphate bridge linkage and a 3′-5′ phosphate bridge linkage).

In some embodiments, the CDN comprises at least one phosphate bridge linkage wherein a non-bridging oxygen atom of the phosphate bridge linkage is substituted with a sulfur atom, thereby forming a phosphorothioate linkage. In some embodiments, the phosphorothioate linkage is a Rp chiral center. In some embodiments, the phosphorothioate linkage is a Sp chiral center. In some embodiments, the CDN comprises two phosphorothioate linkages. In some embodiments, the CDN comprises two phosphorothioate linkages and is a [Rp, Rp] diastereomer. In some embodiments, the CDN comprises two phosphorothioate linkages and is a [Rp, Sp] diastereomer. Methods for preparing CDNs comprising a phosphorothioate linkage are known in the art (see, e.g., Gaffney, et al. (2010) ORG LETT 12:3269-3271).

In some embodiments, the CDN comprises: (i) a first nucleotide base selected from: an adenine or analog thereof, a guanosine or analog thereof, a inosine or analog thereof, or a uridine or analog thereof; (ii) a second nucleotide base selected from: an adenine or analog thereof, a guanosine or analog thereof, a inosine or analog thereof, or a uridine or analog thereof; (iii) a first phosphate bridge linkage selected from: a 2′-5′ phosphate bridge linkage or a 3′-5′ phosphate bridge linkage; and (iv) a second phosphate bridge linkage selected from: a 2′-5′ phosphate bridge linkage or a 3′-5′ phosphate bridge linkage.

In some embodiments, the CDN comprises: (i) a first nucleotide base selected from: an adenine or analog thereof, a guanosine or analog thereof, a inosine or analog thereof, or a uridine or analog thereof; (ii) a second nucleotide base selected from: an adenine or analog thereof, a guanosine or analog thereof, a inosine or analog thereof, or a uridine or analog thereof; (iii) a phosphorothioate bridge linkage selected from: a 2′-5′ phosphorothioate bridge linkage or a 3′-5′ phosphorothioate bridge linkage; and (iv) a phosphate bridge linkage selected from: a 2′-5′ phosphate bridge linkage or a 3′-5′ phosphate bridge linkage. In some embodiments, the phosphorothioate bridge linkage is a Rp chiral center or a Sp chiral center.

In some embodiments, the CDN comprises: (i) a first nucleotide base selected from: an adenine or analog thereof, a guanosine or analog thereof, a inosine or analog thereof, or a uridine or analog thereof; (ii) a second nucleotide base selected from: an adenine or analog thereof, a guanosine or analog thereof, a inosine or analog thereof, or a uridine or analog thereof; (iii) a first phosphorothioate bridge linkage selected from: a 2′-5′ phosphorothioate bridge linkage or a 3′-5′ phosphorothioate bridge linkage; and (iv) a second phosphorothioate bridge linkage selected from: a 2′-5′ phosphorothioate bridge linkage or a 3′-5′ phosphorothioate bridge linkage. In some embodiments, the CDN is a [Rp, Rp] or a [Rp, Sp] diastereomer.

In some embodiments, the CDN is selected from:
(i) cyclic [G(3′,5′)pG(3′,5′)p] (3′3′ c-di-GMP), wherein G is guanosine or an analog thereof;
(ii) cyclic [G(2′,5′)pG(3′,5′)p] (2′3′-c-di-GMP), wherein G is guanosine or an analog thereof;
(iii) cyclic [A(3′,5′)pA(3′,5′)p] (3′3′-c-di-AMP), wherein A is adenosine or an analog thereof;
(iv) cyclic [A(2′,5′)pA(3′,5′)p] (2′3′-c-di-AMP), wherein A is adenosine or an analog thereof;
(v) cyclic [I(3′,5′)pI(3′,5′)p] (3′3′-c-di-IMP), wherein I is inosine or an analog thereof;
(vi) cyclic [I(2′5′)pI(3′,5′)p] (2′3′-c-di-IMP). wherein I is inosine or an analog thereof;
(vii) cyclic [U(3′,5′)pU(3′,5′)p] (3′3′-c-di-UMP), wherein U is uridine or an analog thereof;
(viii) cyclic [U(2′,5′)pU(3′,5′)p] (2′3′-c-di-UMP), wherein U is uridine or an analog thereof;
(ix) cyclic [G(2′,5′)pA(3′,5′)p] (2′3′-cGAMP), wherein G is guanosine or an analog thereof, and wherein A is adenosine or an analog thereof;
(x) cyclic [G(3′,5′)pA(3′,5′)p] (3′3′-cGAMP), wherein G is guanosine or an analog thereof, and wherein A is adenosine or an analog thereof;
(xi) cyclic [A(3′,5′)pI(3′,5′)p] (3′3′-cAIMP), wherein A is adenosine or an analog thereof, and wherein I is inosine or an analog thereof;
(xii) cyclic [A(2′,5′)pI(3′,5′)p] (2′3′-cAIMP), wherein A is adenosine or an analog thereof, and wherein I is inosine or an analog thereof;
(xiii) an analog of any of (i)-(xii), wherein the analog comprises a single phosphorothioate linkage, wherein the phosphorothioate linkage is a Rp chiral center or a Sp chiral center; or
(xiv) an analog of any of (i)-(xii), wherein the analog two phosphorothioate linkages, and wherein the analog is a [Rp, Rp] or a [Rp, Sp] diastereomer.

In some embodiments, a suitable STING agonist for use in the present disclosure is described in, e.g., US 9,770,467; US 10,414,789; US 2016/0287623; Corrales, et al. (2015) CELL REPORTS 11:1018-1030; US 9,840,533; Gao, et al., (2013) MOL CELL 154:748-762; US 2012/0178710; US 10,011,630; US 10,449,211; US2018/0230178; US2019/0062365; US2018/0230178; WO2018/100558; and WO2018/200812, each of which is hereby incorporated by reference.

In some embodiments, the STING agonist is covalently-linked to a polymer-modified lipid via a linker. For example, the STING agonist is covalently attached to the linker and the linker is covalently attached to the polymer of the polymer-modified lipid. In some embodiments, the STING agonist is a CDN or analog thereof, and the STING agonist amphiphile conjugate comprises a CDN or analog thereof covalently-linked to a polymer-modified lipid via a linker. For example, the CDN is covalently attached to the linker and the linker is covalently attached to the polymer of the polymer-modified lipid. In some embodiments, the CDN or analog thereof comprises at least one phosphate bridge linkage, wherein a non-bridging oxygen atom is substituted with a sulfur atom, and wherein the CDN or analog thereof is covalently-linked to the linker via the sulfur atom. In some embodiments, the CDN or analog thereof comprises a sugar ring substituent, wherein the CDN or analog thereof is covalently-linked to the linker via the sugar ring substituent. In some embodiments, the CDN or analog thereof comprises a nucleotide base substituent, wherein the CDN or analog thereof is covalently-linked to the linker via the nucleotide base substituent.

In some embodiments, the STING agonist is a compound disclosed by WO2018/200812, which is hereby incorporated by reference. The STING agonist compounds disclosed by WO2018/200812 are represented by formula (A)-(F):

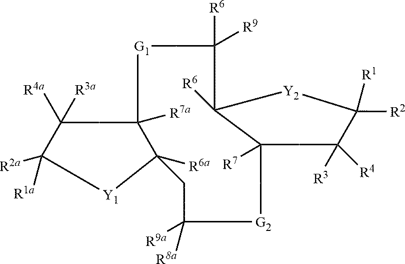

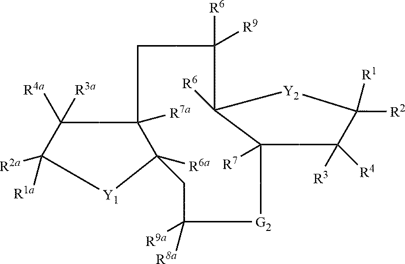

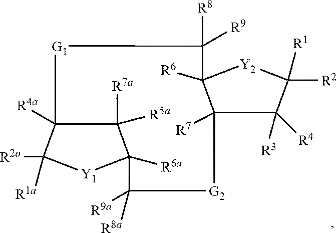

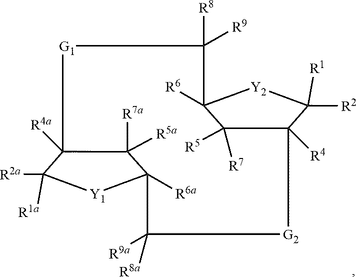

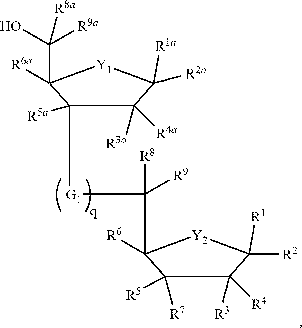

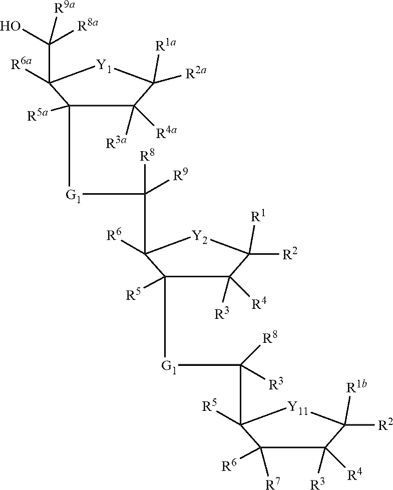

wherein each symbol is defined as in WO2018/200812. In some embodiments, the STING agonist is a compound according to formula (A)-(F) as described by WO2018/200812, wherein the compound is covalently-linked to a polymer-modified lipid via a linker. For example, the compound is covalently-linked to the linker, and the linker is covalently-linked to the polymer of the polymer-modified lipid. Methods for covalent attachment of a compound according to formula (A)-(F) via a linker are known, such as those described in WO2018/200812.

In some embodiments, the STING agonist is a compound disclosed by WO2018/100558, which is hereby incorporated by reference. The STING agonist compounds disclosed by WO2018/100558 are represented by formula (I):

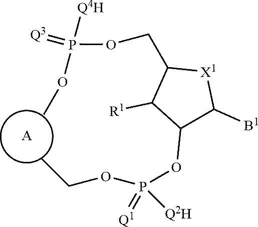

wherein the partial structure represented by formula the partial structure represented by formula (A-1):

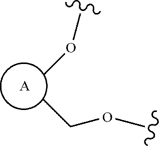

is a partial structure represented by formula (IIA):

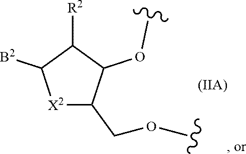

a partial structure represented by formula (IIB):

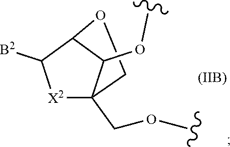

wherein R¹, R², B¹, B², X¹, X², Q¹, Q², Q³, and Q⁴ of formula (I), formula (IIA), and formula (IIB) are each symbols as defined in WO2018/100558. In some embodiments, the STING agonist is a compound according to formula (I) as described by WO2018/100558, wherein the compound according to formula (I) is covalently-linked to a polymer-modified lipid via a linker. For example, the compound according to formula (I) is covalently-linked to the linker and the linker is covalently-linked to the polymer of the polymer-modified lipid. Methods for covalent attachment of a compound according to formula (I) via a linker are known, such as those described in WO2018/100558. Furthermore, methods of covalent attachment of a compound according to formula (I) as described in WO2018/100558 include (i) base attachment, (ii) sugar ring attachment, or (iii) phosphorothioate attachment, as described below.

### Covalent Attachment of STING Agonist

### 1. Base Attachment

In some embodiments, the STING agonist is:
(i) a compound according to formula (I) of WO2018/100558, wherein B¹ is a group represented by

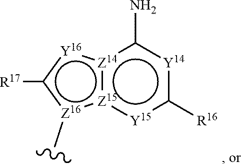

(ii) a compound according to formula (I) of WO2018/100558 wherein B² is a group represented by

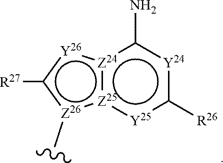

In some embodiments, the compound according to formula (I) of WO2018/100558 is converted to a STING agonist amphiphile conjugate as follows:
(i) B¹ of formula (I) is converted to a group represented by

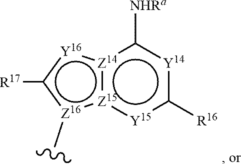

(ii) B² of formula (I) is converted to a group represented by

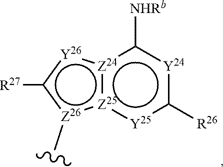

wherein R^{a} and R^{b} are each independently:
   (a) a C₁₋₆ alkyl group,
   (b) an acyl group, or
   (c) a group represented by the formula:

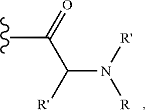

wherein R is a linker providing covalent attachment to the polymer-modified lipid (a linker providing covalent attachment to an available carbon atom, oxygen atom, nitrogen atom, or sulfur atom of the polymer portion of the polymer-modified lipid), and each R′ is a hydrogen atom or a substituent.

### 2. Sugar Ring Attachment

In some embodiments, the compound according to formula (I) of WO2018/100558 is converted to a STING agonist amphiphile conjugate as follows:
(i) R1 and R2 in formula (I) are each independently converted to a group represented by the formula —OR3 wherein R3 is:
   (i.i) a C₁₋₆ alkyl group,
   (i.ii) an acyl group (preferably a C₁₋₆ alkoxy-carbonyl group),
   (i.iii) a group represented by the formula: 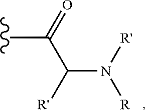
   (i.iv) a group represented by the formula: 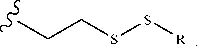
   (i.v) a group represented by the formula: 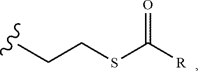
   (i.vi) a group represented by the formula: 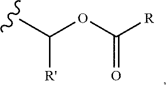
   (i.vii) a group represented by the formula: 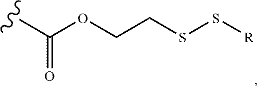
   (i.viii) a group represented by the formula: 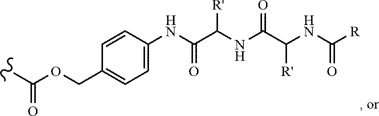
   (i.ix) a group represented by the formula: 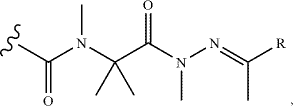
wherein R is a linker providing covalent attachment to the polymer-modified lipid (a linker providing covalent attachment to an available carbon atom, oxygen atom, nitrogen atom, or sulfur atom of the polymer portion of the polymer-modified lipid), and each R′ is a hydrogen atom or a substituent.

### 3. Phosphorothioate Attachment

In some embodiments, the compound according to formula (I) of WO2018/100558 is converted to a STING agonist amphiphile conjugate as follows:
(i) Q²H and Q⁴H in the formula (I) are each independently converted to
   (i.i) a group represented by the formula: -SR⁴, wherein R⁴ is:
      (i.i.i) a C₁₋₆ alkyl group,
      (i.i.ii) an acyl group,
      (i.i.iii) a group represented by the formula: -SR,
      (i.i.iv) a group represented by the formula: 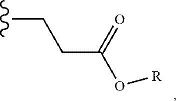
      (i.i.v) a group represented by the formula: 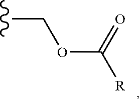
      (i.i.vi) a group represented by the formula: 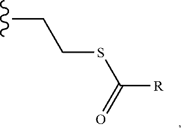
      (i.i.vii) a group represented by the formula: 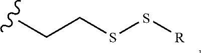
      (i.i.viii) a group represented by the formula: 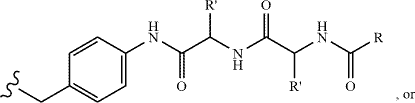
      (i.i.ix) a group represented by the formula: 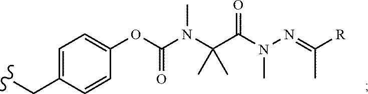
   (i.ii) a group represented by the formula: -OR⁵, wherein R⁵ is:
      (i.ii.i) a C₁₋₆ alkyl group,
      (i.ii.ii) an acyl group,
      (i.ii.iii) a group represented by the formula: 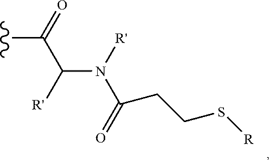
      (i.ii.iv) a group represented by the formula: 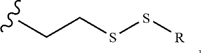
      (i.ii.v) a group represented by the formula; 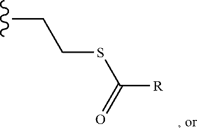
      (i.ii.vi) a group represented by the formula: 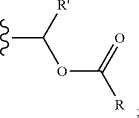
   (i.iii) a group represented by the formula: -NHR⁶, wherein R⁶ is:
      (i.iii.i) a C₁₋₆ alkyl group,
      (i.iii.ii) an acyl group,
      (i.iii.iii) a group represented by the formula: 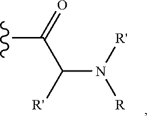
      (i.iii.iv) a group represented by the formula: 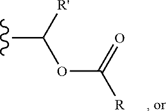
      (i.iii.v) a group represented by the formula: 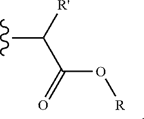
wherein R is a linker providing covalent attachment to the polymer-modified lipid (a linker providing covalent attachment to an available carbon atom, oxygen atom, nitrogen atom, or sulfur atom of the polymer portion of the polymer-modified lipid), and each R′ is a hydrogen atom or a substituent.

In some embodiments, the linker is as described below or as described in Chem. Rev., 114, 9154-9218 (2014), Pharma. Res. 32, 3526-3540 (2015), Bioconjugate Chem. 21, 5-13 (2010), The AAPS journal, 17, 339-351 (2015), WO 2011/005761, which references are hereby incorporated by reference in their entirety.

### (Ii) Linkers of the STING Agonist Amphiphile Conjugate

In some embodiments, a STING agonist amphiphile conjugate of the disclosure comprises a STING agonist (e.g., CDN) covalently-linked to a polymer-modified lipid via a linker. In some embodiments, the linker is any chemical moiety capable of linking a STING agonist to a polymer-modified lipid. In some embodiments, the linker is any chemical moiety capable of linking a CDN to a polymer-modified lipid.

In some embodiments, the linker comprises one or more cleavage elements, wherein the cleavage element is susceptible to cleavage, thereby facilitating release of the STING agonist (e.g., CDN) or cleavage product thereof from the polymer-modified lipid. In some embodiments, the cleavage element is susceptible to acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, disulfide reduction-based cleavage. In some embodiments, the linker is substantially stable in vivo until the STING agonist amphiphile conjugate or lipid composition thereof binds to or enters a cells, whereupon either the one or more cleavage elements of the linker are cleaved due to intracellular enzymes (e.g., proteases, peptidases) and/or intracellular chemical conditions (e.g., low pH, reducing capacity), thereby releasing the STING agonist or cleavage product thereof from the polymer-modified lipid. Methods for measuring efficiency of linker cleavage are known in the art. For example, by measuring rate of formation of cleavage products (e.g., STING agonist or cleavage product thereof) by mass spectrometry, chromatography, or functional assay (e.g., STING activity assay) following exposure to conditions sufficient for cleavage.

In some embodiments, the linker is an esterase-cleavable linker. As used herein, an “esterase cleavable linker” refers to a cleavable linker that is susceptible to cleavage by one or more esterases. Only certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

In some embodiments, the linker is an acid-labile linker. As used herein, an “acid-labile linker” refers to linker comprising a cleavage element susceptible to cleavage at acidic pH. For example, certain intracellular compartments, such as endosomes and lysosomes, have an acidic pH (pH 4-5), and provide conditions suitable for cleavage of acid-labile linkers. In some embodiments, the cleavage element of the acid-labile linker is a hydrazine or silyl ether.

In some embodiments, the linker comprises a cleavage element that is susceptible to cleavage by peptidases. As used herein, a “peptidase cleavable linker” refers to a cleavable linker that is susceptible to cleavage by one or more peptidases. In some embodiments, the cleavage element is a peptide susceptible to peptidase-cleavage. Only certain peptides are readily cleaved by peptidases when present inside or outside of cells. For example, peptides such as those described by Trout et al. (1982) PROC. NATL. ACAD. SCI. USA 79:626-629 and Umemoto, et al. (1989) INT J CANCER 43:677-684. In some embodiments, the peptide is composed of alpha-amino acid units and peptidic bonds, which chemically are amide bonds between carboxylate of one amino acid and the amino group of a second amino acid. It is understood that other amide bonds, such as the bond between a carboxylate and the alpha-amino acid group of lysine, are understood not to be peptidic bonds and are considered non-cleavable.

In some embodiments, the linker comprises a cleavage element that is a peptide comprising at least 1, 2, 3, 4, 5, 6, 7, 8, 9, or 10 amino acid residues. In some embodiments, the peptide is susceptible to cleavage by proteolysis, wherein cleavage of the peptide is sufficient to release the STING agonist (e.g., CDN) from the polymer-modified lipid upon exposure to intracellular proteases, peptidases, proteinases, or enzymes that catalyze proteolysis, such as lysosomal enzymes (see, e.g., Doronina et al. (2003) NAT. BIOTECHNOL. 21:778-784). Non-limiting examples of peptides susceptible to cleavage by proteolysis include dipeptides, tripeptides, tetrapeptides, and pentapeptides. Non-limiting exemplary dipeptides include alanine (ala-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Non-limiting exemplary tripeptides include glycine-valine-citrulline (gly-val-cit) and glycine-glycine-glycine (gly-gly-gly).

In some embodiments, the linker comprises a cleavage element that is a peptide susceptible to cleavage by proteolysis, wherein the peptide comprises naturally-occurring and/or non-natural amino acid residues. In some embodiments, a “naturally-occurring amino acid” includes Ala, Asp, Cys, Glu, Phe, Gly, His, He, Lys, Leu, Met, Asn, Pro, Gin, Arg, Ser, Thr, Val, Trp, and Tyr. In some embodiments, “non-natural amino acids” (i.e., amino acids that do not occur naturally) includes, by way of non-limiting example, homoserine, homoarginine, citrulline, phenylglycine, taurine, iodotyrosine, seleno- cysteine, norleucine (“Nle”), norvaline (“Nva”), beta-alanine, L- or D-naphthalanine, ornithine (“Orn”), and the like. In some embodiments, the peptide is designed and optimized for enzymatic cleavage by a particular enzyme, for example, a tumor-associated protease, cathepsin B, C and D, or a plasmin protease using established methods of peptide design. In some embodiments, the amino acid residue is in the D-form of the natural or non-natural amino acid. “D-” designates an amino acid having the “D” (dextrorotary) configuration, as opposed to the configuration in the naturally occurring (“L-”) amino acids. Natural and non-natural amino acids are available for purchase commercially (Sigma Chemical Co., Advanced Chemtech) or synthesized using methods known in the art.

In some embodiments, the linker is a non-cleavable linker (e.g., a linker substantially resistant to cleavage). In some embodiments, the non-cleavable linker is any chemical moiety capable of linking a STING agonist (e.g., CDN) to a polymer-modified lipid in a stable, covalent manner that is not susceptible to cleavage by any of the foregoing mechanisms of cleavage. In some embodiments, the non-cleavable linker is any chemical moiety capable of linking a STING agonist (e.g., CDN) to a polymer-modified lipid in a manner that is substantially resistant to acid-induced cleavage, photo-induced cleavage, peptidase-induced cleavage, esterase-induced cleavage, and disulfide reduction-based cleavage. In some embodiments, the non-cleavable linker is any chemical moiety that (i) is capable of linking a STING agonist (e.g., CDN) to a polymer-modified lipid, and (ii) is substantially resistant to cleavage induced by an acid, a peptidase, an esterase, a reducing agent, or ultraviolet and/or visible wavelengths of light, at conditions under which the STING agonist and/or polymer-modified lipid are stable and do not lose activity.

In some embodiments, the linker is represented by the formula -X³-T-Z-Q-, wherein X³ is a divalent radical that connects the STING agonist (e.g., CDN) to the rest of the linker, or is absent, wherein T is a peptide, or is absent, wherein Z is a spacer, and wherein Q is a heterobifunctional group or a heterotrifunctional group.

As used herein, the term “heterobifunctional group” or the term “heterotrifunctional group” refers to a chemical moiety that connects a linker and another therapeutically active molecule, e.g., polymer-modified lipid.

In some embodiments, the heterobifunctional group or heterotrifunctional group is a chemical moiety that connects the linker to the polymer of the polymer-modified linker. Heterobi- and trifunctional groups are characterized as having different reactive groups at either end of the chemical moiety. Non-limiting exemplary heterobifunctional groups include:

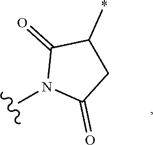

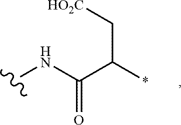

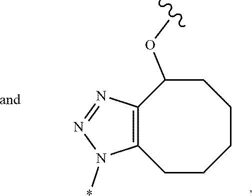

wherein the “*” indicates the attachment point to a carbon atom, nitrogen atom, oxygen atom, or sulfur atom of the polymer. In some embodiments, the heterobifunctional group is

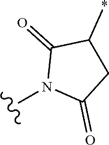

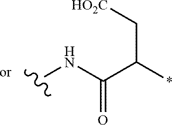

and is attached to a sulfur atom of the polymer.

A non-limiting exemplary heterotrifunctional group is:

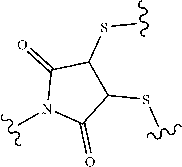

In some embodiments, Z is a chemical moiety that connects the heterobifunctional group or a heterotrifunctional group (Q) to the rest of the linker, e.g., a peptide (T), or, if a heterobifunctional or heterotrifunctional group is absent, connects the rest of the linker or the STING agonist to a carbon atom, nitrogen atom, oxygen atom, or sulfur atom of the polymer. Non-limiting exemplary spacers include —NH—, —S—, —O—, —NHC(═O)CH₂CH₂—, —S(═O)₂—CH₂CH₂—, — C(═O)NHNH_{—}, —C(═O)O—, —C(═O)NH—, —CH₂—, —CH₂CH₂—, —CH₂CH₂CH₂—, —CH₂═CH₂—, —C≡C—, —CH═N—O—, polyethylene glycol (PEG),

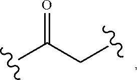

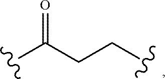

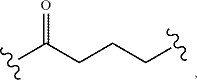

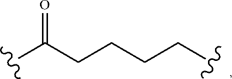

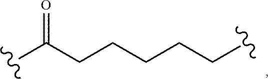

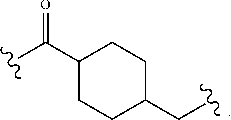

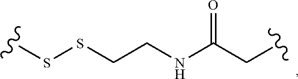

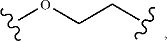

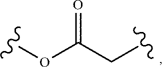

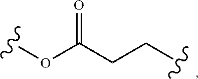

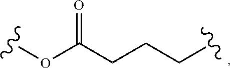

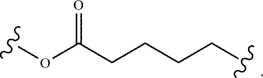

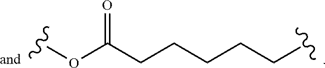

### (Iii) Polymer-Modified Lipids of the STING Agonist Amphiphile Conjugate

In some embodiments, a STING agonist amphiphile conjugate comprises a STING agonist (e.g., CDN) covalently-linked to a polymer-modified lipid. In some embodiments, the STING agonist amphiphile conjugate comprises a STING agonist (e.g., CDN) attached to the polymer of the polymer-modified lipid via a linker. In some embodiments, the polymer-modified lipid comprises a polymer covalently-linked to a lipid.

A suitable polymer for use in the polymer-modified lipid is any hydrophilic polymer. In some embodiments, the polymer comprises a string of hydrophilic amino acids (e.g., serine, threonine, cysteine, tyrosine, asparagine, glutamine, aspartic acid, glutamic acid, lysine, arginine, histidine, or combinations thereof) or a polysaccharide, such as dextran (MW: 1,000 Da to 2,000,000 Da). In some aspects, the polymer comprises polyethylene glycol (PEG) units, polypropylene oxide (PPO) units, polylactide (PLA) units, poly(lactide-co-glycolide) (PLGA) units, poly(lactide-co-caprolactone) (PLCL) units, poly(methacrylate) (PMA) units, polyethylene (PE), polystyrene (PS), polyacrylic acid (PAA), polyacrylamide (PAM), poly(carboxybetaine) (PCB), or a combination thereof.

In some embodiments, a polymer-modified lipid of the disclosure comprises a polymer covalently-linked to a lipid, wherein the lipid is a hydrophobic lipid. In some embodiments, the lipid is a diacyl lipid or two-tailed lipid. In some embodiments, the tails of the diacyl lipid each independently comprise about 12 to about 30 hydrocarbon units, about 14 to about 25 hydrocarbon units, about 16 to about 20 hydrocarbon units, or about 12, about 13, about 14, about 15, about 16, about 17, about 18, about 19, about 20, about 21, about 22, about 23, about 24, about 25, about 26, about 27, about 28, about 29 or about 30 hydrocarbon units. In some embodiments, the diacyl lipid tails are each independently saturated or unsaturated.

In some embodiments, the polymer-modified lipid is a PEG-lipid. PEG-lipids suitable for use in the STING agonist amphiphile conjugate are further detailed below.

In some embodiments, the polymer of the PEG-lipid is represented by the formula —Z²—(CH₂CH₂O)ₙ—Z³—, wherein Z² is a spacer for covalent attachment of the linker to the PEG units of the polymer, n is 25-230, and Z³ is a spacer for covalent attachment of the PEG units of the polymer to the head group of the lipid. A suitable spacer Z² for use in the present disclosure is any chemical moiety that connects the linker (e.g., the heterobifunctional or heterotrifunctional group of the linker) to the PEG units of the polymer. A suitable spacer Z³ for use in the present disclosure is any chemical moiety that connects the PEG units of the polymer to the lipid (e.g., to the head group of the lipid). Non-limiting exemplary spacers include —NH—, —S—, —O—, —NHC(═O)CH₂CH₂—, —S(═O)₂—CH₂CH₂—, —C(═O)NHNH_{—}, —C(═O)O—, —C(═O)NH—, —CH₂—, —CH₂CH₂—, —CH₂CH₂CH₂—, —CH₂═CH₂—, —C≡C—, —CH═N—O—, ethylene glycol, polyethylene glycol (PEG),

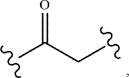

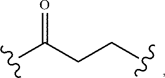

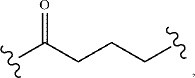

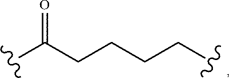

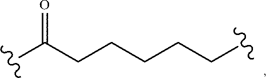

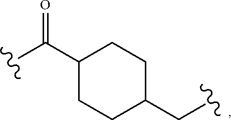

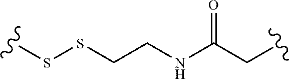

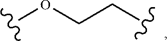

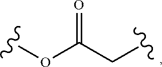

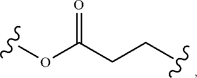

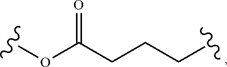

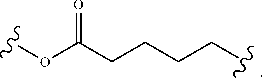

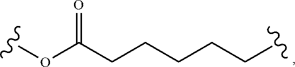

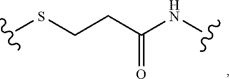

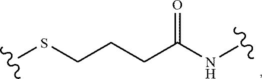

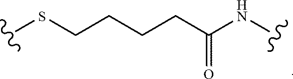

In some embodiments, the lipid of the PEG-lipid is represented by Formula (LI-I):

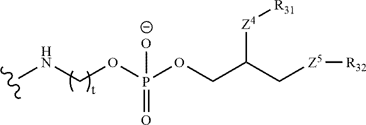

wherein t is 1, 2, 3, 4, 5, 6, 7, 8, 9, or 10; Z⁴ and Z⁵ are spacer groups for attachment to the carbon backbone; and R₃₁ and R₃₂ are aliphatic chains (e.g., optionally substituted alkyl, alkenyl, or alkynyl groups, e.g., optionally substituted C₁₋₃₀ alkyl, alkenyl, or alkynyl groups).

A suitable spacer Z⁴ or Z⁵ for use in the present disclosure are any chemical moieties that connect the aliphatic chains to the carbon backbone. Non-limiting exemplary spacers include: —O—, -N(R^{N})-, —S—, -C(O)—, -C(O)N(R^{N})-, -NR^{N}C(O)-, -C(O)O—, —OC(O)—, —OC(O)O—, -OC(O)N(R^{N})-, -NR^{N}C(O)O-, or -NR^{N}C(O)N(R^{N})-, wherein R^{N} is selected from hydrogen, C₁₋₆ alkyl, or a nitrogen protecting group.

A suitable aliphatic chain R₃₁ and R₃₂ for use in the present disclosure are optionally substituted alkyl, alkenyl, or alkynyl groups comprising 12-30 carbon atoms, 14-25 carbon atoms, 16-20 carbon atoms, or 12, 13, 14, 15, 16, 17, 18, 19, 20, 21, 22, 23, 24, 25, 26, 27, 28, 29 or 30 carbon atoms.

### (Iv) Exemplary STING Agonist Amphiphile Conjugates

In some aspects, the disclosure provides the following STING agonist amphiphile conjugate embodiments:

### Embodiment 1

A STING agonist amphiphile conjugate having the formula (XIV):
CD-L-P-LI (XIV),
wherein CD is a group represented by any one of Formula (XX)-(XXIX):

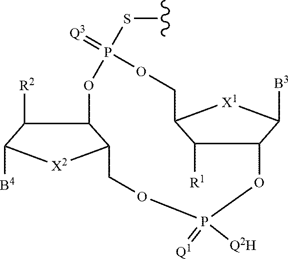

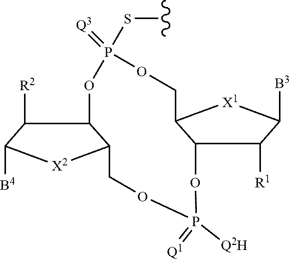

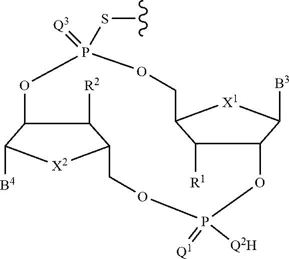

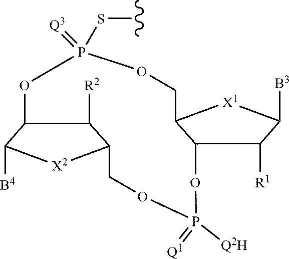

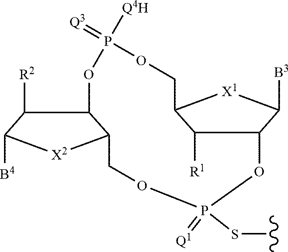

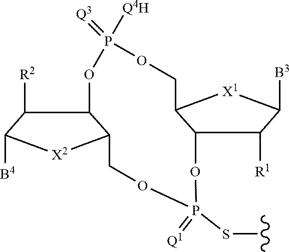

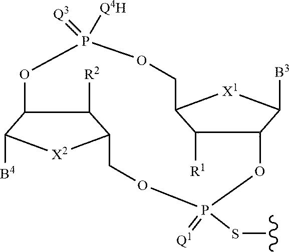

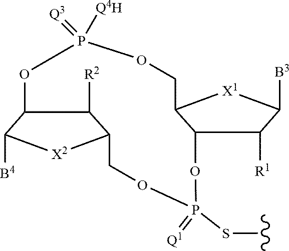

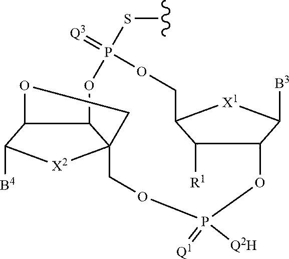

or

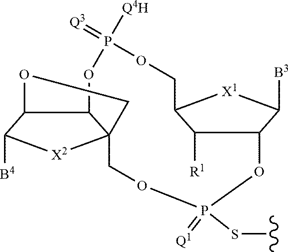

wherein R¹ and R² are each independently a hydroxy group, hydrogen, amino group, or a halogen atom;
wherein B³ and B⁴ are independently an optionally substituted 5- to 14-membered aromatic heterocyclic group;
wherein X¹ and X² are each independently an oxygen atom, CH₂, or a sulfur atom;
wherein Q¹, Q², Q³ and Q⁴ are each independently an oxygen atom or a sulfur atom;
wherein L is a linker;
wherein P is a polymer; and
wherein LI is a diacyl lipid.

### Embodiment 2

The STING agonist amphiphile conjugate of Embodiment 1, wherein R¹ and R² are each independently a hydroxy group or a halogen atom.

### Embodiment 3

The STING agonist amphiphile conjugate of Embodiment 1 or 2, wherein X¹ and X² are each independently an oxygen atom or a sulfur atom.

### Embodiment 4

The STING agonist amphiphile conjugate of any one of Embodiments 1-3, wherein Q¹ and Q³ are an oxygen atom.

### Embodiment 5

The STING agonist amphiphile conjugate of Embodiment 1, wherein CD is a group represented by Formula (XX-A):

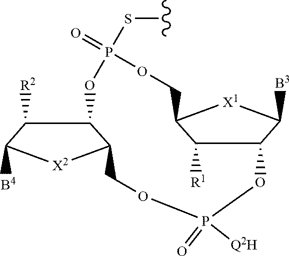

### Embodiment 6

The STING agonist amphiphile conjugate of Embodiment 1, wherein CD is a group represented by Formula (XXI-A):

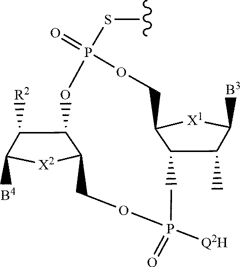

### Embodiment 7

The STING agonist amphiphile conjugate of Embodiment 1, wherein CD is a group represented by Formula (XXII-A):

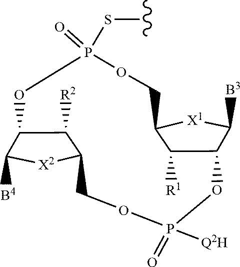

### Embodiment 8

The STING agonist amphiphile conjugate of Embodiment 1, wherein CD is a group represented by Formula (XXIII-A):

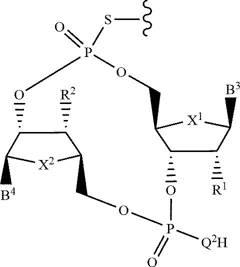

### Embodiment 9

The STING agonist amphiphile conjugate of Embodiment 1, wherein CD is a group represented by Formula (XXIV-A):

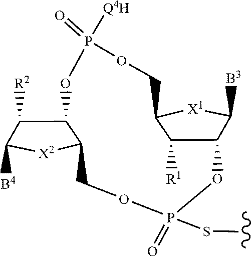

### Embodiment 10

The STING agonist amphiphile conjugate of Embodiment 1, wherein CD is a group represented by Formula (XXV-A):

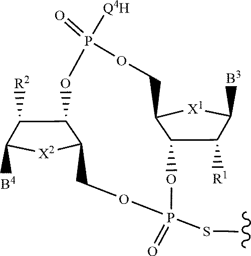

### Embodiment 11

The STING agonist amphiphile conjugate of Embodiment 1, wherein CD is a group represented by Formula (XXVI-A):

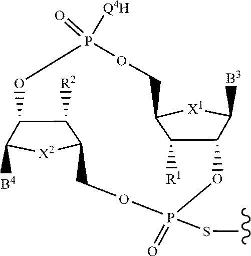

### Embodiment 12

The STING agonist amphiphile conjugate of Embodiment 1, wherein CD is a group represented by Formula (XXVII-A):

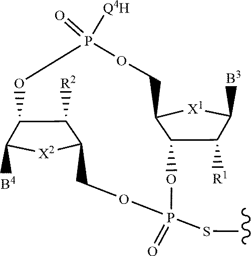

### Embodiment 13

The STING agonist amphiphile conjugate of Embodiment 1, wherein CD is a group represented by Formula (XXVIII-A):

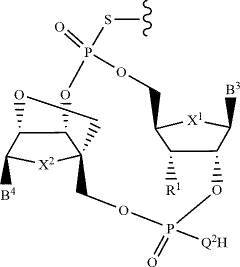

### Embodiment 14

The STING agonist amphiphile conjugate of Embodiment 1, wherein CD is a group represented by Formula (XXIX-A):

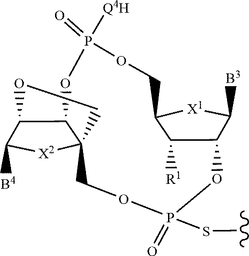

### Embodiment 15

The STING agonist amphiphile conjugate of any one of Embodiments 1-14, wherein:
L is -X³-T-Z-Q-;
X³ is —(CH₂)ₒ—,

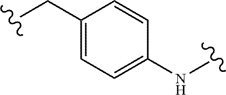

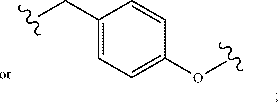

o is 1, 2, or 3; or
X³ is absent;
T is a peptide, or is absent;
Z is a spacer;
Q is a heterobifunctional group or heterotrifunctional group, or
Q is absent.

### Embodiment 16

The STING agonist amphiphile conjugate of Embodiment 15, having the formula (XXX):

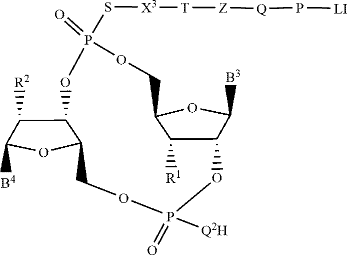

### Embodiment 17

The STING agonist amphiphile conjugate of Embodiment 15, having the formula (XXXI):

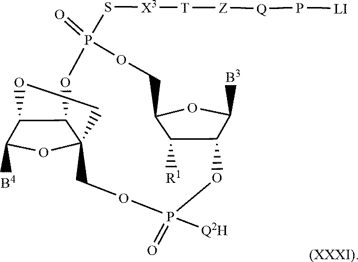

### Embodiment 18

The STING agonist amphiphile conjugate of any one of Embodiments 15-17, wherein:
X³ is

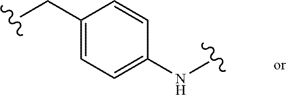

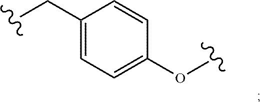

T is

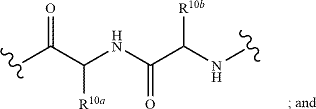

R^{10a} and R^{10b} are independently selected from the group consisting of hydrogen and optionally substituted C₁₋₆ alkyl.

### Embodiment 19

The STING agonist amphiphile conjugate of Embodiment 18, wherein: X³ is

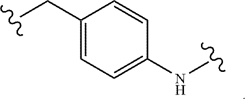

### Embodiment 20

The STING agonist amphiphile conjugate of any one of Embodiments 15-19, wherein:
Z is

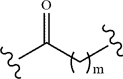

or —(CH₂CH₂O)ⱼ—;
m is 1, 2, 3, 4, 5, or 6; and
j is 1, 2, 3, 4, 5, 6, 7, 8, 9, or 10.

### Embodiment 21

The STING agonist amphiphile conjugate of any one of Embodiments 15-17, wherein:
X³ is —CH₂—;
Z is

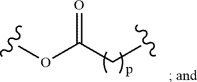

p is 1, 2, 3, 4, 5, 6, 7, 8, 9, or 10.

### Embodiment 22

The STING agonist amphiphile conjugate of any one of Embodiments 15-17, wherein:
X³ is —CH₂CH₂—;
Z is

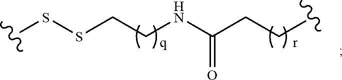

q is 1, 2, 3, 4, 5, or 6; and
r is 1, 2, 3, 4, 5, or 6.

### Embodiment 23

The STING agonist amphiphile conjugate of any one of Embodiments 15-22, wherein Q is a heterobifunctional group selected from the group consisting of:

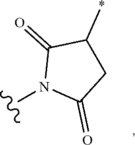

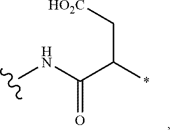

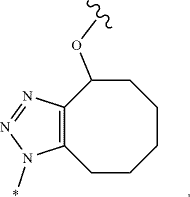

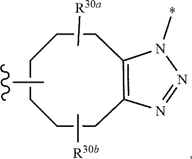

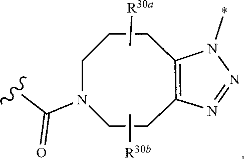

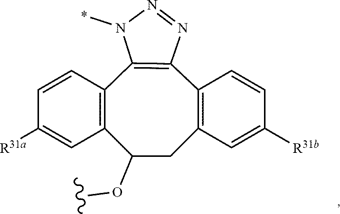

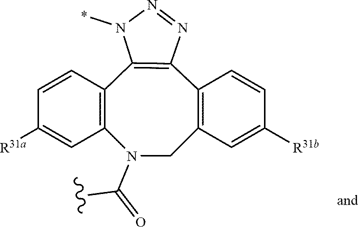

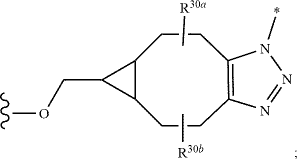

R²⁹ is hydrogen or C₁₋₆ alkyl;
R^{30a} and R^{30b} are independently selected from the group consisting of hydrogen, C₁₋₆ alkyl, halo, —C(═O)OR²⁹, —NH₂, C₁₋₆ alkoxy, —CN, —NO₂, and —OH;
R^{31a} and R^{31b} are independently selected from the group consisting of hydrogen, C₁₋₆ alkyl, halo, —C(═O)OR²⁹, —NH₂, —N(CH₃)₂, C₁₋₆ alkoxy, —CN, —NO₂, and —OH; and
* indicates the attachment point to a carbon atom, nitrogen atom, oxygen atom, or sulfur atom of the polymer (P).

### Embodiment 24

The STING agonist amphiphile conjugate of any one of Embodiments 1-23, wherein P is a group represented by the formula -Z²-(CH₂CH₂O)ₙ-Z³-;
wherein Z² and Z³ are spacers, and
n is 25-230.

### Embodiment 25

The STING agonist amphiphile conjugate of Embodiment 24, wherein
Z² is

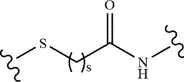

or —(CH₂CH₂O)—;
Z³ is —C(O)— or C₁₋₆ alkyl;
s is 1, 2, 3, 4, 5, or 6.

### Embodiment 26

The STING agonist amphiphile conjugate of any one of Embodiments 1-25, wherein LI is a compound with Formula (LI-I):

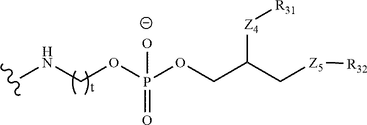

wherein t is 1, 2, 3, 4, 5, 6, 7, 8, 9, or 10;
Z⁴ and Z⁵ are each independently selected from the group consisting of —O—, -N(R^{N})-, —S—, —C(O)—, -C(O)N(R^{N})-, -NR^{N}C(O)-, —C(O)O—, —OC(O)—, —OC(O)O—, -OC(O)N(R^{N})-, -NR^{N}C(O)O-, or -NR^{N}C(O)N(R^{N})-;
R₃₁ and R₃₂ are each independently an optionally substituted alkyl, alkenyl, or alkynyl group comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units;
R^{N} is selected from hydrogen, C₁₋₆ alkyl, or a nitrogen protecting group.

### Embodiment 27

The STING agonist amphiphile conjugate of Embodiment 26, having formula (XXXII):

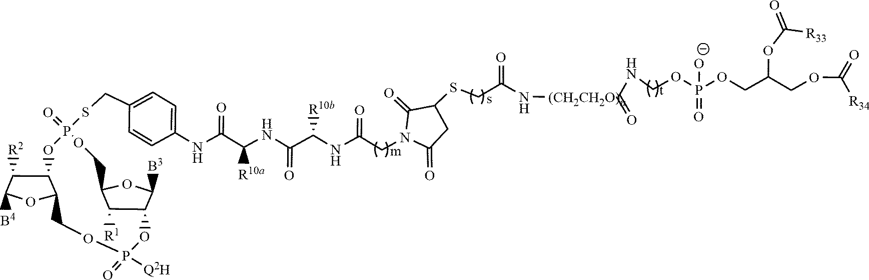

wherein R^{10a} and R^{10b} are independently C₁₋₃ alkyl;
n is 25-230;
m is 2, 3, 4, or 5, optionally m is 5;
s is 1, 2, 3, 4, 5, or 6;
t is 1, 2, 3, 4, 5, or 6; and
R₃₃ and R₃₄ are each independently an optionally substituted alkyl, alkenyl, or alkynyl group comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units.

### Embodiment 28

The STING agonist amphiphile conjugate of Embodiment 26, having formula (XXXII-A):

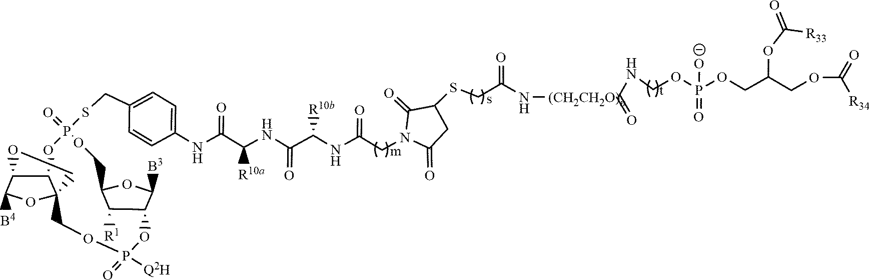

wherein R^{10a} and R^{10b} are independently C₁₋₃ alkyl;
n is 25-230;
m is 2, 3, 4, or 5, optionally m is 5;
s is 1, 2, 3, 4, 5, or 6;
t is 1, 2, 3, 4, 5, or 6; and
R₃₃ and R₃₄ are each independently an optionally substituted alkyl, alkenyl, or alkynyl group comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units.

### Embodiment 29

The STING agonist amphiphile conjugate of Embodiment 26, having formula (XXXIII):

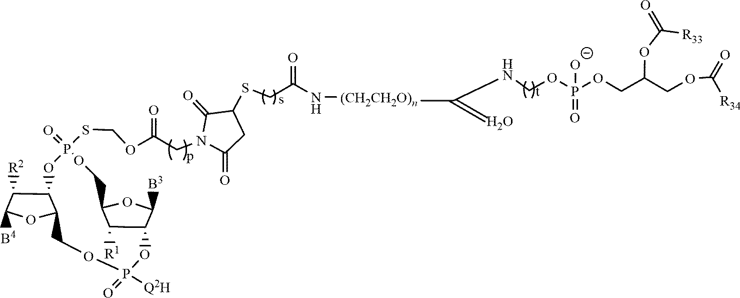

wherein n is 25-230;
p is 4, 5, or 6;
s is 1, 2, 3, 4, 5, or 6;
t is 1, 2, 3, 4, 5, or 6; and
R₃₃ and R₃₄ are each independently an optionally substituted alkyl, alkenyl, or alkynyl group comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units.

### Embodiment 30

The STING agonist amphiphile conjugate of Embodiment 26, having formula (XXXIV):

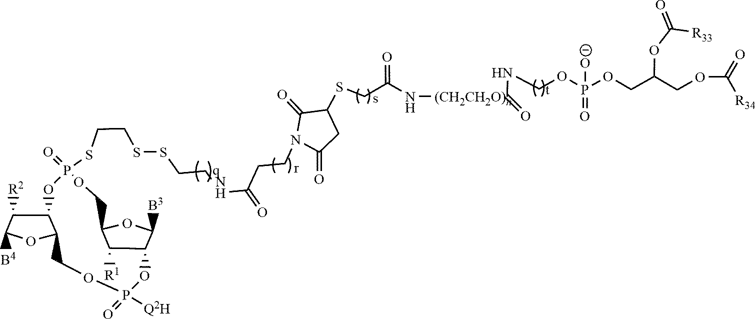

wherein n is 25-230;
q is 1, 2, or 3;
r is 1, 2, or 3;
s is 1, 2, 3, 4, 5, or 6;
t is 1, 2, 3, 4, 5, or 6; and
R₃₃ and R₃₄ are each independently a saturated or unsaturated lipid chain comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units.

### Embodiment 31

The STING agonist amphiphile conjugate of any one of Embodiments 1-30, wherein B³ and B⁴ are independently an optionally substituted 8- to 14-membered fused bicyclic aromatic heterocyclic.

### Embodiment 32

The STING agonist amphiphile conjugate of Embodiment 31, wherein:
B³ is a group represented by formula (B³-A) or formula (B³-B):

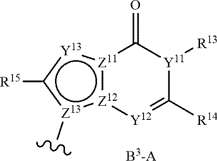

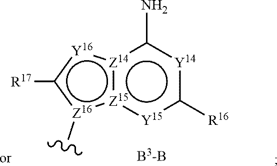

R¹³, R¹⁴, R¹⁵, R¹⁶ and R¹⁷ are each independently a hydrogen atom or a substituent;
Y¹¹, Y¹², Y¹³, Y¹⁴, Y¹⁵ and Y¹⁶ are each independently N or CR^{1a};
Z¹¹, Z¹², Z¹³, Z¹⁴, Z¹⁵ and Z¹⁶ are each independently N or C;
R^{1a} is a hydrogen atom or a substituent;
B⁴ is a group represented by formula (B⁴-A) or formula (B⁴-B):

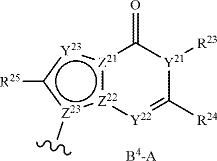

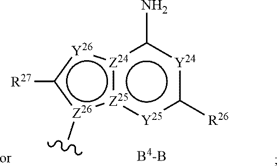

R²³, R²⁴, R²⁵, R²⁶ and R²⁷ are each independently a hydrogen atom or a substituent;
Y²¹, Y²², Y²³, Y²⁴, Y²⁵ and Y²⁶ are each independently N or CR^{2a};
Z²¹, Z²², Z²³, Z²⁴, Z²⁵ and Z²⁶ are each independently N or C;
R^{2a} is a hydrogen atom or a substituent.

### Embodiment 33

The STING agonist amphiphile conjugate of any one of Embodiments 1-32, wherein at least one of B³ or B⁴ is:

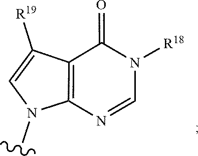

R¹⁸ is hydrogen or C₁₋₆ alkyl; and
R¹⁹ is a halogen atom.

### Embodiment 34

The STING agonist amphiphile conjugate of Embodiment 33, wherein B³ is:

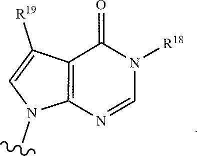

### Embodiment 35

The STING agonist amphiphile conjugate of Embodiment 33, wherein B⁴ is:

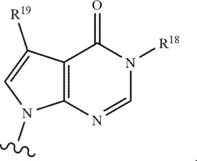

### Embodiment 36

The STING agonist amphiphile conjugate of any one of Embodiments 33-35, wherein R¹⁹ is a fluoro atom.

### Embodiment 37

The STING agonist amphiphile conjugate of any one of Embodiments 33-36, wherein R¹⁸ is hydrogen.

### Embodiment 38

The STING agonist amphiphile conjugate of any one of Embodiments 33-36, wherein R¹⁸ is methyl.

### Embodiment 39

The STING agonist amphiphile conjugate of any one of Embodiments 34 or 36-38, wherein B⁴ is selected from the group consisting of:

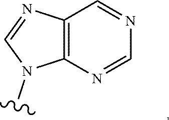

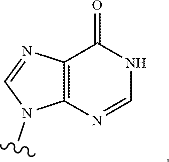

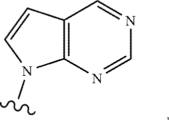

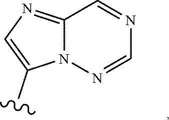

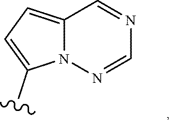

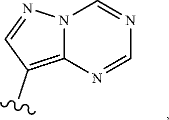

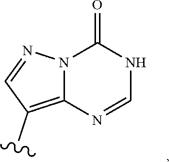

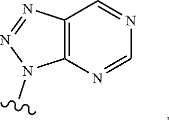

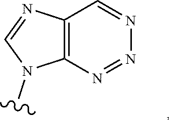

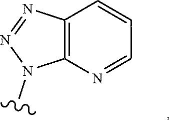

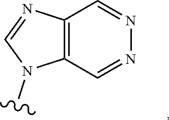

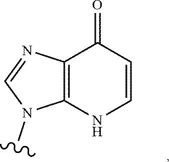

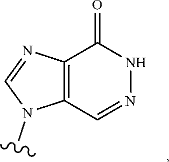

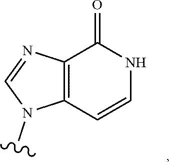

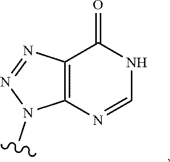

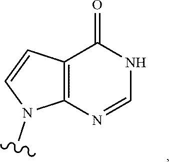

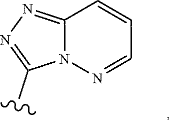

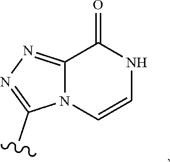

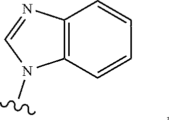

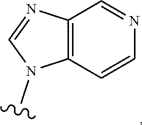

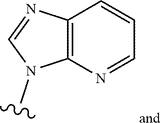

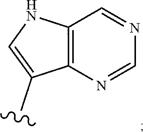

each of which is optionally and independently substituted at:
(i) any available carbon atom with a halo, C₁₋₆ alkyl, C₁₋₆ alkoxy, C₁₋₆ alkylthio, or amino group; and/or
(ii) any available nitrogen atom with a C₁₋₆ alkyl group.

### Embodiment 40

The STING agonist amphiphile conjugate of Embodiment 39, wherein B⁴ is selected from the group consisting of:

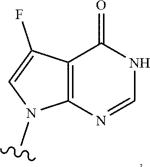

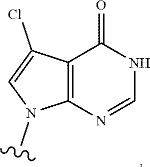

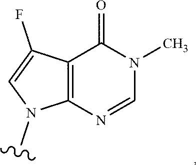

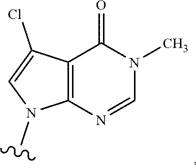

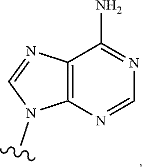

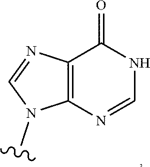

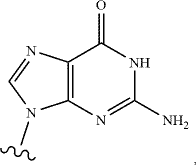

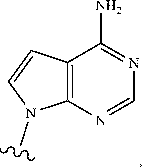

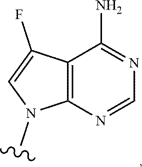

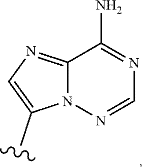

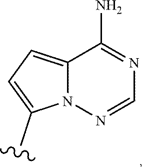

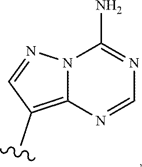

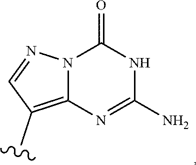

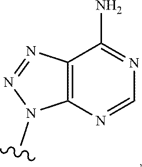

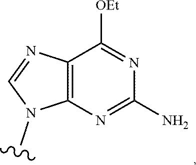

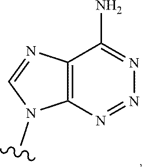

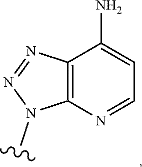

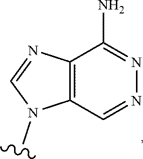

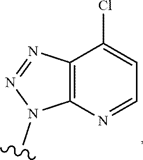

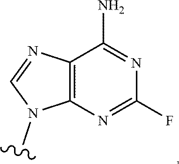

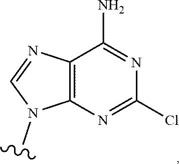

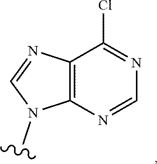

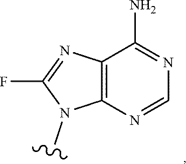

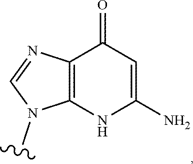

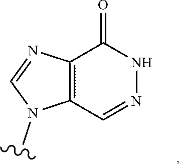

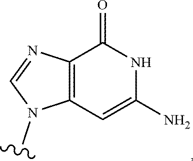

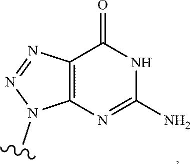

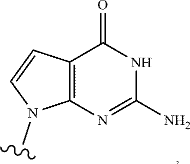

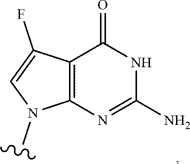

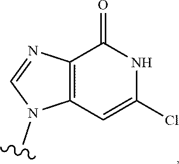

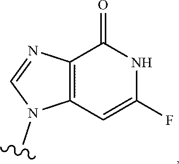

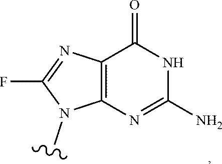

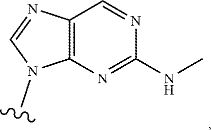

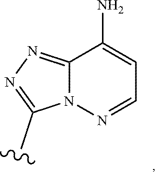

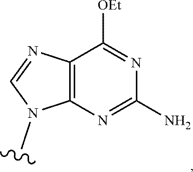

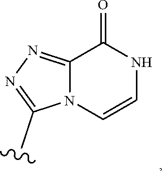

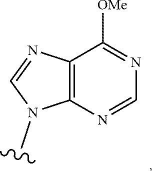

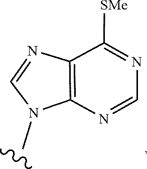

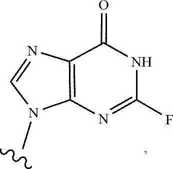

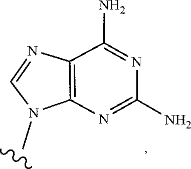

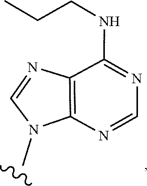

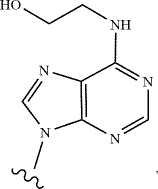

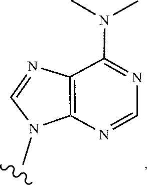

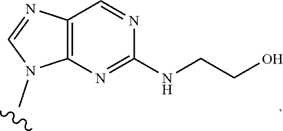

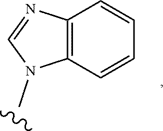

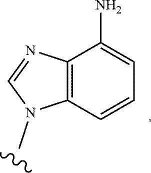

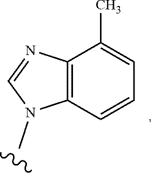

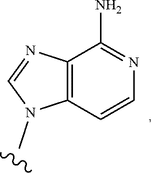

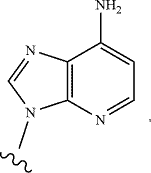

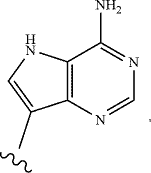

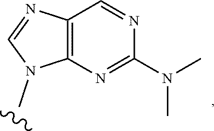

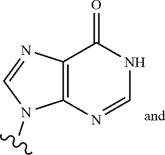

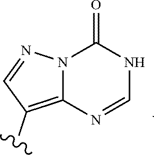

### Embodiment 41

The STING agonist amphiphile conjugate of Embodiment 40, wherein B⁴ is selected from the group consisting of:

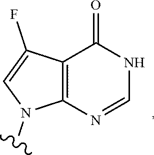

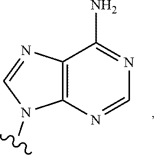

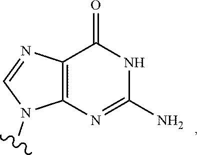

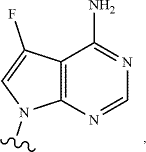

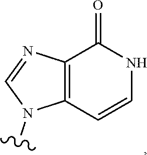

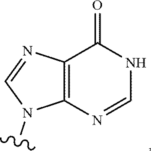

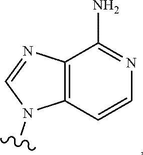

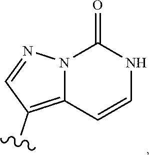

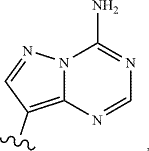

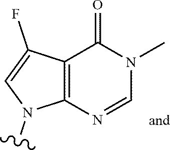

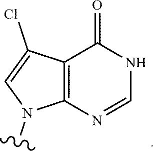

### Embodiment 42

The STING agonist amphiphile conjugate of Embodiment 41, wherein B⁴ is selected from the group consisting of:

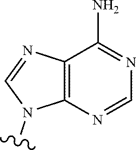

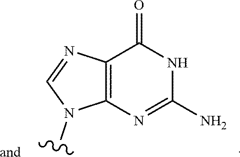

### Embodiment 43

The STING agonist amphiphile conjugate of any one of Embodiments 35-38, wherein B³ is selected from the group consisting of:

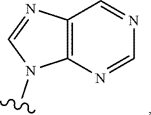

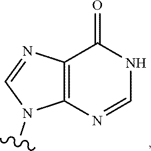

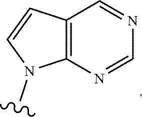

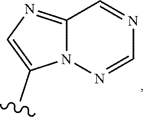

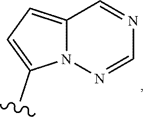

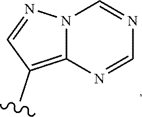

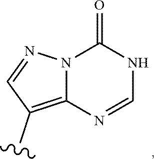

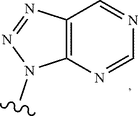

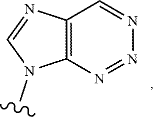

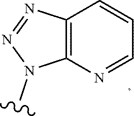

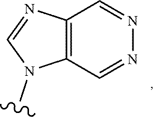

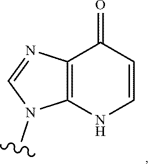

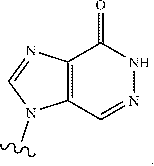

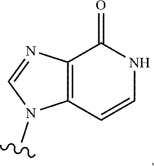

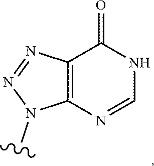

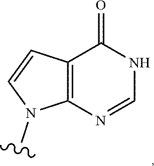

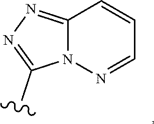

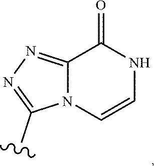

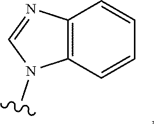

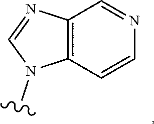

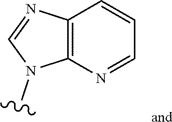

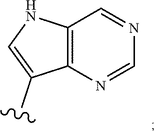

each of which is optionally and independently substituted at:
(i) any available carbon atom with a halo, C₁₋₆ alkyl, C₁₋₆ alkoxy, C₁₋₆ alkylthio, or amino group; and/or
(ii) any available nitrogen atom with a C₁₋₆ alkyl group.

### Embodiment 44

The STING agonist amphiphile conjugate of Embodiment 43, wherein B³ is selected from the group consisting of:

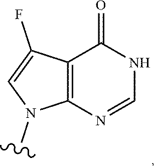

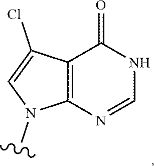

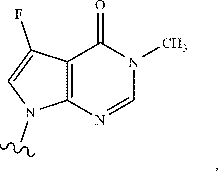

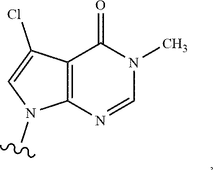

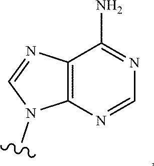

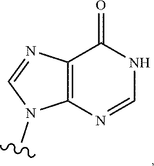

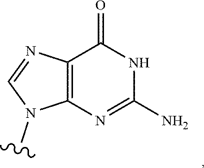

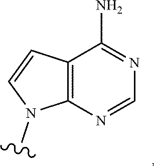

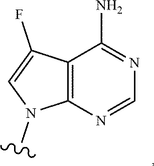

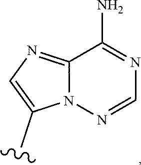

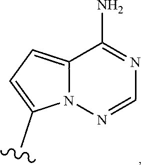

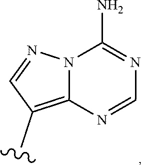

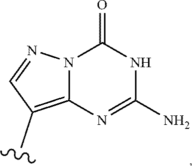

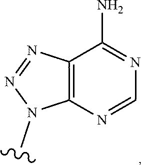

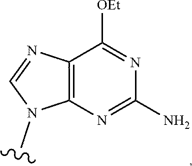

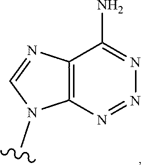

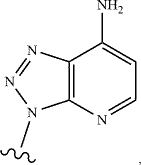

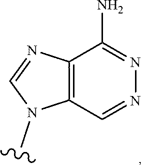

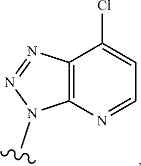

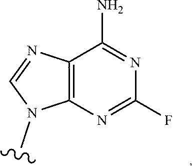

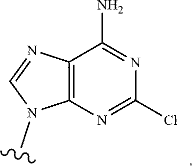

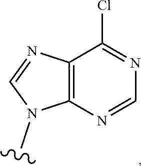

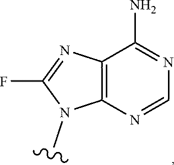

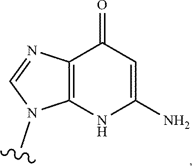

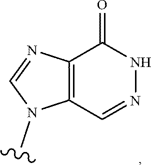

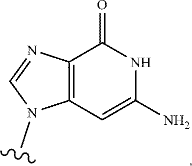

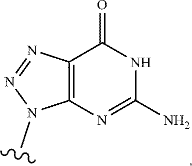

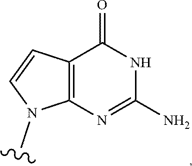

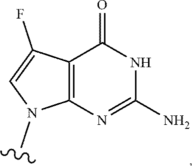

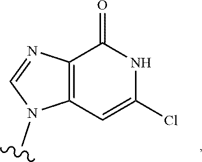

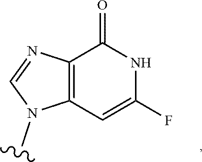

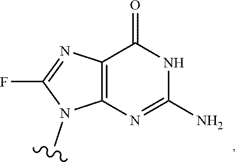

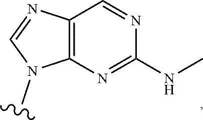

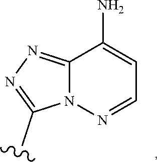

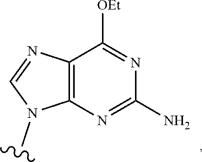

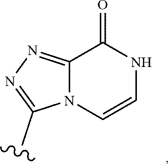

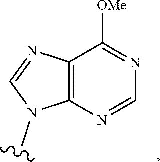

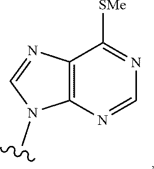

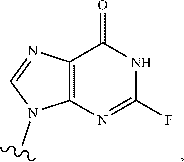

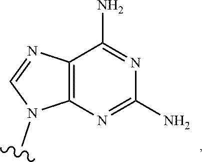

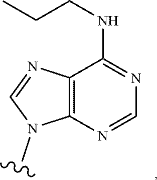

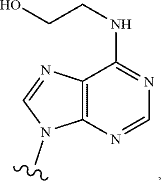

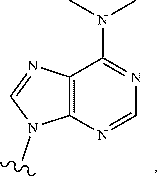

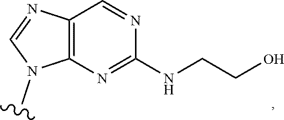

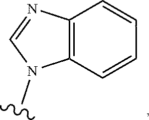

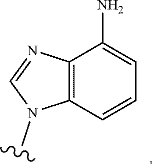

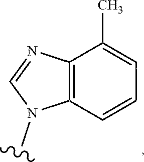

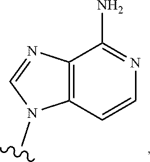

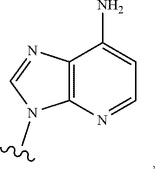

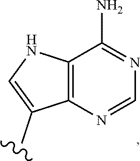

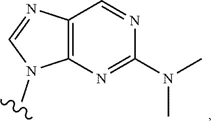

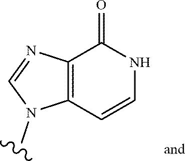

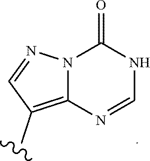

### Embodiment 45

The STING agonist amphiphile conjugate of Embodiment 44, wherein B³ is selected from the group consisting of:

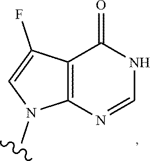

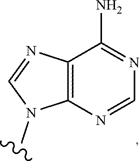

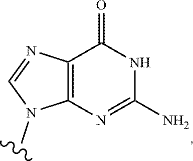

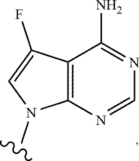

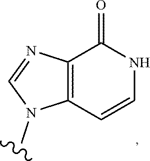

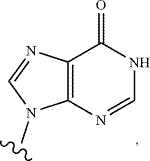

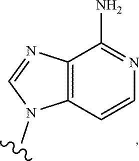

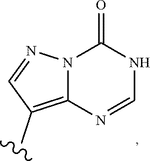

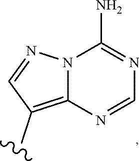

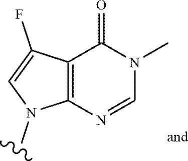

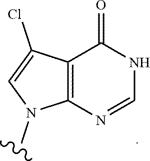

### Embodiment 46

The STING agonist amphiphile conjugate of Embodiment 45, wherein B³ is selected from the group consisting of:

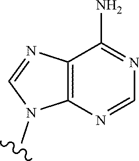

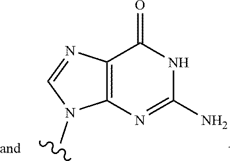

### Embodiment 47

The STING agonist amphiphile conjugate of any one of Embodiments 1-8, 13, and 15-46, wherein Q² is an oxygen atom.

### Embodiment 48

The STING agonist amphiphile conjugate of any one of Embodiments 1-8, 13, and 15-46, wherein Q² is a sulfur atom.

### Embodiment 49

The STING agonist amphiphile conjugate of any one of Embodiments 1-4, 9-12, 14-15, 18-26, and 31-46, wherein Q⁴ is an oxygen atom.

### Embodiment 50

The STING agonist amphiphile conjugate of any one of Embodiments 1-4, 9-12, 14-15, 18-26, and 31-46, wherein Q⁴ is a sulfur atom.

### Embodiment 51

The STING agonist amphiphile conjugate of any one of Embodiments 1-50, wherein X¹ and X² are an oxygen atom and R¹ and R² are independently a hydroxy group, a fluoro atom, or a chloro atom.

### Embodiment 52

A pharmaceutical composition comprising the compound of any one of Embodiments 1-51, or a pharmaceutically acceptable salt thereof, and a pharmaceutically acceptable carrier.

### (V) Methods of Making STING Agonist Amphiphile Conjugates

In some aspects, the disclosure provides methods for making the STING agonist amphiphile conjugates described herein. In some embodiments related to a STING agonist amphiphile conjugate comprising a CDN covalently linked to a polymer-modified lipid via a linker, methods for preparation of a CDN-linker conjugate are known in the art, and described, for example in WO2018/100558 and WO2018/200812, which are both herein incorporated by reference in their entirety.

In some embodiments, a method for preparation of a STING agonist amphiphile conjugate of the disclosure comprises reacting a CDN-linker conjugate with a polymer-modified lipid, wherein the linker comprises a heterobifunctional or heterotrifunctional group for conjugation to a reactive moiety of the polymer. For example, through methods of conjugation such as those described in US 10,464,969, which reference is herein incorporated by reference in its entirety.

Suitable polymer-modified lipids comprising a reactive moiety for conjugation are known, including for example, succinyl, maleimide, amine, orthopyridyl disulfide (PDP), carboxylic acid, carboxy N-hydroxy succinimidyl ester, azide, and bizrylazacyclooctynone functionalized PEG-lipids commercially available from Avanti Polar Lipids or Sigma-Aldrich. In some aspects, the PEG-lipid is a PEG-modified DSPE, a PEG-modified DOPE, a PEG-modified DPPE, a PEG-modified DMPE, a PEG-modified POPE, or a PEG-modified ceramide, wherein the PEG comprises a reactive moiety for covalent attachment to the CDN-linker conjugate. In some aspects, the PEG-lipid is a thiol-functionalized PEG-lipid, wherein the thiol is generated by reduction of a disulfide bond. For example, a PEG-lipid comprising a terminal thiol is generated by reduction of a PEG-lipid comprising a terminal PDP group.

In some embodiments, the linker comprises a heterobifunctional group, wherein the heterobifunctional provides a reactive moiety for conjugation to a reactive moiety of the polymer. In some embodiments, the heterobifunctional group is an azide-reactive group (e.g., bizrylazacyclooctynone) and the polymer comprises an azide, wherein the CDN-linker conjugate is covalently-linked to the polymer-modified lipid by reaction of the azide-reactive group of the linker to the azide of the polymer.

In some embodiments, the heterobifunctional group is a thiol-reactive group (e.g., a maleimide) and the polymer comprises a thiol, wherein the CDN-linker conjugate is covalently-linked to the polymer-modified lipid by reaction of the thiol-reactive group of the linker to the thiol of the polymer. For example, a STING agonist amphiphile conjugate of the disclosure is prepared by reaction of a PEG-lipid comprising a terminal thiol to a CDN-linker conjugate comprising a thiol-reactive group. An exemplary STING agonist amphiphile conjugate generated by reaction of a PEG-lipid comprising a thiol and a CDN-linker comprising a thiol-reactive group is CDN-PEG-Lipid.

### B. Phospholipids

In some embodiments, lipid nanodiscs of the disclosure comprise one or more phospholipids. In some embodiments, the phospholipid is selected from the non-limiting group of: a glycerophospholipid, a sphingophospholipid, a phosphatidylcholine (PC), a phosphatidylethanolamine (PE), a phosphotidylserine (PS), a phosphatidylinositol (PI), a phosphatidylglycerol (PG), or a phosphatidic acid (PA). In some embodiments, the phospholipid is a sphingophospholid, such as a sphingomyelin. In some embodiments, the phospholipid is a glycerophospholipid such as a PC, PE, PS, PI, PG, or PA. In some embodiments, the phospholipid is a ceramide-phosphate.

In some embodiments, the phospholipid comprises one or more aliphatic chains that are coupled to the phospholipid headgroup via ester bond linkages, amide bond linkages, thioester bond linkages, or a combination thereof. In some embodiments, the phospholipid comprises one or more acyl groups such as a fatty acid tail. As used herein, a “fatty acid tail”, alternatively termed a “fatty acid”, refers to a carboxylic acid with a long aliphatic chain which is either saturated or unsaturated. In some embodiments, the fatty acid tail is coupled to the phospholipid head group via an ester linkage. In some embodiments, the phospholipid is a diacyl lipid comprising two fatty acid tails.

In some embodiments, the phospholipid comprises at least one fatty acid tail. In some embodiments, the fatty acid tail is a medium-chain fatty acid comprising about 6 to about 12 carbons. In some embodiments, the fatty acid tail is a long-chain fatty acid comprising about 13 to about 21 carbons. In some embodiments, the fatty acid tail is a very long fatty acid comprising about 22 carbons or more. In some embodiments, the fatty acid tail comprises about 10 to about 30 hydrocarbon units, or preferably about 12, 13, 14, 15, 16, 17, 18, 19, 20, 21, 22, 23, 24, 25, 26, 27, 28, 29 or 30 hydrocarbon units.

In some embodiments, the phospholipid comprises a saturated fatty acid tail, wherein the ratio of number of carbons to number of —C═C— bonds is 14:0, 15:0, 16:0, 17:0, 18:0, 19:0, 20:0, 21:0, or 22:0. In some embodiments, the phospholipid comprises a unsaturated fatty acid tail, wherein the ratio of number of carbons to number of —C═C— bonds is 14:1, 14:2, 15:1, 15:2, 16:1, 16:2, 17:1, 17:2, 18:1, 18:2, 19:1, 19:2, 20:1, 20:2, 20:3, 20:4, 21:1, 21:2, 22:1, 22:2, 22:3, 22:4, 22:5, or 22:6. In some embodiments, the —C═C— bond of the unsaturated lipid tail is a cis bond or a trans bond.

In some embodiments, the fatty acid tail is saturated. In some aspects, the saturated fatty acid tail is selected from, but not limited to: caprylic acid (CH₃(CH₂)₆COOH)), capric acid (CH₃(CH₂)₈COOH)), lauric acid (CH₃(CH₂)₁₀COOH), stearic acid (CH₃(CH₂)₁₆COOH)), arachidic acid (CH₃(CH₂)₁₈COOH)), behenic acid (CH₃(CH₂)₂₀COOH), lignoceric acid CH₃(CH₂)₂₂COOH), or cerotic acid (CH₃(CH₂)₂₄COOH))

In some embodiments, the fatty acid tail is unsaturated. In some embodiments, the fatty acid tail comprises at least one cis alkene. In some embodiments, the fatty acid tail comprises at least one trans alkene. In some embodiments, the unsaturated fatty acid tail is selected from, but not limited to, a fatty acid listed in Table 1. Delta-x refers to a double bond beginning at the xth carbon-carbon bond as counted from the carboxylic end of the fatty acid backbone, with the prefix cis- or trans- indicating the configuration of the double bond.

TABLE 1

Examples of unsaturated fatty acids

| Name | Chemical Structure | Delta-x | # carbon atoms:# double bonds |
|---|---|---|---|
| Myristoleic acid | CH₃(CH₂)₃CH═CH(CH₂)₇COOH | cis-Δ⁹ | 14:1 |
| Palmitoleic acid | CH₃(CH₂)₅CH═CH (CH₂)₇COOH | cis-Δ⁹ | 16:1 |
| Sapienic acid | CH₃(CH₂)₈CH═CH(CH₂)₄COOH | cis-A⁶ | 16:1 |
| Oleic acid | CH₃(CH₂)₇CH═CH(CH₂)₇COOH | cis-Δ⁹ | 18:1 |
| Elaidic acid | CH₃(CH₂)₇CH═CH(CH₂)₇COOH | trans-A⁹ | 18:1 |
| Vaccenic acid | CH₃(CH₂)₅CH═CH(CH₂)₉COOH | trans- Δ¹¹ | 18:1 |
| Linoleic acid | CH₃(CH₂)₄CH═CHCH₂CH═CH(CH₂)₇COOH | cis,cis-Δ⁹, Δ¹² | 18:2 |
| Linoelaidic acid | CH₃(CH₂)₄CH═CHCH₂CH═CH(CH₂)₇COOH | trans, trans-Δ⁹, Δ¹² | 18:2 |
| Alpha-linolenic acid | CH₃CH₂CH═CHCH₂CH═CHCH₂CH═CH (CH2)₇COOH | cis,cis,cis-Δ⁹, Δ¹², Δ¹⁵ | 18:3 |
| Arachidonic acid | CH₃(CH₂)₄CH═CHCH₂CH═CHCH₂CH═CH CH₂CH═CH(CH₂)₃COOH | cis,cis,cis,cis-Δ⁵, Δ⁸, Δ^{11,} Δ¹⁴ | 20:4 |
| Elcosapentaenoic acid | CH₃CH₂CH═CHCH₂CH═CHCH₂CH═CHCH₂ CH═CHCH₂CH═CH(CH₂)₃COOH | cis, cis, cis, cis, cis-Δ⁵, Δ¹⁸, Δ¹¹, Δ¹⁴, Δ¹⁷ | 20:5 |
| Erucic acid | CH₃(CH₂)₇CH═CH(CH₂)₁₁COOH | cis-Δ¹³ | 22:1 |
| Docosahexaenoic acid | CH₃CH₂CH═CHCH₂CH═CHCH₂CH═CHCH₂ CH═CHCH₂CH═CHCH₂CH═CH(CH₂)₂COOH | cis,cis,cis,cis,cis,cis-Δ⁴, Δ⁷, Δ¹⁰, Δ¹³, Δ¹⁶, Δ¹⁹ | 22:6 |

In some embodiments, the phospholipid comprises a modified fatty acid tail. For example, a modified fatty acid tail selected from the non-limiting group of: diphytanoyl lipids, brominated lipids, oxidized lipids, or diacetylene lipids

In some embodiments, the phospholipid is a phosphatidylcholine, such as egg L-α-phosphatidylcholine (egg PC), soy L-α-phosphatidylcholine (soy PC), hydrogenated soy PC (HSPC), or 1-palmitoyl-2-oleoyl-sn-glycero-3-phosphocholine (POPC). In some aspects, the phospholipid is a synthetic phosphatidylcholine lipid, such as 1,2-dimyristoyl-sn-glycero-3-phosphocholine (DMPC), 1,2-distearoyl-sn-glycero-3-phosphocholine (DSPC), 1,2-dilinoleoyl-sn-glycero-3-phosphocholine (DLPC), 1,2-dioleoyl-sn-glycero-3-phosphocholine (DOPC), 1,2-diundecanoyl-sn-glycero-phosphocholine (DUPC), 1-palmitoyl-2-oleoyl-sn-glycero-3-ethylphosphocholine, 1,2-di-O-octadecenyl-sn-glycero-3-phosphocholine (18:0 Diether PC), 1-oleoyl-2 cholesterylhemisuccinoyl-sn-glycero-3-phosphocholine, 1-hexadecyl-sn-glycero-3-phosphocholine, 1-myristoyl-2-stearoyl-sn-glycero-3-phosphocholine (MSPC), or 1,2-dipalmitoyl-sn-glycero-3-phosphocoline (DPPC).

In some embodiments, the phospholipid is a phoshpatidylethanolamine, for example, selected from the non-limiting list of: 1,2-distearoyl-sn-glycero-3-phosphoethanolamine (DSPE), dipalmitoyl-sn-glycero-3-phosphoethanolamine (DPPE), 1,2-dioleoyl-sn-glycero-3-phosphoethanolamine (DOPE), 1,2-dimyristoyl-sn-glycero-3-phosphoethanolamine (DMPE), 1-palmitoyl-2-oleoyl-sn-glycero-3-phosphoethanolamine (POPE), or 1,2-diphytanoyl-sn-glycero-3-phosphoethanolamine.

In some embodiments, the phospholipid is a phosphotidylserine, for example, selected from the non-limiting list of: 1-palmitoyl-2-oleoyl-sn-glycero-3-phospho-L-serine (POPS), 1,2-dioleoyl-sn-glycero-3-phospho-L-serine (DOPS), 1,2-dipalmitoyl-sn-glycer-3-phospho-L-serine, or pharmaceutically acceptable salts thereof.

In some embodiments, the phospholipid is L-α-phosphatidylinositol, 1,2-dioleoyl-sn-glycero-3-phospho-rac-(1-glycerol) sodium salt (DOPG), or a sphingomyelin.

In some embodiments, the phospholipid is a naturally-occurring phospholipid comprising one or more chemical modifications. For example, in some embodiments, the phospholipid comprises a modified fatty acid tail or a modified headgroup. In some embodiments, the phospholipid comprises a chemical modification to allow cross-linking under suitable conditions. For example, the cross-linking of a targeting or imaging moiety (e.g., fluorescent dye) to the phospholipid to enable tracking of the lipid nanodisc.

In some embodiments, the phospholipid is selected according to one or more physical properties, e.g., phase transition temperature. As used herein “phase transition temperature,”“Tm,” or “Tₘ” each refer to the temperature required to induce a change in the liquid physical state from the ordered gel phase (e.g., wherein the hydrocarbon chains are fully extended and closely packed) to the disordered liquid crystalline phase (e.g., wherein the hydrocarbon chains are randomly oriented and fluid). As is understood in the art, one or more factors influences the phase transition temperature, e.g., hydrocarbon length, degree of unsaturation, charge, and headgroup species. For example, as the length of the hydrocarbon chain is increased, the van der Waals interaction is increased, resulting in a requirement for increased energy to disrupt ordered packing of the hydrocarbon chains and an increase in the phase transition temperature. As another example, as an unsaturation is introduced into the hydrocarbon chain (e.g., a cis double bond), the hydrocarbon chain requires lower temperature to induce an ordered packing arrangement, and phase transition temperature is decreased. Methods to measure phase transition temperature are known in the art, and include, for example, differential scanning calorimetry.

In some embodiments, the phospholipid is a saturated diacyl phospholipid having a Tm of at least about 30, 35, 40, 45, 50, 55, 60, 65, 70, 75, or 80° C. In some embodiments, the phospholipid is a phosphatidylcholine having a Tm of at least about 30, 35, 40, 45, 50, 55, 60, 65, 70, 75, or 80° C. In some embodiments, the phospholipid is a phosphatidylglycerol having a Tm of at least about 30, 35, 40, 45, 50, 55, 60, 65, 70, 75, or 80° C. In some embodiments, the phospholipid is a phosphatidylserine having a Tm of at least about 30, 35, 40, 45, 50, 55, 60, 65, 70, 75, or 80° C. In some embodiments, the phospholipid is a phosphatidylethanolamine having a Tm of at least about 30, 35, 40, 45, 50, 55, 60, 65, 70, 75, or 80° C. In some embodiments, the phospholipid has a Tm less than about 60, 55, 50, 45, 40, 35, 30, 25, 20, 15, 10, 5, 0, -5, or -10° C. In some embodiments, the phospholipid is a diacyl phospholipid comprising one or more unsaturated lipid tails, wherein the Tm is less than about 60, 55, 50, 45, 40, 35, 30, 25, 20, 15, 10, 5, 0, -5, or -10° C.

### C. Polyethylene Glycol (PEG)-Lipids

In some embodiments, the disclosure provides lipid nanodiscs comprising a PEG-lipid. In some embodiments, the lipid nanodiscs further comprise a PEG-lipid covalently-linked to a STING agonist, optionally via a linker to form a STING agonist amphiphile conjugate. Suitable PEG-lipids for use in compositions comprising lipid nanodiscs of the disclosure, or for use in covalent-attachment to form a STING agonist amphiphile conjugate of the disclosure, are further described below.

As used herein, the term “PEG-lipid” refers to a lipid modified with a PEG polymer or polymer comprising poly-ethylene glycol (PEG) units. Such species may be alternatively referred to as PEGylated lipids or PEG-modified lipids. For example, in some embodiments, the PEG-lipid comprises a hydrophobic lipid chemically linked to a polymer comprising PEG units. In some embodiments, the polymer comprises about 1 and about 200, between about 20 and about 150, between about 30 and about 100, or between about 40 and about 70 PEG units. In some embodiments, the polymer comprises about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, or 230 PEG units. In some embodiments, the polymer comprises about 40 to about 50 PEG units, or about 40, 41, 42, 43, 44, 45, 46, 47, 48, or 49 PEG units. In some embodiments, the polymer comprises n consecutive PEG units, wherein n is a value between about 20 to about 230, between about 30 and about 100, or between about 40 and about 70. In some embodiments, n is a value of about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, or 230.

PEG polymers are classified according to their molecular weights, for example PEG2000 or PEG2k has an average molecular weight of about 2000 Daltons, PEG5000 or PEG5k has an average molecular weight of about 5000 Daltons, and PEG10,000 or PEG10k has an average molecular weight of about 10,000 Daltons. PEG polymers useful for preparation of PEG-lipids are known in the art and are commercially available from Sigma Aldrich and other companies, which include, for example: monomethoxypolyethylene glycol (MePEG—OH), monomethoxypolyethylene glycol-succinate (MePEG—S), monomethoxypolyethylene glycol-succinimidyl succinate (MePEG—S—NHS), monomethoxypolyethylene glycol-amine (MePEG—NH2), monomethoxypolyethylene glycol-tresylate (MePEG-TRES), and monomethoxypolyethylene glycol-imidazolyl-carbonyl (MePEG-IM). Other PEG polymers useful for preparation of PEG-lipids are such as those described in US6,774,180 and US7,053,150. Additionally, monomethoxypolyethyleneglycol-acetic acid (MePEG—CH2COOH) is particularly useful for preparation of PEG-lipid conjugates, e.g., PEG-DAA conjugates.

In some embodiments, the PEG polymer has an average molecular weight of about 550 Daltons to about 10,000 Daltons, about 750 Daltons to about 5,000 Daltons, about 1,000 Daltons to about 5,000 Daltons, about 2,000 Daltons to about 5,000 Daltons. In some embodiments, the PEG has an average molecular weight of 2,000 Daltons. In some embodiments, the PEG polymer has an average molecular weight of 5,000 Daltons. In some embodiments, the PEG polymer has an average molecular weight of about 10,000 Daltons.

In some embodiments, the PEG polymer is a copolymer comprising (i) PEG units, and (ii) PPO, PLA, PLGA, PLCL, PMA, PE, PS, PAA, or PAM units. In some embodiments, the polymer is a block copolymer. In some embodiments, the polymer is a random copolymer.

In some embodiments, the PEG polymer is a copolymer comprising ethylene glycol (EG) units and propylene oxide (PO) units. In some embodiments, the PEG polymer is a random copolymer comprising EG and PO units. In some embodiments, the PEG polymer is a block copolymer comprising EG and PO units. In some embodiments, the PEG polymer comprises the formula (EG)ₙ(PO)ₖ, wherein n is a value between about 20 to about 150, between about 30 and about 100, or between about 40 and about 70, and k is a value between about 20 to about 150, between about 30 and about 100, or between about 40 and about 70. In some aspects, the PEG polymer comprises the formula (EG)ₙ(PO)ₖ(EG)₁, wherein n is a value between about 20 to about 150, between about 30 and about 100, or between about 40 and about 70, and k is a value between about 20 to about 150, between about 30 and about 100, or between about 40 and about 70, and 1 is a value between about 20 to about 150, between about 30 and about 100, or between about 40 and about 70, and k is a value between about 20 to about 150, between about 30 and about 100, or between about 40 and about 70. In some embodiments, the PEG polymer comprises the formula (PO)ₙ(EG)ₖ(PO)₁, wherein n is a value between about 20 to about 150, between about 30 and about 100, or between about 40 and about 70, and k is a value between about 20 to about 150, between about 30 and about 100, or between about 40 and about 70, and 1 is a value between about 20 to about 150, between about 30 and about 100, or between about 40 and about 70, and k is a value between about 20 to about 150, between about 30 and about 100, or between about 40 and about 70.

In some embodiments, the PEG polymer is directly attached to the lipid or is attached to the lipid via a spacer. Any spacer moiety is suitable for coupling the PEG to a lipid, with non-limiting examples including ester-containing spacer moieties or non-ester containing spacer moieties. Suitable non-ester containing spacer moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), and combinations thereof (e.g., a spacer composed of a carbamate moiety and an amido moiety). In some embodiments, a carbamate is used to conjugate the PEG to the lipid.

In some embodiments, an ester-containing moiety is used to couple the PEG to the lipid. Suitable ester-containing moieties include, e.g., carbonate (—OC(O)O—), succinoyl, phosphate esters (—O—(O)POH—O—), sulfonate esters, and combinations thereof.

In some embodiments, a PEG-lipid is selected from the non-limiting group of a PEG-modified dialkylamines; a PEG-modified dialkylglycerols; PEG coupled to dialkyloxylpropyls (PEG-DAA) such as is described in WO05/026372; PEG coupled to diacylglycerol (PEG-DAG) as described in US20030077829 and US200508689; PEG coupled to phospholipids such as phosphatidylethanolamine (PEG-PE); PEG conjugated to ceramides as described in US5,885,613; PEG conjugated to cholesterol or a derivative thereof.

In some embodiments, a PEG-lipid comprises a PEG conjugated to a phosphatidylethanolamine. Phosphatidylethanolamines are commercially available, or can be isolated or synthesized according to conventional methods know to those skilled in the art. In some embodiments, the phosphatidylethanolamine comprises saturated or unsaturated fatty acids with carbon chain lengths of about 10 to about 30 carbon units. In some embodiments, the phosphatidylethanolamine comprises a mono- or di-unsaturated fatty acid. In some embodiments, a suitable phosphatidylethanolamine is selected from: 1,2-distearoyl-sn-glycero-3-phosphoethanolamine (DSPE), 1,2-dipalmitoyl-sn-glycero-3-phosphoethanolamine (DPPE), 1,2-dioleoyl-sn-glycero-3-phosphoethanolamine (DOPE), 1-palmitoyl-2-oleoyl-sn-glycero-3-phosphoethanolamine (POPE), or 1,2-dimyristoyl-sn-glycero-3-phosphoethanolamine (DMPE).

In some aspects, a PEG-lipid suitable for use in the present disclosure includes, but is not limited to, 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), 1,2-dioleoyl-sn-glycero-3-phosphoethanolamine-N-[amino(polyethylene glycol)] (PEG-DOPE), PEG-dipalmitoyl phosphatidylethanolamine (PEG-DPPE), 1,2-dimyristoyl-sn-glycero-3-phosphoethanolamine-N-[methoxy(polyethyleneglycol)] (PEG-DMPE), 1-palmitoyl-2-oleoyl-sn-glycero-3-phosphoethanolamine-N-[amino(polyethylene glycol)] (PEG-POPE), PEG-disteryl glycerol (PEG-DSG), PEG-dipalmetoleyl, PEG-dioleyl, PEG-distearyl, PEG-diacylglycamide (PEG-DAG), or PEG-1,2-dimyristyloxlpropyl-3-amine (PEG-c-DMA).

In some embodiments, the PEG-lipid is selected from: a PEG-modified DSPE, a PEG-modified DOPE, a PEG-modified DPPE, a PEG-modified DMPE, a PEG-modified POPE, a PEG-modified ceramide, or mixtures thereof. In some embodiments, the PEG-lipid is a PEG2000-DSPE, a PEG3000-DSPE, a PEG5000-DSPE, a PEG7500-DSPE, or a PEG10,000-DSPE.

### D. Additional Components

In some aspects, the lipid nanodiscs disclosed herein comprise a STING agonist amphiphile conjugate, a phospholipid, a PEG-lipid, and at least one additional component. For example, in some embodiments, the lipid nanodisc composition comprises a permeability enhancer molecule, a carbohydrate, a polymer, a surface altering agent (e.g., surfactant), a structural lipid, an ionizable and/or cationic lipid or other components.

### (I) Ionizable Cationic Lipid

In some aspects, a lipid nanodisc comprises an ionizable lipid or an ionizable cationic lipid. As used herein “an ionizable lipid’ has its ordinary meaning in the art and refers to a lipid comprising one or more charged moieties. An “ionizable cationic lipid” refers to an ionizable lipid comprising one or more positively charged moieties. For example, a positively-charged moiety selected from, but not limited to, an amine group (e.g., a primary, secondary, and/or tertiary amine), an ammonium group, a pyridinium group, a guanidine group, and an imidizolium group. The charge of the charged moiety may vary, in some cases, with the environmental conditions, for example, changes in pH may alter the charge of the moiety, and/or cause the moiety to become charged or uncharged. In general, the charge density of the molecule may be selected as desired.

In some embodiments, the ionizable lipid is an ionizable amino lipid, sometimes referred to in the art as an ionizable cationic lipid. In one embodiment, the ionizable amino lipid may have a positively charged hydrophilic head and a hydrophobic tail that are connected via a linker structure. In addition to these, an ionizable lipid may also be a lipid including a cyclic amine group.

In some embodiments, the ionizable lipid is one described in U.S. Pat. No. 9,061,063 or International Publication No. WO2013116126.

In some embodiments, a lipid nanodisc of the disclosure comprises an ionizable lipid selected from, but not limited to, formula CLI-CLXXXXII of U.S. Pat. No. 7,404,969.

In some embodiments, a lipid nanodisc of the disclosure comprises one or more ionizable lipids, such as one selected from, but not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 2-({ 8-[(3β)-cholest-5-en-3-yloxy]octyl}oxy)-N,N-dimethyl-3-[(9Z,12Z)-octadeca-9,12-dien-1-yloxy]prop an-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy ]propan-1-amine (Octyl-CLinDMA (2R)), (2S)-2-({8-[(3β)-cholest-5-en-3-yloxy]octyl}oxy)-N,N-dimethyl-3-[(9Z,12Z)-octadeca-9,12-dien-1-yloxy ]propan-1-amine (Octyl-CLinDMA (2S)).N,N-dioleyl-N,N-dimethylammonium chloride (“DODAC”), N-(2,3-dioleyloxy)propyl-N,N--N-triethylammonium chloride (“DOTMA”); N,N-distearyl-N,N-dimethylammonium bromide (“DDAB”); 1,2-Dioleyloxy-3-trimethylaminopropane chloride salt (“DOTAP.C1”); 3-β-(N--(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”).

In some embodiments, a lipid nanodisc of the disclosure comprises one or more ionizable lipids selected from: 1,2-dioleoyl-3-trimethylammonium-propane (18:1 TAP or DOTAP), 1,2-dipalmitoyl-3-trimethylammonium-propane (16:0 TAP), 1,2-stearoyl-3-trimethylammonium-propane (18:0 TAP), or a pharmaceutically acceptable salt thereof.

### (Ii) Structural Lipid

In some embodiments, a lipid nanodisc comprises one or more structural lipids. As used herein, the term “structural lipid” refers to sterols and also to lipids containing sterol moieties. A suitable structural lipids for use in the disclosure is selected from the group that includes, but is not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol.

### (Iii) Other

In some embodiments, a lipid nanodisc of the disclosure comprises a permeability enhancer molecule, such as one described by U.S. Pat. Application Publication No. 2005/0222064.

In some embodiments, a lipid nanodisc of the disclosure comprises a carbohydrate. A suitable carbohydrate includes a simple sugar (e.g., glucose) and a polysaccharide (e.g., glycogen and derivatives or analogs thereof).

### IV. Formulation of Lipid Compositions of the Disclosure

In some aspects, the disclosure provides a lipid composition (e.g., liposome or lipid nanodisc) comprising a STING amphiphile conjugate, a phospholipid, and a PEG-lipid. In some aspects, the disclosure provides lipid nanodisc compositions, wherein the lipid nanodisc comprises a STING amphiphile agonist conjugate, a phospholipid, and a PEG-lipid.

In some embodiments, a lipid nanodisc comprises a molar ratio of about 5-15% or about 5%, about 6%, about 7%, about 8%, about 9%, about 10%, about 11%, about 12%, about 13%, about 14%, or about 15% STING agonist amphiphile conjugate. In some embodiments, the lipid nanodisc composition comprises a molar ratio of about 5% STING agonist amphiphile conjugate. In some embodiments, the lipid nanodisc composition comprises a molar ratio of about 6% STING agonist amphiphile conjugate. In some embodiments, the lipid nanodisc composition comprises a molar ratio of about 7% STING agonist amphiphile conjugate. In some embodiments, the lipid nanodisc composition comprises a molar ratio of about 8% STING agonist amphiphile conjugate. In some embodiments, the lipid nanodisc composition comprises a molar ratio of about 9% STING agonist amphiphile conjugate. In some embodiments, the lipid nanodisc composition comprises a molar ratio of about 10% STING agonist amphiphile conjugate.

In some embodiments, a lipid nanodisc comprises a molar ratio of 10-30%, about 15-25%, about 15-20%, or about 15%, about 16%, about 17%, about 18%, about 19%, about 20%, about 21%, about 22%, about 23%, about 24%, about 25%, about 26%, about 27%, about 28%, about 29%, or about 30% PEG- lipid.

In some embodiments, a lipid nanodisc comprises a molar ratio of at least about 85-45%, about 80-50%, about 75-55%, about 70-60%, or about 55%, about 60%, about 65%, about 70%, about 75%, or about 80% phospholipid.

In some embodiments, a lipid nanodisc further comprises a molar ratio of about 10-30%, about 10-20%, about 10%, about 15%, about 20%, about 25%, or about 30% ionizable cationic lipid.

In some embodiments, a lipid nanodisc comprises a molar ratio of about 10-50%, about 10-40%, about 10-30%, about 10-20%, about 10%, about 15%, about 20%, about 25%, about 30%, about 35%, about 40%, about 45%, or about 50% sterol.

In some embodiments, a lipid nanodisc comprises a molar ratio of about 5-15% STING agonist amphiphile conjugate, about 10-30% PEG-modified lipid, and about 55-85% phospholipid.

In some embodiments, the lipid nanodisc composition comprises a molar ratio of STING agonist amphiphile conjugate that is at least 5%, molar ratio of STING agonist amphiphile conjugate and PEG-modified lipid when combined that is about 25%, and a molar ratio of phospholipid that is about 75%. In some embodiments, the lipid nanodisc composition comprises a molar ratio of STING agonist amphiphile conjugate, PEG-modified lipid, and phospholipid that is about 5:20:75. In some embodiments, the lipid nanodisc composition comprises a molar ratio of STING agonist amphiphile conjugate, PEG-modified lipid, and phospholipid that is about 10:15:75.

In some embodiments, a lipid nanodisc comprises a molar ratio of:
(i) about 5%, about 6%, about 7%, about 8%, about 9%, about 10%, about 11%, about 12%, about 13%, about 14%, or about 15% STING agonist amphiphile conjugate;
(ii) about 15%, about 16%, about 17%, about 18%, about 19%, about 20%, about 21%, about 22%, about 23%, about 24%, about 25%, about 26%, about 27%, about 28%, about 29%, or about 30% PEG-modified lipid; and
(iii) about 60%, about 61%, about 62%, about 63%, about 64%, about 65%, about 66%, about 67%, about 68%, about 69%, about 70% about 71%, about 72%, about 73%, about 74%, about 75%, about 76%, about 77%, about 78%, about 79%, or about 80% phospholipid.

In some embodiments, a lipid nanodisc comprises a molar ratio of about 5-15% STING agonist amphiphile conjugate; about 15%-30% PEG-modified lipid; about 25-65% phospholipid; and about 15-30% ionizable cationic lipid. In some embodiments, the lipid nanodisc composition comprises a molar ratio of STING agonist amphiphile conjugate that is at least 5%, a molar ratio of STING agonist amphiphile conjugate and PEG-modified lipid that when combined is about 25%, a molar ratio of ionizable cationic lipid that is about 20%, and a molar ratio of phospholipid that is about 55%. In some embodiments, the lipid nanodisc composition comprises a molar ratio of STING agonist amphiphile conjugate that is at least 5%, a molar ratio of STING agonist amphiphile conjugate and PEG-modified lipid that when combined is about 30%, a molar ratio of ionizable cationic lipid that is about 20%, and a molar ratio of phospholipid that is about 50%. In some embodiments, the lipid nanodisc composition comprises a molar ratio of STING agonist amphiphile conjugate that is at least 10%, a molar ratio of STING agonist amphiphile conjugate and PEG-modified lipid that when combined is about 30%, a molar ratio of ionizable cationic lipid that is about 20%, and a molar ratio of phospholipid that is about 50%. In some embodiments, the lipid nanodisc composition comprises a molar ration of STING agonist amphiphile conjugate, PEG-modified lipid, phospholipid, and ionizable cationic lipid that is 5:20:55:20. In some embodiments, the lipid nanodisc composition comprises a molar ration of STING agonist amphiphile conjugate, PEG-modified lipid, phospholipid, and ionizable cationic lipid that is 10:20:50:20.

In some embodiments, a lipid nanodisc comprises a molar ratio of:
(i) about 5%, about 6%, about 7%, about 8%, about 9%, about 10%, about 11%, about 12%, about 13%, about 14%, or about 15% STING agonist amphiphile conjugate;
(ii) about 15%, about 16%, about 17%, about 18%, about 19%, about 20%, about 21%, about 22%, about 23%, about 24%, about 25%, about 26%, about 27%, about 28%, about 29%, or about 30% PEG-modified lipid;
(iii) about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53% about 54%, about 55%, about 56%, about 57%, about 58%, about 59%, about 60%, about 61%, about 62%, about 63%, about 64%, or about 65% phospholipid; and
(iv) about 15%, about 16%, about 17%, about 18%, about 19%, about 20%, about 21%, about 22%, about 23%, about 24%, about 25%, about 26%, about 27%, about 28%, about 29%, or about 30% ionizable cationic lipid.

In some embodiments, a lipid nanodisc comprises a molar ratio of about 5-15% STING agonist amphiphile conjugate; about 15%-30% PEG-modified lipid; about 15-50% phospholipid; and about 10-40% sterol.

In some embodiments, a lipid nanodisc comprises a molar ratio of:
(i) about 5%, about 6%, about 7%, about 8%, about 9%, about 10%, about 11%, about 12%, about 13%, about 14%, or about 15% STING agonist amphiphile conjugate;
(ii) about 15%, about 16%, about 17%, about 18%, about 19%, about 20%, about 21%, about 22%, about 23%, about 24%, about 25%, about 26%, about 27%, about 28%, about 29%, or about 30% PEG-modified lipid;
(iii) about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, phospholipid; and
(iv) about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% sterol.

### A. Exemplary Formulation of Lipid Compositions of the Disclosure

In some embodiments, the STING agonist amphiphile conjugate is a compound set forth in any one of Embodiments 1-52. In some embodiments, the STING agonist amphiphile conjugate is CDN-PEG-Lipid.

In some embodiments, the phospholipid is hydrogenated soy L-α-phosphatidylcholine (HSPC). In some embodiments, the phospholipid is 1,2-dipalmitoyl-sn-glycero-3-phosphocholine (DPPC). In some embodiments, the phospholipid is 1,2-distearoyl-sn-glycero-3-phosphocholine (DSPC).

In some aspects, the PEG-lipid is 1,2-diastearoyl-sn-glycero-3-phosphoethanoalmine-N-[methoxy-(poly(ethylene glycol))-2000] (DSPE-PEG2k) or DSPE-PEG5k.

In some embodiments, the disclosure provides a lipid nanodisc composition, wherein the lipid nanodisc comprises a STING amphiphile agonist conjugate, a phospholipid, a PEG-lipid, and an ionizable cationic lipid. In some embodiments, the ionizable cationic lipid is 1,2-dipalmitoyl-3-trimethylammonium-propane (16:0 TAP).

In some embodiments, a lipid nanodisc composition of the disclosure is as set forth in Table 2.

TABLE 2

Lipid Nanodisc Compositions

| Component 1 (“1”) | Component 2 (“2”) | Component 3 (“3”) | Component 4 (“4”) | Approximate molar ratio (1:2:3:4) |
|---|---|---|---|---|
| HSPC | DSPE-PEG5k | CDN-PEG-Lipid | - | 75:20:5:0 |
| HSPC | DSPE-PEG5k | CDN-PEG-Lipid | - | 70:20:10:0 |
| HSPC | DSPE-PEG5k | CDN-PEG-Lipid | - | 70:25:5:0 |
| HSPC | DSPE-PEG5k | CDN-PEG-Lipid | - | 65:20:15:0 |
| HSPC | DSPE-PEG5k | CDN-PEG-Lipid | - | 65:25:10:0 |
| HSPC | DSPE-PEG5k | CDN-PEG-Lipid | 16:0 TAP | 60:20:5:15 |
| HSPC | DSPE-PEG5k | CDN-PEG-Lipid | 16:0 TAP | 55:20:10:15 |
| HSPC | DSPE-PEG5k | CDN-PEG-Lipid | 16:0 TAP | 55:20:5:20 |
| HSPC | DSPE-PEG5k | CDN-PEG-Lipid | 16:0 TAP | 50:25:5:20 |
| HSPC | DSPE-PEG5k | CDN-PEG-Lipid | 16:0 TAP | 50:20:10:20 |
| HSPC | DSPE-PEG5k | CDN-PEG-Lipid | 16:0 TAP | 45:25:10:20 |
| HSPC | DSPE-PEG5k | CDN-PEG-Lipid | 16:0 TAP | 45:20:15:20 |
| HSPC | DSPE-PEG5k | CDN-PEG-Lipid | 16:0 TAP | 40:25:10:20 |

In some embodiments, the lipid nanodisc composition comprises a particle fraction that are lipid nanodiscs that is at least 0.5, 0.55, 0.6, 0.65, 0.7, 0.75, 0.8, 0.85, 0.9, or higher. Methods to evaluate particle fraction are described further herein. In some embodiments, the lipid nanodisc composition comprises a particle fraction of about 0.5 that are lipid nanodiscs. In some embodiments, the lipid nanodisc composition comprises a particle fraction of about 0.55 that are lipid nanodiscs. In some embodiments, the lipid nanodisc composition comprises a particle fraction of about 0.6 that are lipid nanodiscs. In some embodiments, the lipid nanodisc composition comprises a particle fraction of about 0.65 that are lipid nanodiscs. In some embodiments, the lipid nanodisc composition comprises a particle fraction of about 0.7 that are lipid nanodiscs. In some embodiments, the lipid nanodisc composition comprises a particle fraction of about 0.75 that are lipid nanodiscs. In some embodiments, the lipid nanodisc composition comprises a particle fraction of about 0.8 that are lipid nanodiscs. In some embodiments, the lipid nanodisc composition comprises a particle fraction of about 0.85 that are lipid nanodiscs. In some embodiments, the lipid nanodisc composition comprises a particle fraction of about 0.9 that are lipid nanodiscs. In some embodiments, the lipid nanodisc composition comprises a particle fraction of about 0.95 that are lipid nanodiscs. In some embodiments, the lipid nanodisc composition comprises a particle fraction of about 0.96 that are lipid nanodiscs. In some embodiments, the lipid nanodisc composition comprises a particle fraction of about 0.97 that are lipid nanodiscs. In some embodiments, the lipid nanodisc composition comprises a particle fraction of about 0.98 that are lipid nanodiscs. In some embodiments, the lipid nanodisc composition comprises a particle fraction of about 0.99 that are lipid nanodiscs. In some embodiments, the lipid nanodisc composition comprises a particle fraction that is fully lipid nanodiscs.

In some embodiments, the lipid nanodisc composition comprises a particle fraction that are non-lipid nanodisc particles (e.g., liposomes, spherical micelles, amorphous lipid aggregates) that is not more than about 0.5, 0.45, 0.4, 0.35, 0.3, 0.25, 0.2, 0.15, or 0.1. In some embodiments, the lipid nanodisc composition comprises a particle fraction that is non-lipid nanodisc particles that is about 0.15. In some embodiments, the lipid nanodisc composition comprises a particle fraction that is non-lipid nanodisc particles that is about 0.10. In some embodiments, the lipid nanodisc composition comprises a particle fraction that is non-lipid nanodisc particles that is about 0.05. In some embodiments, the lipid nanodisc composition comprises a particle fraction that is non-lipid nanodisc particles that is about 0.01.

In some embodiments, the lipid nanodisc have one or more physical properties selected from: (i) a disc-like, a bilayer disc, or discoid morphology, e.g., as measured by electron microscopy (e.g., TEM, cryoTEM); (ii) a hydrodynamic diameter of about 10-15 nm, about 10-20 nm, about 15-20 nm, about 15-25 nm, about 20-25 nm, about 20-30 nm, about 25-30 nm, about 25-35 nm, about 30-35 nm, about 35-40 nm, about 35-45 nm, about 40-45 nm, about 40-50 nm, or about 45-50 nm, e.g., as measured by DLS; (iii) a diameter of about 10-15 nm, about 10-20 nm, about 15-20 nm, about 15-25 nm, about 20-25 nm, about 20-30 nm, about 25-30 nm, about 25-35 nm, about 30-35 nm, about 35-40 nm, about 35-45 nm, about 40-45 nm, about 40-50 nm, or about 45-50 nm, e.g., as measured by electron microscopy (e.g., TEM, cryoTEM); (iv) a height of about 3, 4, 5, 6, 7, 8, 9, 10, 11 or 12 nm, e.g., as measured by electron microscopy (e.g., TEM, cryoTEM); (v) an aspect ratio as measured by particle diameter to height of about 10, 9, 8, 7, 6, 5, 4, or 3 to 1, e.g., as measured by electron microscopy (e.g., TEM, cryoTEM); and (vi) a zeta potential of about -3, -2.5, -2, -1.5, -1, or -0.5 mV.

In some embodiments, disclosure provides a lipid composition comprising (i) a STING agonist amphiphile conjugate according to any one of Embodiments 1-52; (ii) a PEG-lipid; and (iii) a phospholipid, wherein the lipid composition comprises lipid particles comprising (i), (ii), and (iii); and wherein a plurality of the particles are lipid nanodiscs having a diameter between about 15 and about 40 nm and a height between about 5 nm and about 6 nm.

In some embodiments, disclosure provides a lipid composition comprising (i) a STING agonist amphiphile conjugate according to any one of Embodiments 1-52; (ii) a PEG-lipid; and (iii) a phospholipid, wherein the lipid composition comprises a molar ratio of (i):(ii):(iii) that is about 5:20:75, wherein the lipid composition comprises lipid particles comprising (i), (ii), and (iii); and wherein a plurality of the particles are lipid nanodiscs having a diameter between about 15 and about 40 nm and a height between about 5 nm and about 6 nm.

In some embodiments, disclosure provides a lipid composition comprising (i) a STING agonist amphiphile conjugate according to any one of Embodiments 1-52; (ii) a PEG-lipid that is DSPE-PEG2k or DSPE-PEG5k; and (iii) a phospholipid, wherein the lipid composition comprises lipid particles comprising (i), (ii), and (iii); and wherein a plurality of the particles are lipid nanodiscs having a diameter between about 15 and about 40 nm and a height between about 5 nm and about 6 nm.

In some embodiments, disclosure provides a lipid composition comprising (i) a STING agonist amphiphile conjugate according to any one of Embodiments 1-52; (ii) a PEG-lipid that is DSPE-PEG5k; and (iii) a phospholipid, wherein the lipid composition comprises a molar ratio of (i):(ii):(iii) that is about 5:20:75, wherein the lipid composition comprises lipid particles comprising (i), (ii), and (iii); and wherein a plurality of the particles are lipid nanodiscs having a diameter between about 15 and about 40 nm and a height between about 5 nm and about 6 nm.

In some embodiments, disclosure provides a lipid composition comprising (i) a STING agonist amphiphile conjugate according to any one of Embodiments 1-52; (ii) a PEG-lipid that is DSPE-PEG5k; and (iii) a phospholipid, wherein the lipid composition comprises lipid particles comprising (i), (ii), and (iii); and wherein a plurality of the particles are lipid nanodiscs having a diameter between about 15 and about 40 nm and a height between about 5 nm and about 6 nm. In some embodiments, (i) is CDN-PEG-Lipid.

In some embodiments, disclosure provides a lipid composition comprising (i) a STING agonist amphiphile conjugate according to any one of Embodiments 1-52; (ii) a PEG-lipid that is DSPE-PEG2k or DSPE-PEG5k; and (iii) a phospholipid, wherein the lipid composition comprises a molar ratio of (i):(ii):(iii) that is about 5:20:75, wherein the lipid composition comprises lipid particles comprising (i), (ii), and (iii); and wherein a plurality of the particles are lipid nanodiscs having a diameter between about 15 and about 40 nm and a height between about 5 nm and about 6 nm.

### V. Methods of Making and Characterizing Lipid Compositions of the Disclosure

In some embodiments, the disclosure provides lipid compositions prepared from a lipid composition comprising a STING agonist amphiphile conjugate, a phospholipid, and a PEG-lipid. In some embodiments, the lipid composition comprises lipid particles, wherein a plurality of lipid particles are lipid nanodiscs. In some embodiments, the plurality of lipid particles are liposomes. In some embodiments, the disclosure provides lipid nanodisc compositions prepared from a lipid composition comprising a STING agonist amphiphile conjugate, a phospholipid, and a PEG-lipid. In some embodiments, the lipid composition comprises an ionizable cationic lipid. In some embodiments, the lipid composition comprises a sterol.

In some embodiments, the disclosure provides lipid nanodisc compositions comprising a STING agonist amphiphile conjugate, a phospholipid, and a PEG-lipid that are prepared using conventional methods for lipid nanodisc preparation. For example, methods for preparing lipid nanodiscs are known in the art and described, e.g., by Viitala, et al (2016) LANGMUIR 32:4554-4563 and Johnsson and Edwards, Biophysical J. 2003, 85, 3839-3847. In some embodiments, the thin film hydration method is used to prepare lipid nanodisc compositions of the disclosure. In this method, the lipid components (e.g., STING agonist amphiphile conjugate, phospholipid, PEG-lipid, sterol, and/or ionizable cationic lipid) are co-dissolved in a suitable organic solvent such as chloroform, ethanol, methanol, acetonitrile, and the like. Following dissolution, the organic solvent is removed to form a lipid film. The lipid film is hydrated by dispersion in a buffered aqueous medium, such as by agitation. The lipid dispersion is then extruded through a membrane to provide a preparation of lipid nanodiscs.

In some embodiments, the ethanol precipitation method is used to prepare lipid nanodisc compositions of the disclosure. In this method, the lipid components (e.g., STING agonist amphiphile conjugate, phospholipid, PEG-lipid, sterol, and/or ionizable cationic lipid) are co-dissolved in a suitable water-miscible organic solvent, such as ethanol. Following dissolution, the organic solvent is slowly added with mixing to a larger volume of buffered aqueous medium. Generally, the final volume of organic solvent is less than or equal to about 20% of the total volume. The organic solvent is removed by multiple rounds of dialysis and replaced by aqueous buffer. The lipid dispersion is then extruded through a membrane to provide a preparation of lipid nanodisc particles.

Methods for characterization of lipid nanodiscs compositions of the disclosure are known in the art. For example, microscopy (e.g., transmission electron microscopy (TEM), cryo-TEM, or scanning electron microscopy) can be used to examine the morphology and size distribution of lipid nanodiscs. Dynamic light scattering or potentiometry (e.g., potentiometric titrations) can be used to measure zeta potentials. Dynamic light scattering can also be used to determine the size (e.g., hydrodynamic radius or diameter) and polydispersity of lipid nanodiscs.

In some embodiments, the disclosure provides compositions comprising lipid nanodiscs, wherein the lipid nanodiscs have disc-like morphology. In some embodiments, the lipid nanodiscs are disc-like micelles. A disc-like micelle may also be referred to as a discoidal micelle or a bilayer disc. Methods of measuring lipid nanodisc morphology are known in the art. For example, the morphology can be observed using transmission electron microscopy (TEM) or cryo-TEM. In the case of cryo-TEM, lipid nanodisc compositions are vitrified using methods such as those described by Kuntsche, et al. (2011) INT. J. PHARM 417:20-137 and Iancu, et al. (2006) NAT. PROTOC. 1:2813-2819. The lipid sample is then transferred to a TEM grid under controlled temperature and humidity conditions. Micrographs are taken using a TEM. The images are analyzed to determine the size and shape of the lipid nanodiscs.

In some embodiments, the disclosure provides composition comprising lipid nanodiscs, wherein the lipid nanodiscs have a diameter of about 20-100 nm, 30-90 nm, 30-80 nm, 30-70 nm, 30-60 nm, 30-50 nm, or 30-40 nm. In some embodiments, the lipid nanodiscs have a diameter of about 10 nm to about 100 nm, about 20 nm to about 90 nm, about 30 nm to about 80 nm, about 30 nm to about 70 nm, about 30 nm to about 60 nm, about 30 nm to about 50 nm, about 30 nm to about 40 nm, as measured by TEM or cryo-TEM. In some embodiments, the lipid nanodiscs have a height of about 5 nm to about 15 nm, about 6 nm, about 7 nm, about 8 nm, about 9 nm or about 10 nm as measured by TEM or cryo-TEM.

In some embodiments, the disclosure provides lipid compositions comprising lipid nanodiscs, wherein the lipid nanodiscs have a hydrodynamic diameter of about 10 nm to about 100 nm, about 20 nm to about 90 nm, about 30 nm to about 80 nm, about 30 nm to about 70 nm, about 30 nm to about 60 nm, about 30 nm to about 50 nm, or about 30 nm to about 40 nm, as measured by DLS.

In some embodiments, the lipid compositions and concentration of lipid components therein used to make lipid nanodiscs affects their phase behavior (see, e.g., Johnsson, et al (2003) BIOPHYSICAL J. 85:3839-3847). For example, in some embodiments, the composition comprises lipid nanodiscs, as well as other types of lipid particles. In some embodiments, the disclosure provides compositions comprising lipid nanodiscs and one or more lipid particles selected from, but not limited to, unilamellar liposomes, bilamellar liposomes, multilamellar liposomes, and spherical micelles

Lipid nanodisc particles are distinctive from other particle types. For example, lipid nanodiscs have a flat circular lipid bilayer surrounded by a highly curved rim, as described by Lundquist, BIOCHIM. BIOPHYS. ACTA-BIOMEMBR. (2008) 1778:2210-2216. Additionally, the lipid and PEG-lipid components used to prepare the lipid nanodisc particles segregate such that the PEG-lipid is enriched at the rim of the particle. Lipid nanodiscs can range in size from a few nanometers to hundreds of nanometers. In contrast, liposomes comprise a lipid bilayer and can also range in size. Liposomes that are multilamellar vesicles (MLVs) have a diameter of hundreds of nanometers and are formed from a series of concentric bilayers separated by narrow aqueous compartments. While unilamellar liposomes have a single lipid bilayer and are either small unicellular vesicles (SUVs) with diameter of approximately 50-100 nm, or large unilamellar vesicles (LUVs) with diameter of approximately 50 to 500 nm.

In some embodiments, the disclosure provides lipid nanodisc compositions, wherein the particle fraction that is lipid nanodiscs is at least 0.4, 0.45, 0.5, 0.55, 0.6, 0.65, 0.7, 0.75, 0.8, 0.85, or 0.9. Methods for measuring particle types present in the lipid nanodisc composition include cryo-transmission electron microscopy (cryo-TEM). For example, as described by Viitala, et al (2019) LANGMUIR 35:3999-4010, the fraction of the mixture that is lipid nanodiscs can be determined using the following formula:

$${x}_{\text{bicelle}}^{\text{TEM}} = \frac{{N}_{\text{bicelle}}}{{N}_{\text{bicelle}} + τ {N}_{\text{liposome}}} ,$$

wherein _{τ} is the area-scaling factor. The area-scaling factor is determined by the following formula:

$$τ = \frac{\left〈{Α}_{\text{liposome}}\right〉}{\left〈{Α}_{\text{bicelle}}\right〉} = 4 \frac{{\left〈{r}_{\text{liposome}}\right〉}^{2} + {\left(\left〈{r}_{\text{liposome}}\right〉 - d\right)}^{2}}{2 {\left〈{r}_{\text{bicelle}}\right〉}^{2} + π \left({r}_{\text{bicelle}}\right) d + {d}^{2}} ,$$

wherein d is the thickness of the bilayer, rₗᵢₚₒₛₒₘₑ and r_{bicelle} are the average radii of the liposome particles and lipid nanodisc particles respectively in the composition.

### Therapeutic Methods of Use

### I. Cancer and Cancer Immunotherapy

In some embodiments, the disclosure provides a STING agonist amphiphile conjugate or lipid composition thereof for use in treating a disorder associated with abnormal apoptosis or a differentiative process (e.g., cellular proliferative disorders (e.g., hyperproliferative disorders) or cellular differentiative disorders, such as cancer). In some embodiments, the lipid composition comprises a lipid particle comprising the STING agonist amphiphile conjugate. In some embodiments, the lipid particle is a lipid nanodisc described herein. In some embodiments, the disclosure provides a method for treating a disorder associated with abnormal apoptosis or a differentiative process (e.g., cellular proliferative disorders (e.g., hyperproliferative disorders) or cellular differentiative disorders, such as cancer) in a subject in need thereof, the method comprising administering a STING agonist amphiphile conjugate or lipid composition thereof described herein. In some embodiments, the disclosure provides a method for treating a disorder associated with abnormal apoptosis or a differentiative process (e.g., cellular proliferative disorders (e.g., hyperproliferative disorders) or cellular differentiative disorders, such as cancer) in a subject in need thereof, the method comprising administering a lipid composition comprising a lipid particle comprising a STING agonist amphiphile conjugate described herein. In some embodiments, the disclosure provides lipid nanodiscs or a composition thereof as described herein for use in treating a disorder associated with abnormal apoptosis or a differentiative process (e.g., cellular proliferative disorders (e.g., hyperproliferative disorders) or cellular differentiative disorders, such as cancer). Non-limiting examples of cancers that are amenable to treatment with the methods of the present disclosure are described below.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions used herein, comprising, e.g., a lipid nanodisc, can be administered to a patient who has cancer.

As used herein, the terms “cancer” (or “cancerous”), “hyperproliferative,” and “neoplastic” refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the lipid nanodiscs or a composition thereof are used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom’s macro globulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin’s disease and Reed-Sternberg disease.

It will be appreciated by those skilled in the art that amounts of the lipid nanodiscs or composition thereof that is sufficient to reduce tumor growth and diameter, or a therapeutically effective amount, will vary not only on the particular compounds (e.g., lipid nanodiscs) or compositions selected (e.g., a composition comprising lipid nanodiscs), but also with the route of administration, the nature of the condition being treated, and the age and condition of the patient, and will ultimately be at the discretion of the patient’s physician or pharmacist. In some embodiments, the amount of STING agonist amphiphile conjugate or lipid composition thereof that is sufficient to reduce tumor growth and diameter, or a therapeutically effective amount, is selected according to similar criteria, including e.g., the route of administration (e.g., systemic administration, parenteral administration, mucosal administration). The length of time during which the compositions used in the instant method will be given varies on an individual basis.

It will be appreciated by those skilled in the art that the colon adenocarcinoma MC38 tumor model and the B16 melanoma tumor model used herein are generalized models for solid tumors. That is, efficacy of treatments in these models is also predictive of efficacy of the treatments in other nonmelanoma or non-adenocarcinoma solid tumors. For example, as described in Baird et al. (J Immunology 2013; 190:469-78; Epub Dec. 7, 2012), efficacy of cps, a parasite strain that induces an adaptive immune response, in mediating anti-tumor immunity against B16F10 tumors was found to be generalizable to other solid tumors, including models of lung carcinoma and ovarian cancer. In another example, results from a line of research into VEGF-targeting lymphocytes also shows that results in B16F10 tumors were generalizable to the other tumor types studied (Chinnasamy et al., JCI 2010;120:3953-68; Chinnasamy et al., Clin Cancer Res 2012;18:1672-83). In yet another example, immunotherapy involving LAG-3 and PD-1 led to reduced tumor burden in a B16F10 tumor model, with generalizable results in a fibrosarcoma and colon adenocarcinoma cell lines (Woo et al., Cancer Res 2012;72:917-27).

In some embodiments, the disclosure provides a STING agonist amphiphile conjugate or lipid composition thereof for use in treating cancer in a subject in need thereof. In some embodiments, the disclosure provides a lipid composition comprising a lipid particle (e.g., lipid nanodisc) comprising the STING agonist amphiphile conjugate for use in treating cancer in a subject in need thereof. In some embodiments, the disclosure provides a method for treating cancer in a subject in need thereof, the method comprising administering a STING agonist amphiphile conjugate or lipid composition thereof described herein. In some embodiments, the disclosure provides a method for treating cancer in a subject in need thereof, the method comprising administering a lipid composition comprising a lipid particle comprising a STING agonist amphiphile conjugate described herein. In some embodiments, the disclosure provides lipid nanodiscs or a composition thereof for use in treating cancer in a subject in need thereof. In some embodiments, the lipid nanodiscs or a composition thereof are administered in combination with one or more additional therapies to treat cancer. In some embodiments, the lipid nanodiscs or a composition thereof are administered to treat melanoma, leukemia, lung cancer, breast cancer, prostate cancer, ovarian cancer, colon cancer, and brain cancer.

In some embodiments, the disclosure provides a STING agonist amphiphile conjugate or lipid composition thereof for use in inhibiting the growth and/or proliferation of tumor cells in a subject in need thereof. In some embodiments, the disclosure provides a lipid composition comprising a lipid particle (e.g., lipid nanodisc) comprising the STING agonist amphiphile conjugate for use in inhibiting the growth and/or proliferation of tumor cells in a subject in need thereof. In some embodiments, the disclosure provides a method for inhibiting the growth and/or proliferation of tumor cells in a subject in need thereof, the method comprising administering a STING agonist amphiphile conjugate or lipid composition thereof described herein. In some embodiments, the disclosure provides a method for inhibiting the growth and/or proliferation of tumor cells in a subject in need thereof, the method comprising administering a lipid composition comprising a lipid particle comprising a STING agonist amphiphile conjugate described herein. In some embodiments, administration of a lipid nanodisc of the disclosure or a composition thereof inhibits the growth and/or proliferation of tumor cells.

In some embodiments, the disclosure provides a STING agonist amphiphile conjugate or lipid composition thereof for use in reducing tumor diameter in a subject in need thereof. In some embodiments, the disclosure provides a lipid composition comprising a lipid particle (e.g., lipid nanodisc) comprising the STING agonist amphiphile conjugate for reducing tumor diameter in a subject in need thereof. In some embodiments, the disclosure provides a method for reducing tumor diameter in a subject in need thereof, the method comprising administering a STING agonist amphiphile conjugate or lipid composition thereof described herein. In some embodiments, the disclosure provides a method for reducing tumor diameter in a subject in need thereof, the method comprising administering a lipid composition comprising a lipid particle comprising a STING agonist amphiphile conjugate described herein. In some embodiments, administration of a lipid nanodisc of the disclosure or a composition thereof reduces tumor diameter.

In some embodiments, the disclosure provides a STING agonist amphiphile conjugate or lipid composition thereof for use in inhibiting metastases in a subject in need thereof. In some embodiments, the disclosure provides a lipid composition comprising a lipid particle (e.g., lipid nanodisc) comprising the STING agonist amphiphile conjugate for use in inhibiting metastases in a subject in need thereof. In some embodiments, the disclosure provides a method for inhibiting metastases in a subject in need thereof, the method comprising administering a STING agonist amphiphile conjugate or lipid composition thereof described herein. In some embodiments, the disclosure provides a method for inhibiting metastases in a subject in need thereof, the method comprising administering a lipid composition comprising a lipid particle comprising a STING agonist amphiphile conjugate described herein. In some embodiments, administration of a lipid nanodisc of the disclosure or a composition thereof inhibits metastases of a primary tumor.

It will be appreciated by those skilled in the art that reference herein to treatment extends to prophylaxis as well as the treatment of the noted cancers and symptoms.

In some embodiments, the disclosure provides a STING agonist amphiphile conjugate or lipid composition thereof for use in cancer immunotherapy in a subject in need thereof. In some embodiments, the disclosure provides a lipid composition comprising a lipid particle (e.g., lipid nanodisc) comprising the STING agonist amphiphile conjugate for use in cancer immunotherapy in a subject in need thereof. In some embodiments, the lipid nanodiscs or a composition thereof are used for cancer immunotherapy. The term “cancer immunotherapy” refers to treatment of a subject afflicted with, or at risk of suffering a recurrence of cancer, by a method comprising inducing, enhancing, suppressing, or otherwise modifying an immune response.

### Combination Therapy

In some embodiments, the disclosure provides a STING agonist amphiphile conjugate or lipid composition thereof for use as a monotherapy in a subject in need thereof. In some embodiments, the disclosure provides a lipid composition comprising a lipid particle (e.g., lipid nanodisc) comprising the STING agonist amphiphile conjugate for use as a monotherapy in a subject in need thereof. In some embodiments, the lipid nanodiscs or a composition thereof are administered to a subject as a monotherapy. In some embodiments, the disclosure provides a STING agonist amphiphile conjugate or lipid composition thereof for use in combination with one or more additional therapies. In some embodiments, the disclosure provides a lipid composition comprising a lipid particle (e.g., lipid nanodisc) comprising the STING agonist amphiphile conjugate for use in combination with one or more additional therapies. In some embodiments, the lipid nanodiscs or a composition thereof are used in combination with one or more additional therapies. As used herein, “combination therapy” embraces (i) administration of each agent or therapy in a sequential manner in a regiment that will provide beneficial effects of the combination, and (ii) co-administration of these agents or therapies in a substantially simultaneous manner, such as in a single capsule or infusion having a fixed ratio of these active agents or in multiple, separate capsules or infusions for each agent.

Combination therapy also includes combinations wherein individual agents may be administered at different times and/or by different routes but which act in combination to provide a beneficial effect by co-action or pharmacokinetic or pharmacodynamic effects of each agent or tumor treatment approaches of the combination therapy. For example, in some embodiments, the lipid nanodiscs or a composition thereof are used in combination with another immunotherapy. Exemplary immunotherapies include, but are not limited to, therapies (e.g., chemotherapy, e.g., radiotherapy) that induce tumor cell immunogenic cell death (immunogenic cell death inducers or ICD inducers) and immune checkpoint inhibitors.

### Immune Checkpoint Blockade

In some embodiments, the disclosure provides a STING agonist amphiphile conjugate or lipid composition thereof for use in conjunction with an immune checkpoint inhibitor or immune checkpoint blocker in a subject in need thereof. In some embodiments, the disclosure provides a lipid composition comprising a lipid particle (e.g., lipid nanodisc) comprising the STING agonist amphiphile conjugate for use in conjunction with an immune checkpoint inhibitor or immune checkpoint blocker in a subject in need thereof. In some embodiments, the lipid nanodiscs or a composition thereof as disclosed herein are used or administered in conjunction with immune checkpoint inhibitors or immune checkpoint blockers.

T cell activation and effector functions are balanced by co-stimulatory and inhibitory signals, referred to as “immune checkpoints.” Inhibitory ligands and receptors that regulate T cell effector functions are overexpressed on tumor cells. Subsequently, agonists of co-stimulatory receptors or antagonists of inhibitory signals, result in the amplification of antigen-specific T cell responses. In contrast to therapeutic antibodies which target tumor cells directly, an immune checkpoint inhibitor enhances endogenous anti-tumor activity. In certain embodiments, the immune checkpoint inhibitor suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardoll, D., Nature (2012), 12:252-264, 2012 and Wei, S. et. Al. Cancer Discovery (2018) 8:1069.

In some embodiments, the immune checkpoint inhibitor targets a component of the PD-1 signaling pathway. In some embodiments, the immune checkpoint inhibitor is an antibody or an antigen-binding portion thereof that disrupts the interaction between the PD-1 receptor and its ligand, PD-L1. Antibodies known in the art which bind to PD-1 or to its ligand PD-L1 and disrupt the interaction between PD-1 and PD-L1, and thereby stimulate an anti-tumor immune response, are suitable for use in the methods disclosed herein. Non-limiting examples of antibodies which bind to PD-1 or to PD-L1 include: nivolumab (OPDIVO® by Bristol-Myers Squibb); pembrolizumab (KEYTRUDA® by Merck), cemiplimab (LIBTAYO® by Sanofi and Regeneron Pharmaceuticals, Inc), avelumab (BAVENCIO® by Merck KGaA and Pfizer), durvalumab (IMFINZI® by AstraZeneca), atezolizumab (TECENTRIQ® by Genentech, Inc.), REGN2810 (in clinical trials, see e.g., clinicaltrials.gov identifiers NCT03409614, NCT03088540), BMS-936559 (Bristol-Myers Squibb, in clinical trials, see e.g., clinicaltrials.gov identifiers NCT01721746 and NCT01721772), SHR1210 (Alphamab and 3D Medicines, Incyte Biosciences and Jiangsu Hengrui medicine Corporation, in clinical trials, see e.g., clinicaltrials.gov identifiers NCT03134872 and NCT03427827), KN035 (in clinical trials, see e.g., clinicaltrials.gov identifier NCT02827968), IB1308 (Innovent Biologics Co, Ltd, in clinical trials, see e.g., clinicaltrials.gov identifier NCT03150875), PDR001 (Novartis Pharmaceuticals, in clinical trials, see e.g., clinicaltrials.gov identifier NCT02967692), BGB-A317 (BeiGene, in clinical trials, see e.g., clinicaltrials.gov identifiers NCT03358875, NCT03430843, NCT03412773), BCD-100 (Biocad, in clinical trials, see e.g., clinicaltrials.gov identifier NCT03288870), and JS001 (Shanghai Junshi Bioscience Co, Ltd., in clinical trials, see e.g., clinicaltrials.gov identifier NCT03430297). Additional examples include anti-PD-1 antibodies disclosed in U.S. Pat. No. 8,008,449, herein incorporated by reference and anti-PD-L1 antibodies disclosed in U.S. Pat. No. 7,943,743, herein incorporated by reference. It will be understood by one of ordinary skill that any antibody which binds to PD-1 or PD-L1, disrupts the PD-1/PD-L1 interaction, and stimulates an anti-tumor immune response, is suitable for use in the methods disclosed herein.

In some embodiments, the immune checkpoint inhibitor targets a component of the CTLA-4 signaling pathway. In some embodiments, the immune checkpoint inhibitor is an antibody or an antigen-binding portion thereof that targets CTLA-4 and disrupts its interaction with CD80 and CD86. Exemplary antibodies that target CTLA-4 include ipilimumab (YERVOY® by Bristol-Myers Squibb) and tremelimumab (formerly ticilimumab, CP-675,206 in development by MedImmune and AstraZeneca). Other suitable antibodies that target CTLA-4 are disclosed in WO 2012/120125, U.S. Pats. No. 6,984720, No. 6,682,7368, and U.S. Pat. Applications 2002/0039581, 2002/0086014, and 2005/0201994, herein incorporated by reference. It will be understood by one of ordinary skill that any antibody which binds to CTLA-4, disrupts its interaction with CD80 and CD86, and stimulates an anti-tumor immune response, is suitable for use in the methods disclosed herein.

### Immunogenic Cell Death Inducer

In some embodiments, the disclosure provides a STING agonist amphiphile conjugate or lipid composition thereof for use in conjunction with an immunogenic cell death inducer in a subject in need thereof. In some embodiments, the disclosure provides a lipid composition comprising a lipid particle (e.g., lipid nanodisc) comprising the STING agonist amphiphile conjugate for use in conjunction with an immunogenic cell death inducer in a subject in need thereof. In some embodiments, lipid nanodiscs or a composition thereof as disclosed herein are used or administered in combination with a therapy that induces immunogenic cell death of tumor cells. In some embodiments, the therapy comprises administration of a chemotherapeutic agent, wherein the chemotherapeutic agent induces tumor cell immunogenic cell death. In some embodiments, the therapy comprises administration of one or more doses of radiation, wherein the radiation induces tumor cell immunogenic cell death. In some embodiments, tumor cells undergoing immunogenic cell death promote an anti-tumor immune response sufficient to eradicate residual tumor cells.

Cell death can be classified according to the morphological appearance of the lethal process (that may be apoptotic, necrotic, autophagic, or associated with mitosis), enzymological criteria (with and without involvement of nucleases or certain classes or proteases, such as caspases), functional aspects (programmed or accidental, physiological, or pathological) or immunological characteristics (immunogenic or non-immunogenic) (see, e.g., Kroemer et al (2009) Nat Rev Immunol 9:353). Cell death that is “immunogenic” is defined in the art by the criteria that the dying cells, if uninfected and expressing a specific antigen (e.g., model antigen), are able to induce a protective immune response against the specific antigen when injected subcutaneously into a subject in the absence of any adjuvant (see, e.g., Casares et al (2005) J Exp Med 202:1691).

A typical method for assessing if an agent or therapy induces immunogenic cell death of tumor cells comprises (i) inducing cell death in a tumor cell sample using the agent or therapy (e.g., sample of mouse tumor cells such as CT26 cells), (ii) inoculating a syngeneic test subject with the dying cells of (i), (iii) inoculating the test subject at a different site with a tumorigenic dose of live tumor cells, (iv) comparing the size of the tumor in the test subject relative to a control subject that received (iii) only. In some embodiments, an agent or therapy induces immunogenic cell death of tumor cells if no or minimal tumor growth is detected in the test subject. Further assays and methods for assessing immunogenic cell death are described in WO2015/017313.

In some embodiments, a suitable therapy for inducing tumor cell immunogenic cell death is one that promotes or increases one or more hallmarks of immunogenic cell death, including, but not limited to, (i) surface exposure of calreticulin on tumor cells, (ii) secretion of ATP by tumor cells, (iii) secretion of HMGB1 by tumor cells, and/or (iv) exposure of heat shock protein by tumor cells.

In some embodiments, a lipid nanodisc composition of the disclosure is co-administered to a subject with cancer with a therapy that induces tumor cell immunogenic cell death, wherein the therapy is described in U.S. Pat. No. 8,828,944, which is hereby incorporated by reference in its entirety.

In some embodiments, a lipid nanodisc composition of the disclosure is co-administered to a subject with cancer with a chemotherapeutic agent that induces tumor cell immunogenic cell death. A suitable chemotherapeutic agent for inducing tumor cell immunogenic cell death is selected from, but not limited to, cyclophosphamide, an anthracycline, a platin, an oxaliplatin, a taxane, a cardiac glycoside, or an antimotic agent. In some embodiments, the anthracycline is selected from, but not limited to: doxorubicine, daunorubicin, epirubicin idarubicine, and mitoxantrone. In some embodiments, the platin is selected from, but not limited to: paclitaxel, cisplatin, carboplatin, or oxaliplatin. In some embodiments, the cardiac glycoside is selected from, but not limited to: digoxin, digitoxin, ouabain, and lanatoside C.

In some embodiments, a lipid nanodisc composition of the disclosure and the one or more additional active agents are administered at the same time. In other embodiments, the lipid nanodisc composition is administered first in time and the one or more additional active agents are administered second in time. In some embodiments, the one or more additional active agents are administered first in time and the lipid nanodisc composition is administered second in time.

A lipid nanodisc composition of the disclosure can replace or augment a previously or currently administered therapy. For example, upon treating with a lipid nanodisc composition, administration of the one or more additional active agents can cease or diminish, e.g., be administered at lower levels or dosages. In some embodiments, administration of the previous therapy can be maintained. In some embodiments, a previous therapy will be maintained until the level of the lipid nanodisc reaches a level sufficient to provide a therapeutic effect. The two therapies can be administered in combination.

### II. Infectious Disease

In some embodiments, the disclosure provides a STING agonist amphiphile conjugate or lipid composition thereof for use treating an acute or chronic infectious disease in a subject in need thereof. In some embodiments, the disclosure provides a lipid composition comprising a lipid particle (e.g., lipid nanodisc) comprising the STING agonist amphiphile conjugate for use in treating an acute or chronic infection disease in a subject in need thereof. In some embodiments, the disclosure provides a lipid nanodisc or composition thereof as described herein for use in treating acute or chronic infectious diseases. Because viral infections are cleared primarily by T-cells, an increase in T-cell activity is therapeutically useful in situations where more rapid or thorough clearance of an infective viral agent would be beneficial to an animal or human subject. Thus, in some embodiments the lipid nanodisc or composition thereof is administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. In some embodiments, pharmaceutical formulations including lipid nanodisc particles are administered topically to treat viral skin diseases such as herpes lesions or shingles, or genital warts. In some embodiments, the synthetic nanoparticles are administered to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoƒormans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni. In certain embodiments, the type of disease to be treated or prevented is a chronic infectious disease caused by a bacterium, virus, protozoan, helminth, or other microbial pathogen that enters intracellularly and is attacked, e.g., by cytotoxic T lymphocytes.

In certain embodiments, the type of disease to be treated or prevented is an infectious disease in the lungs caused by a bacterium. In certain embodiments, the lipid nanodisc composition inhibits biofilm formation of a bacterium. Inhibition of biofilm production by cyclic dinucleotides has been shown, for example, by Zogaj, X., et al., Infection and Immunity, Vol. 80(12): 4239-4247 (2012); and Yan, W., et al., Microbiological Research, Vol. 165: 87-96 (2010), each hereby incorporated by reference. Methods of measuring biofilm production are known in the art. For example, biofilm production can be measured by crystal violet staining and measurement of optical density of extracted crystal violet, or by the methods described in Ahn, S., et al., Journal of Bacteriology, Vol. 187 (9): 3028-3038 (2005), hereby incorporated by reference.

### III. Vaccines

In some embodiments, the disclosure provides a STING agonist amphiphile conjugate or lipid composition thereof for use in an immunogenic composition or as a component of a vaccine. In some embodiments, the disclosure provides a lipid composition comprising a lipid particle (e.g., lipid nanodisc) comprising the STING agonist amphiphile conjugate for use in an immunogenic composition or as a component of a vaccine. In some embodiments, the disclosure provides a lipid nanodisc or a composition thereof as disclosed herein for use in immunogenic compositions or as components of vaccines. In some embodiments, the disclosure provides an immunogenic composition comprising a STING agonist amphiphile conjugate described herein, and an antigen. In some embodiments, the disclosure provides an immunogenic composition comprising (i) a lipid composition described herein, the lipid composition comprising a STING agonist amphiphile conjugate of the disclosure, and (ii) an antigen. In some embodiments, the disclosure provides an immunogenic composition comprising (i) a lipid composition described herein, the lipid composition comprising a lipid particle (e.g., lipid nanodisc) comprising a STING agonist amphiphile conjugate of the disclosure, and (ii) an antigen In some embodiments, immunogenic compositions disclosed herein include (ii) a lipid nanodisc comprising a STING agonist amphiphile conjugate, a phospholipid and a PEG-lipid, (ii) an antigen, or (iii) a combination thereof.

In some embodiments, the disclosure provides a vaccine comprising a lipid composition described herein and an antigen described herein. In some embodiments, the lipid composition comprises a STING agonist amphiphile conjugate described herein. In some embodiments, the lipid composition comprises a lipid particle (e.g., lipid nanodisc) comprising a STING agonist amphiphile conjugate described herein. In some embodiments, the disclosure provides a vaccine comprising a lipid nanodisc comprising a STING agonist amphiphile conjugate described herein and an antigen described herein. In some embodiments, the vaccine is administered to a subject in need thereof, wherein the lipid composition and the antigen are co-administered. In some embodiments, the lipid composition and the antigen are co-administered in the same pharmaceutical composition. In some embodiments, the lipid composition and the antigen are separately administered.

In some embodiments, a vaccine is formed by combining lipid nanodiscs with an antigen. When administered to a subject in combination, the lipid nanodiscs and antigen can be administered in separate pharmaceutical compositions, or they can be administered together in the same pharmaceutical composition. When administered in combination, the antigen can be a lipid conjugate, optionally wherein the antigen is formulated in the lipid nanodiscs.

In some embodiments, an immunogenic composition comprises a lipid nanodisc administered alone, or in combination, with an antigen. Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof.

Suitable antigens are known in the art and are available from commercial government and scientific sources. In one embodiment, the antigens are whole inactivated or attenuated organisms. These organisms may be infectious organisms, such as viruses, parasites and bacteria. These organisms may also be tumor cells. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA

Antigens may be provided as single antigens or may be provided in combination. Antigens may also be provided as complex mixtures of polypeptides or nucleic acids. Exemplary antigens are provided below.

### (i) Peptide Antigens

In some embodiments, the disclosure provides an immunogenic composition or vaccine comprising (i) a STING agonist amphiphile conjugate or lipid composition thereof, and (ii) an antigen protein or polypeptide, such as a tumor-associated antigen or portion thereof. In some embodiments, the disclosure provides an immunogenic composition or vaccine comprising (i) a lipid composition comprising a lipid particle (e.g., lipid nanodisc) comprising the STING agonist amphiphile conjugate, and (ii) an antigen protein or polypeptide, such as a tumor-associated antigen or portion thereof. In some embodiments, an immunogenic composition comprises a lipid nanodisc and an antigenic protein or polypeptide, such as a tumor-associated antigen or portion thereof. In some embodiments, the antigenic protein or polypeptide is conjugated to a lipid (e.g., PEG-Lipid, e.g., phospholipid) of the lipid nanodisc. In some embodiments, the antigenic protein or polypeptide is administered in a separate immunogenic composition from that comprising the lipid nanodisc.

In some embodiments, the peptide is 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids. In some embodiments, a peptide is greater than 50 amino acids. In some embodiments, the peptide is >100 amino acids.

In some embodiments, a protein/peptide is linear, branched or cyclic. The peptide can include D amino acids, L amino acids, or a combination thereof. In some embodiments, the peptide or protein is conjugated to a lipid (e.g., PEG-Lipid, e.g., phospholipid) of the lipid nanodisc at either the N-terminus or the C-terminus of the peptide or protein.

In some embodiments, the protein or polypeptide is any protein or peptide that can induce or increase the ability of the immune system to develop antibodies and T-cell responses to the protein or peptide.

Suitable antigens are known in the art and are available from commercial government and scientific sources. In some embodiments, the antigens are whole inactivated or irradiated tumor cells. The antigens may be purified or partially purified polypeptides derived from tumors. In some embodiments, the antigens are recombinant polypeptides produced by expressing DNA encoding the polypeptide antigen in a heterologous expression system. In some embodiments, the antigens are DNA encoding all or part of an antigenic protein. The DNA may be in the form of vector DNA such as plasmid DNA.

In some embodiments, antigens are provided as single antigens or are provided in combination. In some embodiments, antigens are provided as complex mixtures of polypeptides or nucleic acids.

### (ii) Viral Antigens

In some embodiments, the disclosure provides an immunogenic composition or vaccine comprising (i) a STING agonist amphiphile conjugate or lipid composition thereof, and (ii) a viral antigen. In some embodiments, the disclosure provides an immunogenic composition or vaccine comprising (i) a lipid composition comprising a lipid particle (e.g., lipid nanodisc) comprising the STING agonist amphiphile conjugate, and (ii) a viral antigen. In some embodiments, an immunogenic composition comprises a lipid nanodisc and a viral antigen. In some embodiments, the viral antigen is administered in a separate immunogenic composition from that comprising the lipid nanodisc. In some embodiments, the viral antigen is isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxyiridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

In some embodiments, viral antigens are derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

### (iii) Bacterial Antigens

In some embodiments, the disclosure provides an immunogenic composition or vaccine comprising (i) a STING agonist amphiphile conjugate or lipid composition thereof, and (ii) a bacterial antigen. In some embodiments, the disclosure provides an immunogenic composition or vaccine comprising (i) a lipid composition comprising a lipid particle (e.g., lipid nanodisc) comprising the STING agonist amphiphile conjugate, and (ii) a bacterial antigen. In some embodiments, an immunogenic composition comprises a lipid nanodisc and a bacterial antigen. In some embodiments, the bacterial antigen is administered in a separate immunogenic composition from that comprising the lipid nanodisc. In some embodiments, the bacterial antigen originates from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

### (iv) Parasite Antigens

In some embodiments, the disclosure provides an immunogenic composition or vaccine comprising (i) a STING agonist amphiphile conjugate or lipid composition thereof, and (ii) a parasite antigen. In some embodiments, the disclosure provides an immunogenic composition or vaccine comprising (i) a lipid composition comprising a lipid particle (e.g., lipid nanodisc) comprising the STING agonist amphiphile conjugate, and (ii) a parasite antigen. In some embodiments, an immunogenic composition comprises a lipid nanodisc and a parasite antigen. In some embodiments, the parasite antigen is administered in a separate immunogenic composition from that comprising the lipid nanodisc. In some embodiments, parasite antigens are obtained from parasites such as, but not limited to, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

### (v) Allergens and Environmental Antigens

In some embodiments, the disclosure provides an immunogenic composition or vaccine comprising (i) a STING agonist amphiphile conjugate or lipid composition thereof, and (ii) an allergen or environmental antigen. In some embodiments, the disclosure provides an immunogenic composition or vaccine comprising (i) a lipid composition comprising a lipid particle (e.g., lipid nanodisc) comprising the STING agonist amphiphile conjugate, and (ii) an allergen or environmental antigen. In some embodiments, an immunogenic composition comprises a lipid nanodisc and an allergen or environmental antigen. In some embodiments, the allergen or environmental antigen is administered in a separate immunogenic composition from that comprising the lipid nanodisc. In some embodiments, the allergen or environmental antigen, is an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including e.g., grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

### (vi) Cancer Antigens

In some embodiments, the disclosure provides an immunogenic composition or vaccine comprising (i) a STING agonist amphiphile conjugate or lipid composition thereof, and (ii) a cancer antigen. In some embodiments, the disclosure provides an immunogenic composition or vaccine comprising (i) a lipid composition comprising a lipid particle (e.g., lipid nanodisc) comprising the STING agonist amphiphile conjugate, and (ii) a cancer antigen. In some embodiments, an immunogenic composition comprises a lipid nanodisc and a cancer antigen. In some embodiments, the cancer antigen is administered in a separate immunogenic composition from that comprising the lipid nanodisc. A cancer antigen is an antigen that is typically expressed preferentially by cancer cells (i.e., it is expressed at higher levels in cancer cells than on non-cancer cells) and in some instances it is expressed solely by cancer cells. In some embodiments, the cancer antigen is expressed within a cancer cell or on the surface of the cancer cell. In some embodiments, the cancer antigen is MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

### IV. Increasing an Immune Response

In some embodiments, the disclosure provides a STING agonist amphiphile conjugate or lipid composition thereof described herein for use in inducing, increasing, or enhancing an immune response in a subject in need thereof. In some embodiments, the disclosure provides a lipid composition comprising a lipid particle (e.g., lipid nanodisc) comprising a STING agonist amphiphile conjugate described herein for use in inducing, increasing, or enhancing an immune response in a subject in need thereof. In some embodiments, the disclosure provides a method for inducing, increasing, or enhancing an immune response in a subject in need thereof, the method comprising administering a STING agonist amphiphile conjugate or lipid composition thereof described herein. In some embodiments, the disclosure provides a method for inducing, increasing, or enhancing an immune response in a subject in need thereof, the method comprising administering a lipid composition comprising a lipid particle comprising a STING agonist amphiphile conjugate described herein. In some embodiments, the lipid nanodiscs or a composition thereof as described herein are administered in an effective amount to induce, increase or enhance an immune response. The “immune response” refers to responses that induce, increase, or perpetuate the activation or efficiency of an innate or adaptive immune response.

In some embodiments, the STING agonist amphiphile conjugate or lipid composition thereof is delivered parenterally through the lymphatics. In some embodiments, the STING agonist amphiphile conjugate or lipid composition thereof is administered at a site proximal or adjacent to the target tissue (e.g., tumor tissue). In some embodiments, the STING agonist amphiphile conjugate or lipid composition thereof is administered proximal or adjacent to the site in need of an immune response (e.g., close to a tumor or site of infection) or at a site leading to one or more lymph nodes that drains the site in need of an immune response (e.g., tumor draining lymph node; infection draining lymph node).

In some embodiments, the immune response is induced, increased, or enhanced by parenteral administration of the STING agonist amphiphile conjugate or lipid composition thereof described herein compared to a control, for example parenteral administration of the unconjugated STING agonist. In some embodiments, the immune response is induced, increased, or enhanced by parenteral administration of a lipid composition comprising a lipid particle (e.g., lipid nanodisc) comprising the STING agonist amphiphile conjugate described herein compared to a control, for example parenteral administration of the unconjugated STING agonist, the unformulated STING agonist amphiphile conjugate, or a control lipid formulation of the STING agonist amphiphile conjugate.

In some embodiments, the lipid nanodiscs or a composition thereof is delivered parenterally (e.g., by subcutaneous, intradermal, or intramuscular injection) through the lymphatics. In some embodiments, parenteral administration results in preferential distribution of the lipid nanodiscs to one or more lymph nodes that drain the site of injection, rather than systemic distribution. Accordingly, in some embodiments, the lipid nanodiscs or a composition thereof is administered at a site adjacent to or leading to one or more lymph nodes which is adjacent or near to a site in need of an immune response (i.e., close to a tumor or site of infection).

In some embodiments, the disclosure provides a STING agonist amphiphile conjugate or lipid composition thereof described herein for use in inducing, enhancing, or promoting an immune response following systemic administration through the circulatory system (e.g., by intravenous injection) to a subject in need thereof. In some embodiments, the disclosure provides a lipid composition comprising a lipid particle (e.g., lipid nanodisc) comprising a STING agonist amphiphile conjugate described herein for use in inducing, enhancing, or promoting an immune response following systemic administration through the circulatory system (e.g., by intravenous injection) to a subject in need thereof. In some embodiments, the lipid particle (e.g., lipid nanodisc) comprises one or more properties described herein (e.g., size, shape, deformability, aspect ratio) that provides preferential distribution of the STING agonist amphiphile conjugate to one or more target tissues wherein the immune response is desirable (e.g., tumor site, tumor draining lymphoid tissue). In some embodiments, a single dose of the STING agonist amphiphile conjugate or lipid composition thereof is sufficient to induce, promote, or enhance the immune response in the one or more target tissues wherein the immune response is desirable (e.g., a tumor site). In some embodiments, at least two doses of the STING agonist amphiphile conjugate or lipid composition thereof is used to induce, promote, or enhance the immune response in the one or more target tissues wherein the immune response is desirable (e.g., a tumor site).

In some embodiments, the lipid nanodiscs or a composition thereof is administered by systemic administration through the circulatory system (e.g., by intravenous injection). In some embodiments, systemic administration results in preferential distribution of the lipid nanodiscs to a one or more tissues wherein the immune response is desirable (e.g., a tumor site). In some embodiments, a single dose of the lipid nanodisc or composition thereof is sufficient to induce, promote, or enhance the immune response in the one or more target tissues wherein the immune response is desirable (e.g., a tumor site). In some embodiments, at least two doses of the lipid nanodisc or composition thereof are used to induce, promote, or enhance the immune response in the one or more target tissues wherein the immune response is desirable (e.g., a tumor site).

In some embodiments, the lipid nanodiscs or a composition thereof is administered in multiple doses at various locations throughout the body. In some embodiments, the lipid nanodiscs or a composition thereof is administered directly to a site in need of an immune response (e.g., a tumor or site of infection).

In some embodiments, the immune response is induced, increased, or enhanced by systemic administration of the STING agonist amphiphile conjugate or lipid composition thereof described herein compared to a control, for example systemic administration of the unconjugated STING agonist. In some embodiments, the immune response is induced, increased, or enhanced by systemic administration of a lipid composition comprising a lipid particle (e.g., lipid nanodisc) comprising the STING agonist amphiphile conjugate described herein compared to a control, for example systemic administration of the unconjugated STING agonist, the unformulated STING agonist amphiphile conjugate, or a control lipid formulation of the STING agonist amphiphile conjugate.

In some embodiments, the immune response is induced, increased, or enhanced by administration of the lipid nanodiscs compared to a control, for example an immune response in a subject induced, increased, or enhanced by administration of the STING agonist alone, or the STING agonist delivered using an alternative delivery strategy such as liposomes or spherical micelles. In some embodiments, administration of the lipid nanodiscs or a composition thereof promotes and/or prolongs activation of T cells, increases proliferation of antigen-specific T cells, enhances cytokine production by T cells, stimulates differentiation and effector functions of T cells and/or promote T cell survival. In some embodiments, administration of the lipid nanodiscs or a composition thereof overcomes T cell exhaustion and/or anergy.

In some embodiments, administration of the lipid nanodiscs or a composition thereof is used to induce an immune response, when administering the STING agonist alone, or the STING agonist in combination with an alternative delivery system (e.g., liposome, e.g., spherical micelle), is ineffectual. In some embodiments, administration of the lipid nanodiscs or a composition thereof is used to enhance or improve the immune response compared to administering the STING agonist alone. In some embodiments, administration of the lipid nanodiscs or a composition thereof reduces the dosage of STING agonist required to induce, increase, or enhance an immune response compared to administration of the STING agonist alone. In some embodiments, administration of the lipid nanodiscs or a composition thereof reduces the time needed for the immune system to respond to the STING agonist compared to administration of the STING agonist alone.

In some embodiments, administration of the lipid nanodiscs or a composition thereof induces, increases, or enhances immune cell response by (i) increasing serum half-life of STING agonist, (ii) increasing or promoting accumulation of STING agonist in tissues comprising the immune cells (e.g., tumor tissue, lymphoid tissue), (iii) increasing uptake of the STING agonist by the immune cells, (iv) increasing activation of STING in the immune cells, or (v) a combination of (i)-(iv).

In some embodiments, the STING agonist amphiphile conjugate or lipid composition thereof described herein is administered in an effective amount to induce or increase the activation of STING. In some embodiments, the lipid nanodiscs or a composition thereof is administered in an effective amount to induce or increase the activation of STING in a subject. As described herein, the STING signaling pathway in immune cells is a central mediator of innate immune response and when stimulated, induces expression of various interferons, cytokines and T cell recruitment factors that amplify and strengthen immune activity (e.g., anti-tumor immune activity). Accordingly, in some embodiments, the activation of STING by the lipid nanodiscs or a composition thereof, results in an induced or increased immune response.

In some embodiments, the STING agonist amphiphile conjugate or lipid composition thereof described herein is administered in an effective amount to induce an improved effector cell response, such as a CD4 T-cell and/or CD8 T-cell immune response, against an endogenous antigen (e.g., tumor antigen). In some embodiments, the STING agonist amphiphile conjugate or lipid composition thereof is administered with an exogenous antigen(s) or composition thereof to induce an improved effector cell response against the exogenous antigen. In some embodiments, the effector cell response is improved relative to administration of the unconjugated STING agonist or to administration of STING agonist delivered by an control lipid composition.

In some embodiments, the lipid nanodiscs or a composition thereof are administered to induce an improved effector cell response, such as a CD4 T-cell and/or CD8 T-cell immune response, against an endogenous antigen (e.g., tumor antigen). In some embodiments, the lipid nanodiscs or a composition thereof is administered with an exogenous antigen(s) or composition thereof to induce an improved effector cell response against the exogenous antigen. In some embodiments, the effector cell response is improved relative to administration of STING agonist alone or to administration of STING agonist delivered by an alternate platform (e.g., liposome). The term “improved effector cell response” refers to a heightened effector cell response, such as a CD8 or CD4 response, obtained in a human patient after administration of the lipid nanodiscs or a composition thereof compared to that obtained with administration of STING agonist alone or administration of the STING agonist delivered by an alternate platform (e.g., liposome).

In some embodiments, the lipid nanodiscs or a composition thereof is administered as part of a prophylactic vaccine or immunogenic composition which confer resistance in a subject to subsequent exposure to infectious agents, or as part of a therapeutic vaccine, which can be used to initiate or enhance a subject’s immune response to a pre-existing antigen, such as a viral antigen in a subject infected with a virus or a tumor antigen in a subject with cancer.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

In some embodiments, the improved effector cell response is obtained in an immunologically unprimed patient, i.e. a patient who is seronegative to the antigen. This seronegativity may be the result of the patient having never faced the antigen (so-called “naïve” patient) or, alternatively, having failed to respond to the antigen once encountered. In some embodiments, the improved effector cell response is obtained in an immunocompromised subject.

### V. Increasing STING Agonist Potency

In some embodiments, the disclosure provides a STING agonist amphiphile conjugate or lipid composition thereof for use in increasing or promoting the potency of the STING agonist in a subject following in vivo administration, e.g., as compared to in vivo administration of a control STING agonist (e.g., unconjugated or unformulated STING agonist). In some embodiments, the disclosure provides a lipid composition comprising a lipid particle (e.g., lipid nanodisc) comprising the STING agonist amphiphile conjugate for use in increasing or promoting the potency of the STING agonist in a subject following in vivo administration, e.g., as compared to in vivo administration of a control STING agonist (e.g., unformulated STING agonist).

In some embodiments, the disclosure provides a method for increasing or promoting the potency of a STING agonist in a subject, the method comprising administering to the subject a STING agonist amphiphile conjugate or lipid composition thereof described herein, thereby increasing the potency of the STING agonist in the subject, e.g., relative to a control (e.g., unformulated STING agonist, unconjugated STING agonist, or STING agonist formulated using a control lipid composition). In some embodiments, the disclosure provides a method of increasing or promoting potency of a STING agonist in a subject in vivo, comprising administering to the subject a lipid composition comprising a lipid particle (e.g., lipid nanodisc) comprising a STING agonist amphiphile conjugate described herein, such that potency of the STING agonist is increased relative to a control, e.g., an unconjugated STING agonist, an unformulated STING agonist, a control lipid composition comprising the STING agonist. In some embodiments, the composition increases or promotes the potency of the STING agonist in a subject by (i) increasing serum half-life of the STING agonist; (ii) increasing accumulation of STING agonist in a target tissue (e.g., tumor, tumor lymphoid tissue); (iii) increasing accumulation of STING agonist in a target cell population (e.g., tumor cell); (iv) increasing penetration of STING agonist in a target tissue (e.g., tumor, tumor lymphoid tissue); (v) increasing extravascular accumulation of STING agonist in a target tissue (e.g., tumor, tumor lymphoid tissue); and/or (vi) increasing a STING-induced immune response.

In some embodiments, the disclosure provides a STING agonist amphiphile conjugate or lipid composition thereof for use in increasing the serum half-life of a STING agonist in a subject in need thereof following in vivo administration. In some embodiments, the disclosure provides a lipid composition comprising a lipid particle (e.g., lipid nanodisc) comprising a STING agonist amphiphile conjugate described herein for use in increasing the serum half-life of a STING agonist in a subject in need thereof following in vivo administration. In some embodiments, the disclosure provides a method of increasing serum half-life of a STING agonist following in vivo administration comprising formulating a STING agonist amphiphile conjugate described herein in a lipid composition of the disclosure, and optionally administering the lipid composition to the subject. In some embodiments, the serum half-life of the STING agonist is increased in a subject following systemic administration of a lipid composition comprising a lipid particle (e.g., lipid nanodisc) comprising a STING agonist amphiphile conjugate described herein through the circulatory system (e.g., by intravenous injection). In some embodiments, the systemic administration of the lipid composition provides for a serum half-life of the STING agonist that is at least about 5, 6, 7, 8, 9, 10, 11, 12, 13, 14, 15, 16, 17, 18, 19, or 20 hours. In some embodiments, the systemic administration of the lipid composition provides for a serum half-life of the STING agonist that is about 5-10 hours, about 5-15 hours, about 5-20 hours, about 10-15 hours, about 10-20 hours. In some embodiments, the systemic administration of the lipid composition provides for a serum half-life of the STING agonist that is about 10 hours. In some embodiments, the systemic administration of the lipid composition provides for a serum half-life of the STING agonist that is about 11 hours. In some embodiments, the systemic administration of the lipid composition provides for a serum half-life of the STING agonist that is about 12 hours. In some embodiments, the systemic administration of the lipid composition provides for a serum half-life of the STING agonist that is about 13 hours. In some embodiments, the systemic administration of the lipid composition provides for a serum half-life of the STING agonist that is increased relative to systemic administration of a control STING agonist (e.g., unformulated STING agonist), e.g., by about 1.1, 1.2, 1.3, 1.4, 1.5, 1.6, 1.7, 1.8, 1.9, 2, 3, 4, 5, 6, 7, 8, 9, 10, 15, or 20-fold.

In some embodiments, the disclosure provides a STING agonist amphiphile conjugate or lipid composition thereof for use in increasing accumulation of STING agonist in a target tissue (e.g., tumor, tumor lymphoid tissue) in a subject following in vivo administration. In some embodiments, the disclosure provides a lipid composition comprising a lipid particle (e.g., lipid nanodisc) comprising a STING agonist amphiphile conjugate described herein for use in increasing accumulation of STING agonist in a target tissue (e.g., tumor, tumor lymphoid tissue) in a subject following in vivo administration. In some embodiments, the disclosure provides a method of increasing accumulation of a STING agonist in a target tissue in a subject, the method comprising administering a STING agonist amphiphile conjugate or lipid composition thereof described herein. In some embodiments, the disclosure provides a method of increasing accumulation of a STING agonist in a target tissue in a subject, the method comprising administering a lipid composition of the disclosure comprising a lipid particle (e.g. lipid nanodisc) comprising a STING agonist amphiphile conjugate. In some embodiments, the accumulation of STING agonist in a target tissue is increased following systemic administration of the composition through the circulatory system (e.g., by intravenous injection). In some embodiments, the systemic administration of the composition provides for accumulation of STING agonist in tumor tissue and/or tumor lymphoid tissue that is at least about 5%, 10%, 15%, 20%, 25%, 30%, 35%, 40%, 45%, 50%, 55%, 60%, 65%, 70%, 75%, or higher of the injected dose. In some embodiments, the accumulation of STING agonist in the target tissue is increased relative to systemic administration of a control STING agonist (e.g., unformulated STING agonist), e.g., by about 1.5, 2, 2.5, 3, 3.5, 4, 4.5, 5, 5.5, 6, 6.5, 7, 7.5, 8, 8.5, 9, 9.5, or 10-fold. In some embodiments, the accumulation of STING agonist in the target tissue is increased relative to systemic administration of a composition comprising a control lipid particle comprising the STING agonist amphiphile conjugate (e.g., non-lipid nanodisc, spherical particle, liposome), e.g., by about 1.5, 2, 2.5, 3, 3.5, 4, 4.5, 5, 5.5, 6, 6.5, 7, 7.5, 8, 8.5, 9, 9.5, or 10-fold.

In some embodiments, the disclosure provides a STING agonist amphiphile conjugate or lipid composition thereof for use in increasing accumulation of STING agonist in one or more target cell populations (e.g., tumor cell, tumor endothelial cell, immune cell) in a subject following in vivo administration. In some embodiments, the disclosure provides a lipid composition comprising a lipid particle (e.g., lipid nanodisc) comprising the STING agonist amphiphile conjugate for use in increasing accumulation of STING agonist in one or more target cell populations (e.g., tumor cell, tumor endothelial cell, immune cell) in a subject following in vivo administration. In some embodiments, the disclosure provides a method for increasing accumulation of STING agonist in one or more target cell populations (e.g., tumor cell, tumor endothelial cell, immune cell) in a subject, the method comprising administration of a STING agonist amphiphile conjugate or lipid conjugate thereof described herein. In some embodiments, the disclosure provides a method for increasing accumulation of STING agonist in one or more target cell populations (e.g., tumor cell, tumor endothelial cell, immune cell) in a subject, the method comprising administration of a lipid composition of the disclosure comprising a lipid particle (e.g., lipid nanodisc) comprising the STING agonist amphiphile conjugate. In some embodiments, the disclosure provides a method for increasing accumulation of STING agonist in one or more target cell populations (e.g., tumor cell, tumor endothelial cell, immune cell) in a subject, the method comprising formulating a STING agonist amphiphile conjugate described herein in a lipid composition, and administering the lipid composition to the subject. In some embodiments, the accumulation of STING agonist in one or more target cell populations is increased following systemic administration of the composition through the circulatory system (e.g., by intravenous injection). In some embodiments, the systemic administration of the composition provides for accumulation of STING agonist in one or more target cell populations selected from a population of tumor cell, endothelial cells, myeloid cells, and immune effector cells. In some embodiments the one or more target cell population is present in the tumor microenvironment and/or tumor lymphoid tissue. In some embodiments, the accumulation of STING agonist in the one or more target cell populations is increased relative to systemic administration of a control STING agonist (e.g., unformulated STING agonist), e.g., by about 1.5, 2, 2.5, 3, 3.5, 4, 4.5, 5, 5.5, 6, 6.5, 7, 7.5, 8, 8.5, 9, 9.5, or 10-fold. In some embodiments, the accumulation of STING agonist in the one or more target cell population is increased relative to systemic administration of a composition comprising a control lipid particle comprising the STING agonist amphiphile conjugate (e.g., non-lipid nanodisc, spherical particle, liposome), e.g., by about 1.5, 2, 2.5, 3, 3.5, 4, 4.5, 5, 5.5, 6, 6.5, 7, 7.5, 8, 8.5, 9, 9.5, or 10-fold.

In some embodiments, the disclosure provides a STING amphiphile lipid conjugate or lipid composition thereof for use in increasing penetration of the STING agonist in a target tissue (e.g., tumor, tumor lymphoid tissue) in a subject following in vivo administration. In some embodiments, the disclosure provides a lipid composition comprising a lipid particle (e.g., lipid nanodisc) comprising the STING agonist amphiphile conjugate for use in increasing penetration of the STING agonist in a target tissue (e.g., tumor, tumor lymphoid tissue) in a subject following in vivo administration. In some embodiments, the disclosure provides a method for increasing penetration of the STING agonist in a target tissue (e.g., tumor, tumor lymphoid tissue) in a subject, the method comprising administration of a STING agonist amphiphile conjugate or lipid composition described herein. In some embodiments, the disclosure provides a method for increasing penetration of the STING agonist in a target tissue (e.g., tumor, tumor lymphoid tissue) in a subject, the method comprising administration of a lipid composition of the disclosure comprising a lipid particle (e.g., lipid nanodisc) comprising the STING agonist amphiphile conjugate. In some embodiments, the penetration of STING agonist in the target tissue (e.g., tumor, tumor lymphoid tissue) is increased following systemic administration of the lipid composition through the circulatory system (e.g., by intravenous injection). In some embodiments, the penetration of STING agonist in the target tissue is measured by the accumulation of STING agonist in extravascular regions of the target tissue (e.g., tumor lymphoid tissue). In some embodiments, at least about 5%, 10%, 15%, 20%, 25%, 30%, 35%, 40%, 45%, 50%, or higher of the injected dose accumulates in extravascular regions of the target tissue (e.g., tumor, tumor lymphoid tissue). In some embodiments, the accumulation of STING agonist in the extravascular regions of the target tissue (e.g., tumor, tumor lymphoid tissue) is increased relative to systemic administration of a control STING agonist (e.g., unformulated STING agonist). In some embodiments, the accumulation of STING agonist in the extravascular regions of the target tissue (e.g., tumor, tumor lymphoid tissue) is increased relative to systemic administration of a composition comprising a control lipid particle comprising the STING agonist amphiphile conjugate (e.g., non-lipid nanodisc, spherical particle, liposome).

In some embodiments, the disclosure provides a method for increasing or promoting a STING-induced immune response in a subject, the method comprising a STING agonist amphiphile conjugate or lipid composition thereof described herein. In some embodiments, the disclosure provides a method for increasing or promoting a STING-induced immune response in a subject, the method comprising administration of a lipid composition comprising a lipid particle (e.g., lipid nanodisc) comprising a STING agonist amphiphile conjugate of the disclosure. In some embodiments, the disclosure provides a lipid composition of the disclosure for use in increasing or promoting a STING-induced immune response in a subject following in vivo administration.

As used herein, a “STING-induced immune response” refers to activation and/or triggering of a STING signaling pathway to induce, increases, or perpetuate the activation or efficiency of an innate and/or adaptive immune response. In some embodiments, the STING-induced immune response comprises: (i) increased production of STING-induced immunostimulatory cytokines (e.g., one or more type I interferons, TNFα), (ii) increased tumor necrosis, e.g., as measured by the abundance of necrotic tumor cells and/or tumor endothelial cells in the tumor microenvironment, (iii) increased uptake of tumor antigens by cross-presenting dendritic cells, (iv) increased trafficking of tumor antigen to tumor lymphoid tissue by cross-presenting dendritic cells, (v) increased presentation of tumor antigen by cross-presenting dendritic cells, (vi) increased activation of tumor-reactive CD8+ T cells, and/or (vii) increased tumor infiltration of immune effector cells (e.g., tumor reactive CD8+ T cells).

In some embodiments, the STING-induced immune response comprises increased production of one or more inflammatory cytokines, e.g., a type I interferon (e.g., IFN-alpha, IFN-beta), IL-6, TNF-alpha. In some embodiments, the STING-induced immune response comprises increased cell death of tumor cells and/or tumor endothelial cells. In some embodiments, the STING-induced immune response comprises increased tumor necrosis. In some embodiments, the STING-induced immune response comprises increased tumor antigen uptake and/or presentation by antigen presenting cells (e.g., cross-presenting dendritic cells). In some embodiments, the STING-induced immune response comprises increased trafficking of antigen presenting cells to lymphatic tissues. In some embodiments, the STING-induced immune response comprises increased infiltration of effector cells (e.g., cytotoxic T cells) into tumor and/or tumor lymphatic tissues. In some embodiments, the STING-induced immune response comprises increased proliferation of antigen-specific effector cells (e.g., cytotoxic T cells). In some embodiments, the STING-induced immune response comprises increased activation and/or effector function of antigen-specific T cells.

In some embodiments, systemic administration of the composition of lipid particles (e.g., lipid nanodiscs) provides increased production type I interferons (e.g., IFN-alpha, IFN-beta), e.g., as compared to systemic administration of a control STING agonist (e.g., unformulated STING agonist) or a composition comprising a control lipid particle comprising the STING agonist amphiphile conjugate (e.g., non-lipid nanodisc, spherical particle, liposome). In some embodiments, systemic administration of the composition of lipid particles (e.g., lipid nanodiscs) provides increased production of TNF-alpha, e.g., as compared to systemic administration of a control STING agonist (e.g., unformulated STING agonist) or a composition comprising a control lipid particle comprising the STING agonist amphiphile conjugate (e.g., non-lipid nanodisc, spherical particle, liposome). In some embodiments, systemic administration of the composition of lipid particles (e.g., lipid nanodiscs) provides increased production of IL-6, e.g., as compared to systemic administration of a control STING agonist (e.g., unformulated STING agonist) or a composition comprising a control lipid particle comprising the STING agonist amphiphile conjugate (e.g., non-lipid nanodisc, spherical particle, liposome).

In some embodiments, systemic administration of the composition of lipid particles (e.g., lipid nanodiscs) increases, promotes, or enhances the immune response of antigen presenting cells (e.g., cross-presenting cells), e.g., as compared to systemic administration of a control STING agonist (e.g., unformulated STING agonist) or a composition comprising a control lipid particle comprising the STING agonist amphiphile conjugate (e.g., non-lipid nanodisc, spherical particle, liposome). In some embodiments, the antigen presenting cells (e.g., cross-presenting dendritic cells) have increased uptake of tumor antigens, increased trafficking to tumor lymphatic tissue, and increased presentation to immune effector cells. In some embodiments, systemic administration of the composition of lipid particles (e.g., lipid nanodiscs) results in increased uptake of tumor antigen by antigen presenting cells (e.g., cross-presenting dendritic cells) in the tumor microenvironment. In some embodiments, systemic administration of the composition of lipid particles (e.g., lipid nanodiscs) results in increased uptake of STING agonist by antigen presenting cells (e.g., cross-presenting dendritic cells) in the tumor microenvironment

In some embodiments, systemic administration of the composition of lipid particles (e.g., lipid nanodiscs) increases, promotes, or enhances the response of immune effector cells (e.g., cytotoxic T cells), e.g., as compared to systemic administration of a control STING agonist (e.g., unformulated STING agonist) or a composition comprising a control lipid particle comprising the STING agonist amphiphile conjugate (e.g., non-lipid nanodisc, spherical particle, liposome). In some embodiments, the immune effector cells (e.g., cytotoxic T cells) have increased infiltration of tumor and/or tumor lymphoid tissue, e.g., as measured by histology or flow cytometry. In some embodiments, the immune effector cells (e.g., cytotoxic T cells) have increased survival, increased differentiation, increased effector function, and/or increased survival.

In some embodiments, systemic administration of the composition of lipid particles (e.g., lipid nanodiscs) results in increased tumor necrosis, e.g., as compared to systemic administration of a control STING agonist (e.g., unformulated STING agonist) or a composition comprising a control lipid particle comprising the STING agonist amphiphile conjugate (e.g., non-lipid nanodisc, spherical particle, liposome). In some embodiments, the increased tumor necrosis is measured by increased abundance of dead or dying tumor cells and/or tumor endothelial cells, e.g., using a method of measuring cell survival known in the art (e.g., flow cytometry, histology). Without being bound by theory, increased tumor necrosis provides tumor antigens for uptake and presentation by antigen presenting cells (e.g., cross-presenting dendritic cells) to induce activation of antigen-specific effector cells (e.g., cytotoxic T cells).

### Pharmaceutical Compositions and Modes of Administration

In some embodiments, the disclosure provides a pharmaceutical composition comprising a STING agonist amphiphile conjugate or lipid composition thereof, as described herein, and a pharmaceutically acceptable carrier. In some embodiments, the disclosure provides a pharmaceutical composition of a lipid composition described herein, wherein the lipid composition comprises a lipid particle (e.g., lipid nanodisc) comprising the STING agonist amphiphile conjugate described herein.

In certain embodiments, the disclosure provides for a pharmaceutical composition comprising lipid nanodiscs, as described herein, with a pharmaceutically acceptable diluents, carrier, solubilizer, emulsifier, preservative and/or adjuvant. In certain embodiments, the disclosure provides for a pharmaceutical composition comprising lipid nanodiscs and an antigen, with a pharmaceutically acceptable diluents, carrier, solubilizer, emulsifier, preservative and/or adjuvant.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen- sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta- cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington’s Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington’s Pharmaceutical Sciences, supra. In certain embodiments, such compositions may influence the physical state, stability, biophysical distribution, and rate of in vivo clearance of the lipid nanodiscs, with or without antigen.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising lipid nanodiscs, with or without antigen, can be prepared for storage by mixing the selected composition having the desired degree of purity with optional formulation agents (Remington’s Pharmaceutical Sciences, supra) in the form of a lyophilized cake or an aqueous solution. Further, in certain embodiments, a composition comprising lipid nanodiscs, with or without antigen, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising an amphiphile and immunomodulatory compound, or nanoparticle described herein, with or without antigen, in a pharmaceutically acceptable vehicle. In certain embodiments, a vehicle for parenteral injection is sterile distilled water in which the composition comprising lipid nanodiscs, with or without antigen, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, the composition comprising lipid nanodiscs, with or without antigen, can be formulated as a dry powder for inhalation. In certain embodiments, an inhalation solution comprising the lipid nanodiscs, with or without antigen, can be formulated with a propellant for aerosol delivery. In certain embodiments, solutions can be nebulized. Pulmonary administration is further described in PCT application No. PCT/US94/001875, which describes pulmonary delivery of chemically modified proteins.

In certain embodiments, it is contemplated that formulations can be administered orally. In certain embodiments, the lipid nanodiscs, with or without antigen, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of the lipid nanodiscs, with or without antigen. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of the lipid nanodiscs, with or without antigen, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving the lipid nanodiscs, with or without antigen, in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98- 105 (1982)), ethylene vinyl acetate (Langer et al., supra) or poly-D(-)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising the lipid nanodiscs, with or without antigen, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the lipid nanodiscs, with or without antigen, are being used, the route of administration, and the diameter (body weight, body surface or organ diameter) and/or condition (the age and general health) of the patient. In certain embodiments, the clinician can titer the dosage and modify the route of administration to obtain the optimal therapeutic effect.

In certain embodiments, the frequency of dosing will take into account the pharmacokinetic parameters of the lipid nanodiscs, with or without antigen, in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition of the lipid nanodiscs, with or without antigen, in an ex vivo manner. In such instances, cells, tissues and/or organs that have been removed from the patient are exposed to a pharmaceutical composition comprising the lipid nanodiscs, with or without antigen, after which the cells, tissues and/or organs are subsequently implanted back into the patient.

### Kits

In some embodiments, the disclosure provides a kit comprising a STING agonist amphiphile conjugate or lipid conjugate thereof, as described herein, and instructions for use. In some embodiments, the disclosure provides a kit comprising a lipid composition described herein, wherein the lipid composition comprises a lipid particle (e.g., lipid nanodisc) comprising a STING agonist amphiphile conjugate described herein, and instructions for use.

In certain embodiments, a kit can include lipid nanodiscs or a composition thereof, as described herein, and instructions for use. The kits may comprise, in a suitable container, lipid nanodiscs or a composition thereof, one or more controls, and various buffers, reagents, enzymes and other standard ingredients well known in the art. The container can include at least one vial, well, test tube, flask, bottle, syringe, or other container means, into which the lipid nanodiscs or a composition thereof may be placed, and in some instances, suitably aliquoted.

Where an additional component is provided, the kit can contain additional containers into which this component may be placed. In some embodiments, the kit further comprises one or more antigens. In some embodiments, the kit further comprises one or more immunogenic cell death inducers. In some embodiments, the kit further comprises one or more immune checkpoint inhibitors. In some embodiments, the kit comprises the lipid nanodisc or composition thereof and one or more additional agent(s). Accordingly, in some embodiments, the lipid nanodiscs or compositions thereof and the additional agent(s) are in the same vial. In some embodiments, the lipid nanodiscs or compositions thereof and the additional agent(s) are in separate vials. In some embodiments, the kit comprises the STING agonist amphiphile conjugate or lipid composition thereof, and one or more additional agent(s). In some embodiments, the STING agonist amphiphile conjugate or lipid composition thereof and the additional agent(s) are in separate vials. In some embodiments, the STING agonist amphiphile conjugate or lipid composition thereof and the additional agent(s) are in the same vial.

In some embodiments, the kit comprises the lipid nanodisc or composition thereof. The kits can also include a means for containing the lipid nanodiscs or a composition thereof and any other reagent containers in close confinement for commercial sale. In some embodiments, the kit comprises the STING agonist amphiphile conjugate or lipid composition thereof, wherein the STING agonist amphiphile conjugate or lipid composition thereof and any other reagent contains are provided in close confinement for commercial sale. Such containers may include injection or blow-molded plastic containers into which the desired vials are retained. Containers and/or kits can include labeling with instructions for use and/or warnings.

In some embodiments, the disclosure provides a kit comprising a container comprising STING agonist amphiphile conjugate or lipid composition thereof described herein, an optional pharmaceutically acceptable carrier, and a package insert comprising instructions for administration of the STING agonist amphiphile conjugate or lipid composition thereof for treating or delaying progression of cancer in a subject in need thereof. In some embodiments, the kit further comprises one or more antigens and instructions for administration of the antigen(s) for treating or delaying progression of cancer in a subject in need thereof. In some embodiments, the kit further comprises one or more immunogenic cell death inducers and instructions for administration of the immunogenic cell death inducer(s) for treating or delaying progression of cancer in a subject in need thereof. In some embodiments, the kit further comprises one or more immune checkpoint inhibitors and instructions for administration of the immune checkpoint inhibitor(s) for treating or delaying progression of cancer in a subject in need thereof.

In some embodiments, the disclosure provides a kit comprising a container comprising lipid nanodiscs or a composition thereof described herein, an optional pharmaceutically acceptable carrier, and a package insert comprising instructions for administration of the lipid nanodiscs or composition thereof for treating or delaying progression of cancer in a subject in need thereof. In some embodiments, the kit further comprises one or more antigens and instructions for administration of the antigen(s) for treating or delaying progression of cancer in a subject in need thereof. In some embodiments, the kit further comprises one or more immunogenic cell death inducers and instructions for administration of the immunogenic cell death inducer(s) for treating or delaying progression of cancer in a subject in need thereof. In some embodiments, the kit further comprises one or more immune checkpoint inhibitors and instructions for administration of the immune checkpoint inhibitor(s) for treating or delaying progression of cancer in a subject in need thereof.

In some embodiments, the disclosure provides a kit comprising a medicament comprising a STING agonist amphiphile conjugate or lipid composition thereof described herein, an optional pharmaceutically acceptable carrier, and a package insert comprising instructions for administration of the medicament alone or in combination with a composition comprising one or more antigens, immunogenic cell death inducers, or immune checkpoint inhibitors and an optional pharmaceutically acceptable carrier, for treating or delaying progression of cancer in a subject in need thereof.

In some embodiments, the disclosure provides a kit comprising a medicament comprising lipid nanodiscs or a composition thereof described herein, an optional pharmaceutically acceptable carrier, and a package insert comprising instructions for administration of the medicament alone or in combination with a composition comprising one or more antigens, immunogenic cell death inducers, or immune checkpoint inhibitors and an optional pharmaceutically acceptable carrier, for treating or delaying progression of cancer in a subject in need thereof.

In some embodiments, the disclosure provides a kit comprising a container comprising a STING agonist amphiphile conjugate or lipid composition thereof described herein, an optional pharmaceutically acceptable carrier, and a package insert comprising instructions for administration of the STING agonist amphiphile conjugate or lipid composition thereof for reducing or inhibiting tumor growth in a subject in need thereof. In some embodiments, the kit further comprises one or more antigens and instructions for administration of the antigen(s) for reducing or inhibiting tumor growth in a subject in need thereof. In some embodiments, the kit further comprises one or more immunogenic cell death inducers and instructions for administration of the immunogenic cell death inducer(s) for reducing or inhibiting tumor growth in a subject in need thereof. In some embodiments, the kit further comprises one or more immune checkpoint inhibitors and instructions for administration of the immune checkpoint inhibitor(s) for reducing or inhibiting tumor growth in a subject in need thereof.

In some embodiments, the disclosure provides a kit comprising a container comprising lipid nanodiscs or a composition thereof described herein, an optional pharmaceutically acceptable carrier, and a package insert comprising instructions for administration of the lipid nanodiscs or composition thereof for reducing or inhibiting tumor growth in a subject in need thereof. In some embodiments, the kit further comprises one or more antigens and instructions for administration of the antigen(s) for reducing or inhibiting tumor growth in a subject in need thereof. In some embodiments, the kit further comprises one or more immunogenic cell death inducers and instructions for administration of the immunogenic cell death inducer(s) for reducing or inhibiting tumor growth in a subject in need thereof. In some embodiments, the kit further comprises one or more immune checkpoint inhibitors and instructions for administration of the immune checkpoint inhibitor(s) for reducing or inhibiting tumor growth in a subject in need thereof.

In some embodiments, the disclosure provides a kit comprising a medicament comprising a STING agonist amphiphile conjugate or lipid composition thereof described herein, an optional pharmaceutically acceptable carrier, and a package insert comprising instructions for administration of the medicament alone or in combination with a composition comprising one or more antigens, immunogenic cell death inducers, or immune checkpoint inhibitors and an optional pharmaceutically acceptable carrier, for reducing or inhibiting tumor growth in a subject in need thereof.

In some embodiments, the disclosure provides a kit comprising a medicament comprising lipid nanodiscs or a composition thereof described herein, an optional pharmaceutically acceptable carrier, and a package insert comprising instructions for administration of the medicament alone or in combination with a composition comprising one or more antigens, immunogenic cell death inducers, or immune checkpoint inhibitors and an optional pharmaceutically acceptable carrier, for reducing or inhibiting tumor growth in a subject in need thereof.

### Definitions

Terms used in the claims and specification are defined as set forth below unless otherwise specified.

As used herein, a “lipid nanodisc” refers to lipid aggregates with disc-like morphology (e.g., discoidal micelle or disc-like micelle) comprising a PEG-lipid and a phospholipid, wherein the micellar particle is formed by self-assembly of the PEG-lipid and phospholipid. In some embodiments, the lipid nanodisc further comprises a STING agonist amphiphile conjugate.

As used herein, the term “STING agonist amphiphile conjugate” refers to an amphiphilic compound comprising a STING agonist covalently-linked to a polymer-modified lipid, optionally via a linker. In some embodiments, the STING agonist is a cyclic dinucleotide (CDN). In some embodiments, the STING agonist amphiphile conjugate is represented by the formula (XIV):

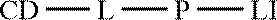

wherein CD is a compound represented by any one of Formula (XX) - (XXIX), L is a linker, P is a polymer, and LI is a diacyl lipid. In some embodiments, an exemplary STING agonist amphiphile conjugate is CDN-PEG-lipid. As used herein, the term “CDN-PEG-Lipid” refers to a compound having the following structure, comprising a CDN covalently-linked to a PEG-lipid via a dipeptide linker as shown in FIG. 1B, or a pharmaceutically acceptable salt thereof.

As used herein, the term “amphiphile” refers to a conjugate comprising a hydrophilic head group and a hydrophobic tail, thereby forming an amphiphilic conjugate. In some embodiments, an amphiphile comprises a conjugate comprising a STING agonist covalently-linked to a polymer-modified lipid, optionally via a lipid. In some embodiments, an amphiphile comprises a hydrophobic lipid tail and a hydrophilic polymer and/or STING agonist, thereby creating an amphiphilic molecule. The amphiphiles described herein are capable of self-assembly with one or more additional lipid components to form lipid particles, including lipid nanodisc particles.

As used herein, the terms “linked,” “fused,” “conjugated,” “conjugate” or “fusion,” in the context of joining together of two more elements or components or domains by suitable means including chemical conjugation are used interchangeably. Methods of chemical conjugation (e.g., using heterobifunctional crosslinking agents) are known in the art.

As used herein, the term “linker” refers to any chemical moiety capable of linking a polymer-modified lipid (e.g., a PEG-modified lipid) or a functional equivalent thereof to a STING agonist (e.g., a CDN, e.g., a compound represented by any one of Formula (XX) - (XXIX)). Linkers may be susceptible to cleavage (a “cleavable linker”) thereby facilitating release of the STING agonist or a cleavage product thereof. For example, such cleavable linkers may be susceptible to acid-induced cleavage, photo-induced cleavage, peptidase-induced cleavage, esterase-induced cleavage, and disulfide bond cleavage, at conditions under which the STING agonist and/or the polymer-modified lipid remains active. Alternatively, linkers may be substantially resistant to cleavage (a “noncleavable linker”).

As used herein, a “non-cleavable linker” refers to any chemical moiety capable of linking a STING agonist (e.g., a CDN, e.g., a compound represented by any one of Formula (XX) - (XXIX)) to a polymer-modified lipid (e.g., a PEG-modified lipid) or a functional equivalent thereof in a stable, covalent manner and does not fall off under the categories listed above for cleavable linkers. Thus, non-cleavable linkers are substantially resistant to acid-induced cleavage, photo-induced cleavage, peptidase-induced cleavage, esterase-induced cleavage and disulfide bond cleavage. Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which a STING agonist and/or the polymer-modified lipid does not lose its activity.

As used herein, the term “spacer” refers to a chemical moiety that connects two or more elements or components or domains. For example, in some embodiments, a spacer refers to a chemical moiety that connects the heterobi- and tri-functional group of a linker to the remaining portion of the linker. In some embodiments, a spacer refers to a chemical moiety that connects the polymer portion of a polymer-modified lipid to the lipid portion. In some embodiments, a spacer refers to a chemical moiety that connects the heterobi- and tri-functional group of a linker to the polymer portion of a polymer-modified lipid.

As used herein, the term “compound,” is meant to include all isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, “self-assembling” refers to spontaneous or induced assembly of a molecule into defined, stable, noncovalently bonded assemblies that are held together by intermolecular forces. Self-assembling molecules include protein, peptides, nucleic acids, virus-like particles, lipids and carbohydrates. In some embodiments, a STING agonist amphiphile conjugate self-assembles via noncovalent interactions with a PEG-lipid and a phospholipid, to form a lipid nanodisc.

As used herein, “nucleobase” refers to naturally occurring heterocyclic bases such as adenine, guanine, thymine, cytosine, and uracil, and also non-naturally occurring nucleobase analogs, homologs, and modified nucleobases such as those bearing removable protecting groups.

It must be noted that, as used in the specification and the appended claims, the singular forms “a,” “an” and “the” include plural referents unless the context clearly dictates otherwise.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C2-14 alkenyl” means an optionally substituted linear or branched hydrocarbon including 2-14 carbon atoms and at least one carbon-carbon double bond. An alkenyl group may include one, two, three, four, or more carbon-carbon double bonds. For example, C₁₈ alkenyl may include one or more double bonds. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C₁₈ alkynyl may include one or more carbon-carbon triple bonds. Unless otherwise specified, an alkynyl group described herein refers to both unsubstituted and substituted alkynyl groups.

As used herein, examples of the “halogen atom” includes fluorine, chlorine, bromine and iodine.

As used herein, examples of the “C₁₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

As used herein, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

As used herein, examples of the “C₃₋₁₀ cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

As used herein, examples of the “optionally halogenated C₃₋₁₀ cycloalkyl group” include a C₃₋₁₀ cycloalkyl group optionally having 1 to 7, preferably 1 to 5, halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

As used herein, examples of the “C₃₋₁₀ cycloalkenyl group” include cyclopropenyl, cyclobutenyl, cyclopentenyl, cyclohexenyl, cycloheptenyl and cyclooctenyl.

As used herein, examples of the “C₆₋₁₄ aryl group” include phenyl, 1-naphthyl, 2-naphthyl, 1-anthryl, 2-anthryl and 9-anthryl.

As used herein, examples of the “C₇₋₁₆ aralkyl group” include benzyl, phenethyl, naphthylmethyl and phenylpropyl.

As used herein, examples of the “C₁₋₆ alkoxy group” include methoxy, ethoxy, propoxy, isopropoxy, butoxy, isobutoxy, sec-butoxy, tert-butoxy, pentyloxy and hexyloxy.

As used herein, examples of the “optionally halogenated C₁₋₆ alkoxy group” include a C₁₋₆ alkoxy group optionally having 1 to 7, preferably 1 to 5, halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

As used herein, examples of the “C₃₋₁₀ cycloalkyloxy group” include cyclopropyloxy, cyclobutyloxy, cyclopentyloxy, cyclohexyloxy, cycloheptyloxy and cyclooctyloxy.

As used herein, examples of the “C₁₋₆ alkylthio group” include methylthio, ethylthio, propylthio, isopropylthio, butylthio, sec-butylthio, tert-butylthio, pentylthio and hexylthio.

As used herein, examples of the “optionally halogenated C₁₋₆ alkylthio group” include a C₁₋₆ alkylthio group optionally having 1 to 7, preferably 1 to 5, halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

As used herein, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

As used herein, examples of the “optionally halogenated C₁₋₆ alkyl-carbonyl group” include a C₁₋₆ alkyl-carbonyl group optionally having 1 to 7, preferably 1 to 5, halogen atoms. Specific examples thereof include acetyl, chloroacetyl, trifluoroacetyl, trichloroacetyl, propanoyl, butanoyl, pentanoyl and hexanoyl.

As used herein, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

As used herein, examples of the “C₆₋₁₄ aryl-carbonyl group” include benzoyl, 1-naphthoyl and 2-naphthoyl.

As used herein, examples of the “C₇₋₁₆ aralkyl-carbonyl group” include phenylacetyl and phenylpropionyl.

As used herein, examples of the “5- to 14-membered aromatic heterocyclylcarbonyl group” include nicotinoyl, isonicotinoyl, thenoyl and furoyl.

As used herein, examples of the “3- to 14-membered non-aromatic heterocyclylcarbonyl group” include morpholinylcarbonyl, piperidinylcarbonyl and pyrrolidinylcarbonyl.

As used herein, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

As used herein, examples of the “mono- or di-C₇₋₁₆ aralkyl-carbamoyl group” include benzylcarbamoyl and phenethylcarbamoyl.

As used herein, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

As used herein, examples of the “optionally halogenated C₁₋₆ alkylsulfonyl group” include a C₁₋₆ alkylsulfonyl group optionally having 1 to 7, preferably 1 to 5, halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

As used herein, examples of the “C₆₋₁₄ arylsulfonyl group” include phenylsulfonyl, 1-naphthylsulfonyl and 2-naphthylsulfonyl.

As used herein, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

As used herein, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

As used herein, examples of the “optionally substituted hydrocarbon group” include a hydrocarbon group optionally having substituent(s) selected from the following Substituent group A.

“Substituent group A:”
(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl)(C₁₋₆ alkyl-carbonyl) amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group, and
(62) a C₆₋₁₄ aryl group.

The number of the above-mentioned substituents in the “optionally substituted hydrocarbon group” is, for example, 1 to 5, preferably 1 to 3. When the number of the substituents is two or more, the respective substituents may be the same or different.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 heteroatoms selected from a nitrogen atom, a sulfur atom and an oxygen atom.

As used herein, examples of the “aromatic heterocyclic group” (including “5- to 14-membered aromatic heterocyclic group”) include a 5- to 14-membered (preferably 5- to 10-membered) aromatic heterocyclic group containing, as a ring-constituting atom besides carbon atom, 1 to 4 heteroatoms selected from a nitrogen atom, a sulfur atom and an oxygen atom.

Preferable examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and 8- to 14-membered fused polycyclic (preferably bi or tricyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, β-carbolinyl, phenanthridinyl, acridinyl, phenazinyl, phenothiazinyl, phenoxazinyl and the like.

As used herein, examples of the “non-aromatic heterocyclic group” (including “3- to 14-membered non-aromatic heterocyclic group”) include a 3- to 14-membered (preferably 4- to 10-membered) non-aromatic heterocyclic group containing, as a ring-constituting atom besides carbon atom, 1 to 4 heteroatoms selected from a nitrogen atom, a sulfur atom and an oxygen atom.

Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (preferably bi or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

As used herein, preferable examples of the “7- to 10-membered bridged heterocyclic group” include quinuclidinyl and 7-azabicyclo[2.2.1]heptanyl.

As used herein, examples of the “nitrogen-containing heterocyclic group” include a “heterocyclic group” containing at least one nitrogen atom as a ring-constituting atom.

As used herein, examples of the “optionally substituted heterocyclic group” include a heterocyclic group optionally having substituent(s) selected from the above-mentioned “Substituent group A.

The number of the substituents in the “optionally substituted heterocyclic group” is, for example, 1 to 3. When the number of the substituents is two or more, the respective substituents may be the same or different.

As used herein, examples of the “acyl group” include a formyl group, a carboxy group, a carbamoyl group, a thiocarbamoyl group, a sulfino group, a sulfo group, a sulfamoyl group and a phosphono group, each optionally having 1 or 2 substituents selected from a C₁₋₆ alkyl group, a C₁₋₃₀ alkyl group, a C₂₋₆ alkenyl group, a C₂₋₃₀ alkenyl group, a C₃₋₁₀ cycloalkyl group, a C₃₋₁₀ cycloalkenyl group, a C₆₋₁₄ aryl group, a C₇₋₁₆ aralkyl group, a C₂₋₃₀ alkynyl group, a 5- to 14-membered aromatic heterocyclic group and a 3- to 14-membered non-aromatic heterocyclic group, each of which is optionally substituted (e.g., optionally has 1 to 3 substituents, e.g., optionally has 1 to 10 substituents, e.g., optionally has 1 to 20 substituents) selected from a halogen atom, an optionally halogenated C₁₋₆ alkoxy group, a hydroxy group, a nitro group, a cyano group, an amino group and a carbamoyl group.

Examples of the “acyl group” also include a hydrocarbon-sulfonyl group, a heterocyclylsulfonyl group, a hydrocarbon-sulfinyl group and a heterocyclylsulfinyl group.

Here, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

As used herein, examples of the “optionally substituted amino group” include an amino group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5-to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from “Substituent group A”. Preferable examples of the optionally substituted amino group include an amino group, a mono- or di-(optionally halogenated C₁₋₆ alkyl) amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆ alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl) amino group (e.g., methylcarbamoylamino), a (mono- or di-C₇₋₁₆ aralkyl-carbamoyl) amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl)(C₁₋₆ alkyl-carbonyl) amino group (e.g., N-acetyl-N-methylamino) and a (C₁₋₆ alkyl)(C₆₋₁₄ aryl-carbonyl) amino group (e.g., N-benzoyl-N-methylamino).

As used herein, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5-to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from “Substituent group A”.

Preferable examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ aryl-carbonyl-carbamoyl group (e.g., benzoylcarbamoyl) and a 5- to 14-membered aromatic heterocyclylcarbamoyl group (e.g., pyridylcarbamoyl).

As used herein, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋ ₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from “Substituent group A”.

Preferable examples of the optionally substituted thiocarbamoyl group include a thiocarbamoyl group, a mono- or di-C₁₋₆ alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

As used herein, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5-to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from “Substituent group A”.

Preferable examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or di-C₁₋₆ alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

As used herein, examples of the “optionally substituted hydroxy group” include a hydroxy group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from “Substituent group A”.

Preferable examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋ ₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇₋₁₆ aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

As used herein, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from “Substituent group A” and a halogenated sulfanyl group.

Preferable examples of the optionally substituted sulfanyl group include a sulfanyl (-SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

As used herein, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from “Substituent group A”. Examples of the optionally substituted silyl group include a tri-C₁₋₆ alkylsilyl group (e.g., trimethylsilyl, tert-butyl(dimethyl)silyl).

As used herein, “vaccine” refers to a formulation which contains a lipid nanodisc as described herein, combined with an antigen, which is in a form that is capable of being administered to a vertebrate and which induces a protective immune response sufficient to induce immunity to prevent and/or ameliorate an infection or disease and/or to reduce at least one symptom of an infection or disease. Typically, the vaccine comprises a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine provokes an immune response including, but not limited to, the production of antibodies and/or cytokines and/or the activation of cytotoxic T cells, antigen presenting cells, helper T cells, dendritic cells and/or other cellular responses.

In certain embodiments, the vaccine is a “cancer vaccine,” which refers to a treatment that induces the immune system to attack cells with one or more tumor associated antigens. The vaccine can treat existing cancer (e.g., therapeutic cancer vaccine) or prevent the development of cancer in certain individuals (e.g., prophylactic cancer vaccine). The vaccine creates memory cells that will recognize tumor cells with the antigen and therefore prevent tumor growth. In certain embodiments, the cancer vaccine comprises a lipid nanodisc or composition thereof as described herein, and a tumor-associated antigen.

As used herein, the term “immunogen” or “antigen” refers to a substance such as a protein, peptide, or nucleic acid that is capable of eliciting an immune response. Both terms also encompass epitopes, and are used interchangeably.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, a “Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, a “Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “infectious agent” refers to microorganisms that cause an infection in a vertebrate. Usually, the organisms are viruses, bacteria, parasites, protozoa and/or fungi.

As used herein, the term “antigenic formulation” or “antigenic composition” or “immunogenic composition” refers to a preparation which, when administered to a vertebrate, especially a mammal, will induce an immune response.

The term “ameliorating” refers to any therapeutically beneficial result in the treatment of a disease state, e.g., cancer, including prophylaxis, lessening in the severity or progression, remission, or cure thereof.

The term “in vivo” refers to processes that occur in a living organism.

The term “mammal” or “subject” or “patient” as used herein includes both humans and non-humans and includes, but is not limited to, humans, non-human primates, canines, felines, murines, bovines, equines, and porcines.

As used herein, “cancer antigen” refers to (i) tumor- specific antigens, (ii) tumor- associated antigens, (iii) cells that express tumor- specific antigens, (iv) cells that express tumor- associated antigens, (v) embryonic antigens on tumors, (vi) autologous tumor cells, (vii) tumor- specific membrane antigens, (viii) tumor- associated membrane antigens, (ix) growth factor receptors, (x) growth factor ligands, and (xi) any other type of antigen or antigen-presenting cell or material that is associated with a cancer.

As used herein, the term “sufficient amount” or “amount sufficient to” means an amount sufficient to produce a desired effect, e.g., an amount sufficient to reduce the diameter of a tumor.

As used herein, the term “therapeutically effective amount” is an amount that is effective to ameliorate a symptom of a disease. A therapeutically effective amount can be a “prophylactically effective amount” as prophylaxis can be considered therapy.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

As used herein, the term “T cell” refers to a CD4+ T cell or a CD8+ T cell. The term T cell encompasses TH1 cells, TH2 cells and TH17 cells.

As used herein, the term “T cell cytotoxicity” includes any immune response that is mediated by CD8+ T cell activation. Exemplary immune responses include cytokine production, CD8+ T cell proliferation, granzyme or perforin production, and clearance of an infectious agent.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the terms “approximately” and “about,” as applied to one or more values of interest, refer to a value that is similar to a stated reference value. In some embodiments, “approximately” or “about” refers to a range of values within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, in some embodiments, when used in the context of an amount of a given component for use in a lipid nanodisc formulation disclosed herein, “about” or “approximately” means ±10% of the recited value. For example, a lipid nanodisc composition including about 10 mole percent of a STING agonist amphiphile conjugate (e.g., CDN-PEG-Lipid) may be interpreted as a composition including 9 to 11 mole percent of the STING agonist amphiphile conjugate.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Preparation of Amphiphilic CDN-PEG-Lipid Conjugates

To enable systemic administration of CDNs, which are hydrophilic small molecules that agonize STING (Stimulator of Interferon Genes) and have a short serum half-life, a CDN amphiphilic conjugate was generated for formulation in lipid-based particles (e.g., lipid nanodisc (LND), liposome, micelle). Specifically, a CDN-PEG-Lipid amphiphile conjugate was prepared by first generating a CDN-Linker as shown in FIG. 1A. A parent CDN compound was synthesized and conjugated to a cleavable alanine-alanine dipeptide linker (“Linker”) to yield the CDN-Linker compound. The CDN-Linker compound was used to covalently link the CDN to a PEG2k-lipid, resulting in the CDN-PEG-Lipid amphiphile conjugate shown in FIG. 1B. The resulting CDN-PEG-lipid was designed to facilitate formulation in lipid-based drug carriers, with release of the active STING agonist (parent CDN shown in FIG. 1A) upon peptidase cleavage in endosomes following cellular uptake (see, e.g., Miller, M. L. et al. Mol Cancer Ther 17, molcanther.0940.2017 (2018)).

Methods for preparing the parent CDN and its covalent attachment to a linker to generate a CDN-linker compound are described in PCT Publication No. WO/2018/100558, the entire contents of which are incorporated herein by reference.

### Preparation of Parent CDN

2-amino((5R,7R,8R,12aR,14R,15R,15aS,18R)-14-(6-amino-9H-purinyl)-10,18-dihydroxy-2,10-oxido-2-sulfanylhexahydro-14H-15,12a-(epoxymethano)-5,8-methanofuro[3,2-1][1,3,6,9,11,2,10]pentaoxadiphosphacyclotetradecin-7(12H)-yl)-1,9-dihydro-6H-purin-6-onesesqui-triethylamine salt (optical isomer; parent CDN) was generated according to the following procedure.

### (A) (1R,3R,4R, 7S)-3-(6-benzamido-9H-purin-9-yl)-1-((((((2R,3R,4R,5R)-4-((tert-butyldimethylsilyl)oxy)-5-(hydroxymethyl)-2-(2-isobutylamido-6-oxo-1H-purin-9(6H)-yl)tetrahydrofuran-3-yl)oxy)(2-cyanoethoxy)phosphoryl)oxy)methyl)-2,5-dioxabicyclo[2.2.1]heptan-7-yl Hydrogen Phosphonate

(1S,3R,4R,7S)-3-(6-Benzamido-9H-purin-9-yl)-1-(hydroxymethyl)-2,5-dioxabicyclo[2.2.1]heptan-7-yl hydrogen phosphonate (700 mg) and 5′-O-[bis(4-methoxyphenyl)(phenyl)methyl]-3′-O-[tert-butyl(dimethyl)silyl]-2′-O-{ (2-cyanoethoxy)[diisopropylamino]phosphanyl)-N-(2-methylpropanoyl)guanosine (2280 mg) were subjected to azeotropic dehydration with anhydrous acetonitrile, and anhydrous acetonitrile (15 mL) and anhydrous tetrahydrofuran (5 mL) were added thereto. To the mixture was added a mixture of 5-(ethylsulfanyl)-2H-tetrazole (611 mg) (which was subjected to azeotropic dehydration with anhydrous acetonitrile) and anhydrous acetonitrile (10 mL), and the mixture was stirred overnight under argon atmosphere at room temperature. 70% tert-Butyl hydroperoxide (643 µL) was added thereto, and the mixture was stirred at room temperature for 20 min. To the reaction mixture was added a mixture of sodium thiosulfate (5920 mg) and water (3 mL), and the mixture was concentrated under reduced pressure. To the residue was added 80% acetic acid (30 mL), and the mixture was stirred at room temperature for 20 min. The reaction mixture was concentrated under reduced pressure, and the residue was purified by silica gel column chromatography (methanol/ethyl acetate) to give the title compound (980 mg). MS: [M+H]⁺ 1030.2

### (B) 2-amino-9-[(5R,7R,8R,12aR,14R,15R,15aS,18R)-14-(6-amino-9H-purin-9-yl)-18-[[tert-butyl(dimethyl)silyl]oxy}-2,10-dihydroxy-10-oxido-2-sulfidohexahydro-14H-15,12a-(epoxymethano)-5,8-methanofuro[3,2-1][1,3,6,9,11,2,10]pentaoxadiphosphacyclotetradecin-7(12H)-yl)-1,9-dihydro-6H-purin-6-one (Optical Isomer)

(1R,3R,4R,7S)-3-(6-Benzamido-9H-purin-9-yl)-1-((((((2R,3R,4R,5R)-4-((tertbutyldimethylsilyl)oxy)-5-(hydroxymethyl)-2-(2-isobutylamido-6-oxo-1H-purin-9(6H)-yl)tetrahydrofuran-3-yl)oxy)(2-cyanoethoxy)phosphoryl)oxy)methyl)-2,5-dioxabicyclo[2.2.1]heptan-7-yl hydrogen phosphonate (980 mg) was subjected to azeotropic dehydration with anhydrous acetonitrile and anhydrous pyridine, and anhydrous pyridine (50 mL) was added thereto. To the mixture was added 2-chloro-5,5-dimethyl-1,3,2-dioxaphosphinane 2-oxide (615 mg) at room temperature, and the mixture was stirred under argon atmosphere at room temperature for 1 hour. Water (600 µL) and 3H-benzo[c][1,2]dithiol-3-one (240 mg) were added thereto, and the mixture was stirred at room temperature for additional 30 minutes. To the reaction mixture was added a mixture of sodium thiosulfate (1180 mg) and water (3 mL), and the mixture was concentrated under reduced pressure. To the residue were added anhydrous acetonitrile (15 mL) and 2-methylpropan-2-amine (5.0 mL), and the mixture was stirred at room temperature for 1.5 hours, and concentrated under reduced pressure. The residue was purified by silica gel column chromatography (methanol/ethyl acetate), and to the obtained residue was added 40% methylamine ethanol solution (30 mL). The mixture was stirred overnight under argon atmosphere at room temperature, and the reaction mixture was concentrated under reduced pressure. The residue was purified by silica gel column chromatography (methanol/ethyl acetate). The obtained residue was resolved into two diastereomers (tR1 and tR2, retention times of which by LC/MS are from shorter to longer in this order) by HPLC (L-column2 ODS, 50x150 mm, mobile phase: 5 mM aqueous ammonium acetate solution/acetonitrile) to give the title compound (38 mg, tR1) and the title compound (322 mg, tR2). MS (tR1): [M+H]⁺ 817.1. MS (tR2): [M+14]⁺ 817.1

### (C) 2-amino-9-[(5R,7R,8R,12aR,14R,15R,15aS,18R)-14-(6-amino-9H-purin-9-yl)-2,10,18-trihydroxy-10-oxido-2-sulfidohexahydro-14H-15,12a-(epoxymethano)-5,8-methanofuro[3,2-1][1,3,6,9,11,2,10]pentaoxadiphosphacyclotetradecin-7(12H)-yl]-1,9-dihydro-6H-purin-6-one sesqui-triethylamine Salt (Optical Isomer)

To 2-amino-9-[(5R,7R,8R,12aR,14R,15R,15aS,18R)-14-(6-amino-9H-purin-9-yl)-18-{[tert-butyl(dimethyl)silyl]oxy}-2,10-dihydroxy-10-oxido-2-sulfidohexahydro-14H-15,12a-(epoxymethano)-5,8-methanofuro[3,2-l][1,3,6,9,11,2,10]pentaoxadiphosphacyclotetradecin-7(12H)-yl]-1,9-dihydro-6H-purin-6-one (optical isomer) (38 mg, tR1) were added methanol (3.0 mL) and triethylamine trihydrofluoride (0.76 mL). The reaction mixture was concentrated to remove the methanol, and the residue was stirred at 55° C. for 1 hour. The mixture was cooled to room temperature, ethoxy(trimethyl)silane (4.2 mL) was added thereto, and the mixture was stirred at room temperature for 2 hours. The reaction mixture was concentrated under reduced pressure, and the residue was purified by C18 column chromatography (acetonitrile/10 mM triethylammonium acetate buffer solution) to give the title compound (27 mg). ¹H NMR (400 MHz, D₂O) δ 1.23 (13H, t, J = 7.3 Hz), 3.15 (9H, q, J = 7.3 Hz), 4.04 (1H, d, J = 8.3 Hz), 4.08-4.19 (3H, m), 4.28 (1H, d, J = 12.2 Hz), 4.37-4.52 (2H, m), 4.65 (1H, d, J = 4.2 Hz), 4.90 (1H, d, J = 4.6 Hz), 5.36 (1H, s), 5.55 (1H, td, J = 8.5,4.0 Hz), 5.98 (1H, d, J = 8.3 Hz), 6.16 (1H, s), 7.94 (1H, s), 8.21 (1H, s), 8.25 (1H, s). ³¹P NMR (162 MHz, D₂O) 8-1.45, 53.78.

### (D) Synthesis of 2-amino-9-[(5R,7R,8R,12aR,14R,15R,15aS,18R)-14-(6-amino-9H-purin-9-yl)-10,18-dihydroxy-2,10-dioxido-2-sulfanylhexahydro-14H-15,]2a-(epoxymethano)-5, 8-methanofuro[3,2-l][1,3,6,9,11,2,10]pentaoxadiphosphacyclotetradecin-7(12H)-yl]-1,9-dihydro-6H-purin-6-one sesqui-triethylamine Salt (Optical Isomer; Parent CDN)

To 2-amino-9-[(5R,7R,8R,12aR,14R,15R,15aS,18R)-14-(6-amino-9H-purin-9-yl)-18-{[tert-butyl(dimethyl)silyl]oxy)-2,10-dihydroxy-10-oxido-2-sulfidohexahydro-14H-15,12a-(epoxymethano)-5,8-methanofuro[3,2-1][1,3,6,9,11,2,10]pentaoxadiphosphacyclotetradecin-7(12H)-yl]-1,9-dihydro-6H-purin-6-one (optical isomer) (322 mg, tR2) were added methanol (3.0 mL) and triethylamine trihydrofluoride (3.2 mL). The reaction mixture was concentrated to remove the methanol, and the residue was stirred at 55° C. for 1 hour. The mixture was cooled to room temperature, ethoxy(trimethyl)silane (14 mL) was added thereto, and the mixture was stirred at room temperature for 2 hours. The reaction mixture was concentrated under reduced pressure, and the residue was purified by C18 column chromatography (acetonitrile/10 mM triethylammonium acetate buffer solution) to give the title compound (266 mg). ¹H NMR (400 MHz, D₂O) δ 1.23 (14H, t, J = 7.3 Hz), 3.15 (10H, q, J = 7.3 Hz), 4.02 (1H, d, J = 8.1 Hz), 4.13-4.24 (2H, m), 4.27-4.42 (4H, m), 4.59 (1H, d, J = 4.4 Hz), 5.01 (1H, s), 5.11 (1H, d, J = 4.2 Hz), 5.61-5.73 (1H, m), 5.95 (1H, d, J = 8.3 Hz), 6.15 (1H, s), 7.87 (1H, s), 8.00 (1H, s), 8.25 (1H, s). ³¹P NMR (162 MHz, D₂O) 8-1.93, 55.44.

### Preparation of CDN-Linker

(A) tert-butyl ((S)-1-(((S)-1-((4-(hydroxymethyl)phenyl)amino)-1-oxopropan-2-yl)amino)-1-oxopropan-2-yl)carbamate, intermediate-1

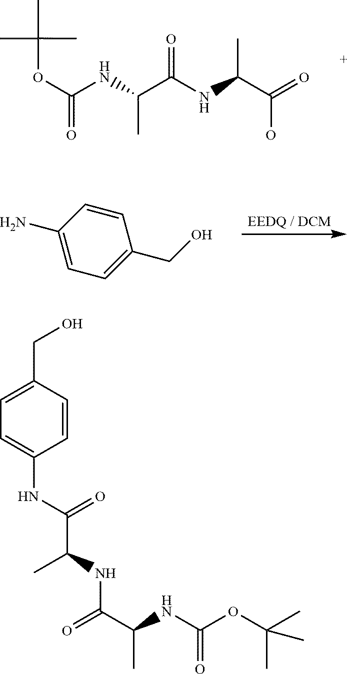

(tert-Butoxycarbonyl)-L-alanyl-L-alanine (2440 mg, 9.09 mmol, Chem-Impex) and 4-aminobenzyl alcohol (1154 mg, 9.09 mmoL) in DCM (50 mL) was treated with N-ethoxycarbonyl-2ethoxy-1,2-dihydroquinoline (EEDQ, 2498 mg, 10.0 mmol) at rt for 40 h. The solid was collected by filtration and washed with DCM (3 x 10 mL) and dried over vacuum to get the pure product. The filtrate was purified by chromatography on silica gel (0-10% MeOH in DCM) to get another batch of product. Two batches of product were combined as the intermediate-1 (2600 mg, 78.3%). LCMS (AA): m/z = 366.2 (M+H). ¹H NMR (MeOH-d4) 8 7.57 (d, J = 8.0 Hz, 2H), 7.30 (d, J = 8.0 Hz, 2H), 4.56 (s, 2H), 4.49-4.45 (m, 1H), 4.09-4.04 (m, 1H), 1.45 (s, 3H), 1.44 (s, 9H), 1.33 (d, J = 8.0 Hz, 3H).

(B) (S)Amino-N-((S)-1-((4-(chloromethyl)phenyl)amino)-1-oxopropanyl)propanamide, intermediate-2

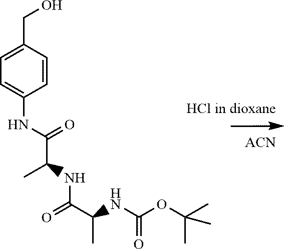

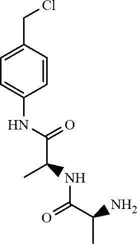 [SMILES: C[C@H](N)C(=O)N[C@@H](C)C(=O)Nc1ccc(CCl)cc1]

To a round bottom flask charged with Intermediate-1 (700 mg, 1.92 mmol) and acetonitrile (20 mL) was added a solution of hydrogen chloride in dioxane (4 M, 25 mL, 100 mmol). The solution was stirred at rt for 90 min. The solvent was removed using rotavapor. The remaining residue was suspended in acetonitrile (5 mL) and filtrated. The solid was washed with diethyl ether (10 mL x 2) as the intermediate-2 and used for next step without further purification (730 mg, >100% due to residue HCl/solvent). LCMS (AA): m/z = 284.1 (M+H). ¹H NMR (DMSO-d6) δ 10.29 (s, br, 1H), 8.74 (s, br, 1H), 8.18 (s, br, 2H), 7.61 (d, J = 8.0 Hz, 2H), 7.37 (d, J = 8.0 Hz, 2H), 4.72 (s, 2H), 4.52-4.47 (m, 1H), 3.90-3.87 (m, 1H), 1.38-1.35 (m, 6H).

(C) N-((S)-1-(((S)-1-((4-(Chloromethyl)phenyl)amino)-1-oxopropan-2-yl)amino)-1-oxopropan-2-yl)-6-(2,5-dioxo-2,5-dihydro-1H-pyrrol-1-yl)hexanamide, intermediate-3

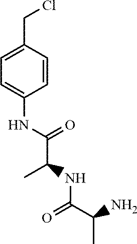 [SMILES: C[C@H](N)C(=O)N[C@@H](C)C(=O)Nc1ccc(CCl)cc1]

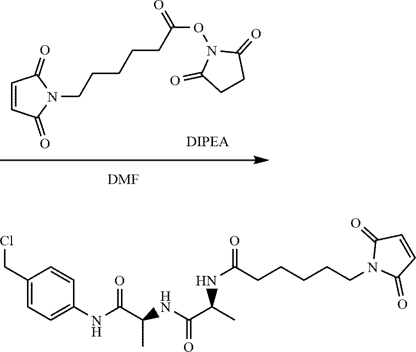

Crude Intermediate-2 (569.9 mg, 1.78 mmol) was suspended in DMF (6 mL) and treated with DIPEA (0.93 mL, 5.34 mmol). To the above mixture was added a solution of N-succinimidyl 6-maleimidohexanoate (657 mg, 2.13 mmol) in DMF (4 mL) and kept at rt for 30 min. The reaction mixture was then poured into ice-cold buffer (PBS, 100 mL, pH 7.4). The precipitate was collected by centrifugation and washed with water (60 mL x 3), diethyl ether (5 mL x 2) and EtOAc (2 mL) and dried to get intermediate-3 (560 mg, 66%). LCMS (AA): m/z = 477.2 (M+H). ¹H NMR (DMSO-d6) δ 9.93 (s, 1H), 8.07 (d, J = 8.0 Hz, 1H), 7.99 (d, J = 8.0 Hz, 1H), 7.62 (d, J = 8.0 Hz, 2H), 7.36 (d, J = 8.0 Hz, 2H), 7.00 (s, 2H), 4.71 (s, 2H), 4.39-4.37 (m, 1H), 4.25-4.24 (m, 1H), 3.38-3.34 (m, 2H), 2.10 (t, J = 8.0 Hz, 2H), 1.52-1.43 (m, 4H), 1.30 (d, J = 8.0 Hz, 3H), 1.22-1.15 (m, 5H).

(D) N-[(2S)-1-{[(2S)-1-{[4-({[(2S,5R,7R,8R,12aR,14R,15R,15aS,18R)-7-(2-amino-6-oxo-1,6-dihydro-9H-purin-9-yl)-14-(6-amino-9H-purin-9-yl)-10,18-dihydroxy-2,10-dioxidohexahydro-14H-15,12a-(epoxymethano)-5,8-methanofuro[3, 2-l][1,3,6,9,11,2,10]pentaoxadiphosphacyclotetradecin-2(12H)-yl]sulfanyl}methyl)phenyl]amino}-1-oxopropan-2-yl]amino}-1-oxopropan-2-yl]-6-(2,5-dioxo-2,5-dihydro-1H-pyrrol-1-yl)hexanamide or N-[(2S)-1-{[(2S)-1-{[4-({[(2R,5R,7R,8R,12aR,14R,15R,15aS,18R)-7-(2-amino-6-oxo-1,6-dihydro-9H-purin-9-yl)-14-(6-amino-9H-purin-9-yl)-10,18-dihydroxy-2,10-dioxidohexahydro-14H-15,12a-(epoxymethano)-5, 8-methanofuro[3,2-l][1,3,6,9,11,2,10]pentaoxadiphosphacyclotetradecin-2(12H)-yl]sulfanyl}methyl)phenyl]amino}-1-oxopropan-2-yl]amino}-1-oxopropan-2-yl]-6-(2,5-dioxo-2,5-dihydro-1H-pyrrol-1-yl)hexanamide, CDN-Linker

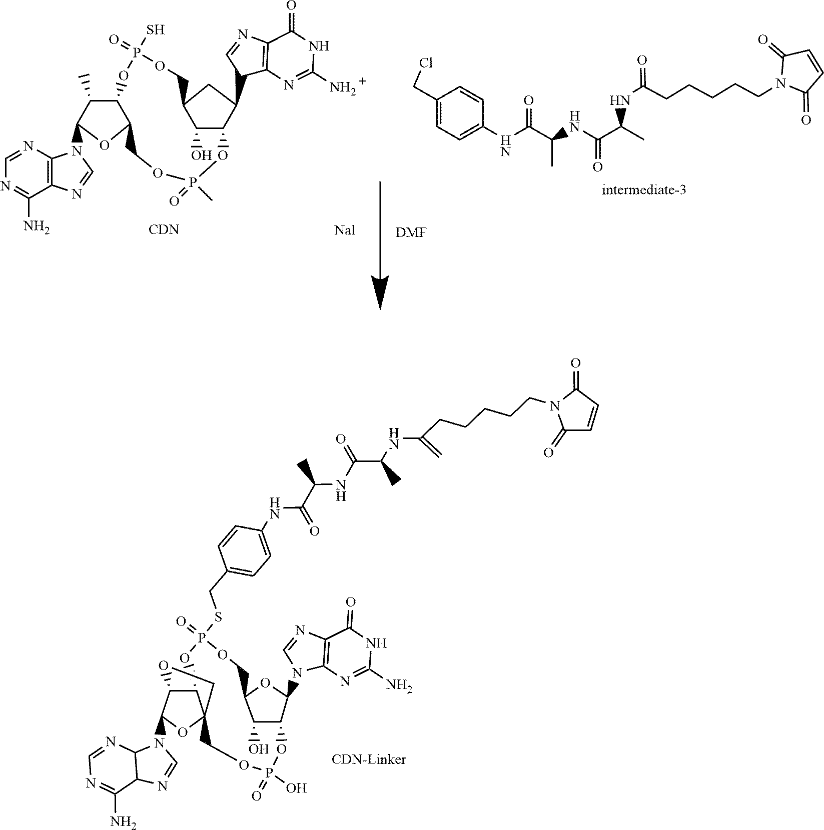

2-Amino[(2S,5R,7R,8R,12aR,14R,15R,15aS,18R)-14-(6-amino-9H-purinyl)-10,18-dihydroxy-2,10-dioxido-2-sulfanylhexahydro-14H-15,12a-(epoxymethano)-5,8-methanofuro[3,2-1][1,3,6,9,11,2,10]pentaoxadiphosphacyclotetradecin-7(12H)-yl]-1,9-dihydro-6H-purin-6-one or 2-amino[(2R,5R,7R,8R,12aR,14R,15R,15aS,18R)-14-(6-amino-9H-purinyl)-10,18-dihydroxy-2,10-dioxido-2-sulfanylhexahydro-14H-15,12a-(epoxymethano)-5,8-methanofuro[3,2-1][1,3,6,9,11,2,10]pentaoxadiphosphacyclotetradecin-7(12H)-yl]-1,9-dihydro-6H-purin-6-one (parent CDN) di-triethylamine salt (85 mg, 0.094 mmol) was co-evaporated with anhydrous acetonitrile (1.5 mL x 3) and dissolved in DMF (1 mL). To this solution was added intermediate-3 (89.6 mg, 0.188 mmol) followed by sodium iodide (2.82 mg, 0.0188 mmol). The reaction was sealed and heated at 50° C. for 50 min. The mixture was diluted with DMF (1 mL) and directly purified using reverse phase C18 column on ISCO system (0-30-100% acetonitrile/water with 10 mM ammonium acetate as modifier). The pure fractions were collected and lyophilized to get white solid as target CDN-Linker ammonium salt (45 mg, 42%). LCMS (AA): m/z = 1143.3 (M+H). ¹H NMR (DMSO-d6/D20) 8 8.18 (s, 1H), 8.08-8.03 (m, 1H), 7.99 (s, 1H), 7.56 (d, J = 8.0 Hz, 2H), 7.30 (d, J = 8.0 Hz, 1H), 6.94-6.83 (br, 2H), 6.06 (s, 1H), 5.91 (d, J = 8.0 Hz, 1H), 5.38 (br, s, 1H), 5.10-5.09 (m, J = 8.0 Hz, 2H), 4.93 (s, 1H), 4.81-4.78 (m, 1H), 4.54-4.55 (m, 1H), 4.36-4.32 (m, 1H), 4.24-4.02 (m, 8H), 3.95 (d, J = 8.0 Hz, 2H), 3.34 (t, J = 8.0 Hz, 2H), 2.09 (t, J = 8.0 Hz, 2H), 1.50-1.41 (m, 4H), 1.29 (d, J = 8.0 Hz, 3H), 1.20-1.13 (m, 5H).

### Preparation of CDN-PEG-Lipid

The CDN-PEG-Lipid amphiphile conjugate as shown in FIG. 1B was prepared by coupling the CDN-Linker to a PEG2k-Lipid having a terminal thiol using a maleimide coupling reaction as further described below.

In order to prepare DSPE-PEG2k-SH by reducing the disulfide bond in the orthopyridyl disulfide (PDP) group, 100.0 mg (33.6 µmole) of DSPE-PEG2k-PDP (Avanti) was dissolved in 50.0 mM ammonium acetate buffer (pH 5) with 5.0 mM EDTA (6.0 mL total volume) and then 5.0 equivalent of tris(2-carboxyethyl)phosphine (TCEP) (168 µmole, 48 mgs) was added. The solution was incubated at 25° C. for one hour and then transferred to amicon ultra centrifugal filters with a 3 kDa molecular weight cut-off membrane and the sample was washed using fresh reaction buffer and three spin steps to remove the released pyridine-2-thione. The flow-through was monitored by reading the absorbance at 343 nm to ensure the pyridine-2-thione was removed. The solution containing DSPE-PEG2k-SH was transferred to a glass vial, the volume was adjusted to 5.0 mL total and buffered to pH 7.6 by adding triethanolamine buffer to a final concentration of 100 mM, and then degassed by bubbling with argon gas. The CDN-linker maleimide (33.6 µmole, 38.4 mg) was added (67.2 µL of a 0.500 M stock in dimethylsulfoxide) and the reaction was sealed under argon and stirred overnight at 25° C. The CDN-PEG-Lipid was purified by reverse-phase HPLC using a C8 column and mixtures of acetonitrile and 0.100 M triethylamine acetate buffered water (pH 7), starting with 40% acetonitrile and ramping to a final mixture with 98% acetonitrile. The CDN-PEG-lipid was isolated in 76% yield (102 mg). As shown in Table 3, the identity was confirmed by MALDI-TOF mass spectrometry where a series of peaks separated by 44 amu (the repeat unit mass of polyethylene glycol) were observed between approximately 3900 to 4300 m/z. The three strongest peaks at 4070, 4115, and 4159 correspond within the margin of error of the technique of +/- 4 amu to the [M + Na+] species with PEG molecular weights of 2024, 2068, and 2112, which have the expected masses of 4069, 4113, and 4157. The purity was confirmed by observation of a single peak on reverse phase HPLC when monitored at 260 nm.

TABLE 3

Characterization of CDN-PEG-Lipid by MALDI-TOF Mass Spectrometry

| PEG repeat units (OC₂H₂) | PEG MW | Species MW | [M+H]⁺ | Observed peak | [M+Na]⁺ | Observed peak |
|---|---|---|---|---|---|---|
| 42 | 1848 | 3870 | 3871 | n.o. | 3893 | 3894 |
| 43 | 1892 | 3914 | 3915 | n.o. | 3937 | 3939 |
| 44 | 1936 | 3958 | 3959 | 3960 | 3981 | 3983 |
| 45 | 1980 | 4002 | 4003 | 4003 | 4025 | 4027 |
| 46 | 2024 | 4046 | 4047 | 4047 | 4069 | 4070 |
| 47 | 2068 | 4090 | 4091 | 4091 | 4113 | 4115 |
| 48 | 2112 | 4134 | 4135 | 4134 | 4157 | 4159 |
| 49 | 2156 | 4178 | 4179 | 4179 | 4201 | 4202 |
| 50 | 2200 | 4222 | 4223 | 4222 | 4245 | 4246 |
| 51 | 2244 | 4266 | 4267 | 4266 | 4289 | 4291 |
| 52 | 2288 | 4310 | 4311 | 4310 | 4333 | 4335 |

### Example 2: Evaluation of Cellular Uptake and STING Activation in Vitro

The CDN-PEG-Lipid generated as described in Example 1 was evaluated for delivery of the CDN payload and activation of the STING signaling pathway in vitro. Specifically, the potency of CDN-PEG-Lipid for inducing STING activation was compared to parent CDN using a STING reporter assay. Briefly, a mouse macrophage cell line with stable integration of an interferon regulatory factor (IRF)-inducible Lucia luciferase reporter construct (Invivogen, RAW-Lucia™ ISG cells) was used. The cells express the Lucia luciferase gene under control of the ISG54 minimal promoter in conjunction with five IFN-stimulated response elements (ISRE). The presence of activated STING induces IRF3 (interferon regulatory factor 3) which binds to ISREs that activate the expression of Lucia luciferase such that increased luciferase activity correlates with activation of STING. To measure STING activation, the manufacturer’s protocol was followed. Briefly, test solutions comprising CDN-PEG-Lipid or parent CDN were prepared in PBS at 10-times the desired assay concentration and 20 µL was added to each assay well of a 96-well plate. Subsequently, 1x10⁵ RAW-ISG cells in 180 µL of complete DMEM media were added and the assay was incubated for 20 hours. Following incubation, 10 µL aliquots of the cell supernatant were transferred to an opaque white 96-well plate and combined with 50 µL of Quanti-Luc (Invivogen) substrate. The luciferase activity was immediately measured using a plate reader to detect luminescence.

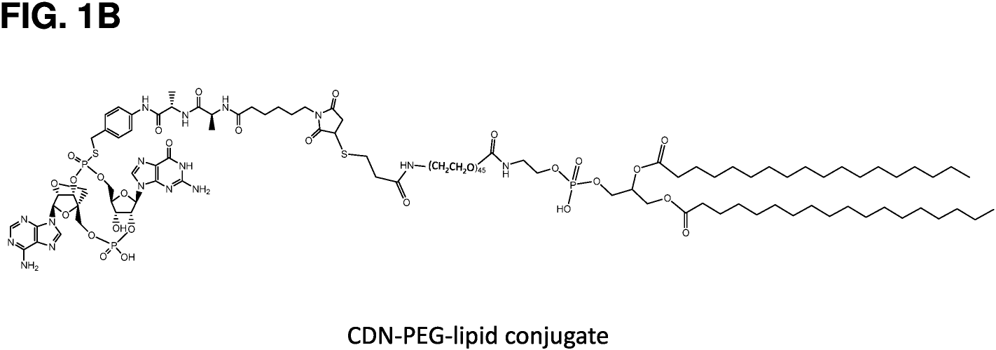

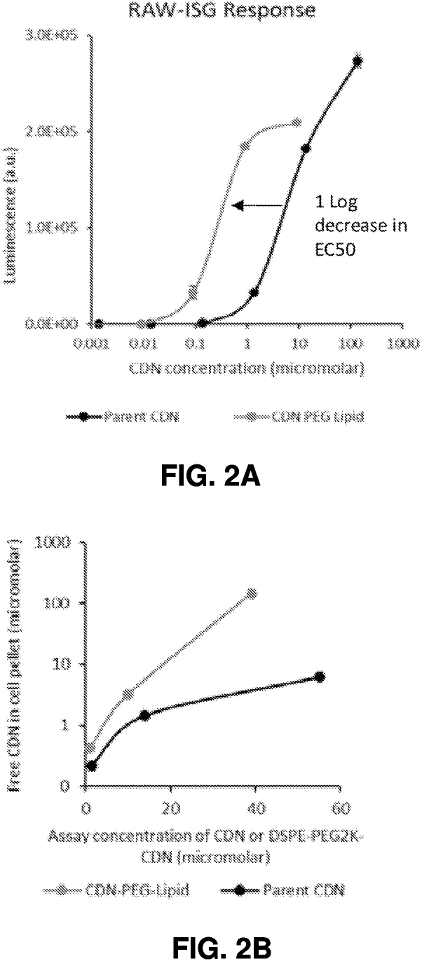

The parent CDN or CDN-PEG-Lipid were added to the STING reporter cell line at a final concentration of 0.001-100 µM. As shown in FIG. 2A, cells stimulated with the CDN-PEG-Lipid had an approximately one log decrease in EC50 compared to cells stimulated with parent CDN. EC50 measured for the parent CDN is approximate given that the response did not plateau at the high concentrations evaluated. This result indicates that the CDN-PEG-Lipid amphiphilic conjugate induces more potent STING activation than the soluble parent CDN.

To further evaluate the CDN-PEG-Lipid amphiphilic conjugate, the intracellular level of soluble CDN was determined following treatment with CDN-PEG-Lipid or parent CDN. Specifically, 10x10⁶ RAW-ISG cells were treated at three concentrations of parent CDN (1.4, 14, 55 µM) or CDN-PEG-Lipid (1, 10, 39 µM) for 18 hours in non-tissue culture treated 6-well plates, the supernatant was removed and saved, and then the cells were dispersed by addition of 1 mL of PBS containing 0.1 wt% bovine serum albumin and 1 mM EDTA followed by pipetting. The cell suspension was transferred to 1.5 mL tubes and centrifuged at 600 g to obtain a cell pellet. The cell pellet was resuspended in 1 mL of PBS containing 0.1 wt% bovine serum albumin and 1 mM EDTA in order to wash the cells of surface bound drug, the cells were pelleted again as before, the supernatant was removed, and the cell pellet was lysed and the level of intracellular soluble CDN was quantified using LC/MS/MS. As shown in FIG. 2B, cells treated with CDN-PEG-Lipid had higher intracellular levels of soluble CDN than cells treated with parent CDN. This result indicates the CDN-PEG-Lipid conjugate induces cellular uptake and release of CDN. Indeed, the increased intracellular levels of CDN due to stimulation with CDN-PEG-Lipid may attribute to increased STING activation compared to parent CDN.

### Example 3: Preparation of Synthetic Lipid Nanodiscs for STING Agonist Delivery

Synthetic PEGylated lipid nanodiscs (LNDs) comprising CDN-PEG-Lipid as described in Example 1 were prepared. Methods for preparation of LNDs are known, in which LNDs form spontaneously when PEGylated lipids are combined at 20-30 mol% with high-Tₘ phospholipids in aqueous buffers (see, e.g., Johnsson and Edwards, Biophysical J. 2003, 85, 3839-3847), and were used with minor modification to prepare LNDs comprising CDN-PEG-Lipid as detailed below. LND particles comprising CDN-PEG-Lipid are alternatively referred to as “LND-formulated CDN” or “LND-CDN” herein.

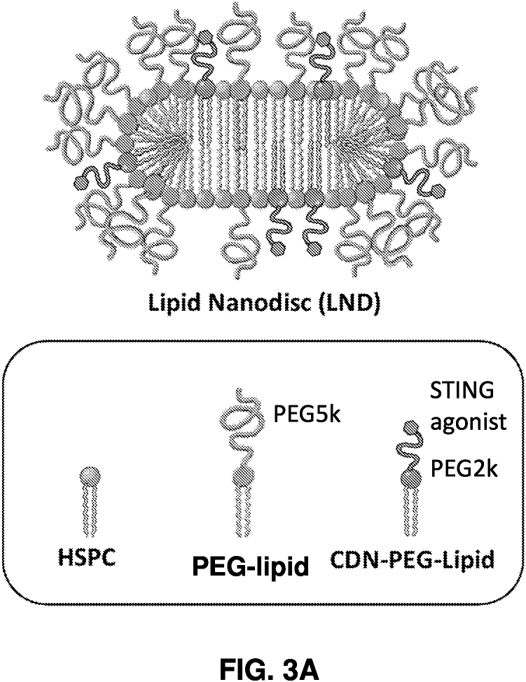

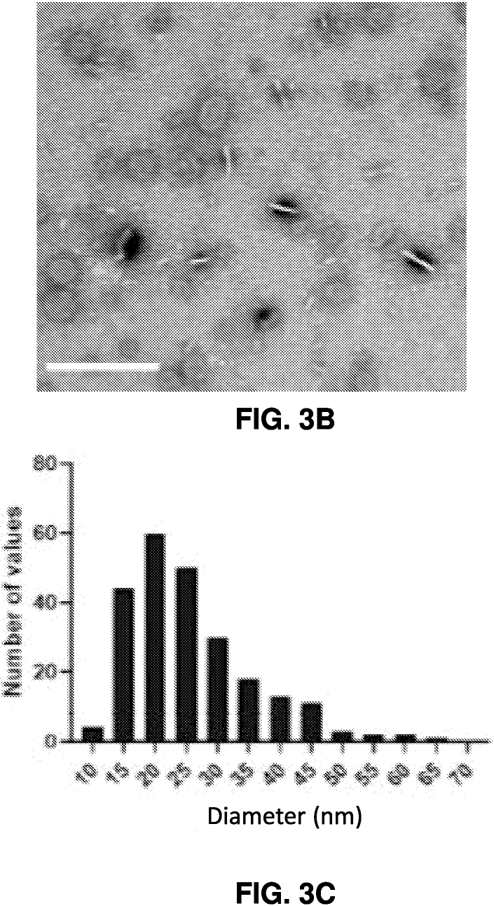

The lipid nanodiscs were prepared using lipid compositions comprising hydrogenated soy L-α-phosphatidylcholine (HSPC), 1,2-distearoyl-sn-glycero-3-phosphoethanolamine-N-[methoxy(polyethylene glycol)-5000] ammonium (DSPE-PEG5k), CDN-PEG-Lipid, and optionally 1,2-dipalmitoyl-3-trimethylammonium-propane chloride (16:0 TAP). Phospholipids were purchased from Avanti Polar Lipids. Three different lipid compositions comprising the CDN-PEG-Lipid amphiphilic conjugate were used to generate LND particles (with the LND particles derived from each composition referred to as LND 1, LND 2, or LND 3). The lipid components and mole percentage used in each composition are indicated in Table 4. As shown by the schematic in FIG. 3A, LND 1 nanoparticles comprised PEG-Lipid (DSPE-PEG5k), CDN-PEG-Lipid, and phospholipid (HSPC).

TABLE 4

Lipid Nanodisc Formulations

| Lipid Nanodisc | HSPC mol% | DSPE-PEG5k mol% | CDN-PEG-Lipid mol% | 16:0 TAP mol% | Average hydrodynamic diameter by number (d.nm) | PDI (polydispersity index) |
|---|---|---|---|---|---|---|
| LND 1 | 75 | 20 | 5 | 0 | 31 | 0.17 |
| LND 2 | 55 | 20 | 5 | 20 | 34 | 0.22 |
| LND3 | 50 | 20 | 10 | 20 | 28 | 0.26 |

LNDs were prepared via ethanol precipitation. Briefly, to prepare LND 1, a total of 10.0 µmole of lipid (75.0 mol% HSPC (Avanti), 20.0 mol% DSPE-PEG5k-OMe (Avanti), 5.0 mol% CDN-PEG-Lipid) was dissolved in 0.400 mL of ethanol and the solution was warmed to 40° C. until it was completely clear. Subsequently, 20.0 µL aliquots of the ethanol solution were added to a vial containing 1.600 mL of PBS buffer, which was also maintained at 40° C. The vial with the aqueous solution was vortexed briefly following each addition and the process continued until all 0.400 mL of ethanol was added to the aqueous solution. The ethanol was then removed by dialysis using amicon ultra centrifugal filters with a 3 kDa molecular weight cut-off membrane as per the manufacturer’s instructions until the solution contained less than 0.2% by volume of ethanol. The solution was filtered through a syringe filter with a 0.2 micron Supor® membrane. The concentration of CDN conjugate was determined by diluting an aliquot of the PBS stock solution with methanol to a methanol concentration of 95% by volume and then measuring the absorbance at 260 nm using the molar attenuation coefficient of the CDN-linker (30.3 mM⁻¹cm⁻¹).

LND 2 and LND 3 particles were formed by a thin-film hydration method followed by thermal annealing. Briefly, depending on the specific formulation, lipid stocks in either chloroform or ethanol were combined in a glass vial and the organic solvent was evaporated. The components were then completely solubilized in ethanol, and subsequently the ethanol was removed by evaporation under a stream of nitrogen. The resulting film was further dried under high vacuum and then hydrated with PBS at a lipid concentration of 10.0 mg/mL with heating to 65° C. for 20 minutes followed by cooling to 25° C., and this heating/cooling cycle was repeated a total of three times with frequent mixing of the solution using a vortexer. Following hydration, the solution was treated in the same way as for LND 1.

The resulting LNDs were characterized by dynamic light scattering (DLS) and were found to have a hydrodynamic radius of approximately 30 nm with a PD1 of approximately 0.2 as shown in Table 4. Based upon comparison of LND 2 and LND 3 particles, particle assembly and size were not significantly impacted by increasing the mol % of CDN-PEG-Lipid.

LNDs formulated as LND 1 particles were further characterized by transmission electron microscopy (TEM). As shown in FIGS. 3B-3C, the particles were found to have well-defined discoid morphologies (FIG. 3B) with mean diameters of about 26 nm (FIG. 3C). A subset of discs oriented perpendicular to the plane appear as rectangles with a width of 5-6 nm, as expected for a single lipid bilayer.

Unless indicated otherwise in the following Examples, “LND-CDN” or “LND-formulated CDN” refer to CDN-PEG-Lipid formulated as LND 1 particles.

### Example 4: LND-Formulated STING Agonist Is More Effective Than Unformulated STING Agonist in a MC38 Tumor Mouse Model

CDN agonists of the intracellular STING receptor have been shown to induce potent anti-tumor immunity, resulting in rapid tumor shrinkage in mouse tumor models when administered by intratumoral injection (see, e.g., Corrales, L, et al (2015) Cell Reports 11:1018). However, the therapeutic anti-tumor effects of systemically administered soluble CDN are limited, likely due to rapid clearance of the CDN (i.e., short serum half-life) (see, e.g., Shae, D. et al (2019) Nat Nanotech 14:269-278; Cheng, N. et al (2018) JCI Insight 3(22)) Thus, it was evaluated whether delivery of CDN using a LND carrier would improve anti-tumor efficacy following systemic administration.

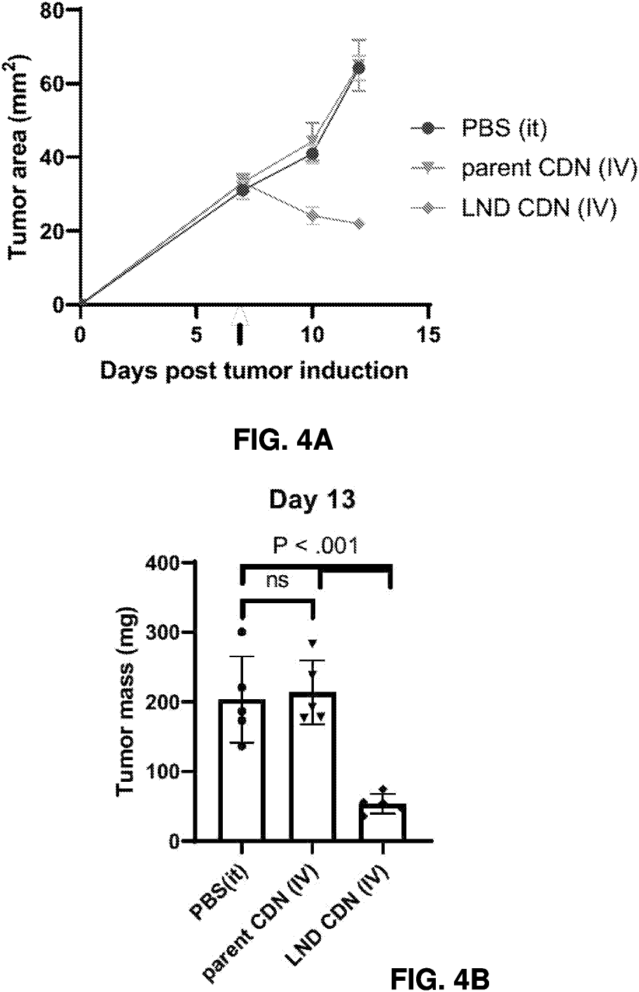

Specifically, a single intravenous injection of LND 1 prepared as described in Example 3 was compared to intravenous injection of parent CDN for effects on tumor growth in a mouse model of colon adenocarcinoma. Tumors were established in 8-10 week old C57BL/6 mice by inoculating the right flank with a subcutaneous injection of 5x10⁵ MC38 cells suspended in PBS. On day 7 post-inoculation, the mice were administered an intravenous injection of either parent CDN or LND-formulated CDN at a dose of 5 nmol CDN. Tumor area was measured on days 7, 10, and 12 and compared to untreated mice that received an intratumoral injection of PBS on day 7. The mice were then sacrificed on day 13, and excised tumors were weighed. As shown in FIGS. 4A-4B, a single intravenous dose of LND-formulated CDN significantly reduced tumor area and mass, while an equivalent dose of the parent CDN had no effect relative to tumors in untreated mice.

The effect of systemic administration of LND-formulated CDN versus soluble CDN was further evaluated in the MC38 tumor model. Specifically, a single intravenous dose of CDN formulated as LND 1 particles was compared to intravenous administration of soluble CDN, either parent CDN or ADU-S100. ADU-S100 (dithio-(Rₚ, Rₚ)-[cyclic[A(2′,5′)pA(3′,5′)p]]; alternatively referred to as ML RR-S2 CDA or MIW815) is a synthetic CDN agonist known to be a potent activator of murine and human STING (see, e.g., Corrales, et al (2015) Cell Rep. 11:1018; Yi, et al (2013) PLoS One 8:e77846) and is under clinical investigation in methods for treatment of cancer (see, e.g., NCT02675439, NCT03937141, and NCT03172936).

Mice were inoculated with MC38 tumors as described above. On day 7 post tumor inoculation, mice were administered a single intravenous injection of parent CDN at a dose of 5 nmol (n=5) or 100 nmol (n=10), ADU-S100 at a dose of 100 nmol (n=10), or LND 1 at a dose of 5 nmol CDN (n=20). Following administration, tumor growth and animal survival was monitored and compared to untreated mice that received an injection of PBS.

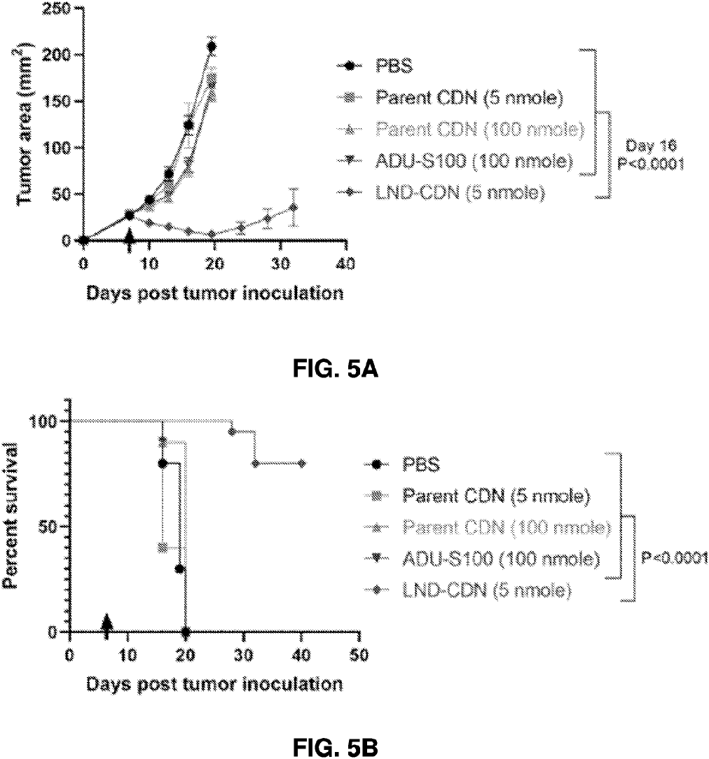

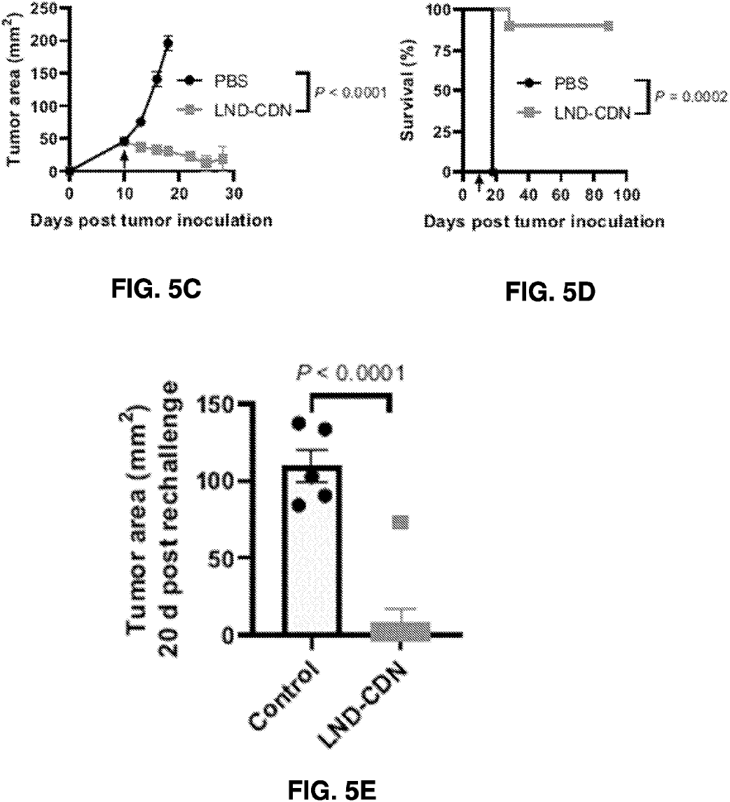

As shown in FIGS. 5A-5B, a single intravenous dose of LND-formulated CDN resulted in rapid tumor shrinkage (FIG. 5A) and improved long-term survival (FIG. 5B) compared to mice administered parent CDN or ADU-S100. Indeed, while 15 of 20 mice (75%) administered LND-formulated CDN eliminated primary tumor and had long-term survival, mice administered either dose of parent CDN or ADU-S100 showed no reduction of tumor burden compared to untreated mice and required euthanasia by day 20. The surviving mice in FIG. 5B remained long-term survivors for the duration of the study (about 80 days; data not shown).

It was further investigated whether intravenous administration of LND-formulated CDN was effective in treating well-established MC38 tumors. Specifically, MC38 tumors were allowed to grow for 10 days post-inoculation (mean size of about 45 mm²), whereupon a single intravenous dose of LND 1 was administered (5 nmol CDN). Control mice were administered PBS only. As shown in FIGS. 5C-5D, a majority of animals rejected their tumors and had long-term survival following a single dose. Though mice administered LND-formulated CDN experience some weight loss (about 10% body weight), they recovered full body weight within about 5 days of administration (data not shown).

To determine if administration of LND-formulated CDN resulted in a memory response, cured mice indicated in FIG. 5D were subsequently re-challenged at day 90 following the original tumor inoculation by subcutaneous injection with 5x10⁵ MC38 cells on the opposite flank. Control mice were naive age-matched mice. As shown in FIG. 5E, at day 20 post inoculation, all control mice developed tumors. In contrast, 8 of 9 re-challenged mice remained tumor-free, demonstrating formation of anti-tumor immune memory.

### Example 5: LND-Formulated STING Agonist is Effective in Multiple Tumor Models

The therapeutic efficacy of intravenous LND-formulated CDN administration was further evaluated in mouse tumor models of melanoma (B16F10), lung (TC-1), and breast (4T1) cancer.

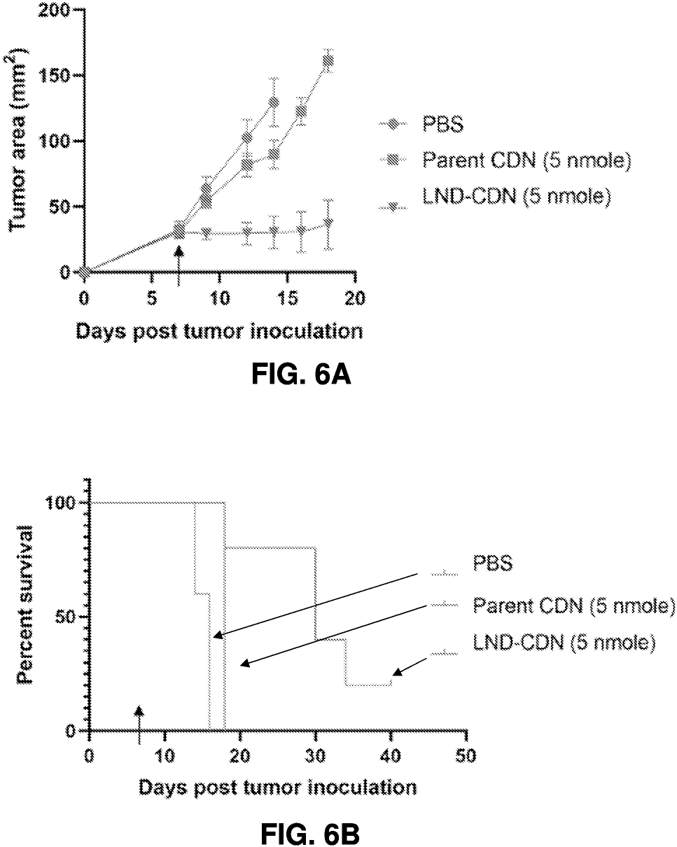

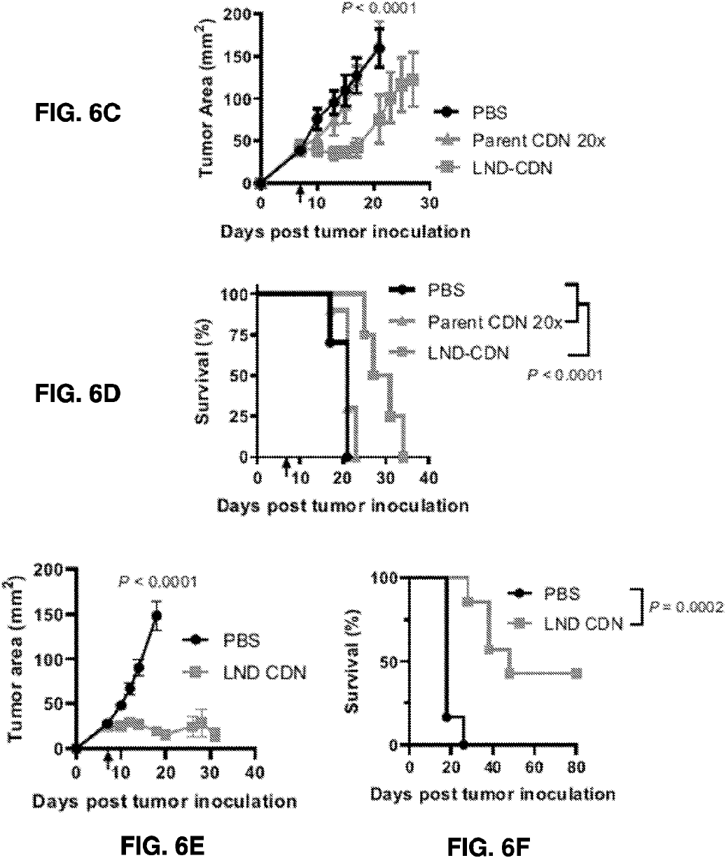

Briefly, 8-10 week old C57BL/6J mice were inoculated in the right flank by a subcutaneous injection of 5x10⁵ B16F10 tumor cells suspended in PBS. On day 7 post-inoculation, the mice were administered parent CDN or CDN formulated as LND 2 particles as described in Example 3 (n=5). In both groups the mice received a dose of 5 nmol CDN given by intravenous injection. Following administration, tumor growth and animal survival was monitored and compared to untreated mice injected with PBS alone. As shown in FIGS. 6A-6B, a single dose of LND-formulated CDN eliminated tumor growth and extended survival compared to untreated mice or mice treated with parent CDN.

To evaluate therapeutic efficacy in the aggressive 4T-1 orthotopic tumors, BALB/c mice were inoculated with 5 x 10⁵ 4T1-Luc tumor cells that were injected into the fourth mammary fat-pad. At 7 days post tumor inoculation, mice were administered CDN formulated as LND 1 at a dose of 10 nmol CDN or parent CDN at a dose of 200 nmol CDN per mouse by intravenous injection. Control mice were administered PBS only. As shown in FIGS. 6C-6D, a single-dose of the LND-formulated CDN resulted in delayed tumor outgrowth and significantly increased median survival compared to untreated tumors. In contrast, administration of the parent CDN, even at a 20-fold higher dose, had no impact on tumor growth or overall survival.

To evaluate the effect of systemic administration of LND-formulated CDN in TC-1 tumors (a model of HPV-driven cancers), C57BL6 mice were inoculated in the right flank with a subcutaneous injection of 3 x 10⁵ TC-1 cells in 100 µL sterile PBS. At day 7 post tumor inoculation, the mice were intravenously administered LND-CDN at a dose of 5 nmol CDN per mouse. Control mice were administered PBS only. As shown in FIGS. 6E-6F, administration of a single intravenous dose of LND-formulated CDN resulted in tumor regression and curative effect in about 43% of mice.

Overall, these results indicate that a single intravenous dose of CDN delivered by LND provides effective tumor control and extended survival in multiple solid tumor models, while intravenous administration of soluble CDN in the absence of the LND carrier has minimal or no effect.

### Example 6: Immune Effects of LND-Formulated STING Agonist

Stimulation of intratumoral STING by CDN agonists has been shown to induce potent anti-tumor immunity that is dependent upon activation of tumor-specific CD8+ effector T cells (see, e.g., Sivick, et al (2018) Cell Reports 25:3074). Additionally, type I interferon and TNFα cytokines induced by activation of the STING pathway may contribute to anti-tumor immunity (see, e.g., Demaria, O. et al. Proc National Acad Sci 112, 15408-15413 (2015); Francica, B. J. et al. Cancer Immunol Res 6, canimm.0263.2017 (2018)). Thus, it was evaluated whether CD8+ effector T cells or STING-induced immunostimulatory cytokines contribute to the anti-tumor immune effects induced by a single intravenous dose of LND-formulated CDN.

### (A) Requirement for Host STING Expression

It has been demonstrated that anti-tumor effects of STING agonists are dependent on the expression of STING in host cells (see, e.g., Corrales, L. et al. Cell Reports 11, 1018-1030 (2015); Baird, J. R. et al. Cancer Res 76, 50-61 (2016)). It was evaluated whether LND-formulated CDN would have anti-tumor effects in STING^{-/-} mice. Briefly, MC38 tumors were established in STING-deficient Goldenticket mice (Tmem173^{gt}, C57BL/6J-Sting1^{gt}/J) (Jackson Laboratory) by inoculating the flank with a subcutaneous injection of 5x10⁵ MC38 cells suspended in PBS. On day 7 post-inoculation, the mice were administered LND 1 at a dose of 5 nmol CDN per mouse by intravenous injection. Control mice were administered saline only. Each group included 5 mice. Tumor growth and overall survival were evaluated. LND-formulated CDN had no effect on reducing tumor growth or improving survival relative to control animals (data not shown), indicating a requirement for STING expression in host cells.

### (B) Role of Effector Cells

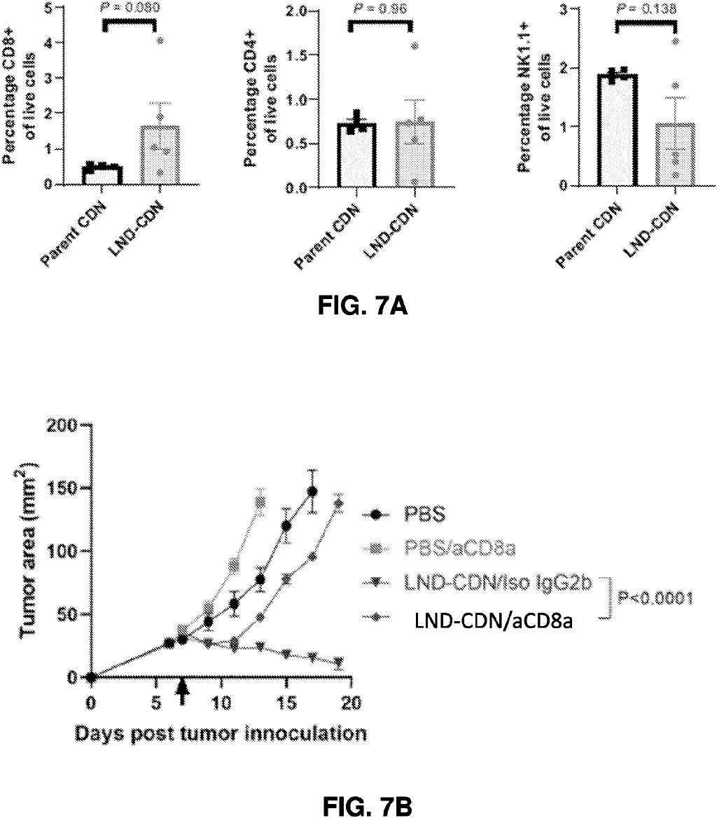

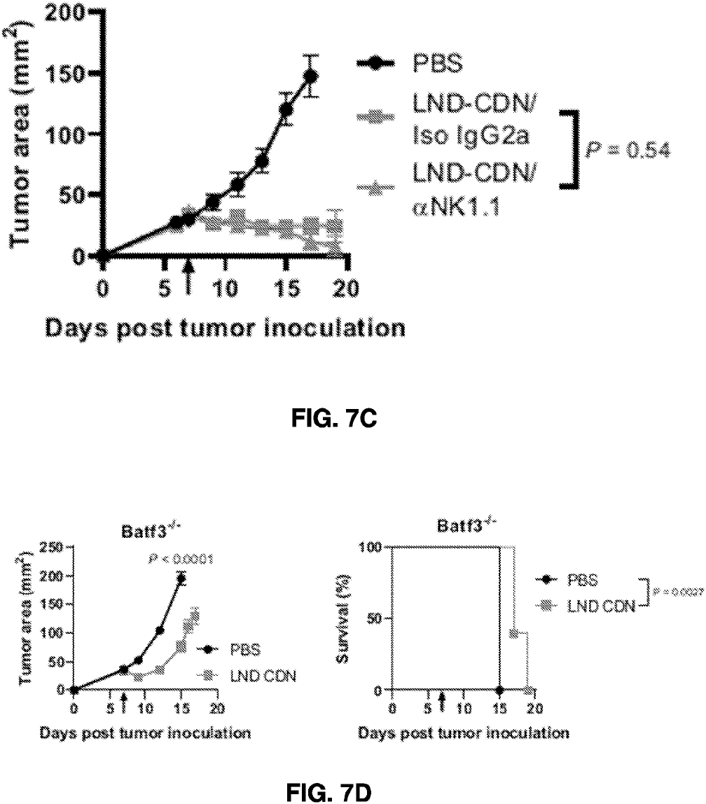

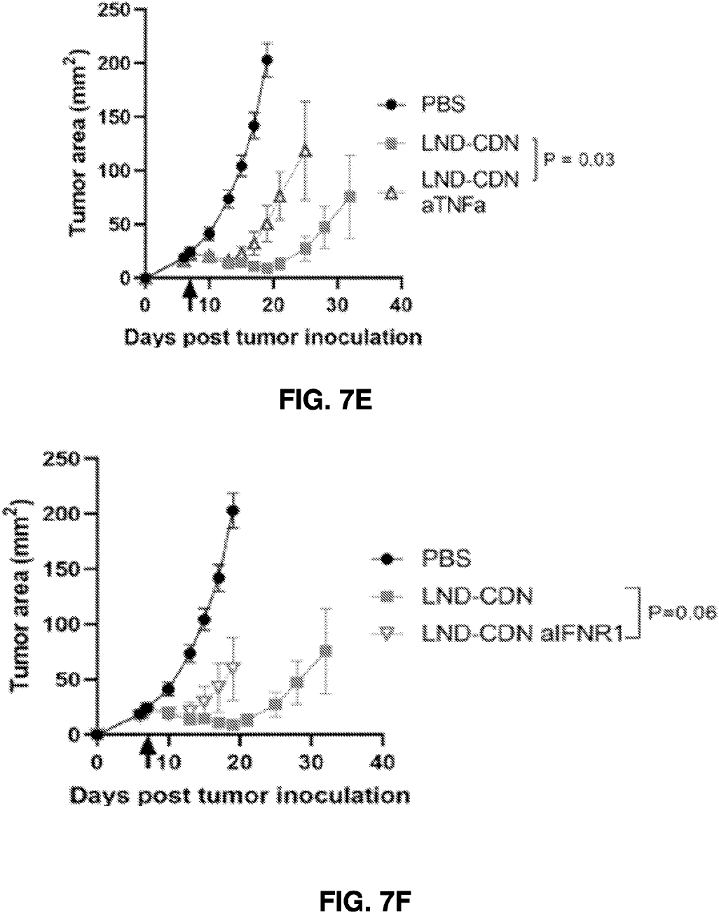

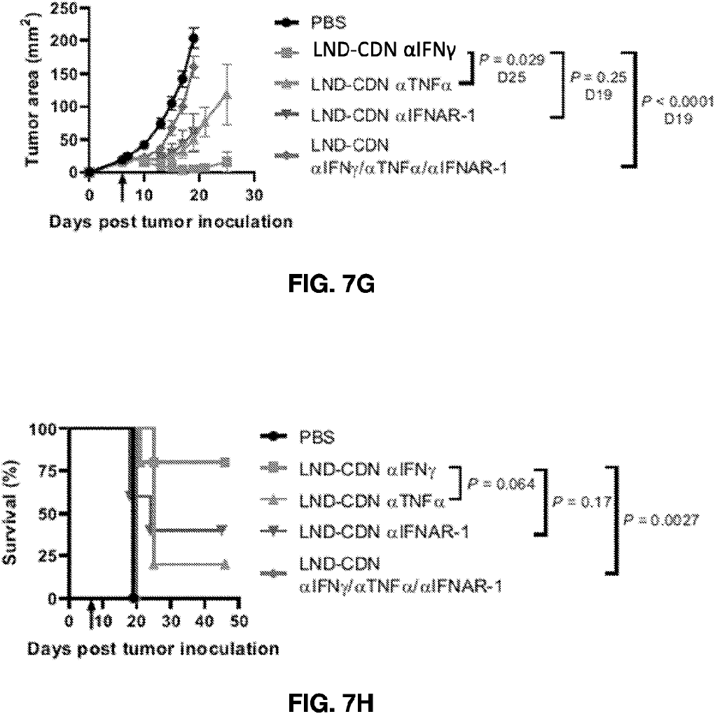

The abundance of CD8+ T cells and NK cells was evaluated in tumors following intravenous administration of LND-formulated CDN. Briefly, MC38 tumor bearing mice were intravenously administered LND 1 at 5 nmol CDN per mouse or parent CDN at 5 nmol per mouse on day 7 post tumor inoculation. Six days later, tumors were collected, weighed, cut into small pieces using scissors, and then digested in Hanks Balanced Salt Solution with Ca²⁺ and Mg²⁺ containing 10.0 mg/mL collagenase II (ThermoFisher) and 0.5 mg/ml DNAse I (Roche) for 1 hour at 37° C. with gentle shaking. The dissociated tumors were passed through a 70 µm cell strainer using a syringe plunger to force the material through with frequent rinsing with PBS buffer, isolated by centrifugation at 300 g. The resulting cell suspension was stained with antibodies against CD3e (clone 145-2C11, BV421), CD45 (clone 30-F11, FITC), NK1.1 (clone PK136, PerCP/Cyanine5.5), CD8a (clone 53-6.7, PE-Cy7), and CD4 (clone RM4-4, APC-Fire750). CD8 T cells were defined as CD45+ CD3e+ CD8+. CD4 T cells were defined as CD45+ CD3e+ CD4+. NK cells were defined as CD45+ CD3e- NK1.1+. As shown in FIG. 7A, tumors from mice administered LND-formulated CDN has significantly increased infiltration of CD8+ T cells relative to tumors from mice administered parent CDN. In contrast, tumor infiltration of NK cells was reduced in mice administered LND-CDN particles, while tumor infiltration of CD4+ T cells was similar to parent CDN.

The role of CD8+ T cells and NK effector cells was further evaluated in depletion studies in MC38 tumors. The MC38 tumors were established in C57BL/6J mice as described in Example 4. For CD8+ T cell depletion, the mice were administered an anti-CD8α depleting antibody or an isotype control antibody by intraperitoneal injection on day 6, 8, 11, and 15 post tumor inoculation at a dose of 250 µg per mouse. On day 7 post-inoculation, mice received an intravenous injection of LND 1 formulated as described in Example 3 at a dose of 5 nmol CDN or an injection of PBS only. As shown in FIG. 7B, while administration of LND-formulated CDN resulted in tumor regression in mice administered the isotype control, depletion of CD8+ T cells significantly decreased the ability of the LND-formulated CDN to control tumor growth.

For NK cell depletion, the MC38-tumor bearing mice were administered 0.200 mg of an NK cell-depleting antibody (αNK1.1 (clone PK136, rat IgG2a)) by intraperitoneal injection on day 6, 8, 11, and 15 following tumor inoculation. A separate cohort of mice were administered an isotype control antibody (clone C1.18.4, rat IgG2a, BioXCell). On day 7 post-inoculation, mice received an intravenous injection of LND 1 at a dose of 5 nmol CDN. Control mice that received no depleting antibody or isotype control antibody were administered saline only. As shown in FIG. 7C, administration of LND-formulated CDN resulted in tumor regression in mice administered either the isotype control or the NK cell-depletion antibody. These results indicated that whereas CD8+ T cells are critical as effector cells in inducing the anti-tumor immune response resulting from administration LND-CDN, the contribution of NK cells as effector cells is not as essential.

### (C) Role of Antigen Presenting Cells

Activation of anti-tumor T cells requires cross-presenting dendritic cells (DCs). Thus, it was evaluated whether therapeutic efficacy is dependent on cross-presenting DCs. Briefly, MC38 tumors were established in 8-10 week old Batf3^{-/-} mice (B6.129S(C)-Batf3tm1Kmm/J; The Jackson Laboratory) by subcutaneous flank injection of 5 x 10⁵ MC38 tumor cells (5 mice per group). On day 7 post tumor inoculation, the mice were administered LND 1 at a dose of 5 nmol CDN per mouse by intravenous injection. Control mice were administered saline only. Tumor growth and survival was monitored. As shown in FIG. 7D, LND-CDN therapy in BatF3^{-/-} mice lacking these DCs was ineffective, with tumors growth and survival being comparable to control animals.

Together these data demonstrate CD8+ T cells and DCs capable of cross presenting tumor antigen to these T cells are critical to the therapeutic efficacy of LND-formulated CDN.

### (D) Role of Immunostimulatory Cytokines

The role of STING-induced immunostimulatory cytokines was further evaluated in MC38 tumors. For neutralization of TNFα and blockade of the type I interferon receptor (IFNAR-1), mice were administered an anti-TNFα monoclonal antibody (clone XT3.11, BioXCell) or anti-IFNR1 monoclonal antibody (IFNAR-1, clone MAR1-5A3) respectively. The antibodies were given by intraperitoneal injection on day 6 and 7 post-inoculation at a dose of 250 µg per mouse. On day 7, mice received PBS alone or an intravenous dose of LND 1 at a dose of 5 nmol CDN. As shown in FIGS. 7E-7F, neutralization of TNFα or blockade of the type I interferon receptor significantly reduced the efficacy of CDN delivered by LND.

In addition, the effect of neutralization of IFN-γ was compared to neutralization/blockade of TNFα and IFNAR-1. To do so, the anti-TNFα monoclonal antibody, the anti-IFNR1 monoclonal antibody, and a monoclonal antibody targeting IFN-γ (clone XMG1.2, BioXCell) were used. Antibodies were administered by intraperitoneal injection on day 6 and 7 post tumor inoculation at a dose of 0.200 mg antibody per mouse. Experimental groups were administered the anti-TNFα monoclonal antibody, the anti-IFNR1 monoclonal antibody, the anti-IFN-γ monoclonal antibody, or a combination of the antibodies. On day 7 post tumor inoculation, the mice were administered with LND 1 at a dose of 5.0 nmol CDN per mouse by intravenous injection. Control mice were administered saline only. Tumor growth and survival were monitored. As shown in FIGS. 7G-7H, whereas neutralization of TNFα or blockade of the type I interferon receptor resulted in reduced therapeutic efficacy of the LND-formulated CDN, neutralization of IFN-γ had no effect. Blockade of TNFα, type I interferon receptor, and IFNγ simultaneously resulted in a complete failure of the therapy.

Together, these results demonstrate that the anti-tumor effects induced by LND-formulated CDN is dependent on CD8+ T cell as effectors, cross-presenting DCs, and the production of STING-induced immunostimulatory cytokines.

### Example 7: Evaluation of Lipid Particle Formulation for Delivery of STING Agonist

Having established that a single intravenous dose of LND-formulated CDN promotes eradication of murine tumors as described above, it was further evaluated if CDN delivered by other lipid particle formulations would have a similar effect. Accordingly, CDN-carrying vesicles with compositions similar to commercial long-circulating PEGylated liposomes were prepared (see, e.g., Barenholz, Y. C. J. Controlled Release 160, 117-134 (2012)).

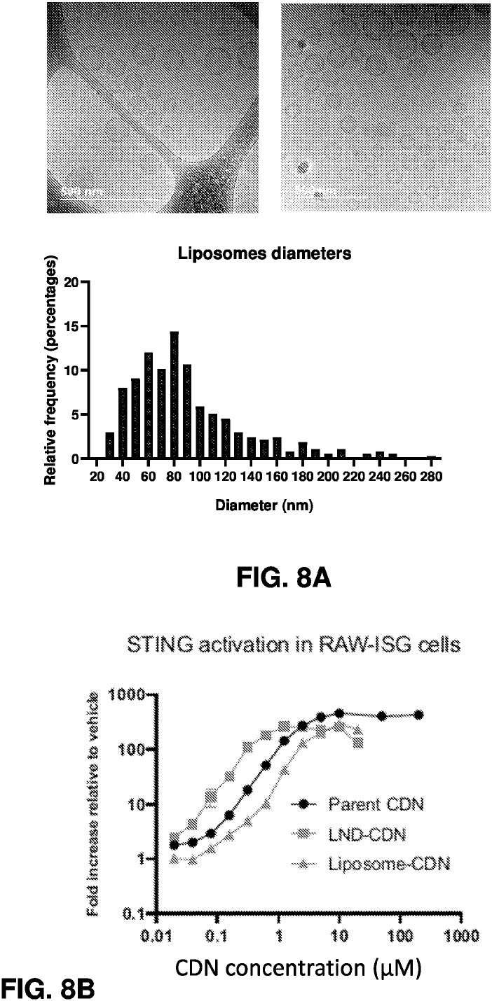

Specifically, CDN-PEG-Lipid prepared as described in Example 1 was formulated as an LND or a liposome. The LND 1 formulation described in Example 3 was used. Liposomes comprising CDN-PEG-Lipid were prepared according to the following method (referred to as “liposome-CDN”), which was selected to provide small-size liposomes that were stable. Liposomes were prepared using a lipid formulation of 60 mol% HSPC, 35.0 mol% cholesterol, 4 mol% DSPE-PEG5k, and 1.0 mol% CDN-PEG-Lipid using the ethanol precipitation method described for LND 1 in Example 3. The resulting particles were evaluated by electron microscopy as DLS. As shown in FIG. 8A, the liposome-CDN particles had typical spherical shape (top panel) and were found to have an average dimeter of 90.5 nm (bottom panel). Average diameter as measured by DLS and TEM for both LND-CDN and liposome-CDN particles are provided in Table 5.

TABLE 5

LND 1 and liposome-CDN compositions and characterization

| Particle | Lipid composition (mol%) | | | | Mean number diameter by DLS (nm) / PDI | Mean diameter by electron microscopy (nm) ± S.D. | Zeta potential (mV) |
|---|---|---|---|---|---|---|---|
| | HSPC | Cholesterol | DSPE-PEG₅₀₀₀- OMe | CDN-PEG-Lipid | | | |
| LND 1 | 75 | 0 | 20 | 5 | 33.7/0.12 | 25.7 ± 10.0 | -1.26 |
| Liposome-CDN | 60 | 35 | 4 | 1 | 61.6/0.20 | 90.5 ± 44.9 | -2.59 |

To quantify cellular uptake, the formulation of LND-CDN and liposome-CDN particles as described above was modified to include a fluorescent label that was either DSPE-PEG₂₀₀₀-sulfo-Cy5 or DSPE-PEG₂₀₀₀-IR800cw at a CDN-to-dye ratio of 5:1 at the time of formulation. DSPE-PEG₂₀₀₀-sulfo-Cy5 and DSPE-PEG₂₀₀₀-IR800cw were prepared by coupling DSPE-PEG₂₀₀₀-NH₂ (Avanti) to disulfo-Cy5-NHS (Tocris) or IR800cw-NHS (Licor). Fluorescently-labeled LND-CDN or liposome-CDNs were readily taken up by mouse macrophages (RAW-ISG) or MC38 colon adenocarcinoma cells when cultured in complete media for 24 hours (data not shown).

The potency of CDN-PEG-Lipid for inducing STING activation was compared to parent CDN using the RAW-ISG reporter cell assay described in Example 2. As shown in FIG. 8B, the LND-CDN particles were found to be moderately more potent than the liposome-CDN and the parent free CDN for inducing STING activation. However, at micromolar concentrations, all 3 forms of the drug fully activated the reporter cells.

### Example 8: LND Formulated STING Agonist Has Improved Pore Penetration Compared to Liposome Formulated STING Agonist

The size, shape, charge, surface chemistry, and rigidity (elasticity) of particles are all parameters that influence their ability to penetrate small pores present in the tumor ECM (see, e.g., Zhu, X, et al. Mater Horizons 6, 1094-1121 (2019); Wang, Z., et al. Expert Opin Drug Del 15, 379-395 (2017); Albanese, A., Annual review of biomedical engineering 14, 1-16 (2012)). Recent studies have emphasized the improved tumor penetration capacity of nanomaterials with sizes < 100 nm and high aspect ratio morphologies (Chauhan, V. P. et al. Angewandte Chemie (International ed in English) 50, 11417-11420 (2011); Niora, M. et al. Acs Omega 5, 21162-21171 (2020); Tang, L. et al.Proc National Acad Sci 111, 15344-15349 (2014); Ding, J. et al. Nano Today 29, 100800 (2019); Cabral, H. et al. Nature Nanotechnology 6, 815-823 (2011)). Without being bound by theory, based on their small size and deformable morphology, it was predicted that LND-formulated CDN particles would be particularly effective at penetrating structural barriers present in tumors.

Diffusion experiments were performed to assess the comparative ability of LND and liposomes particles to pass through pores of defined sizes. Briefly, the experiments used QuikPrep® Fast Micro-Equilibrium Dialyzers (Harvard Apparatus) with polycarbonate track-etch membranes of 50.0 nm and 200.0 nm pore size. Membranes were passivated by soaking in PBS buffer with 1.0 wt% bovine serum albumin for 1 hour followed by washing with PBS buffer. LND-CDN or liposome-CDN prepared with DSPE-PEG₂₀₀₀-sulfo-Cy5 as described in Example 7. The particles were loaded in PBS buffer on one side and PBS buffer without particle was loaded on the opposite side. The chambers were sealed and incubated at 25° C. with shaking (100-200 rpm) for 24 hours. The solutions from each side of the dialyzer were transferred to a black 96-well plate and the fluorescence was quantified using a plate reader. Percent diffusion was calculated as: Percent Diffusion = F_{Unloaded} /( 0.5 x (F_{Unloaded} + F_{loaded})) x 100, where F_{loaded} and F_{unloaded} are the fluorescence intensities of the chambers loaded with particle and loaded with only PBS, respectively. The assay was performed in triplicate.

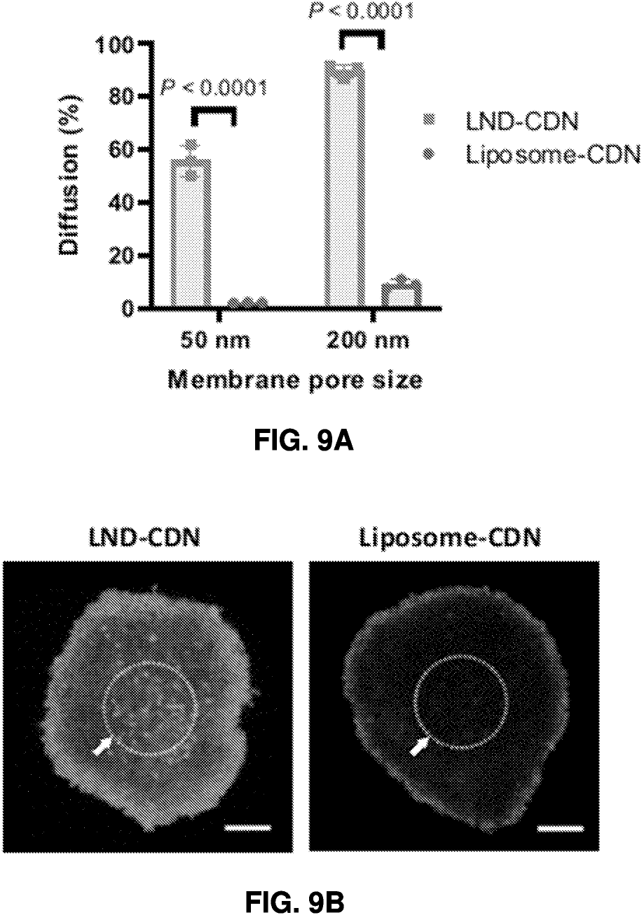

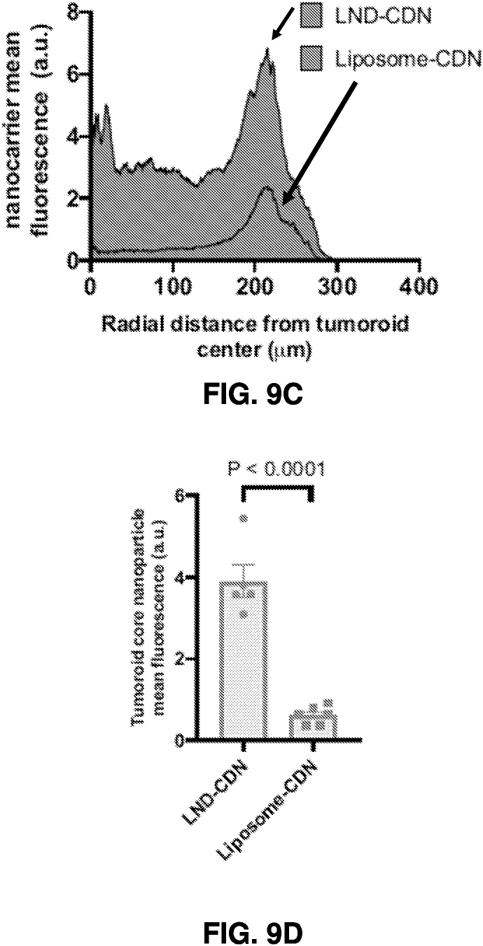

As shown in FIG. 9A, LND-CDNs were found to efficiently crossed membranes with well-defined pore sizes of 50 or 200 nm. In contrast, although liposome-CDNs readily passed through 200 nm syringe filters under gentle pressure, they showed inefficient passage across either pore size by passive diffusion.

It was next evaluated whether LND-CDN and liposome-CDN would penetrate the extracellular matrix of solid tumors. LND-CDNs or liposome-CDNs were added to the culture medium of MC38 tumor spheroids, and penetration into spheroids in vitro was tracked by confocal microscopy. Tumor spheroids, referred to as tumoroids, were formed by seeding 10,000 MC38 cells per well in a 96-well round bottom ultra-low attachment plate (Corning) and used after 5 days of growth. Tumoroids were incubated in complete cell media (DMEM with 10% FBS) with sulfo-Cy5 labeled LND-CDN or liposome-CDN particles at a concentration of 5.0 µM CDN and 1.0 µM dye for 24 hours. Following incubation, tumoroids were washed with complete media 2 times, followed by a PBS buffer wash, and then fixed using 4% paraformaldehyde in PBS for 20 minutes at 4° C. Glass slides were prepared for mounting samples by adding two layers of double-sided tape strips (approximately 5 mm wide) along the edges of the slide so that addition of the coverslip did cause the tumoroids to be flattened. Tumoroids were washed with PBS and then transferred to glass slide, excess PBS was blotted away, Vectashield vibrance antifade mounting medium was added, and a coverslip was added. Samples were imaged on a Leica SP8 spectral confocal microscope and analyzed using imageJ.

Optical sections at the center of the tumoroids were evaluated for particle fluorescence. As shown in FIG. 9B, distinct particle fluorescence patterns were observed for LND-CDN and liposome-CDN. Upon plotting the average fluorescence as a function of radial distance from the tumoroid center, fluorescence overall and at the tumoroid center was substantially higher for the LND-CDN particles (FIG. 9C). Additionally, the mean particle signal was measured in the central 100 µm radius core of spheroids (corresponding to the core region denoted by dotted line and arrow in FIG. 9B), and was found to be significantly increased for the LND-CDN particles (FIG. 9D).

Altogether, the data indicates tumoroids treated with LND-CDN display significantly higher signals in their inner core compared to liposomes. Based on the data, LND-CDN are capable of improved penetration across a dense matrix compared to liposome-CDN.

### Example 9: LND Formulated STING Agonist Has Improved Anti-Tumor Efficacy Compared to Liposome Formulated STING Agonist

Systemic administration of LND or liposome particles comprising CDN-PEG-Lipid was compared in the MC38 tumor model. MC38 tumors were established as described in Example 4. On day 7 post-inoculation, mice received a single intravenous injection of LND-formulated CDN or liposome-formulated CDN at a dose of 5 nmol CDN. The particles were formulated as described in Example 7. The effect on tumor area and survival was evaluated and compared to untreated mice receiving an injection of PBS alone. Tumor size was measured by area (longest dimension x perpendicular dimension) and the mice were euthanized when the tumor area exceeded 150 mm² or tumor ulceration became severe.

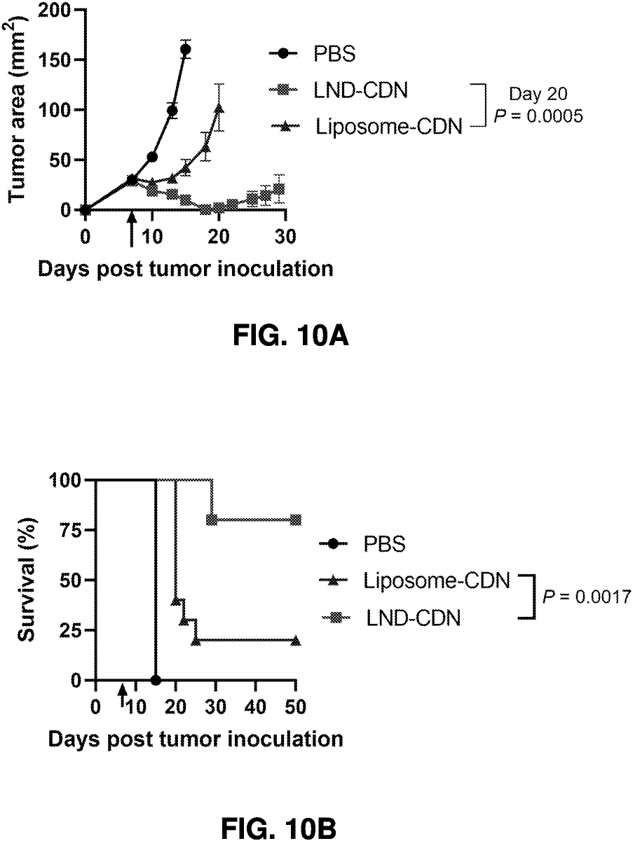

In an initial study (data not shown), LND-formulated CDN resulted in significant reduction of tumor growth and improved long-term survival compared to untreated mice. In contrast, liposome-formulated CDN had minimal effect. A comparable outcome was observed in a repeat study that evaluated the effect of a single intravenous dose of liposome-CDN and LND-CDN on tumor growth and survival in MC38-tumor bearing mice (5 nmol CDN per mouse; n=10 per treatment group), as shown in FIGS. 10A-10B. Control mice were administered PBS only (n=9). Administration of liposome-CDN triggered tumor regression that lasted a few days, but the tumors rebounded and grew out in a majority of animals starting around one week post dosing with liposomes. In contrast, administration of LND-CDNs triggered steady regression and rejection of a majority of tumors.

### Example 10: LND-Formulated STING Agonist Has Improved Tumor Uptake Compared to Liposome Formulated STING Agonist

Lipid particle accumulation in tumor and tumor draining lymph node (TDLN) tissue was measured to determine if improved delivery of CDN to the tumor and TDLN may contribute to the increased efficacy of LND-formulated CDN compared to liposome-formulated CDN. Given that LND-CDN particles are effective for penetrating a dense non-tumor matrix, it was expected they would likewise be effective for entry and penetration of a tumor bed.

To do so, LND and liposome particles were prepared comprising CDN-PEG-Lipid and 1,2-diastearoyl-sn-glycero-3-phosphoethanolamine-N-[amino(polyethylene glycol)-2000]N-(Cyanine 5) (DSPE-PEG2k-Cy5; Avanti Polar Lipids, Inc). The LND particles were prepared according to Example 3 with a lipid composition of 74 mol% HSPC, 5 mol% CDN-PEG-Lipid, 20 mol% DSPE-PEG5k, and 1.0 mol% DSPE-PEG2k-Cy5. The liposome particles were prepared as described in Example 7 with a lipid composition of 60.0 mol% HSPC, 34.0 mol% cholesterol, 4.0 mol% DSPE-PEG5k, 1.0 CDN-PEG-Lipid mol% and 1.0 mol% DSPE-PEG2k-Cy5.

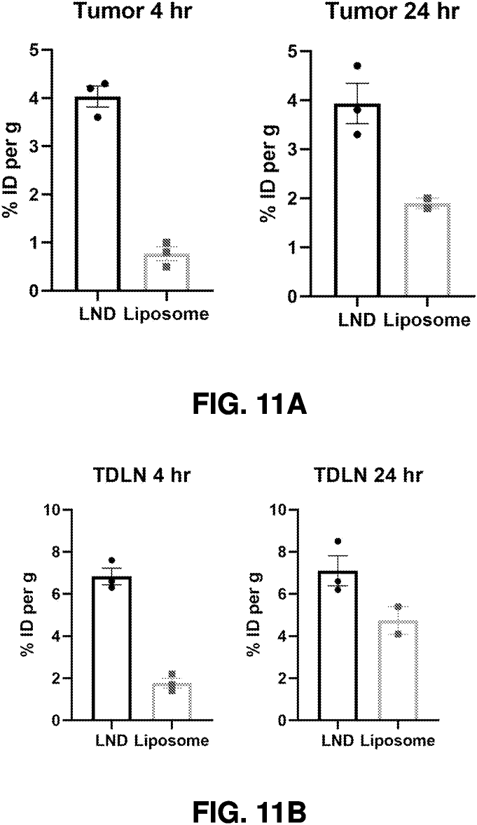

MC38 tumors were established as described in Example 4. Mice were given a single intravenous injection of fluorescently-labeled LND or liposome at a dose of 5 nanomoles. Tumors and TDLNs were collected at 4 hours and 24 hours post-injection. The percent injected dose per gram (% ID per g) of tissue was determined by measuring tissue fluorescence of the Cy5-label. As shown in FIGS. 11A-11B, accumulation of LND particles was greater than accumulation of liposomes in both tumor (FIG. 11A) and TDLN (FIG. 11B) at both time points evaluated. Thus, the improved accumulation of LNDs in tumor tissue likely contributes to improved delivery of CDN to the tumor microenvironment if CDN-PEG-Lipid is formulated as LND particles rather than liposome particles.

### Example 11: Effect of STING Agonist Formulation as LND or Liposome Particles on In Vivo Pharmacodynamics and Biodistribution

Pharmacokinetic and biodistribution profiles of fluorescently-labeled CDN, fluorophore-labeled LND-CDN, and liposome-CDN were compared following intravenous administration in vivo.

### (A) Pharmacokinetics

Briefly, the LND-CDN and liposome-CDN particles were labeled with DSPE-PEG₂₀₀₀-sulfo-Cy5 as described in Example 7. A Cy5-conjugated cGAMP (BioLog, c[3′-[sCya5]-AHC-G(2′,5′)pA(3′,5′)p])) was used as a labeled surrogate of the parental CDN. C57B1/6 mice were i.v.-administered cGAMP-sulfo-Cy5 at a dose of 1.0 nmol dye per mouse or the LND-CDN or liposome-CDN at a dose of 5.0 nmol CDN and 1.0 nmol sulfo-Cy5 dye per mouse (n = 9 per each experimental group). At pre-defined timepoints following injection, groups of three mice were bled retro-orbitally to collect 50 µL of blood. The plasma fraction was collected after centrifugation, diluted 5-fold with PBS buffer containing 5 mM EDTA, and the fluorescence was quantified using a plate reader. Sample concentration was determined against a standard curve prepared in PBS buffer containing 5 mM EDTA and 20% by volume naive mouse plasma. Data were evaluated as percent injected dose (%ID) and plasma collected at one minute was taken to represent the maximum injected dose (100% ID). Curves were fit using nonlinear regression and a two-phase decay on GraphPad Prism software.

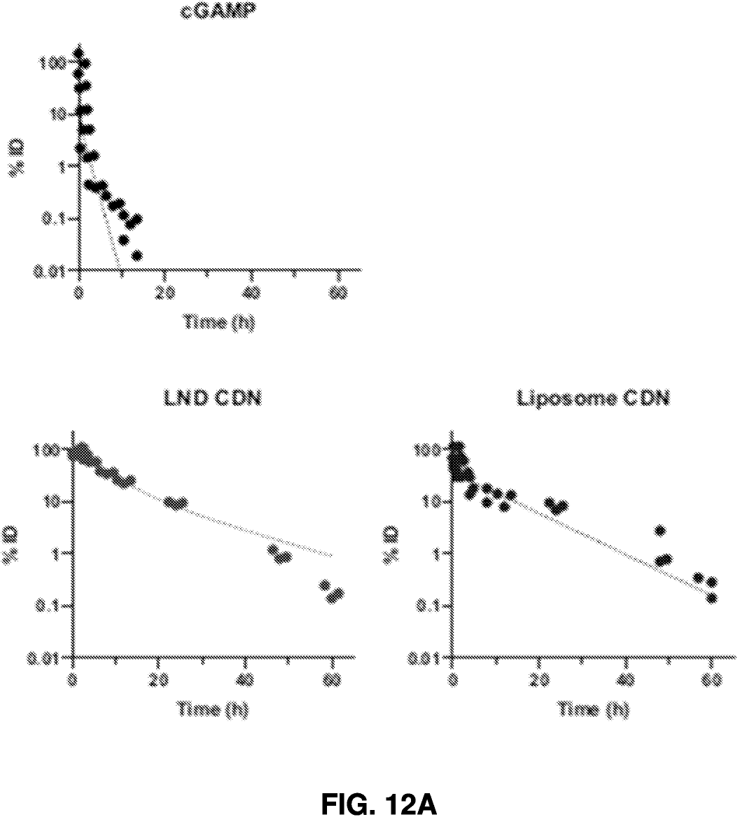

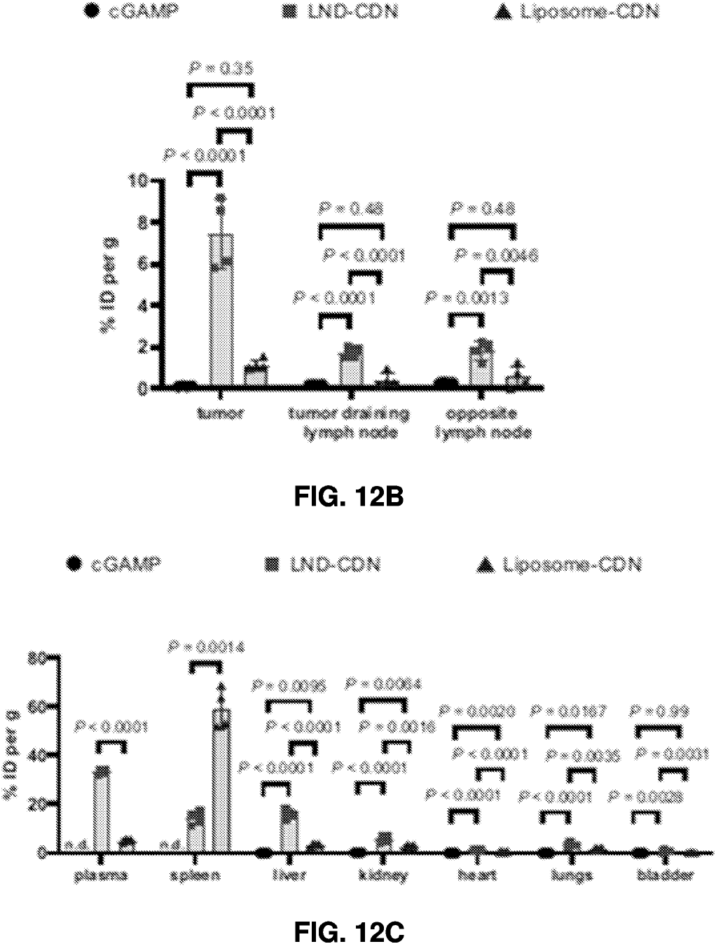

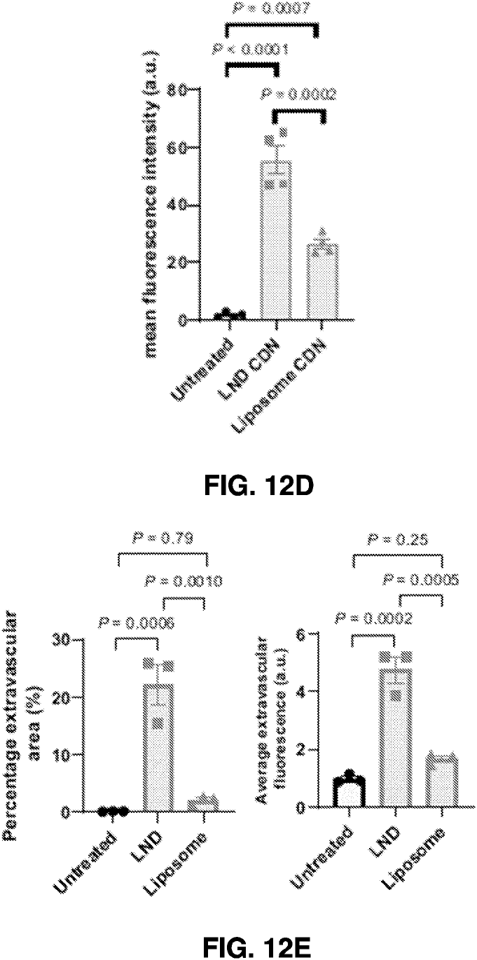

As shown in FIG. 12A, cGAMP-Cy5 cleared rapidly with a terminal plasma clearance half-life (τ_{½}) of approximately 1 hour. In contrast, both LND-CDN and liposome-CDN displayed extended circulation half-lives of 12.6 and 7.6 hours, respectively.

### (B) Biodistribution

Biodistribution in tumor-bearing mice was also evaluated following intravenous administration of LND-CDN or liposome-CDN. Briefly, C57B1/6 mice were inoculated with MC38 flank tumors as described in Example 4. At 10 days following tumor inoculation, cGAMP-sulfo-Cy5 (2.0 nmol dye/mouse), LND-CDN (5.0 nmol CDN/mouse, 2.0 nmol dye/mouse), or liposome-CDN (5.0 nmol CDN/mouse, 2.0 nmol dye/mouse) were intravenously administered via retro-orbital injection (n = 4 per experimental group). Mice were euthanized after 24 hours and tissues were harvested. The tissues were weighed and mechanically dissociated until homogenous in lysis buffer (100 mM HEPES pH 7.0, 2 wt-% Triton-X, 5 mM EDTA) using disposable tissue grinder tubes (Kimble Biomasher). Subsequently, tubes were vortexed for 1 m, centrifuged at 300 g for 2 m, and the supernatants were transferred to a black 96-well plate for quantification using a fluorescence plate reader (excitation 640 nm, emission 680 nm). The concentration of fluorophore was determined using a tissue-specific standard curve prepared with tissue digests from untreated mice. Organ uptake was reported as the percentage of injected dose per gram of tissue.

As shown in FIG. 12B, tumor accumulation of LND-CDNs was substantially higher compared to liposome-CDNs (7.4 % ID/g vs. 1.1 % ID/g). LND-CDN also accumulated in both tumor-draining and non-tumor-draining inguinal lymph nodes, but resulted in low uptake in the heart, lungs, bladder, and kidneys.

As shown in FIG. 12C, accumulation in the spleen and liver was high for both LND and liposome formulations. However, liposome-CDNs accumulated to a higher levels in the spleen, whereas LND-CDN had greater uptake in the liver. At the 24 hour time point, no cGAMP was detected in any of the tissues.

### (C) Cryofluorescence Tomography (CFT)

Whole-animal CFT was performed to visualize whole-body biodistribution of unformulated CDN, LND-CDN, and liposome-CDN following intravenous administration.

Briefly, C57B1/6 mice bearing MC38 flank tumors inoculated for 10 days were intravenously injected via the tail vein with LND-CDN or liposome-CDN at a dose of 5.0 nmol CDN per mouse. Control mice were administered saline. Each group included 4 mice. The LND-CDN and liposome-CDN were both labeled with IR800cw as described in Example 7. Mice were treated with equivalent amounts of dye on a molar basis (1.0 nmol of dye per mouse) and solutions of LND-CDN and liposome-CDN particles displayed equivalent fluorescence as measured by a plate reader before injection. Control mice were untreated. Mice were euthanized after 4 hours and immediately frozen by immersion in hexane cooled with dry ice. Mice were maintained below -80° C. until they were processed by EMIT Imaging (Boston, MA) using the Xerra system. Imaging was performed using 50.0 µM sections. For each section, in addition to a white-light image, a fluorescence image was acquired with laser excitation at 780 nm and an 835 nm emission filter with an optimized exposure time. Images were processed using ImageJ. To quantify tumor uptake, a region of interest was manually drawn around the tumor using the white light image on the section corresponding to the coronal center of the tumor and average fluorescence intensity per pixel was measured. Additional regions of interest were draw in sections 1.0 mm ventral and 1.0 mm dorsal to the center section, and the average value of the three sections was used. Fluorescence intensities were normalized based on exposure times.

Based on whole-animal maximum intensity projections and 3D tomography reconstructions, LND-CDN was observed to accumulate in the liver and throughout tumor cross-sections, whereas liposome-CDN concentrated in the spleen and bone marrow with limited accumulation in tumors (data not shown). As shown in FIG. 12D, the total tumor accumulation of liposome-CDN was notably lower than for LND-CDN.

### (D) Histology

Traditional histology was performed to corroborate observations made by CFT. Briefly, C57BL/6 mice bearing MC38 flank tumors inoculated for 7 days were intravenously administered LND-CDN or liposome-CDN. The particles were labeled with sulfo-Cy5 as described in Example 7, and were administered by retro-orbital injection (n = 3 per group). Mice were administered equivalent amounts of dye on a molar basis (2.0 nmol sulfo-Cy5 dye per mouse) and solutions of particles displayed equivalent fluorescence as measured by a plate reader before injection. Control mice were untreated. After 24 hours, mice were administered 0.30 mg/mouse of fluorescein-labeled, anionic, fixable dextran (2,000,000 MW, ThermoFisher) via retro-orbital injection to label tumor blood vessels, and the mice were euthanized after 10 minutes. Tumors were excised and fixed with 4% paraformaldehyde in PBS buffer for 16 h at 4° C. Subsequently, tumors were embedded in 2.5% agarose and sliced into 100 µm sections using a vibratome (Leica VT1000S). Sections were mounted under a glass coverslip using Vectashield vibrance antifade mounting medium on positively charged glass slides and then imaged using a Leica SP8 spectral confocal microscope using the same laser intensities and gain settings for all samples. Images were analyzed in ImageJ. Patent blood vessels were identified using the fluorescein signal with a vascular mask by setting a fixed value threshold above background. The vascular mask was adjusted with the ImageJ functions remove outliers, dilate, and fill holes, using the same settings for each image. The vascular mask was then subtracted from a region of interest defined by the tumor margin to produce an extravascular region of interest, from which the average fluorescence intensity of the particle signal was measured. To quantify the percentage of extravascular area containing particle signal, the extravascular region of interest was set at a constant threshold above background, made binary, and the percentage of pixels with non-zero signal was measured. Each point represents the average of 2 unique tumor sections.

Based on the image analysis of tumor cross sections, LND-CDN was found dispersed throughout the tumor, while liposome-CDN were more confined to the tumor periphery with little penetration away from blood vessels. As shown in FIG. 12E, the percentage of the extravascular tumor area with particle fluorescence (left panel) and the average fluorescence intensity of the extravascular tumor area (right panel) was substantially higher for LND-CDN compared to liposome-CDN. Thus, LND-CDN particles exhibited both greater total accumulation in tumors and greater penetration through the tumor bed than liposome-CDN particles.

### Example 12: Effect of STING Agonist Formulation on Production of a STING-Induced Immune Response

It was further investigated whether LND-CDN and liposome-CDN were effective in eliciting the initial steps of a STING-induced immune response.

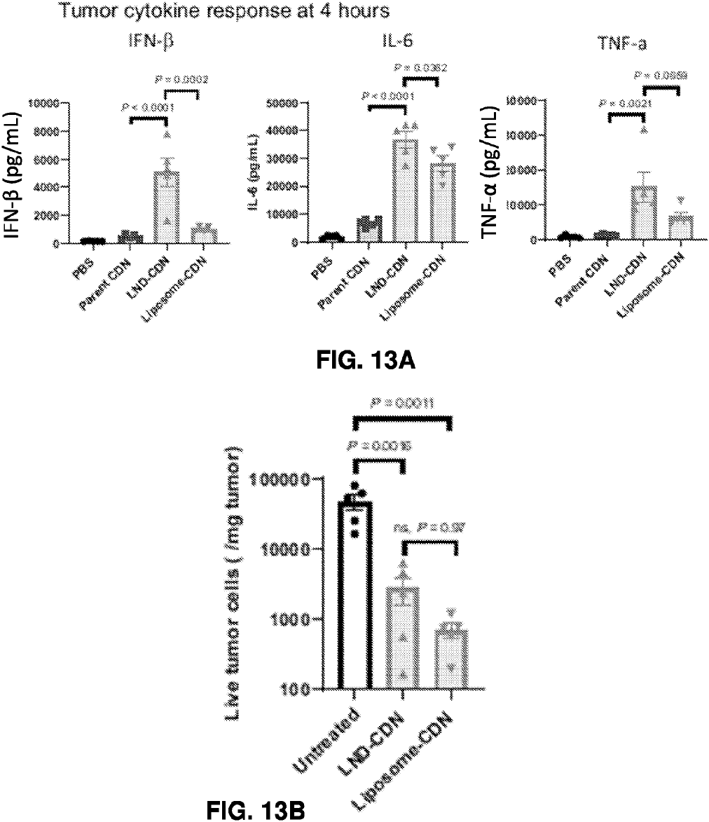

The effect of intravenous administration of LND-CDN or liposome-CDN on early production of STING-associated cytokines in tumors was evaluated using a bead-based ELISA. Briefly, at 10 days post-tumor inoculation, MC38 tumors-bearing mice were administered parent CDN at a dose of 5 nmol per mouse or LND-CDN or liposome-CDN at a dose of 5 nmol CDN per mouse. Control mice were administered saline. Four hours after treatment, tumors and the tumor-draining inguinal lymph nodes were collected and weighed. Tissues were transferred to disposable tissue grinder tubes (Kimble Biomasher) and lysis buffer (0.1X PBS, 20 mM HEPES pH 7.0, 1 wt% Triton-X, HALT Protease Cocktail (ThermoFisher), 5 mM EDTA) was added (100 µL per lymph node, 2 µL per mg of tumor). The tissues were homogenized and then analyzed or flash frozen and stored at -80° C. until later analysis. Tumor and lymph node lysates were analyzed using the legendplex mouse anti-virus response panel (Biolegend) following the manufacturer’s suggested protocol. As shown in FIG. 13A, high levels of IL-6 and TNF-α were measured in tumors by either formulation, but LND-CDNs triggered much higher levels of IFN-β production.

In addition to inducing cytokine production, STING activation is also known to trigger rapid death of tumor endothelial cells, leading to profound early tumor necrosis (Demaria, O. et al. Proc Natl Acad Sci 112, 15408-15413 (2015); Yang, H. et al. J Clin Invest 129, 43 50-4364 (2019); Francica, B. J. et al. Cancer Immunol Res 6, canimm.0263.2017 (2018); Bagu ley, B. C. et al. Int J Radiat Oncol Biology Phys 54, 1503-1511 (2002)). Thus, the degree of cell death in tumors was evaluated following intravenous administration of LND-CDN or liposome-CDN. MC38 tumors-bearing mice were administered LND-CDN or liposome-CDN at a dose of 5 nmol CDN per mouse. Control mice were untreated. At 24 hours following administration, tumors were collected and dissociated into single cells suspension as described in Example 6. The number of live tumor cells per milligram of tumor was quantified by flow cytometry. As shown in FIG. 13B, administration of either LND-CDN or liposome-CDN resulted in substantial cell death in tumors compared to control conditions, suggesting that both lipid formulations were effective at eliciting this first step in the STING-driven immune response.

### Example 13: Effect of Lipid Particle Formulation on Cell Uptake in the Tumor Microenvironment

The effect of lipid particle formulation on cellular uptake by particular cells subsets in the tumor microenvironment was evaluated in MC38 tumors. Specifically, three particle formats were prepared (LNDs, liposomes, and micelles) and their uptake by tumor cells, endothelial cells, and immune cells in the tumor microenvironment was compared. The particles were not prepared with STING agonist, in order to allow evaluation of cellular uptake in the absence of STING-induced endothelial cell death.

To quantify cellular uptake, the particles were prepared with fluorescently labeled PEG lipid, namely 1,2-diastearoyl-sn-glycero-3-phosphoethanolamine-N-[amino(polyethylene glycol)-2000]N-(Cyanine 5) (DSPE-PEG2k-Cy5; Avanti Polar Lipids, Inc). Briefly, LNDs were prepared using a lipid formulation of 74 mol% HSPC, 5 mol% DSPE-PEG2k, 20 mol% DSPE-PEG5k, and 1.0 mol% DSPE-PEG2k-Cy5 according to the ethanol-precipitation method of Example 3. Liposomes were prepared using a lipid formulation of 60 mol% HSPC, 35.0 mol% cholesterol, 4 mol% DSPE-PEG5k, and 1.0 mol% DSPE-PEG2k-Cy5 according to the method of Example 7. Micelles were prepared from a dispersion of DSPE-PEG2k-Cy5 in PBS buffer using the ethanol-precipitation method of Example 3.

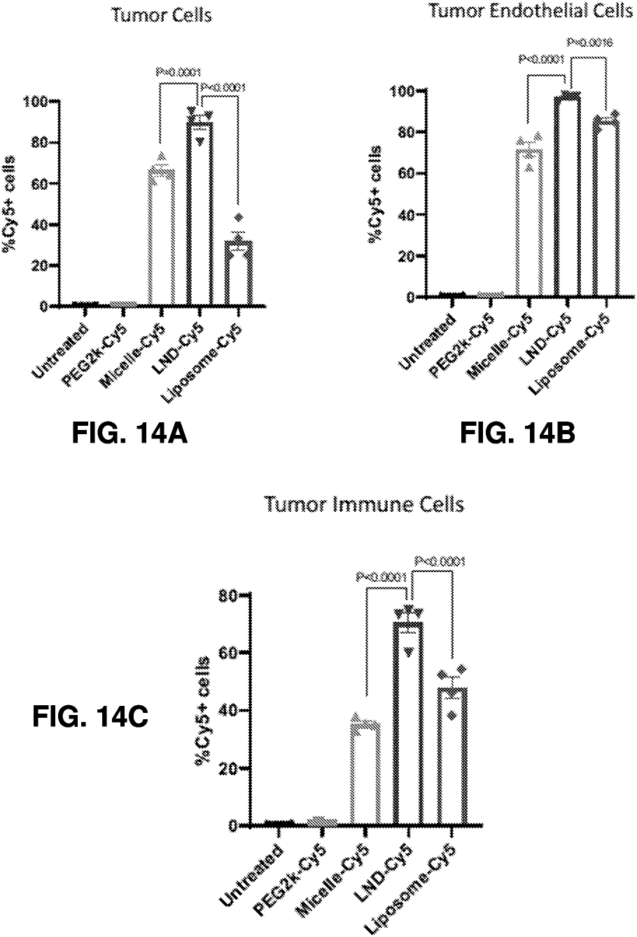

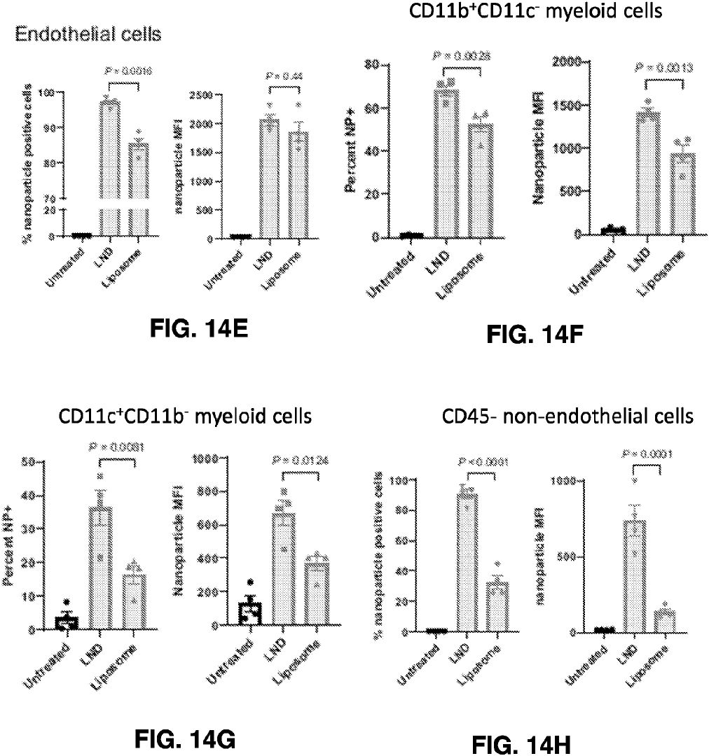

MC38 tumors were established in mice as detailed in Example 4. On day 7 post-inoculation, the mice were injected with LND-Cy5, liposome-Cy5, or micelle-Cy5 at a dose of 2 nmol Cy5 dye. As controls, mice were either untreated or administered PEG2k-Cy5 at a dose of 2 nmol Cy5 dye. At 24 hours post-administration, the tumors were excised and processed for flow cytometry, with staining for viability, CD45, CD31, and CD146. The percentage of tumor cells (Live, CD45-, CD31-, CD146-), endothelial cells (Live, CD45-, CD31+, CD146+), and immune cells (Live, CD45+) positive for Cy5 dye was determined. As shown in FIGS. 14A-14C, cellular uptake by each population was increased for Cy5 formulated as an LND compared to Cy5 formulated as a liposome or micelle. Overall, these results demonstrate that LNDs achieve superior uptake by cells in the tumor microenvironment compared to other lipid particle formulations.

In addition, the distribution of LND and liposome particles was evaluated in specific cell subsets in the tumor microenvironment following intravenous administration of sulfo-Cy5-labeled LND and liposome. Briefly, at 10 days following tumor inoculation, MC38-tumor bearing mice were administered labeled LND or liposome via retro-orbital injection. Control mice were untreated. Mice were administered equivalent amounts of dye on a molar basis (2.0 nmol sulfo-Cy5 dye per mouse) and solutions of particles displayed equivalent fluorescence as measured by a plate reader before injection. After 24 hours, tumors were excised and placed on PBS buffer on ice. Tumors were processed to a single cell suspension as described in Example 6. The cells were labeled with a panel to identify tumor endothelial cells (see Taguchi, K. et al. J Immunol Methods 464, 105-113 (2019)) and included antibodies against mouse CD31 (clone 390, BV421), CD45 (clone 30-F11, FITC), and CD146 (clone ME-9F1, PE). The cells were also labeled with a panel to identify myeloid cell subtypes and included antibodies against mouse Ly6G (clone 1A8, BV421), CD45 (clone 30-F11, FITC), CD19 (clone 1D3/CD19, PerCP/Cyanine5.5), CD3e (clone 145-2C11, PerCP/Cyanine5.5), NK1.1 (clone PK136, PerCP/Cyanine5.5), CDllb (clone M1/70, PE), Ly6C (clone HK1.4, PE-Cy7), CDllc (clone N418, APC-Fire 750). Cells were also stained with LIVE/DEAD Fixable Aqua (Life Technologies) as per the manufacturer’s instructions and only live cells were analyzed. Particle signal was detected in the APC channel. Endothelial cells were defined as CD45⁻ CD31⁺ CD146⁺ and tumor cells were identified as CD45⁻ CD31⁻ CD146⁻. The myeloid subsets of interest, CD11b+ CD11c- and CD11c⁺ CD11b⁻, were gated from CD45⁺ Ly6G⁻ and CD45⁺ Ly6G⁻ CD19⁻ CD3e NK1.1- cells, respectively.

As shown in FIGS. 14E-14F, LND and liposome were internalized by a substantial majority of tumor endothelial cells and tumor myeloid cells. However, accumulation of LNDs in CD11c+ DCs was about 2-fold higher than for liposomes (FIG. 14G). Additionally, uptake of LND was higher in non-endothelial cells. The majority of non-endothelial cells are cancer cells, and of these about 90% had internalized LNDs compared to only about 33% for liposomes (FIG. 14H). The MFI of LND-positive cells was about 5.5-fold higher than the MFI of liposome-positive cells. Based on these results, while both LND and liposomes reached tumor endothelial cells at relatively high levels, only LNDs effectively penetrated throughout the extravascular areas of the tumor to reach the majority of cancer cells.

### Example 14: LND Formulated STING Agonist Provides Superior Antigen Uptake and Activation of DCs and Induction of Antigen-Specific T Cells Compared to Liposome Formulated STING Agonist

When dying tumor cells carrying CDNs are phagocytosed by antigen presenting cells, STING is activated in trans in the APC, leading to enhanced antigen presentation (see, e.g., Ahn, J., et al. Cancer Cell 33, 862-873.e5 (2018); Corrales, L., et. al. Cell Res 27, 96-108 (2017)). It was thus evaluated whether LND-CDN has superior therapeutic efficacy compared to liposome-CDN by virtue of efficient CDN delivery to cancer cells throughout the tumor, thereby ensuring that DCs engulfing dying cancer cells become fully activated.

Briefly, MC38 tumor cells expressing a stabilized green fluorescent protein (ZsGreen) were used, with the ZsGreen functioning as a surrogate tumor antigen (see Roberts, E. W. et al. Cancer Cell 30, 324-336 (2016)). To characterize tumor antigen and particle trafficking and uptake by dendritic cells in the proximal lymph node, LND-CDN or liposome-CDN was administered intravenously to C57BL6 mice bearing MC38-ZsGreen flank tumors via retro-orbital injection at a dose of 5 nmol CDN and 1 nmol sulfo-Cy5 dye per mouse. Control mice were administered saline only. Each group had 5 mice per time point. Tumors were established at 10 days prior to the administration by subcutaneous flank injection of 5x10⁵ MC38-ZsGreen cells. Lymph nodes were harvested at one, two, and three days after administration. The harvested lymph nodes were mechanically dissociated and analyzed by flow cytometry. Cells were stained with antibodies against CD45 (clone 30-F11, BUV395, BD), IA/IE (clone M5/114.15.2, BV 421), CD19 (clone 1D3/CD19, PerCP/Cyanine5.5), CD3e (clone 145-2C11, PerCP/Cyanine5.5), NK1.1 (clone PK136, PerCP/Cyanine5.5), Ly6G (1A8, PerCP/Cyanine5.5), CD11c (clone N418, PE), CD86 (clone GL-1, PE-Cy7), and CD11b (clone M1/70, APC). DCs were defined as CD45⁺CD11c⁺ cells that were Ly6G CD19⁻CD3e⁻NK1.1⁻. Tumor antigen (ZsGreen) signal was detected in the FITC channel and particle signal was detected in the APC channel.

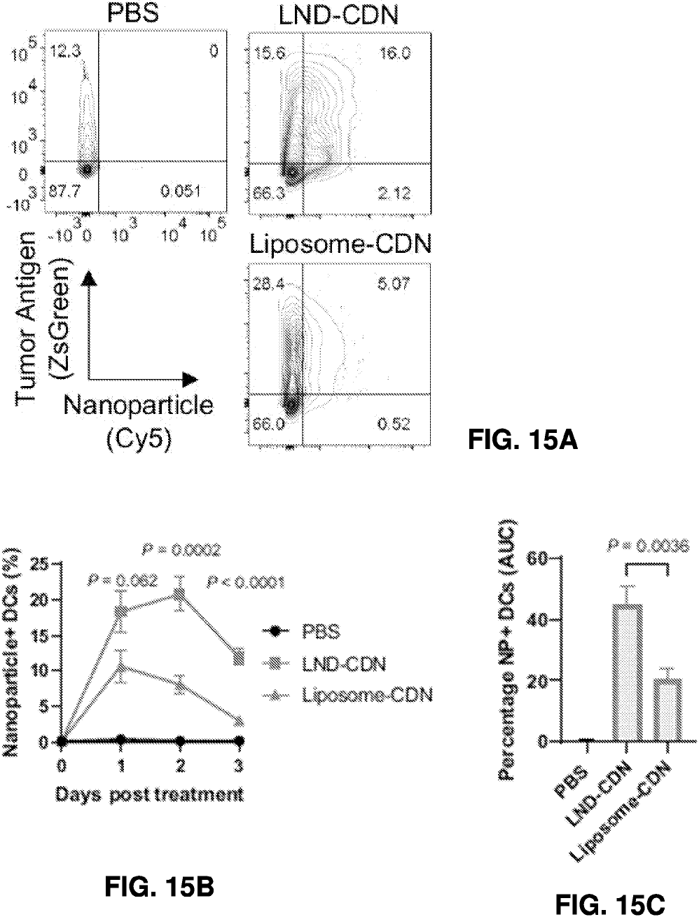

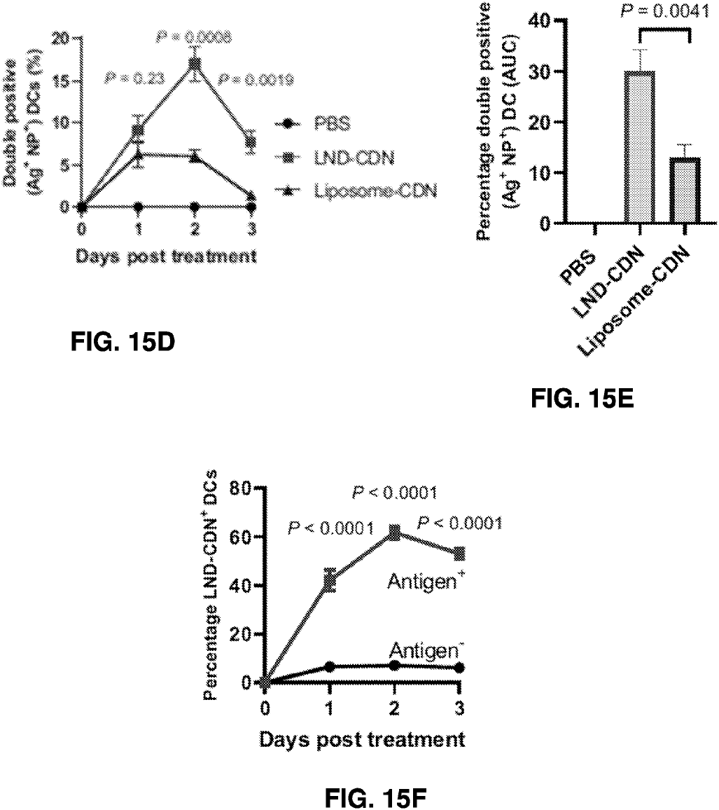

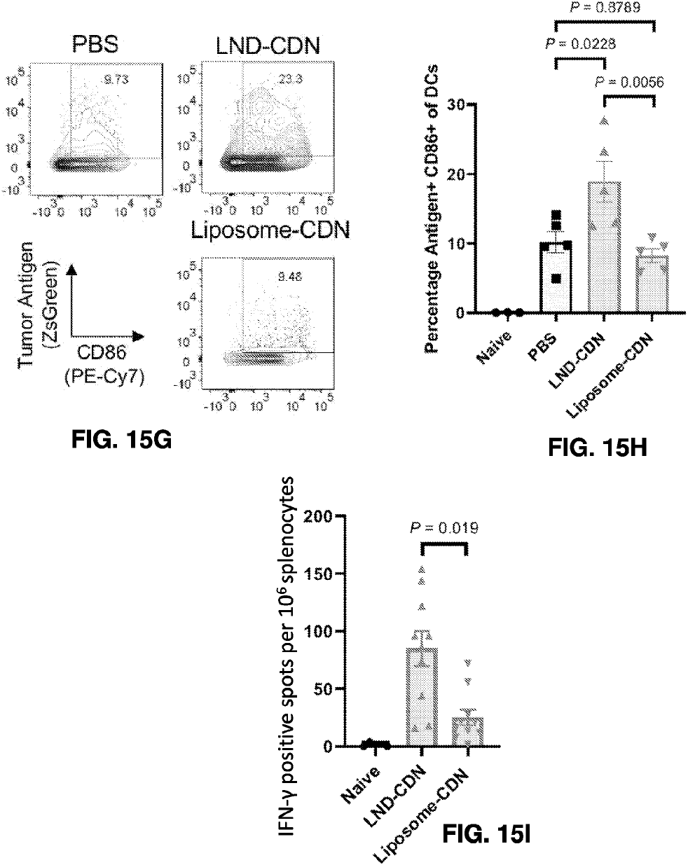

As shown in FIG. 15A, the flow analysis allowed quantification of DCs that had internalized Cy5-labeled particle, tumor antigen (ZsGreen), or were double-positive for both. Significantly more DCs took up LND-CDN particles as compared to liposome-CDN particles as measured by the percentage of particle-positive DCs at each time point (FIG. 15B) and quantification of the corresponding area under the curve (FIG. 15C). Additionally, the quantity of DCs that were double positive for both particle and antigen was nearly 3-fold higher for LND-CDN as measured by the percentage of double-positive DCs at each time point (FIG. 15D) and quantification of area under the curve (FIG. 15E).

Additionally, it was evaluated whether administration of LND-CDN particles would result in particle-positive DCs that were equally likely to be antigen-positive and antigen-negative. This would likely be the case if DCs were acquiring LND-CDN particles that directly entered the lymph node as free particles or if DCs captured the particles in the blood and carried them into the lymph node. As shown in FIG. 15F, the majority of antigen-positive DCs were also found to be positive for LND-CDN particle, whereas as antigen-negative DCs had marginal particle fluorescence. This suggests that the majority of LND-CDN uptake occurs by DC phagocytosis of dying tumor cells in the tumor that are LND-CDN-positive (followed by DC migration to the TDLN) or via DC phagocytosis of LND-CDN-positive tumor debris that trafficked in lymph to the TDLN.

Antigen-positive DCs were also evaluated for expression of activation markers. As shown in FIGS. 15G-15H, the percentage of DCs that were double-positive for antigen uptake and the activation marker CD86 was significantly higher in mice administered LND-CDN. In contrast, there was no difference in the proportion of DCs that were tumor antigen-positive and activated for mice administered liposome-CDN as compared to the untreated control animals.

The effect of LND-CDN administration on activation of an antigen-specific T cell response was evaluated by ELISPOT. Briefly, MC38-tumor bearing mice were administered LND-CDN or liposome-CDN at a dose of 5 nmol CDN per mouse at 7 days following tumor inoculation. Control mice were untreated. Effector cells were splenocytes that were harvested from the mice at 14 days following the administration. The effector cells were co-cultured with target MC38 cells. The target were treated with 50.0 U/ml mouse IFN-γ (Peprotech) for 16 hours, then irradiated (120 Gy) prior to the co-culture. A mouse IFN-γ ELISPOT Kit (BD) was used. Targets cells were seeded at 25,000 cells per well. Effector cells were seeded at 500,000 and 250,000 splenocytes per well. Plates were wrapped in foil and cultured for 24 hours, then developed according to manufacturer’s protocol. Plates were scanned using a CTL-ImmunoSpot Plate Reader, and the data were analyzed using CTL ImmunoSpot Software. As shown in FIG. 151, administration of LND-CDN induced a robust tumor antigen-specific T cell response that was significantly greater than that elicited following liposome-CDN administration.

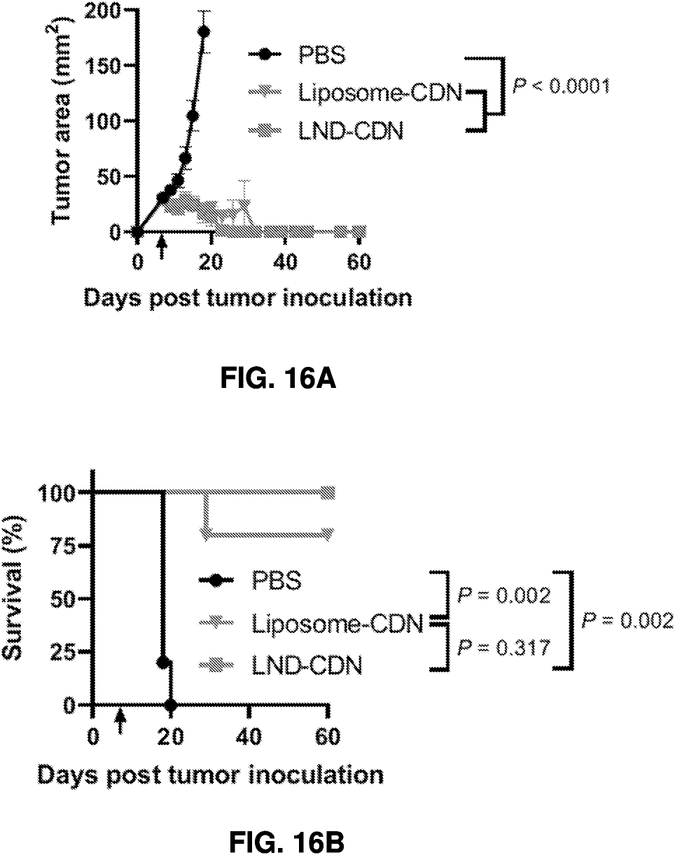

Together these data indicated the enhanced efficacy of LND-CDN particles compared to liposome-CDN particles was caused by more effective initial distribution of CDNs to cancer cells throughout the tumor bed, ensuring that DCs become optimally activating upon acquiring tumor debris in the wave of tumor cell death caused by STING-induced vascular collapse. To further evaluate this aspect, comparison was made between LND-CDN and liposome-CDN administered intratumorally rather than by intravenous administration, in order to alleviate transport barriers for the liposome-CDN (e.g., exiting the vasculature to enter the tumor bed and infiltrating the tumor bed. In this setting, both LND-CDN particles and liposome-CDN particles were highly effective, curing 5/5 and ⅘ mice, respectively (FIGS. 16A-16B). These data indicate LND particles are superior to liposome particles for delivery of STING agonist by enabling effective transport of the agonist into and throughout the tumor bed following systemic (e.g., intravenous) administration.

1. A lipid nanodisc comprising:
(i) a STING agonist amphiphile conjugate, wherein the STING agonist amphiphile conjugate comprises an agonist of STING (Stimulator of Interferon Gene) covalently linked to a polymer-modified lipid, optionally via a linker;
(ii) a phospholipid; and
(iii) a polyethylene glycol (PEG)-lipid.

2. The lipid nanodisc of claim 1, wherein the lipid nanodisc is a disc-like micelle, a discoidal micelle, a bilayer disc, or a particle with disc morphology as measured by transmission electron microscopy (TEM).

3. The lipid nanodisc of claim 1, wherein the lipid nanodisc comprises:
(i) a hydrodynamic diameter of about 10 nm to about 100 nm, about 20 nm to about 90 nm, about 30 nm to about 80 nm, about 30 nm to about 70 nm, about 30 nm to about 60 nm, about 30 nm to about 50 nm, or about 30 nm to about 40 nm as measured by dynamic light scattering (DLS);
(ii) a diameter of about 10 nm to about 100 nm, about 20 nm to about 90 nm, about 30 nm to about 80 nm, about 30 nm to about 70 nm, about 30 nm to about 60 nm, about 30 nm to about 50 nm, about 30 nm to about 40 nm as measured by TEM;
(iii) a height of about 5 nm to about 15 nm, about 6 nm, about 7 nm, about 8 nm, about 9 nm or about 10 nm as measured by TEM; and
(iv) a combination of (i)-(iii).

4. The lipid nanodisc of claim 1, wherein the lipid nanodisc comprises a hydrodynamic diameter of about 10-15 nm, about 10-20 nm, about 15-20 nm, about 15-25 nm, about 20-25 nm, about 20-30 nm, about 25-30 nm, about 25-35 nm, about 30-35 nm, about 35-40 nm, about 35-45 nm, about 40-45 nm, about 40-50 nm, or about 45-50 nm as measured by DLS.

5. The lipid nanodisc of claim 1, wherein the lipid nanodisc comprises a diameter of about 10-15 nm, about 10-20 nm, about 15-20 nm, about 15-25 nm, about 20-25 nm, about 20-30 nm, about 25-30 nm, about 25-35 nm, about 30-35 nm, about 35-40 nm, about 35-45 nm, about 40-45 nm, about 40-50 nm, or about 45-50 nm as measured by TEM.

6. The lipid nanodisc of claim 1, wherein the lipid nanodisc comprises a height of about 3, 4, 5, 6, 7, 8, 9, 10, 11 or 12 nm as measured by TEM.

7. The lipid nanodisc of claim 1, wherein the lipid nanodisc remains assembled in the presence of serum albumin under physiological conditions.

8. The lipid nanodisc of claim 1, wherein the polymer-modified lipid comprises a polymer covalently-linked to a lipid that is a diacyl lipid.

9. The lipid nanodisc of claim 8, wherein the diacyl lipid comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units.

10. The lipid nanodisc of claim 8, wherein the diacyl lipid is selected from: a phosphatidylethanolamine (PE), 1,2-distearoyl-sn-glycero-3-phosphoethanolamine (DSPE), 1,2-dipalmitoyl-sn-glycero-3-phosphoethanolamine (DPPE), 1,2-dioleoyl-sn-glycero-3-phosphoethanolamine (DOPE), 1-palmitoyl-2-oleoyl-sn-glycero-3-phosphoethanolamine (POPE), or 1,2-dimyristoyl-sn-glycero-3-phosphoethanolamine (DMPE).

11. The lipid nanodisc of claim 8, wherein the polymer is selected from: a hydrophilic polymer, a string of hydrophilic amino acids, a polysaccharide, or a combination thereof.

12. The lipid nanodisc of claim 11, wherein the hydrophilic polymer comprises PEG, polypropylene oxide) (PPO), poly(methacrylate), or a combination thereof.

13. The lipid nanodisc of claim 11, wherein the hydrophilic polymer comprises “n” consecutive PEG units, wherein n is about 25 to about 230.

14. The lipid nanodisc of claim 1, wherein the polymer-modified lipid comprises a hydrophilic polymer covalently-linked to a DSPE head-group, wherein the hydrophilic polymer comprises “n” consecutive PEG units, and wherein n is about 25 to about 230.

15. The lipid nanodisc of claim 1, wherein the STING agonist is a compound that binds to mouse STING receptor, human STING receptor, or both.

16. The lipid nanodisc of claim 15, wherein the STING agonist increases or promotes production of one or more STING-dependent cytokines in a STING-expressing cell, wherein the STING-dependent cytokine is selected from: a cytokine that binds interferon receptor, interferon, type 1 interferon, IFN-α, IFN-β, IL-6, or TNF-α.

17. The lipid nanodisc of claim 1, wherein the STING agonist is selected from: a cyclic dinucleotide (CDN) or a non-nucleotide small molecule, optionally wherein the non-nucleotide small molecule is an amidobenzimidazole (ABZI)-based compound or a di-ABZI-based compound.

18. The lipid nanodisc of claim 17, wherein the CDN comprises:
(i) a pyrimidine nucleotide base or analog thereof, a purine nucleotide base or analog thereof, or both; and
(ii) a 2′,5′ phosphate bridge linkage, a 3′5′ phosphate bridge linkage, or both.

19. The lipid nanodisc of claim 17, wherein the CDN is selected from: cyclic di-guanosine 5′-monophosphate (cyclic di-GMP), cyclic di-inosine monophosphate, cyclic di-adenosine 5′-monophosphate (cyclic di-AMP or CDA), cyclic GMP-AMP (cGAMP), cyclic[G(2′,5′)pA(3′,5′)p] (2′-3′ cGAMP), or cyclic[A(2′,5′)pA(3′5′)p] (2′-3′ CDA).

20. The lipid nanodisc of claim 17, wherein the CDN comprises at least one phosphate bridge linkage wherein a non-bridging oxygen atom is substituted with a sulfur atom, and wherein the CDN is covalently linked to the polymer-modified lipid by the sulfur atom.

21-204. (canceled)
